(19) United States
(12) Patent Application Publication
(10) Pub. No.: US 2022/0380341 A1
(43) Pub. Date: Dec. 1, 2022

(54) INHIBITORS OF HIV-1 NEF FOR THE TREATMENT OF HIV DISEASE

(71) Applicants: University of Pittsburgh - Of the Commonwealth System of Higher Education, Pittsburgh, PA (US); Fox Chase Chemical Development Center, Inc., Doylestown, PA (US)

(72) Inventors: Thomas Smithgall, Wexford, PA (US); Allen Reitz, Doylestown, PA (US); Jay Wrobel, Doylestown, PA (US); Colin Tice, Doylestown, PA (US); Thomas Haimowitz, Doylestown, PA (US); Marianne Carlsen, Doylestown, PA (US); Marie Loughran, Doylestown, PA (US); Hong Ye, Doylestown, PA (US)

(73) Assignees: University of Pittsburgh - Of the Commonwealth System of Higher Education, Pittsburgh, PA (US); Fox Chase Chemical Development Center, Inc., Doylestown, PA (US)

(21) Appl. No.: 17/285,389

(22) PCT Filed: Oct. 17, 2019
(86) PCT No.: PCT/US2019/056800

§ 371 Date: Apr. 14, 2021

Related U.S. Application Data
(60) Provisional application No. 62/747,603, filed on Oct. 18, 2018.

(51) Int. Cl. C07D 401/14; C07D 403/04; C07D 405/14; A61K 45/06

(52) U.S. Cl. CPC C07D 401/14; A61K 45/06; C07D 405/14; C07D 403/04

(57) ABSTRACT

A compound of formula I, or a stereoisomer, tautomer, or pharmaceutically acceptable salt thereof:

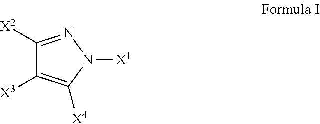

wherein X¹ is benzimidazolyl, substituted benzimidazolyl, azabenzimidazolyl, substituted azabenzimidazolyl, pyrazolyl, or substituted pyrazolyl;
X² is aryl, substituted aryl, arylalkyl, substituted arylalkyl, heteroaryl, substituted heteroaryl, heteroarylalkyl, substituted heteroarylalkyl, cycloalkyl, substituted cycloalkyl, (cycloalkyl)alkyl, substituted (cycloalkyl)alkyl, heterocycloalkyl, substituted heterocycloalkyl, (heterocycloalkyl)alkyl, substituted (heterocycloalkyl)alkyl, alkyl, substituted alkyl, alkoxycarbonyl, substituted alkoxycarbonyl, dialkylaminocarbonyl, substituted dialkylaminocarbonyl, (heterocycloalkyl)carbonyl, or substituted (heterocycloalkyl)carbonyl;
X³ is alkyl, substituted alkyl, alkenyl, substituted alkenyl, heterocycloalkyl, substituted heterocycloalkyl, (heterocycloalkyl)alkyl, substituted (heterocycloalkyl)alkyl, aryl, substituted aryl, arylalkyl, substituted arylalkyl, heteroaryl, substituted heteroaryl, heteroarylalkyl, substituted heteroarylalkyl, cycloalkyl, substituted cycloalkyl, (cycloalkyl)alkyl, or substituted (cycloalkyl)alkyl; and
X⁴ is hydroxy, amino, alkyl, or hydrogen.

This application claims the benefit of U.S. Provisional Application No. 62/747,603, filed Oct. 18, 2018, which is incorporated herein by reference in its entirety.

### ACKNOWLEDGMENT OF GOVERNMENT SUPPORT

This invention was made with government support under Grant Numbers GM112516 and AI102724 awarded by the National Institutes of Health. The government has certain rights in the invention.

### BACKGROUND

Combination antiretroviral drug therapy has transformed HIV infection from a life-threatening illness to a chronic condition for those with drug access. However, current antiretroviral drugs do not clear the virus from the patient and require lifelong administration to prevent relapse. Emerging data show that chronic antiretroviral drug exposure over decades causes metabolic disturbances, organ damage, and promotes drug resistance. These issues underscore the urgent need for new pharmaceutical agents to combat HIV disease, which may also afford the possibility of functional cure through clearance of latent viral reservoirs.

Current AIDS drugs block the activity of HIV-1 enzymes critical to the viral life cycle, including reverse transcriptase, integrase and protease, as well as fusion of the virus to host cell receptors. The HIV genome also encodes four accessory factors (Vpr, Vpu, Vif, and Nef) essential for viral pathogenicity that represent alternative targets for drug discovery. HIV-1 Nef is particularly attractive in this regard, because it is critical to the HIV life cycle in vivo and also promotes immune escape of HIV-infected cells. HIV-1 Nef is a relatively small, polymorphic protein (27-30 kDa) that is packaged in the virion and is also expressed at high levels early in the viral life cycle. Nef is myristoylated on its N-terminus, which localizes it to cellular membrane compartments essential for function. Nef lacks any known biochemical activities, functioning instead through interactions with a diverse range of host cell proteins. These interactions provide the mechanistic basis for many Nef activities, including downregulation of cell-surface immune (MHC-I/II) and viral (CD4/CXCR4/CCR5) receptors, remodeling of the actin cytoskeleton, and stimulation of host cell signaling pathways to favor viral replication. These functions of Nef allow HIV-infected cells to avoid immune surveillance by the host, prevent viral superinfection, and enhance virion release.

Foundational studies in non-human primates provide strong evidence that Nef is required for SIV and HIV pathogenesis and the development of AIDS. Infection of rhesus macaques with Nef-defective SIV resulted in low viral loads and caused a substantial delay in the onset of disease. These findings are consistent with reports of rare individuals infected with Nef-defective HIV. In these patients, viral loads remain low or undetectable, and in many cases, CD4+ T-cell counts remain stable for years, even in the absence of antiretroviral therapy.

A recent study has demonstrated an essential role for Nef in HIV infection and T-cell loss in vivo using humanized mice, in which immunodeficient animals are reconstituted with the human immune system through transplantation of human fetal liver and thymus. These mice display a full range of human immune cells, including B and T cells, myelomonocytic cells, and dendritic cells. Infection of these animals with wild-type HIV-1 results in viremia and rapid depletion of CD4+ T-cells from both the blood and tissue compartments. In striking contrast, Nef-defective HIV replicates poorly in these animals and does not cause CD4+ T-cell loss, supporting a role for Nef in thymocyte killing in vivo. Taken together, animal and patient data support a dominant role for Nef in HIV pathogenesis, and strongly support the development of small molecule Nef antagonists as new weapons against HIV/AIDS.

### SUMMARY

Disclosed herein is a compound of formula I, or a stereoisomer, tautomer, or pharmaceutically acceptable salt thereof:

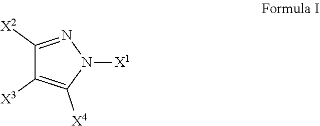

wherein X¹ is benzimidazolyl, substituted benzimidazolyl, azabenzimidazolyl, substituted azabenzimidazolyl, pyrazolyl, or substituted pyrazolyl;

X² is aryl, substituted aryl, arylalkyl, substituted arylalkyl, heteroaryl, substituted heteroaryl, heteroarylalkyl, substituted heteroarylalkyl, cycloalkyl, substituted cycloalkyl, (cycloalkyl)alkyl, substituted (cycloalkyl)alkyl, heterocycloalkyl, substituted heterocycloalkyl, (heterocycloalkyl)alkyl, substituted (heterocycloalkyl)alkyl, alkyl, substituted alkyl, alkoxycarbonyl, substituted alkoxycarbonyl, dialkylaminocarbonyl, substituted dialkylaminocarbonyl, (heterocycloalkyl)carbonyl, or substituted (heterocycloalkyl)carbonyl;

X³ is alkyl, substituted alkyl, alkenyl, substituted alkenyl, heterocycloalkyl, substituted heterocycloalkyl, (heterocycloalkyl)alkyl, substituted (heterocycloalkyl)alkyl, aryl, substituted aryl, arylalkyl, substituted arylalkyl, heteroaryl, substituted heteroaryl, heteroarylalkyl, substituted heteroarylalkyl, cycloalkyl, substituted cycloalkyl, (cycloalkyl)alkyl, or substituted (cycloalkyl)alkyl; and

X⁴ is hydroxy, amino, alkyl, or hydrogen;

provided that if X¹ is benzimidazolyl, substituted benzimidazolyl, pyrazolyl, or substituted pyrazolyl, then X² is aryl, substituted aryl, arylalkyl, substituted arylalkyl, heteroaryl, substituted heteroaryl, heteroarylalkyl, substituted heteroarylalkyl, cycloalkyl, substituted cycloalkyl, (cycloalkyl)alkyl, substituted (cycloalkyl)alkyl, heterocycloalkyl, substituted heterocycloalkyl, (heterocycloalkyl)alkyl, or substituted (heterocycloalkyl)alkyl; X³ is heterocycloalkyl, substituted heterocycloalkyl, (heterocycloalkyl)alkyl, substituted (heterocycloalkyl)alkyl, aryl, substituted aryl, arylalkyl, substituted arylalkyl, heteroaryl, substituted heteroaryl, heteroarylalkyl, substituted heteroarylalkyl, cycloalkyl, substituted cycloalkyl, (cycloalkyl)alkyl, or substituted (cycloalkyl)alkyl; and further provided that at least one of X² or X³ is substituted aryl, substituted arylalkyl, substituted heteroaryl, substituted heteroarylalkyl, substituted cycloalkyl, substituted (cycloalkyl)alkyl, substituted heterocycloalkyl, or substituted (heterocycloalkyl)alkyl.

Also disclosed herein is a compound of formula I, or a stereoisomer, tautomer, or pharmaceutically acceptable salt thereof:

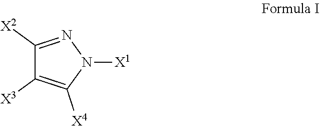

wherein X¹ is benzimidazolyl, substituted benzimidazolyl, azabenzimidazolyl, substituted azabenzimidazolyl, pyrazolyl, or substituted pyrazolyl;

X² is aryl, substituted aryl, arylalkyl, substituted arylalkyl, heteroaryl, substituted heteroaryl, heteroarylalkyl, substituted heteroarylalkyl, cycloalkyl, substituted cycloalkyl, (cycloalkyl)alkyl, substituted (cycloalkyl)alkyl, heterocycloalkyl, substituted heterocycloalkyl, (heterocycloalkyl)alkyl, substituted (heterocycloalkyl)alkyl, alkoxycarbonyl, substituted alkoxycarbonyl, dialkylaminocarbonyl, substituted dialkylaminocarbonyl, (heterocycloalkyl)carbonyl, or substituted (heterocycloalkyl)carbonyl;

X³ is alkyl, substituted alkyl, alkenyl, substituted alkenyl, heterocycloalkyl, substituted heterocycloalkyl, (heterocycloalkyl)alkyl, substituted (heterocycloalkyl)alkyl, aryl, substituted aryl, arylalkyl, substituted arylalkyl, heteroaryl, substituted heteroaryl, heteroarylalkyl, substituted heteroarylalkyl, cycloalkyl, substituted cycloalkyl, (cycloalkyl)alkyl, or substituted (cycloalkyl)alkyl; and

X⁴ is hydroxy, amino, alkyl, or hydrogen;

provided that if X¹ is benzimidazolyl, substituted benzimidazolyl, pyrazolyl, or substituted pyrazolyl, then at least one of X² or X³ is heteroaryl, substituted heteroaryl, heteroarylalkyl, substituted heteroarylalkyl, cycloalkyl, substituted cycloalkyl, (cycloalkyl)alkyl, substituted (cycloalkyl)alkyl, heterocycloalkyl, substituted heterocycloalkyl, (heterocycloalkyl)alkyl, or substituted (heterocycloalkyl)alkyl.

Further disclosed herein is a compound of formula I, or a stereoisomer, tautomer, or pharmaceutically acceptable salt thereof:

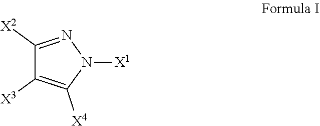

wherein X¹ is benzimidazolyl, substituted benzimidazolyl, azabenzimidazolyl, substituted azabenzimidazolyl, pyrazolyl, or substituted pyrazolyl;

X² is aryl, substituted aryl, arylalkyl, substituted arylalkyl, heteroaryl, substituted heteroaryl, heteroarylalkyl, substituted heteroarylalkyl, cycloalkyl, substituted cycloalkyl, (cycloalkyl)alkyl, substituted (cycloalkyl)alkyl, heterocycloalkyl, substituted heterocycloalkyl, (heterocycloalkyl)alkyl, substituted (heterocycloalkyl)alkyl, alkyl, substituted alkyl, alkoxycarbonyl, substituted alkoxycarbonyl, dialkylaminocarbonyl, substituted dialkylaminocarbonyl, (heterocycloalkyl)carbonyl, or substituted (heterocycloalkyl)carbonyl;

X³ is

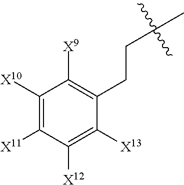

wherein each of X⁹, X¹⁰, X¹¹, X¹² and X¹³ is independently selected from hydrogen, halogen cyano, alkyl, alkoxy, haloalkyl or alkylsulfonyl; or

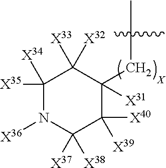

wherein x is 1 or 2; each of X³¹, X³², X³³, X³⁴, X³⁵, X³⁷, X³⁸, X³⁹, and X⁴⁰ is independently selected from hydrogen, hydroxy, cyano, aminocarbonyl, alkyl or haloalkyl; and X³⁶ is H, haloalkyl, alkoxycarbonyl, arylalkoxycarbonyl, substituted arylalkoxycarbonyl, alkylcarbonyl, hydroxyalkylcarbonyl, cycloalkylcarbonyl, arylcarbonyl, substituted arylcarbonyl, alkylsulfonyl, heteroaryl, or substituted heteroaryl; or

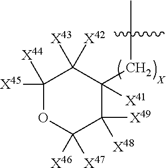

wherein x is 1 or 2; and each of X⁴¹, X⁴², X⁴³, X⁴⁴, X⁴⁵, X⁴⁶, X⁴⁷, X⁴⁸, and X⁴⁹ is independently selected from hydrogen, hydroxy, cyano, aminocarbonyl, alkyl or haloalkyl; and

X⁴ is hydroxy, amino, alkyl, or hydrogen.

Also disclosed herein is a pharmaceutical composition comprising at least one compound disclosed herein and at least one pharmaceutically acceptable excipient.

Further disclosed herein is a pharmaceutical composition comprising at least one compound disclosed herein and at least one antiretroviral agent.

Additionally, disclosed herein is a method comprising administering to a subject having, suspected of having, or at risk of developing, HIV an effective amount of at least one compound disclosed herein.

Also disclosed herein is a method of treating an HIV-related condition comprising administering to a subject in need thereof an effective amount of a compound disclosed herein or a pharmaceutical composition disclosed herein.

Further disclosed herein is a method for inhibiting a biological function of Nef, comprising contacting Nef with an effective amount of a compound disclosed herein.

The foregoing will become more apparent from the following detailed description, which proceeds with reference to the accompanying figures.

### BRIEF DESCRIPTION OF THE DRAWINGS

FIG. 1. Structures and by surface plasmon resonance (SPR) sensorgrams for several compounds disclosed herein. Each of the compounds shown was assayed for interaction with HIV-1 Nef_{NL4-3} by SPR. In particular, Recombinant HIV-1 Nef protein (strain NL4-3) was expressed in E. coli and purified to homogeneity. The Nef protein was immobilized on the surface of a carboxymethyl dextran hydrogel biosensor chip, and each compound was injected over a range of concentrations in duplicate (concentrations shown at lower left) for 90 seconds, followed by a 120 second dissociation phase. The resulting sensorgrams were best fit by either a 1:1 Langmuir binding model or by a two-step induced-fit model, and the K_{D} values shown were calculated from the resulting kinetic rate constants. Compounds were tested over the range of concentrations shown, and the resulting sensorgrams were best-fit by a two-step induced-fit model. The resulting K_{D} values are shown below each compound number.

FIG. 2. Structures and SPR data for several compounds disclosed herein. Each of the compounds shown was assayed for interaction with HIV-1 Nef_{NL4-3} by SPR. Compounds were tested over the range of concentrations shown, and the resulting sensorgrams were best-fit by a two-step induced-fit model. The resulting K_{D} values are shown below each compound.

FIGS. 3A and 3B. HIV-1 Nef enhances HIV-1 replication in donor PBMCs. PBMCs were isolated from HIV-negative donor blood and activated with PHA and IL-2. FIG. 3A) PBMCs from a representative donor were infected with wild-type and Nef-defective (ΔNef) HIV-1_{NL4-3} over the range of viral inputs shown, and replication was assessed by HIV-1 p24 Gag using and AlphaLISA assay (Perkin Elmer) after 4 days in culture. FIG. 3B) PBMCs from seven additional donors were infected with wild-type or ΔNef HIV-1 with either 625 or 1,250 p24 equivalents per mL as shown. Replication was assessed by p24 Gag AlphaLISA 4 days later. Each symbol represents the average of three endpoints ±SE for an individual donor, with the wild-type (WT) and ΔNef values connected by a solid line. These experiments establish that Nef enhances HIV-1 replication under appropriate cell culture conditions in primary cells in vitro.

FIG. 4. Inhibition of HIV-1 replication by compounds disclosed herein in PBMCs. Donor PBMCs were infected with HIV-1 in the presence of inhibitor concentrations ranging from 1 to 1,000 nM or the DMSO carrier solvent as a negative control (0.1% DMSO final). Replication was assessed by p24 Gag AlphaLISA (Perkin Elmer) five days later. Each donor was also infected with Nef-defective HIV-1 to establish the baseline for Nef-dependent enhancement of replication in each culture. Cytotoxicity was assessed in uninfected PBMCs over the same incubation period using the CellTiter-Blue assay (Promega). Data were fit by non-linear regression analysis (GraphPad Prism v.7.04) to determine the IC₅₀ and CC₅₀ values shown.

FIGS. 5A and 5B. Compounds disclosed herein rescue cell-surface MHC-I in Nef-transfected cells. The CEM-SS T cell line, which is engineered to over-express an HLA-A2 allele, was transfected with an expression plasmid for HIV-1 Nef plus GFP as a gating marker. Cells transfected with GFP and Nef were incubated in the presence of B9 and the compounds shown. After 48 hours, cells were fixed and stained for cell-surface HLA-A2 and analyzed by flow cytometry. FIG. 5A) Representative flow cytometry plots showing the percentage of GFP⁺/MHC-I⁻ cells in the presence and absence of select compounds (box). FIG. 5B) Cell-surface MHC-I rescue by compounds based on median fluorescence intensity of MHC-I⁺ cells and the following formula: [(Nef⁺MHC⁺/Nef⁻MHC-I⁺)_{drug}−(Nef⁺MHC⁺/Nef⁻MHC-I⁺)_{no drug}]×100, where Nef⁺ cells are defined by GFP expression. Bars represent mean values ±S.E. with 3 to 5 biological replicates in each case.

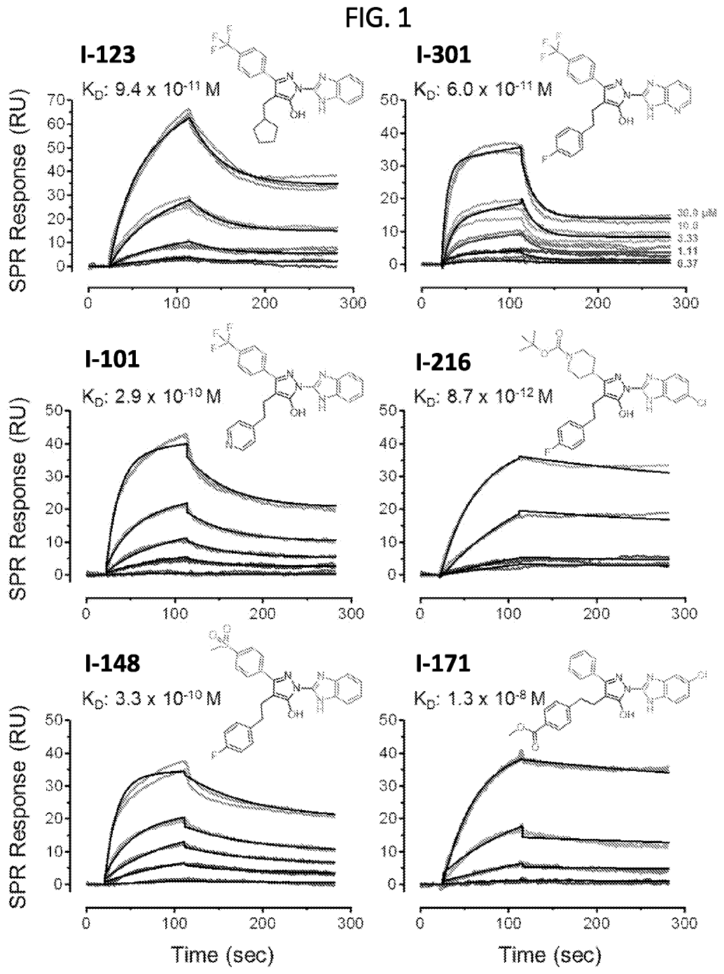

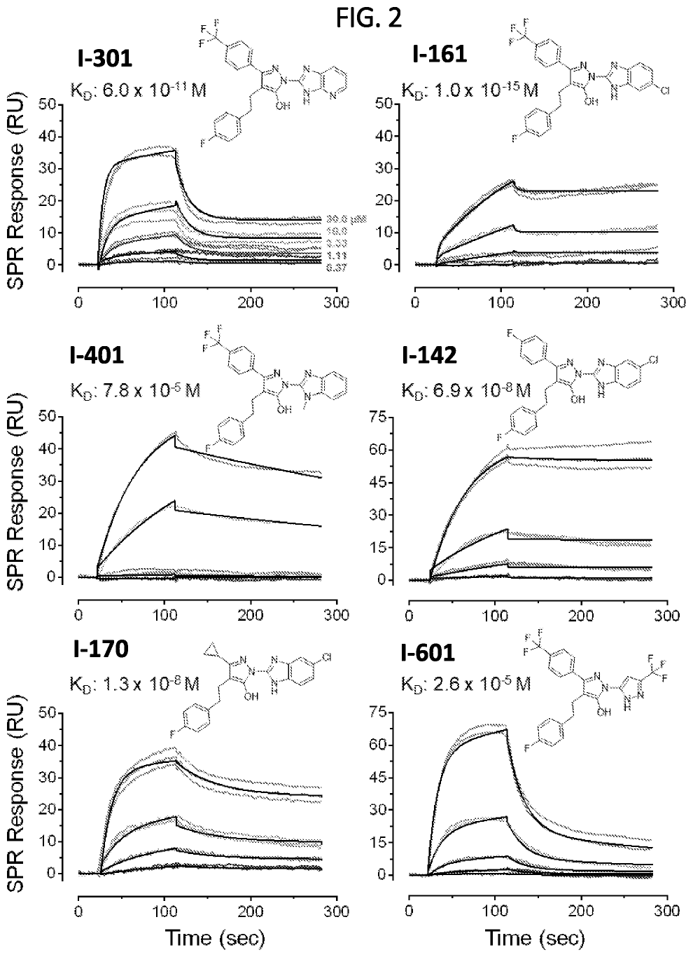

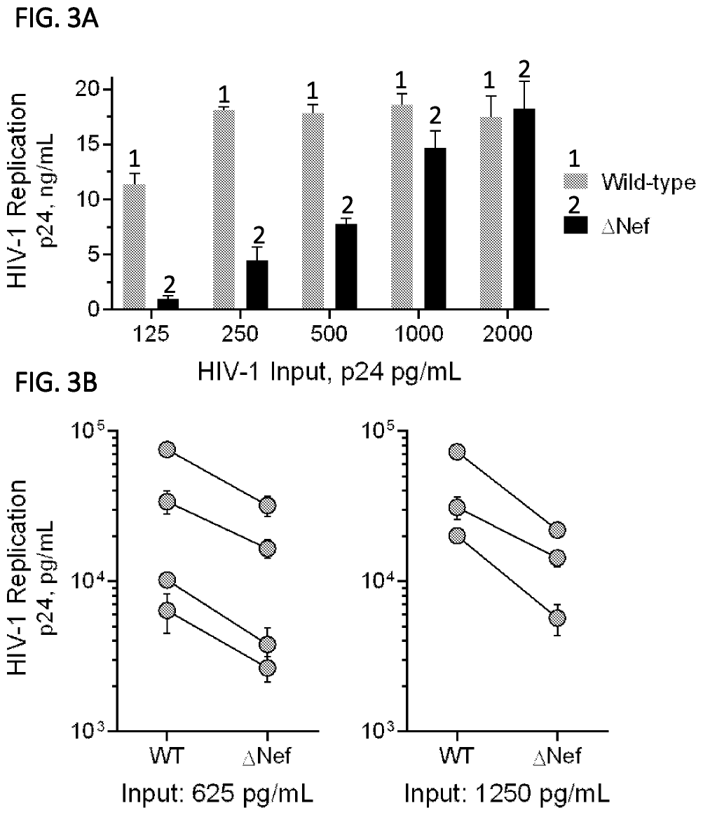

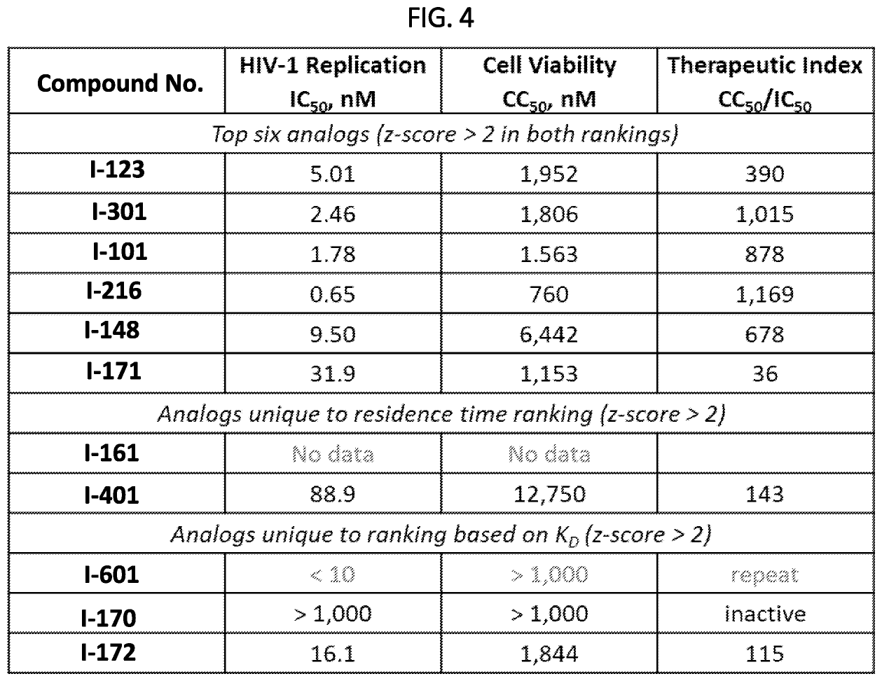

### DETAILED DESCRIPTION

Disclosed herein are embodiments of a compound for treating HIV. In particular disclosed embodiments, the compound is capable of inhibiting Nef, such as by acting as a HIV-Nef function antagonist. Nef inhibitors represent an innovative approach to antiretroviral therapy, and when combined with existing antiretroviral agents and/or therapeutic vaccines, may provide a path to eradication of HIV-infected cells and functional cure of HIV disease. The compound is a small molecule compound that may be capable of inhibiting both HIV-1 infectivity and replication. Further, the compound may be capable of reversing Nef-mediated interference with cell-surface display of MHC-I in complex with HIV-1 antigens, thereby restoring natural immune responses to HIV-infected cells. The compound may be active against HIV-1 replication supported by Nef alleles representative of all major subtypes of HIV-1. The disclosed compound is capable of binding to Nef and thereby altering or inhibiting its activity. In particular disclosed embodiments, the compound may bind electrostatically, via hydrogen bonding, or covalently. In certain embodiments, disclosed herein are compounds that bind directly to the Nef protein in vitro with nM to pM potency, and show antiretroviral activity in cell-based assays.

### Terminology

The following explanations of terms and methods are provided to better describe the present compounds, compositions and methods, and to guide those of ordinary skill in the art in the practice of the present disclosure. It is also to be understood that the terminology used in the disclosure is for the purpose of describing particular embodiments and examples only and is not intended to be limiting.

“Acyl” refers to a group having the structure—C(O)R, where R may be, for example, optionally substituted alkyl, optionally substituted aryl, or optionally substituted heteroaryl. “Lower acyl” groups are those that contain one to six carbon atoms.

“Acyloxy” refers to a group having the structure —OC(O)R—, where R may be, for example, optionally substituted alkyl, optionally substituted aryl, or optionally substituted heteroaryl. “Lower acyloxy” groups contain one to six carbon atoms.

“Administration” as used herein is inclusive of administration by another person to the subject or self-administration by the subject.

The term “aliphatic” is defined as including alkyl, alkenyl, alkynyl, halogenated alkyl and cycloalkyl groups. A “lower aliphatic” group is a branched or unbranched aliphatic group having from 1 to 10 carbon atoms.

“Alkanediyl,” “cycloalkanediyl,” “aryldiyl,” “alkanearyldiyl” refers to a divalent radical derived from aliphatic, cycloaliphatic, aryl, and alkanearyl hydrocarbons.

“Alkenyl” refers to a cyclic, branched or straight chain group containing only carbon and hydrogen, and contains one or more double bonds that may or may not be conjugated. Alkenyl groups may be unsubstituted or substituted. “Lower alkenyl” groups contain one to six carbon atoms.

The term “alkoxy” refers to a straight, branched or cyclic hydrocarbon configuration and combinations thereof, including from 1 to 20 carbon atoms, preferably from 1 to 8 carbon atoms (referred to as a “lower alkoxy”), more preferably from 1 to 4 carbon atoms, that include an oxygen atom at the point of attachment. An example of an “alkoxy group” is represented by the formula —OR, where R can be an alkyl group, optionally substituted with an alkenyl, alkynyl, aryl, aralkyl, cycloalkyl, halogenated alkyl, alkoxy or heterocycloalkyl group. Suitable alkoxy groups include methoxy, ethoxy, n-propoxy, i-propoxy, n-butoxy, i-butoxy, sec-butoxy, tert-butoxy cyclopropoxy, cyclohexyloxy, and the like.

“Alkoxycarbonyl” refers to an alkoxy substituted carbonyl radical, —C(O)OR, wherein R represents an optionally substituted alkyl, aryl, aralkyl, cycloalkyl, cycloalkylalkyl or similar moiety.

The term “alkyl” refers to a branched or unbranched saturated hydrocarbon group of 1 to 24 carbon atoms, such as methyl, ethyl, n-propyl, isopropyl, n-butyl, isobutyl, t-butyl, pentyl, hexyl, heptyl, octyl, decyl, tetradecyl, hexadecyl, eicosyl, tetracosyl and the like. A “lower alkyl” group is a saturated branched or unbranched hydrocarbon having from 1 to 6 carbon atoms. Preferred alkyl groups have 1 to 4 carbon atoms. Alkyl groups may be “substituted alkyls” wherein one or more hydrogen atoms are substituted with a substituent such as halogen, cycloalkyl, alkoxy, amino, hydroxyl, aryl, alkenyl, or carboxyl. For example, a lower alkyl or (C₁-C₆)alkyl can be methyl, ethyl, propyl, isopropyl, butyl, iso-butyl, sec-butyl, pentyl, 3-pentyl, or hexyl; (C₃-C₆)cycloalkyl can be cyclopropyl, cyclobutyl, cyclopentyl, or cyclohexyl; (C₃-C₆)cycloalkyl(C₁-C₆)alkyl can be cyclopropylmethyl, cyclobutylmethyl, cyclopentylmethyl, cyclohexylmethyl, 2-cyclopropylethyl, 2-cyclobutylethyl, 2-cyclopentylethyl, or 2-cyclohexylethyl; (C₁-C₆)alkoxy can be methoxy, ethoxy, propoxy, isopropoxy, butoxy, iso-butoxy, sec-butoxy, pentoxy, 3-pentoxy, or hexyloxy; (C₂-C₆)alkenyl can be vinyl, allyl, 1-propenyl, 2-propenyl, 1-butenyl, 2-butenyl, 3-butenyl, 1,-pentenyl, 2-pentenyl, 3-pentenyl, 4-pentenyl, 1-hexenyl, 2-hexenyl, 3-hexenyl, 4-hexenyl, or 5-hexenyl; (C₂-C₆)alkynyl can be ethynyl, 1-propynyl, 2-propynyl, 1-butynyl, 2-butynyl, 3-butynyl, 1-pentynyl, 2-pentynyl, 3-pentynyl, 4-pentynyl, 1-hexynyl, 2-hexynyl, 3-hexynyl, 4-hexynyl, or 5-hexynyl; (C₁-C₆)alkanoyl can be acetyl, propanoyl or butanoyl; halo(C₁-C₆)alkyl can be iodomethyl, bromomethyl, chloromethyl, fluoromethyl, trifluoromethyl, 2-chloroethyl, 2-fluoroethyl, 2,2,2-trifluoroethyl, or pentafluoroethyl; hydroxy(C₁-C₆)alkyl can be hydroxymethyl, 1-hydroxyethyl, 2-hydroxyethyl, 1-hydroxypropyl, 2-hydroxypropyl, 3-hydroxypropyl, 1-hydroxybutyl, 4-hydroxybutyl, 1-hydroxypentyl, 5-hydroxypentyl, 1-hydroxyhexyl, or 6-hydroxyhexyl; (C₁-C₆)alkoxycarbonyl can be methoxycarbonyl, ethoxycarbonyl, propoxycarbonyl, isopropoxycarbonyl, butoxycarbonyl, pentoxycarbonyl, or hexyloxycarbonyl; (C₁-C₆)alkylthio can be methylthio, ethylthio, propylthio, isopropylthio, butylthio, isobutylthio, pentylthio, or hexylthio; (C₂-C₆)alkanoyloxy can be acetoxy, propanoyloxy, butanoyloxy, isobutanoyloxy, pentanoyloxy, or hexanoyloxy.

“Alkynyl” refers to a cyclic, branched or straight chain group containing only carbon and hydrogen, and unless otherwise mentioned typically contains one to twelve carbon atoms, and contains one or more triple bonds. Alkynyl groups may be unsubstituted or substituted. “Lower alkynyl” groups are those that contain one to six carbon atoms.

The term “amine” or “amino” refers to a group of the formula —NRR′, where R and R′ can be, independently, hydrogen or an alkyl, alkenyl, alkynyl, acyl, aryl, arylalkyl, cycloalkyl, halogenated alkyl, or heterocycloalkyl group. For example, an “alkylamino” or “alkylated amino” refers to —NRR′, wherein at least one of R or R′ is an alkyl. A suitable amine or amino group is acetamido.

The term “aminoalkyl” refers to alkyl groups as defined above where at least one hydrogen atom is replaced with an amino group (e.g, —CH₂—NH₂).

“Aminocarbonyl” alone or in combination, means an amino substituted carbonyl (carbamoyl) radical, wherein the amino radical may optionally be mono- or di-substituted, such as, for example, with alkyl, aryl, acyl, aralkyl, cycloalkyl, cycloalkylalkyl, alkanoyl, alkoxycarbonyl, aralkoxycarbonyl and the like. For example, an aminocarbonyl may be represented by the formula —C(O)NRR′, where R and R′ independently can be, for example, a hydrogen, alkyl, alkenyl, alkynyl, acyl, aryl, aralkyl, cycloalkyl, halogenated alkyl, or heterocycloalkyl group.

An “animal” refers to living multi-cellular vertebrate organisms, a category that includes, for example, mammals and birds. The term mammal includes both human and non-human mammals. Similarly, the term “subject” includes both human and non-human subjects, including birds and non-human mammals, such as non-human primates, companion animals (such as dogs and cats), livestock (such as pigs, sheep, cows), as well as non-domesticated animals, such as the big cats. The term subject applies regardless of the stage in the organism's life-cycle. Thus, the term subject applies to an organism in utero or in ovo, depending on the organism (that is, whether the organism is a mammal or a bird, such as a domesticated or wild fowl).

The term “arylalkyl” refers to an alkyl group wherein an aryl group is substituted for a hydrogen of the alkyl group. An example of an arylalkyl group is a benzyl group.

“Aryl” refers to a monovalent unsaturated or aromatic (including pseudoaromatic). carbocyclic group having a single ring (e.g., phenyl) or multiple condensed rings (e.g., naphthyl or anthryl), which can optionally be unsubstituted or substituted. The term “pseudoaromatic” refers to a ring system which is not strictly aromatic, but which is stabilized by means of delocalization of electrons and behaves in a similar manner to aromatic rings. A “heteroaryl group,” is defined as an unsaturated or aromatic (including pseudoaromatic) group that has at least one heteroatom incorporated within the ring of the aromatic group. Examples of heteroatoms include, but are not limited to, nitrogen, oxygen, sulfur, and phosphorous. Heteroaryl includes, but is not limited to, pyridinyl, pyrazinyl, pyrimidinyl, pyrrolyl, pyrazolyl, imidazolyl, thiazolyl, oxazolyl, isooxazolyl, thiadiazolyl, oxadiazolyl, thiophenyl, furanyl, quinolinyl, isoquinolinyl, benzimidazolyl, benzooxazolyl, quinoxalinyl, and the like. The aryl or heteroaryl group can be substituted with one or more groups including, but not limited to, alkyl, alkynyl, alkenyl, aryl, halide, nitro, amino, ester, ketone, aldehyde, hydroxy, carboxylic acid, or alkoxy, or the aryl or heteroaryl group can be unsubstituted. The term heteroaryl includes hydroxy-substituted heteroaryls that may exist in tautomeric keto forms, such as 2-hydroxypyridine and pyridine-2-one, and their N-substituted derivatives that necessarily exist in the keto form, such as N-methylpyridin-2-one.

“Aryloxy” or “heteroaryloxy” refers to a group of the formula —OAr, wherein Ar is an aryl group or a heteroaryl group, respectively.

A “carbonylamino” group may be —N(R)—C(O)—R (wherein each R is independently a substitution group such as, for example, alkyl, alkenyl, alkynyl, acyl, aryl, arylalkyl, cycloalkyl, halogenated alkyl, or heterocycloalkyl group, or H). A suitable carbonylamino group is acetamido.

The term “carboxylate” or “carboxyl” refers to the group —COO⁻ or —COOH. The carboxyl group can form a carboxylic acid. “Substituted carboxyl” refers to —COOR where R is alkyl, alkenyl, alkynyl, aryl, arylalkyl, cycloalkyl, halogenated alkyl, or heterocycloalkyl group. For example, a substituted carboxyl group could be a carboxylic acid ester or a salt thereof (e.g., a carboxylate).

The term “co-administration” or “co-administering” refers to administration of a compound disclosed herein with at least one other therapeutic or diagnostic agent within the same general time period, and does not require administration at the same exact moment in time (although co-administration is inclusive of administering at the same exact moment in time). Thus, co-administration may be on the same day or on different days, or in the same week or in different weeks.

The term “cycloalkyl” refers to a non-aromatic carbon-based ring composed of at least three carbon atoms. A cycloalkyl may be a mono or bicyclic ring or ring system. Examples of cycloalkyl groups include, but are not limited to, cyclopropyl, cyclobutyl, cyclopentyl, cyclohexyl, and the like. The term “heterocycloalkyl group” is a cycloalkyl group as defined above where at least one of the carbon atoms of the ring is substituted with a heteroatom such as, but not limited to, nitrogen, oxygen, sulfur, or phosphorous. A heterocycloalkyl may be mono or bicyclic ring or ring system.

The term “ester” refers to a carboxyl group-containing moiety having the hydrogen replaced with, for example, a C₁₋₆alkyl group (“carboxylC₁₋₆alkyl” or “alkylester”), an aryl or aralkyl group (“arylester” or “aralkylester”) and so on. CO₂C₁₋₃alkyl groups are preferred, such as for example, methylester (CO₂Me), ethylester (CO₂Et) and propylester (CO₂Pr) and includes reverse esters thereof (e.g. —OCOMe, —OCOEt and OCOPr).

The terms “halogenated alkyl” or “haloalkyl group” refer to an alkyl group with one or more hydrogen atoms present on these groups substituted with a halogen (F, Cl, Br, I).

The term “hydroxyl” is represented by the formula —OH.

The term “hydroxyalkyl” refers to an alkyl group that has at least one hydrogen atom substituted with a hydroxyl group. The term “alkoxyalkyl group” is defined as an alkyl group that has at least one hydrogen atom substituted with an alkoxy group described above.

“Inhibiting” refers to inhibiting the full development of a disease or condition. “Inhibiting” also refers to any quantitative or qualitative reduction in biological activity or binding, relative to a control.

“N-heterocyclic” refers to mono or bicyclic rings or ring systems that include at least one nitrogen heteroatom. The rings or ring systems generally include 1 to 9 carbon atoms in addition to the heteroatom(s) and may be saturated, unsaturated or aromatic (including pseudoaromatic). The term “pseudoaromatic” refers to a ring system which is not strictly aromatic, but which is stabilized by means of delocalization of electrons and behaves in a similar manner to aromatic rings. Aromatic includes pseudoaromatic ring systems, such as pyrrolyl rings.

Examples of 5-membered monocyclic N-heterocycles include pyrrolyl, H-pyrrolyl, pyrrolinyl, pyrrolidinyl, oxazolyl, oxadiazolyl, (including 1,2,3 and 1,2,4 oxadiazolyls) isoxazolyl, furazanyl, thiazolyl, isothiazolyl, pyrazolyl, pyrazolinyl, pyrazolidinyl, imidazolyl, imidazolinyl, triazolyl (including 1,2,3 and 1,3,4 triazolyls), tetrazolyl, thiadiazolyl (including 1,2,3 and 1,3,4 thiadiazolyls), and dithiazolyl. Examples of 6-membered monocyclic N-heterocycles include pyridyl, pyrimidinyl, pyridazinyl, pyrazinyl, piperidinyl, morpholinyl, thiomorpholinyl, piperazinyl, and triazinyl. The heterocycles may be optionally substituted with a broad range of substituents, and preferably with C₁₋₆ alkyl, C₁₋₆ alkoxy, C₂₋₆ alkenyl, C₂₋₆ alkynyl, halo, hydroxy, mercapto, trifluoromethyl, amino, cyano or mono or di(C₁₋₆alkyl)amino. The N-heterocyclic group may be fused to a carbocyclic ring such as phenyl, naphthyl, indenyl, azulenyl, fluorenyl, and anthracenyl.

Examples of 8, 9 and 10-membered bicyclic heterocycles include 1H thieno[2,3-c]pyrazolyl, indolyl, isoindolyl, benzoxazolyl, benzothiazolyl, benzisoxazolyl, benzisothiazolyl, benzimidazolyl, indazolyl, isoquinolinyl, quinolinyl, quinoxalinyl, purinyl, cinnolinyl, phthalazinyl, quinazolinyl, quinoxalinyl, benzotriazinyl, and the like. These heterocycles may be optionally substituted, for example with C₁₋₆ alkyl, C₁₋₆ alkoxy, C₂₋₆ alkenyl, C₂₋₆ alkynyl, halo, hydroxy, mercapto, trifluoromethyl, amino, cyano or mono or di(C₁₋₆alkyl)amino Unless otherwise defined optionally substituted N-heterocyclics includes pyridinium salts and the N-oxide form of suitable ring nitrogens.

The term “subject” includes both human and non-human subjects, including birds and non-human mammals, such as non-human primates, companion animals (such as dogs and cats), livestock (such as pigs, sheep, cows), as well as non-domesticated animals, such as the big cats. The term subject applies regardless of the stage in the organism's life-cycle. Thus, the term subject applies to an organism in utero or in ovo, depending on the organism (that is, whether the organism is a mammal or a bird, such as a domesticated or wild fowl).

“Substituted” or “substitution” refers to replacement of a hydrogen atom of a molecule or an R-group with one or more additional R-groups. Unless otherwise defined, the term “optionally-substituted” or “optional substituent” as used herein refers to a group which may or may not be further substituted with 1, 2, 3, 4 or more groups, preferably 1, 2 or 3, more preferably 1 or 2 groups. The substituents may be selected, for example, from C₁₋₆alkyl, C₂₋₆ alkenyl, C₂₋₆ alkynyl, C₃₋₈ cycloalkyl, hydroxyl, oxo, C₁₋₆alkoxy, aryloxy, C₁₋₆alkoxyaryl, halo, C₁₋₆alkylhalo (such as CF₃ and CHF₂), C₁₋₆alkoxyhalo (such as OCF₃ and OCHF₂), carboxyl, esters, cyano, nitro, amino, substituted amino, disubstituted amino, acyl, ketones, amides, aminoacyl, substituted amides, disubstituted amides, thiol, alkylthio, thioxo, sulfates, sulfonates, sulfinyl, substituted sulfinyl, sulfonyl, substituted sulfonyl, sulfonylamides, substituted sulfonamides, disubstituted sulfonamides, aryl, arylC₁₋₆alkyl, heterocyclyl and heteroaryl wherein each alkyl, alkenyl, alkynyl, cycloalkyl, aryl and heterocyclyl and groups containing them may be further optionally substituted. Optional substituents in the case N-heterocycles may also include but are not limited to C₁₋₆alkyl i.e. N—C₁₋₃alkyl, more preferably methyl particularly N-methyl.

“Sulfinyl” refers to the group —S(═O)H.

The term “substituted sulfinyl” or “sulfoxide” refers to a sulfinyl group having the hydrogen replaced with, for example a C₁₋₆alkyl group (“C₁₋₆alkylsulfinyl” or “C₁₋₆alkylsulfoxide”), an aryl (“arylsulfinyl”), an arylalkyl (“arylalkyl sulfinyl”) and so on. C₁₋₃alkylsulfinyl groups are preferred, such as for example, —SOmethyl, —SOethyl and —SOpropyl.

The term “sulfonyl” refers to the group —SO₂H. The sulfonyl group can be further substituted with a variety of groups to form, for example, sulfonic acids, sulfonamides, sulfonate esters and sulfones.

The term “substituted sulfonyl” refers to a sulfonyl group having the hydrogen replaced with, for example a C₁₋₆alkyl group (“sulfonylC₁₋₆alkyl”), an aryl (“arylsulfonyl”), an arylalkyl (“arylalkylsulfonyl”) and so on. SulfonylC₁₋₃alkyl groups are preferred, such as for example, —SO₂Me, —SO₂Et and —SO₂Pr.

The term “sulfonylamido” or “sulfonamide” refers to the group —SO₂NH₂.

The term “tautomer” refers to constitutional isomers of organic compounds that readily interconvert by migration of a proton, for example, the three tautomers of 3-hydroxypyrazole:

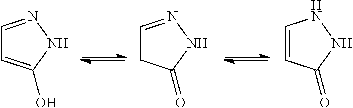 [SMILES: Oc1ccn[nH]1 ; O=C1CC=NN1 ; O=c1cc[nH][nH]1]

A “therapeutically effective amount” refers to a quantity of a specified agent sufficient to achieve a desired effect in a subject being treated with that agent. Ideally, a therapeutically effective amount of an agent is an amount sufficient to inhibit or treat the disease or condition without causing a substantial cytotoxic effect in the subject. The therapeutically effective amount of an agent will be dependent on the subject being treated, the severity of the affliction, and the manner of administration of the therapeutic composition.

“Treatment” refers to a therapeutic intervention that ameliorates a sign or symptom of a disease or pathological condition after it has begun to develop, or administering a compound or composition to a subject who does not exhibit signs of a disease or exhibits only early signs for the purpose of decreasing the risk of developing a pathology or condition, or diminishing the severity of a pathology or condition. As used herein, the term “ameliorating,” with reference to a disease or pathological condition, refers to any observable beneficial effect of the treatment. The beneficial effect can be evidenced, for example, by a delayed onset of clinical symptoms of the disease in a susceptible subject, a reduction in severity of some or all clinical symptoms of the disease, a slower progression of the disease, an improvement in the overall health or well-being of the subject, or by other parameters well known in the art that are specific to the particular disease. The phrase “treating a disease” refers to inhibiting the full development of a disease, for example, in a subject who is at risk for a disease. “Preventing” a disease or condition refers to prophylactic administering a composition to a subject who does not exhibit signs of a disease or exhibits only early signs for the purpose of decreasing the risk of developing a pathology or condition, or diminishing the severity of a pathology or condition.

“Pharmaceutical compositions” are compositions that include an amount (for example, a unit dosage) of one or more of the disclosed compounds together with one or more non-toxic pharmaceutically acceptable additives, including carriers, diluents, and/or adjuvants, and optionally other biologically active ingredients. Such pharmaceutical compositions can be prepared by standard pharmaceutical formulation techniques such as those disclosed in Remington's Pharmaceutical Sciences, Mack Publishing Co., Easton, Pa. (19th Edition).

The terms “pharmaceutically acceptable salt or ester” refers to salts or esters prepared by conventional means that include salts, e.g., of inorganic and organic acids, including but not limited to hydrochloric acid, hydrobromic acid, sulfuric acid, phosphoric acid, methanesulfonic acid, ethanesulfonic acid, malic acid, acetic acid, oxalic acid, tartaric acid, citric acid, lactic acid, fumaric acid, succinic acid, maleic acid, salicylic acid, benzoic acid, phenylacetic acid, mandelic acid and the like. “Pharmaceutically acceptable salts” of the presently disclosed compounds also include those formed from cations such as sodium, potassium, aluminum, calcium, lithium, magnesium, zinc, and from bases such as ammonia, ethylenediamine, N-methyl-glutamine, lysine, arginine, ornithine, choline, N,N′-dibenzylethylenediamine, chloroprocaine, diethanolamine, procaine, N-benzylphenethylamine, diethylamine, piperazine, tris(hydroxymethyl)aminomethane, and tetramethylammonium hydroxide. These salts may be prepared by standard procedures, for example by reacting the free acid with a suitable organic or inorganic base. Any chemical compound recited in this specification may alternatively be administered as a pharmaceutically acceptable salt thereof. “Pharmaceutically acceptable salts” are also inclusive of the free acid, base, and zwitterionic forms. Descriptions of suitable pharmaceutically acceptable salts can be found in Handbook of Pharmaceutical Salts, Properties, Selection and Use, Wiley VCH (2002). When compounds disclosed herein include an acidic function such as a carboxy group, then suitable pharmaceutically acceptable cation pairs for the carboxy group are well known to those skilled in the art and include alkaline, alkaline earth, ammonium, quaternary ammonium cations and the like. Such salts are known to those of skill in the art. For additional examples of “pharmacologically acceptable salts,” see Berge et al., J. Pharm. Sci. 66:1 (1977).

“Pharmaceutically acceptable esters” includes those derived from compounds described herein that are modified to include a carboxyl group. An in vivo hydrolysable ester is an ester, which is hydrolysed in the human or animal body to produce the parent acid or alcohol. Representative esters thus include carboxylic acid esters in which the non-carbonyl moiety of the carboxylic acid portion of the ester grouping is selected from straight or branched chain alkyl (for example, methyl, n-propyl, t-butyl, or n-butyl), cycloalkyl, alkoxyalkyl (for example, methoxymethyl), aralkyl (for example benzyl), aryloxyalkyl (for example, phenoxymethyl), aryl (for example, phenyl, optionally substituted by, for example, halogen, C.sub.1-4 alkyl, or C.sub.1-4 alkoxy) or amino); sulphonate esters, such as alkyl- or aralkylsulphonyl (for example, methanesulphonyl); or amino acid esters (for example, L-valyl or L-isoleucyl). A “pharmaceutically acceptable ester” also includes inorganic esters such as mono-, di-, or tri-phosphate esters. In such esters, unless otherwise specified, any alkyl moiety present advantageously contains from 1 to 18 carbon atoms, particularly from 1 to 6 carbon atoms, more particularly from 1 to 4 carbon atoms. Any cycloalkyl moiety present in such esters advantageously contains from 3 to 6 carbon atoms. Any aryl moiety present in such esters advantageously comprises a phenyl group, optionally substituted as shown in the definition of carbocycylyl above. Pharmaceutically acceptable esters thus include C₁-C₂₂ fatty acid esters, such as acetyl, t-butyl or long chain straight or branched unsaturated or omega-6 monounsaturated fatty acids such as palmoyl, stearoyl and the like. Alternative aryl or heteroaryl esters include benzoyl, pyridylmethyloyl and the like any of which may be substituted, as defined in carbocyclyl above. Additional pharmaceutically acceptable esters include aliphatic L-amino acid esters such as leucyl, isoleucyl and especially valyl.

For therapeutic use, salts of the compounds are those wherein the counter-ion is pharmaceutically acceptable. However, salts of acids and bases which are non-pharmaceutically acceptable may also find use, for example, in the preparation or purification of a pharmaceutically acceptable compound.

The pharmaceutically acceptable acid and base addition salts as mentioned hereinabove are meant to comprise the therapeutically active non-toxic acid and base addition salt forms which the compounds are able to form. The pharmaceutically acceptable acid addition salts can conveniently be obtained by treating the base form with such appropriate acid. Appropriate acids comprise, for example, inorganic acids such as hydrohalic acids, e.g. hydrochloric or hydrobromic acid, sulfuric, nitric, phosphoric and the like acids; or organic acids such as, for example, acetic, propanoic, hydroxyacetic, lactic, pyruvic, oxalic (i.e. ethanedioic), malonic, succinic (i.e. butanedioic acid), maleic, fumaric, malic (i.e. hydroxybutanedioic acid), tartaric, citric, methanesulfonic, ethanesulfonic, benzenesulfonic, p-toluenesulfonic, cyclamic, salicylic, p-aminosalicylic, pamoic and the like acids. Conversely said salt forms can be converted by treatment with an appropriate base into the free base form.

The compounds containing an acidic proton may also be converted into their non-toxic metal or amine addition salt forms by treatment with appropriate organic and inorganic bases. Appropriate base salt forms comprise, for example, the ammonium salts, the alkali and earth alkaline metal salts, e.g. the lithium, sodium, potassium, magnesium, calcium salts and the like, salts with organic bases, e.g. the benzathine, N-methyl-D-glucamine, hydrabamine salts, and salts with amino acids such as, for example, arginine, lysine and the like.

Prodrugs of the disclosed compounds also are contemplated herein. A prodrug is an active or inactive compound that is modified chemically through in vivo physiological action, such as hydrolysis, metabolism and the like, into an active compound following administration of the prodrug to a subject. The term “prodrug” as used throughout this text means the pharmacologically acceptable derivatives such as esters, amides and phosphates, such that the resulting in vivo biotransformation product of the derivative is the active drug as defined in the compounds described herein. Prodrugs preferably have excellent aqueous solubility, increased bioavailability and are readily metabolized into the active inhibitors in vivo. Prodrugs of a compounds described herein may be prepared by modifying functional groups present in the compound in such a way that the modifications are cleaved, either by routine manipulation or in vivo, to the parent compound. The suitability and techniques involved in making and using prodrugs are well known by those skilled in the art. F or a general discussion of prodrugs involving esters see Svensson and Tunek, Drug Metabolism Reviews 165 (1988) and Bundgaard, Design of Prodrugs, Elsevier (1985).

The term “prodrug” also is intended to include any covalently bonded carriers that release an active parent drug of the present invention in vivo when the prodrug is administered to a subject. Since prodrugs often have enhanced properties relative to the active agent pharmaceutical, such as, solubility and bioavailability, the compounds disclosed herein can be delivered in prodrug form. Thus, also contemplated are prodrugs of the presently disclosed compounds, methods of delivering prodrugs and compositions containing such prodrugs. Prodrugs of the disclosed compounds typically are prepared by modifying one or more functional groups present in the compound in such a way that the modifications are cleaved, either in routine manipulation or in vivo, to yield the parent compound. Prodrugs include compounds having a phosphonate and/or amino group functionalized with any group that is cleaved in vivo to yield the corresponding amino and/or phosphonate group, respectively. Examples of prodrugs include, without limitation, compounds having an acylated amino group and/or a phosphonate ester or phosphonate amide group. In particular examples, a prodrug is a lower alkyl phosphonate ester, such as an isopropyl phosphonate ester.

Protected derivatives of the disclosed compounds also are contemplated. A variety of suitable protecting groups for use with the disclosed compounds are disclosed in Greene and Wuts, Protective Groups in Organic Synthesis; 3rd Ed.; John Wiley & Sons, New York, 1999.

In general, protecting groups are removed under conditions that will not affect the remaining portion of the molecule. These methods are well known in the art and include acid hydrolysis, hydrogenolysis and the like. One preferred method involves the removal of an ester, such as cleavage of a phosphonate ester using Lewis acidic conditions, such as in TMS-Br mediated ester cleavage to yield the free phosphonate. A second preferred method involves removal of a protecting group, such as removal of a benzyl group by hydrogenolysis utilizing palladium on carbon in a suitable solvent system such as an alcohol, acetic acid, and the like or mixtures thereof. A t-butoxy-based group, including t-butoxy carbonyl protecting groups can be removed utilizing an inorganic or organic acid, such as HCl or trifluoroacetic acid, in a suitable solvent system, such as water, dioxane and/or methylene chloride. Another exemplary protecting group, suitable for protecting amino and hydroxy functions amino is trityl. Other conventional protecting groups are known and suitable protecting groups can be selected by those of skill in the art in consultation with Greene and Wuts, Protective Groups in Organic Synthesis; 3rd Ed.; John Wiley & Sons, New York, 1999. When an amine is deprotected, the resulting salt can readily be neutralized to yield the free amine. Similarly, when an acid moiety, such as a phosphonic acid moiety is unveiled, the compound may be isolated as the acid compound or as a salt thereof.

### Compounds

A compound of formula I, or a stereoisomer, tautomer, or pharmaceutically acceptable salt thereof:

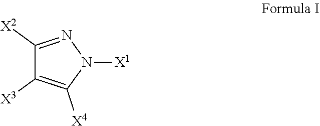

wherein X¹ is benzimidazolyl, substituted benzimidazolyl, azabenzimidazolyl, substituted azabenzimidazolyl, pyrazolyl, or substituted pyrazolyl;

X² is aryl, substituted aryl, arylalkyl, substituted arylalkyl, heteroaryl, substituted heteroaryl, heteroarylalkyl, substituted heteroarylalkyl, cycloalkyl, substituted cycloalkyl, (cycloalkyl)alkyl, substituted (cycloalkyl)alkyl, heterocycloalkyl, substituted heterocycloalkyl, (heterocycloalkyl)alkyl, substituted (heterocycloalkyl)alkyl, alkyl, substituted alkyl, alkoxycarbonyl, substituted alkoxycarbonyl, dialkylaminocarbonyl, substituted dialkylaminocarbonyl, (heterocycloalkyl)carbonyl, or substituted (heterocycloalkyl)carbonyl;

X³ is alkyl, substituted alkyl, alkenyl, substituted alkenyl, heterocycloalkyl, substituted heterocycloalkyl, (heterocycloalkyl)alkyl, substituted (heterocycloalkyl)alkyl, aryl, substituted aryl, arylalkyl, substituted arylalkyl, heteroaryl, substituted heteroaryl, heteroarylalkyl, substituted heteroarylalkyl, cycloalkyl, substituted cycloalkyl, (cycloalkyl)alkyl, or substituted (cycloalkyl)alkyl; and

X⁴ is hydroxy, amino, alkyl, or hydrogen.

In certain embodiments, X¹ is a benzimidazolyl or a substituted benzimidazolyl.

In certain embodiments, X¹ is

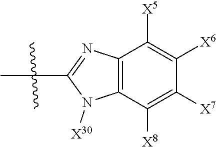

wherein each of X⁵, X⁶, X⁷, and X⁸ is independently selected from hydrogen, halogen, cyano, alkyl, haloalkyl, alkoxy, haloalkoxy or cycloalkyl; and X³⁰ is hydrogen, alkyl, haloalkyl, or cycloalkyl. In certain embodiments, X³⁰ is aminoalkyl, alkylaminoalkyl, dialkylaminoalkyl, heterocycloalkyl, or heteroarylalkyl.

In certain embodiments, each of X⁵, X⁶, X⁷, X⁸ and X³⁰ is hydrogen.

In certain embodiments, X³⁰ is hydrogen.

In certain embodiments, X³⁰ is morpholinylalkyl.

In certain embodiments, X³⁰ is 2-(morpholin-4-yl)ethyl.

In certain embodiments, X⁶ is halogen, particularly —Cl. In certain embodiments, X⁶ is halogen, particularly —Cl, and X⁵, X⁷ and X⁸ are each hydrogen. In certain embodiments X⁶ and X⁷ are both —F.

In certain embodiments, X¹ is azabenzimidazolyl or a substituted azabenzimidazolyl.

In certain embodiments, X¹ is:

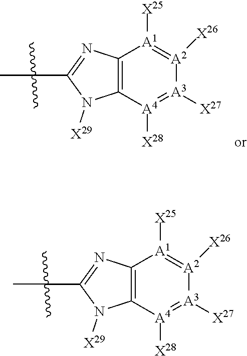

wherein each of A¹, A², A³ and A⁴ is independently C or N, provided at least one of A¹, A², A³ and A⁴ is N; each of X²⁵, X²⁶, X²⁷, and X²⁸ is independently selected from hydrogen, halogen, cyano, alkyl, haloalkyl, alkoxy, haloalkoxy or cycloalkyl; and X²⁹ is hydrogen, alkyl, haloalkyl, or cycloalkyl (in certain embodiments, X²⁹ is aminoalkyl, alkylaminoalkyl, dialkylaminoalkyl, heterocycloalkyl, or heteroarylalkyl); and also provided that if A¹ is N then X²⁵ is not present, if A² is N then X²⁶ is not present, if A³ is N then X²⁷ is not present, and if A⁴ is N then X²⁸ is not present.

In certain embodiments, A⁴ is N and X²⁸ is not present.

In certain embodiments, A⁴ is N and X²⁸ is not present, and A′, A² and A³ are each C.

In certain embodiments, each of X²⁵, X²⁶, X²⁷, and X²⁹ is H (X²⁸ is not present when A⁴ is N).

In certain embodiments, X¹ is pyrazolyl or substituted pyrazolyl.

In certain embodiments, X¹ is:

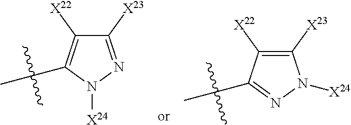

wherein each of X²² and X²³ and X²⁴ is independently selected from hydrogen, halogen, cyano, alkyl, haloalkyl, alkoxy, haloalkoxy, and cycloalkyl; and X²⁴ is selected from hydrogen, alkyl, haloalkyl, alkylsulfonyl and cycloalkyl.

In certain embodiments, X²² and X²⁴ are each hydrogen, and X²³ is alkyl, haloalkyl or cycloalkyl.

In certain embodiments, X²³ is haloalkyl, particularly —CF₃. In certain embodiments, X²² and X²⁴ are each hydrogen, and X²³ is —CF₃.

In certain embodiments, X¹ is substituted benzimidazolyl, substituted azabenzimidazolyl or substituted pyrazolyl, each of which is substituted with 1 to 5 substituents independently selected from halogen, cyano, alkyl, haloalkyl, alkoxy haloalkoxy, cycloalkyl, or a combination thereof.

In certain embodiments, X² is

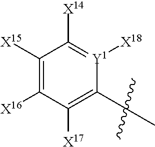

wherein each of X¹⁴, X¹⁵, X¹⁶, X¹⁷ and X¹⁸ is independently selected from hydrogen, halogen, cyano, alkyl, haloalkyl, alkoxy, haloalkoxy, alkylsulfonyl or substituted alkyl, or X¹⁵ and X¹⁶ taken together, or X¹⁶ and X¹² taken together, comprise a methylenedioxy or difluoromethylenedioxy group; and
Y¹ is C or N, provided that if Y¹ then X¹⁸ is not present.

In certain embodiments, X¹⁵ is a haloalkyl, particularly —CF₃.

In certain embodiments, X¹⁵ is alkylsulfonyl, particularly MeSO₂—.

In certain embodiments, X¹⁶ is alkylsulfonyl, particularly MeSO₂—.

In certain embodiments, at least one of X¹⁴, X¹⁵, X¹⁶, X¹⁷ or X¹⁸ is not hydrogen.

In certain embodiments, each of X¹⁴, X¹⁶, X¹⁷ and X¹⁸ is hydrogen, and X¹⁵ is not hydrogen.

In certain embodiments, each of X¹⁴, X¹⁵, X¹⁷ and X¹⁸ is hydrogen, and X¹⁶ is not hydrogen.

In certain embodiments, X² is a N-heterocycloalkyl.

In certain embodiments, X² is

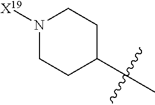

wherein X¹⁹ is alkoxycarbonyl, heteroaryl, alkylcarbonyl, alkoxycarbonylamino, alkyl, haloalkyl, cycloalkyl, arylalkyl, heteroarylalkyl, (heterocycloalkyl)alkyl, cycloalkylcarbonyl, hydroxycycloalkylcarbonyl, hydroxyalkylcarbonyl, alkoxyalkylcarbonyl, arylalkoxycarbonyl, arylcarbonyl, heterocycloalkyl, heterocycloalkylcarbonyl, heteroarylcarbonyl, (arylalkyl)carbonyl, heterocycloalkyl)alkylcarbonyl, heteroarylalkylcarbonyl, heteroarylalkoxyalkylcarbonyl, alkylsulfonyl, cycloalkylsulfonyl, arylsulfonyl, heteroarylsulfonyl, alkylaminocarbonyl, dialkylaminocarbonyl, or dialkylaminosulfonyl, each of which is optionally substituted with 1 to 5 groups independently selected from halogen, amino, hydroxy, alkyl and alkoxy.

In certain embodiments, X² is

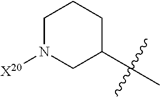

wherein X¹⁹ is alkoxycarbonyl, heteroaryl, alkylcarbonyl, alkoxycarbonylamino, alkyl, haloalkyl, cycloalkyl, arylalkyl, heteroarylalkyl, (heterocycloalkyl)alkyl, cycloalkylcarbonyl, hydroxycycloalkylcarbonyl, hydroxyalkylcarbonyl, alkoxyalkylcarbonyl, arylalkoxycarbonyl, arylcarbonyl, heterocycloalkyl, heterocycloalkylcarbonyl, heteroarylcarbonyl, (arylalkyl)carbonyl, heterocycloalkyl)alkylcarbonyl, heteroarylalkylcarbonyl, heteroarylalkoxyalkylcarbonyl, alkylsulfonyl, cycloalkylsulfonyl, arylsulfonyl, heteroarylsulfonyl, alkylaminocarbonyl, dialkylaminocarbonyl, or dialkylaminosulfonyl, each of which is optionally substituted with 1 to 5 groups independently selected from halogen, amino, hydroxy, alkyl and alkoxy.

In certain embodiments, X¹⁹ or X²⁰ is alkoxycarbonyl, particularly (C₂-C₅) alkoxycarbonyl (for example, n-propoxycarbonyl, i-propoxycarbonyl, n-butoxycarbonyl, i-butoxycarbonyl, t-butoxycarbonyl, or sec-butoxycarbonyl), dialkylaminosulfonyl, particularly N(CH₃)₂SO₂—, or cycloalkylsulfonyl, particularly cyclohexyl-SO₂—.

In certain embodiments, X² is O-heterocycloalkyl, particularly tetrahydropyran-4-yl.

In certain embodiments, X² is cycloalkyl, particularly cyclopropyl or cyclobutyl.

In certain embodiments, X² is substituted aryl, substituted arylalkyl, substituted heteroaryl, substituted heteroarylalkyl, substituted cycloalkyl, substituted (cycloalkyl)alkyl, substituted heterocycloalkyl, substituted (heterocycloalkyl)alkyl, substituted alkyl, substituted alkoxycarbonyl, substituted dialkylaminocarbonyl, or substituted (heterocycloalkyl)carbonyl, each of which is substituted with 1 to 5 substituents independently selected from hydroxy, halogen, cyano, carboxy, carboxamide, alkyl, haloalkyl, alkoxy, haloalkoxy, alkylsulfonyl, alkoxycarbonylamino, heteroarylsulfonylamino, heteroarylamino, cycloalkyl, arylalkyl, heteroaryl, heteroarylalkyl, heterocycloalkyl, (heterocycloalkyl)alkyl, alkylcarbonyl, cycloalkylcarbonyl, hydroxycycloalkylcarbonyl, hydroxyalkylcarbonyl, alkoxycarbonyl, alkoxyalkylcarbonyl, arylalkoxycarbonyl, arylcarbonyl, heterocycloalkylcarbonyl, (heterocycloalkyl)alkylcarbonyl, heteroarylcarbonyl, (arylalkyl)carbonyl, heteroarylalkylcarbonyl, heteroarylalkoxyalkylcarbonyl, cycloalkylsulfonyl, arylsulfonyl, heteroarylsulfonyl, arylalkoxycarbonyl, alkylaminocarbonyl, (cycloalkyl)alkylaminocarbonyl, dialkylaminocarbonyl, dialkylaminosulfonyl, methylenedioxy, difluoromethylenedioxy, or a combination thereof; each of which is further optionally independently substituted with 1 to 5 groups independently selected from halogen, amino, hydroxy, alkyl, alkoxy, or a combination thereof.

In certain embodiments, X³ is alkenyl, arylalkyl, substituted arylalkyl, heteroarylalkyl, substituted heteroarylalkyl, (heterocycloalkyl)alkyl, substituted (heterocycloalkyl)alkyl, (cycloalkyl)alkyl, substituted (cycloalkyl)alkyl or hydroxyalkyl.

In certain embodiments, X³ is arylmethyl, substituted arylmethyl, heteroarylmethyl, substituted heteroarylmethyl, (heterocycloalkyl)methyl, substituted (heterocycloalkyl)methyl, (cycloalkyl)methyl or substituted (cycloalkyl)methyl.

In certain embodiments, X³ is arylethyl, substituted arylethyl, heteroarylethyl, substituted heteroarylethyl, (heterocycloalkyl)ethyl, substituted (heterocycloalkyl)ethyl, (cycloalkyl)ethyl or substituted (cycloalkyl)ethyl.

In certain embodiments, X³ is

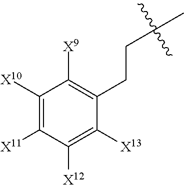

wherein each of X⁹, X¹⁰, X¹¹, X¹² and X¹³ is independently selected from hydrogen, halogen cyano, alkyl, alkoxy, haloalkyl or alkylsulfonyl.

In certain embodiments, X¹¹ is halogen, particularly —F. In certain embodiments, X¹¹ is halogen, particularly —F, and each of X⁹, X¹⁰, X¹² and X¹³ is hydrogen. In certain embodiments, X¹¹ is not hydrogen.

In certain embodiments, X³ is

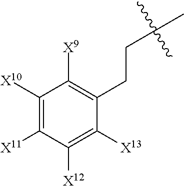

wherein each of X⁹, X¹⁰, X¹² and X¹³ is independently selected from hydrogen, halogen cyano, alkyl, alkoxy, haloalkyl or alkylsulfonyl; and X¹¹ is —C(O)R⁷⁰, wherein R⁷⁰ is hydroxy, alkoxy, substituted alkoxy, alkyl, substituted alkyl, aryl, substituted aryl, heteroaryl, or substituted heteroaryl. In certain embodiments, X¹¹ is —C(O)—O-alkyl, particularly —C(O)—O-methyl.

In certain embodiments, X³ is:

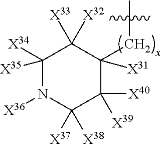

wherein x is 1 or 2; each of X³¹, X³², X³³, X³⁴, X³⁵, X³⁷, X³⁸, X³⁹ and X⁴⁰ is independent selected from hydrogen, hydroxy, cyano, aminocarbonyl, alkyl or haloalkyl; and X³⁶ is H, haloalkyl, alkoxycarbonyl, arylalkoxycarbonyl, alkylcarbonyl, hydroxyalkylcarbonyl, cycloalkylcarbonyl, arylcarbonyl, arylcarbonyl, alkylsulfonyl and heteroaryl, wherein the aryl and heteroaryl groups are each optionally substituted with halogen and alkoxy.

In certain embodiments, each of X³¹, X³², X³³, X³⁴, X³⁵, X³⁷, X³⁸, X³⁹ and X⁴⁰ is hydrogen, and X³⁶ is alkoxycarbonyl, particularly (C₂-C₅) alkoxycarbonyl (for example, n-propoxycarbonyl, i-propoxycarbonyl, n-butoxycarbonyl, i-butoxycarbonyl, t-butoxycarbonyl, or sec-butoxycarbonyl).

In certain embodiments, X³ is:

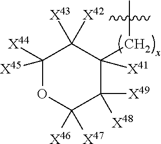

wherein x is 1 or 2; and each of X⁴¹, X⁴², X⁴³, X⁴⁴, X⁴⁵, X⁴⁶, X⁴⁷, X⁴⁸, and X⁴⁹ is independently selected from hydrogen, hydroxy, cyano, aminocarbonyl, alkyl or haloalkyl.

In certain embodiments, each of X⁴¹, X⁴², X⁴³, X⁴⁴, X⁴⁵, X⁴⁶, X⁴⁷, X⁴⁸, and X⁴⁹ is hydrogen.

In certain embodiments, X³ is substituted alkyl, substituted alkenyl, substituted heterocycloalkyl, substituted (heterocycloalkyl)alkyl, substituted aryl, substituted arylalkyl, substituted heteroaryl, substituted heteroarylalkyl, substituted cycloalkyl, or substituted (cycloalkyl)alkyl, each of which is independently substituted with 1 to 5 substituents selected from hydroxy, halogen, cyano, carboxy, carboxamide, alkyl, haloalkyl, alkoxy, haloalkoxy, cycloalkyl, alkoxycarbonyl, arylalkoxycarbonyl, alkylcarbonyl, hydroxyalkylcarbonyl, cycloalkylcarbonyl, arylcarbonyl, haloarylcarbonyl, alkylsulfonyl, heteroaryl, or a combination thereof.

In certain embodiments, X³ is:

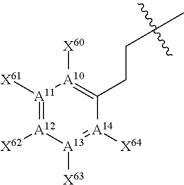

wherein each of A¹⁰, A¹¹, A¹², A¹³ and A¹⁴ is independently C or N, provided at least one of A¹, A¹¹, A¹², A¹³ and A¹⁴ is N; each of X⁶⁰, X⁶¹, X⁶², X⁶³ and X⁶⁴ is independently selected from hydrogen, halogen, cyano, alkyl, haloalkyl, alkoxy, haloalkoxy or cycloalkyl; and X²⁹ is hydrogen, alkyl, haloalkyl, or cycloalkyl; and also provided that if A¹⁰ is N then X⁶⁰ is not present, if A¹¹ is N then X⁶¹ is not present, if A¹² is N then X⁶² is not present, if A¹³ is N then X⁶³ is not present, and if A¹⁴ is N then X⁶⁴ is not present. In certain embodiments, A¹² is N; A¹⁰, A¹¹, A¹³ and A¹⁴ are each C; and X⁶⁰, X⁶¹, X⁶³ and X⁶⁴ are each H.

In certain embodiments, X⁴ is hydroxy.

Particular examples of the presently disclosed compounds may include one or more asymmetric centers; thus these compounds can exist in different stereoisomeric forms. Accordingly, compounds and compositions may be provided as individual pure enantiomers or as stereoisomeric mixtures, including racemic mixtures. In certain embodiments the compounds disclosed herein are synthesized in or are purified to be in substantially enantiopure form, such as in a 90% enantiomeric excess, a 95% enantiomeric excess, a 97% enantiomeric excess or even in greater than a 99% enantiomeric excess, such as in enantiopure form.

The presently disclosed compounds can have at least one asymmetric center or geometric center, cis-trans center(C═C, C═N). All chiral, diasteromeric, racemic, meso, rotational and geometric isomers of the structures are intended unless otherwise specified. The compounds can be isolated as a single isomer or as mixture of isomers. All tautomers of the compounds are also considered part of the disclosure. The presently disclosed compounds also includes all isotopes of atoms present in the compounds, which can include, but are not limited to, deuterium, tritium, ¹⁸F, etc

Illustrative compounds are listed below:

| Cpd No | Name |
|---|---|
| I-101 | 1-(1H-1,3-benzodiazol-2-yl)-4-[2-(pyridin-4-yl)ethyl]-3-[4- |
| | (trifluoromethyl)phenyl]-1H-pyrazol-5-ol |
| I-102 | 1-(1H-1,3-benzodiazol-2-yl)-3-cyclopropyl-4-[2-(4-fluorophenyl)ethyl]-1H- |
| | pyrazol-5-ol |
| I-103 | 1-(1H-1,3-benzodiazol-2-yl)-4-[2-(4-fluorophenyl)ethyl]-3-(pyrazin-2-yl)-1H- |
| | pyrazol-5-ol |
| I-104 | 1-(1H-1,3-benzodiazol-2-yl)-4-[2-(4-fluorophenyl)ethyl]-3-(4-methylphenyl)- |
| | 1H-pyrazol-5-ol |
| I-105 | 1-(1H-1,3-benzodiazol-2-yl)-3-(4-chlorophenyl)-4-[2-(4-fluorophenyl)ethyl]- |
| | 1H-pyrazol-5-ol |
| I-106 | 1-(1H-1,3-benzodiazol-2-yl)-4-[2-(4-fluorophenyl)ethyl]-3-[4- |
| | (trifluoromethyl)phenyl]-1H-pyrazol-5-ol |
| I-107 | 1-(1H-1,3-benzodiazol-2-yl)-4-[2-(4-fluorophenyl)ethyl]-3-(4-methoxyphenyl)- |
| | 1H-pyrazol-5-ol |
| I-108 | 1-(1H-1,3-benzodiazol-2-yl)-4-[2-(4-fluorophenyl)ethyl]-3-phenyl-1H-pyrazol- |
| | 5-ol |
| I-109 | 1-(1H-1,3-benzodiazol-2-yl)-4-[2-(4-fluorophenyl)ethyl]-3-(pyridin-2-yl)-1H- |
| | pyrazol-5-ol |
| I-110 | 1-(1H-1,3-benzodiazol-2-yl)-3-(3-fluorophenyl)-4-[2-(4-fluorophenyl)ethyl]- |
| | 1H-pyrazol-5-ol |
| I-111 | 1-(1H-1,3-benzodiazol-2-yl)-4-[2-(3,4-difluorophenyl)ethyl]-3-(pyrazin-2-yl)- |
| | 1H-pyrazol-5-ol |
| I-112 | 1-(1H-1,3-benzodiazol-2-yl)-4-[2-(3-chloro-4-fluorophenyl)ethyl]-3-(pyrazin-2- |
| | yl)-1H-pyrazol-5-ol |
| I-113 | 1-(1H-1,3-benzodiazol-2-yl)-4-[2-(4-fluorophenyl)ethyl]-3-(pyridin-4-yl)-1H- |
| | pyrazol-5-ol |
| I-114 | 1-(1H-1,3-benzodiazol-2-yl)-4-[2-(4-fluorophenyl)ethyl]-3-(oxan-4-yl)-1H- |
| | pyrazol-5-ol |
| I-115 | 1-(1H-1,3-benzodiazol-2-yl)-4-(prop-2-en-1-yl)-3-[4-(trifluoromethyl)phenyl]- |
| | 1H-pyrazol-5-ol |
| I-116 | 1-(1H-1,3-benzodiazol-2-yl)-3-(3-chlorophenyl)-4-[2-(4-fluorophenyl)ethyl]- |
| | 1H-pyrazol-5-ol |
| I-117 | 1-(1H-1,3-benzodiazol-2-yl)-3-(2-chlorophenyl)-4-[2-(4-fluorophenyl)ethyl]- |
| | 1H-pyrazol-5-ol |
| I-118 | 1-(1H-1,3-benzodiazol-2-yl)-3-(5-bromopyridin-3-yl)-4-[2-(4- |
| | fluorophenyl)ethyl]-1H-pyrazol-5-ol |
| I-119 | 1-(1H-1,3-benzodiazol-2-yl)-4-[2-(4-fluorophenyl)ethyl]-3-[5- |
| | (trifluoromethyl)pyridin-2-yl]-1H-pyrazol-5-ol |
| I-120 | 1-(1H-1,3-benzodiazol-2-yl)-4-[2-(4-fluorophenyl)ethyl]-3-[3-(4- |
| | fluorophenyl)propyl]-1H-pyrazol-5-ol |
| I-121 | 1-(1H-1,3-benzodiazol-2-yl)-4-[2-(4-fluorophenyl)ethyl]-3-(pyridin-3-yl)-1H- |
| | pyrazol-5-ol |
| I-122 | 1-(1H-1,3-benzodiazol-2-yl)-4-[(oxan-2-yl)methyl]-3-[4- |
| | (trifluoromethyl)phenyl]-1H-pyrazol-5-ol |
| I-123 | 1-(1H-1,3-benzodiazol-2-yl)-4-(cyclopentylmethyl)-3-[4- |
| | (trifluoromethyl)phenyl]-1H-pyrazol-5-ol |
| I-124 | 1-(1H-1,3-benzodiazol-2-yl)-4-[2-(4-fluorophenyl)ethyl]-3-(methoxymethyl)- |
| | 1H-pyrazol-5-ol |
| I-125 | 1-(1H-1,3-benzodiazol-2-yl)-4-[2-(4-fluorophenyl)ethyl]-3-(2-methoxyethyl)- |
| | 1H-pyrazol-5-ol |
| I-126 | 1-(1H-1,3-benzodiazol-2-yl)-4-[2-(4-fluorophenyl)ethyl]-3-[2- |
| | (trifluoromethyl)pyrimidin-5-yl]-1H-pyrazol-5-ol |
| I-127 | 3-[1-(1H-1,3-benzodiazol-2-y1)-4-[2-(4-fluorophenyl)ethyl]-5-hydroxy-1H- |
| | pyrazol-3-yl]-1-methyl-1,2-dihydropyridin-2-one |
| I-128 | 1-(1H-1,3-benzodiazol-2-yl)-4-[2-(3-methyl-1H-pyrazol-1-yl)ethyl]-3-[4- |
| | (trifluoromethyl)phenyl]-1H-pyrazol-5-ol |
| I-129 | 1-(1H-1,3-benzodiazol-2-yl)-3-cyclobutyl-4-[2-(4-fluorophenyl)ethyl]-1H- |
| | pyrazol-5-ol |
| I-130 | 1-(1H-1,3-benzodiazol-2-yl)-3-tert-butyl -4-[2-(4-fluorophenyl)ethyl]-1H- |
| | pyrazol-5-ol |
| I-131 | 1-(1H-1,3-benzodiazol-2-yl)-4-[2-(morpholin-4-yl)ethyl]-3-[4- |
| | (trifluoromethyl)phenyl]-1H-pyrazol-5-ol |
| I-132 | 1-{2-[1-(1H-1,3-benzodiazol-2-yl)-5-hydroxy-3-[4-(trifluoromethyl)phenyl]- |
| | 1H-pyrazol-4-yl]ethyl}pyrrolidin-2-one |
| I-133 | 4-[1-(1H-1,3-benzodiazol-2-y1)-4-[2-(4-fluorophenyl)ethyl]-5-hydroxy-1H- |
| | pyrazol-3-yl]-1-methyl-1,2-dihydropyridin-2-one |
| I-134 | 5-[1-(1H-1,3-benzodiazol-2-y1)-4-[2-(4-fluorophenyl)ethyl]-5-hydroxy-1H- |
| | pyrazol-3-yl]-1-methyl-1,2-dihydropyridin-2-one |
| I-135 | 1-(1H-1,3-benzodiazol-2-yl)-4-[2-(4-fluorophenyl)ethyl]-3-[6- |
| | (trifluoromethyl)pyridin-3-yl]-1H-pyrazol-5-ol |
| I-136 | 1-(1H-1,3-benzodiazol-2-yl)-4-[2-(4-fluorophenyl)ethyl]-3-(oxolan-2-yl)-1H- |
| | pyrazol-5-ol |
| I-137 | 3-{2-[1-(1H-1,3-benzodiazol-2-yl)-5-hydroxy-3-[4-(trifluoromethyl)phenyl]- |
| | 1H-pyrazol-4-yl]ethyl}-1-methyl-1,2-dihydropyridin-2-one |
| I-138 | 1-(1H-1,3-benzodiazol-2-yl)-4-[2-(4-fluorophenyl)ethyl]-3-(6-methoxypyridin- |
| | 2-yl)-1H-pyrazol-5-ol |
| I-139 | 1-(1H-1,3-benzodiazol-2-yl)-4-[2-(oxan-4-yl)ethyl]-3-[4- |
| | (trifluoromethyl)phenyl]-1H-pyrazol-5-ol |
| I-140 | 1-(1H-1,3-benzodiazol-2-yl)-4-[2-(4-fluorophenyl)ethyl]-3-(6-methylpyridin-3- |
| | yl)-1H-pyrazol-5-ol |
| I-141 | 1-(1H-1,3-benzodiazol-2-yl)-4-[2-(2H-1,2,3-triazol-2-yl)ethyl]-3-[4- |
| | (trifluoromethyl)phenyl]-1H-pyrazol-5-ol |
| I-142 | 1-(1H-1,3-benzodiazol-2-yl)-4-[2-(2-methyl-1H-imidazol-1-yl)ethyl]-3-[4- |
| | (trifluoromethyl)phenyl]-1H-pyrazol-5-ol |
| I-143 | 1-(1H-1,3-benzodiazol-2-yl)-4-[2-(4-chloro-1H-pyrazol-1-yl)ethyl]-3-[4- |
| | (trifluoromethyl)phenyl]-1H-pyrazol-5-ol |
| I-144 | 1-(1H-1,3-benzodiazol-2-yl)-4-[2-(3-methyl-1H-pyrazol-1-yl)ethyl]-3-[4- |
| | (trifluoromethyl)phenyl]-1H-pyrazol-5-ol |
| I-145 | 1-(1H-1,3-benzodiazol-2-yl)-3-(2,2-difluoro-2H-1,3-benzodioxol-5-yl)-4-[2-(4- |
| | fluorophenyl)ethyl]-1H-pyrazol-5-ol |
| I-146 | 1-(1H-1,3-benzodiazol-2-yl)-4-[2-(pyridin-2-yl)ethyl]-3-[5- |
| | (trifluoromethyl)pyridin-2-yl]-1H-pyrazol-5-ol |
| I-147 | 1-(1H-1,3-benzodiazol-2-yl)-4-[2-(4-bromophenyl)ethyl]-3-[5- |
| | (trifluoromethyl)pyridin-2-yl]-1H-pyrazol-5-ol |
| I-148 | 1-(1H-1,3-benzodiazol-2-yl)-4-[2-(4-fluorophenyl)ethyl]-3-(4- |
| | methanesulfonylphenyl)-1H-pyrazol-5-ol |
| I-149 | 1-(1H-1,3-benzodiazol-2-yl)-3-(4-methanesulfonylphenyl)-4-[2-(oxan-4- |
| | yl)ethyl]-1H-pyrazol-5-ol |
| I-150 | 1-(1H-1,3-benzodiazol-2-yl)-3-(3-fluorophenyl)-4-[2-(oxan-4-yl)ethyl]-1H- |
| | pyrazol-5-ol |
| I-151 | 1-(1H-1,3-benzodiazol-2-yl)-3-(4-fluorophenyl)-4-[(oxan-4-yl)methyl]-1H- |
| | pyrazol-5-ol |
| I-152 | 1-(1H-1,3-benzodiazol-2-yl)-4-[2-(4-fluorophenyl)ethyl]-3-[4- |
| | (trifluoromethyl)phenyl]-1H-pyrazol-5-ol |
| I-153 | 1-(1H-1,3-benzodiazol-2-yl)-4-(2-hydroxyethyl)-3-[4-(trifluoromethyl)phenyl]- |
| | 1H-pyrazol-5-ol |
| I-154 | 1-(1H-1,3-benzodiazol-2-yl)-3-(4-methanesulfonylphenyl)-4-{2-[1-(2,2,2- |
| | trifluoroethyl)piperidin-4-yl]ethyl}-1H-pyrazol-5-ol |
| I-155 | 4-{[1-(1H-1,3-benzodiazol-2-yl)-4-[2-(4-fluorophenyl)ethyl]-5-hydroxy-1H- |
| | pyrazol-3-yl]methyl}-1-phenylpiperazin-2-one |
| I-156 | 1-(1H-1,3-benzodiazol-2-yl)-4-[(4-chloro-2-fluorophenyl)methyl]-3-methyl- |
| | 1H-pyrazol-5-ol |
| I-157 | 1-(1H-1,3-benzodiazol-2-yl)-4-[2-(4-fluorophenyl)ethyl]-3-[(morpholin-4- |
| | yl)methyl]-1H-pyrazol-5-ol |
| I-158 | benzyl 4-[1-(1H-1,3-benzodiazol-2-yl)-4-[2-(4-fluorophenyl)ethyl]-5-hydroxy- |
| | 1H-pyrazol-3-yl]piperidine-1-carboxylate |
| I-159 | benzyl 4-[1-(1H-1,3-benzodiazol-2-yl)-4-[(3,4-difluorophenyl)methyl]-5- |
| | hydroxy-1H-pyrazol-3-yl]piperidine-1-carboxylate |
| I-160 | benzyl 3-[1-(1H-1,3-benzodiazol-2-yl)-4-[2-(4-fluorophenyl)ethyl]-5-hydroxy- |
| | 1H-pyrazol-3-yl]piperidine-1-carboxylate |
| I-161 | 1-(6-chloro-1H-1,3-benzodiazol-2-yl)-4-[2-(4-fluorophenyl)ethyl]-3-[4- |
| | (trifluoromethyl)phenyl]-1H-pyrazol-5-ol |
| I-162 | benzyl 4-{[1-(1H-1,3-benzodiazol-2-yl)-5-hydroxy-3-[4- |
| | (trifluoromethyl)phenyl]-1H-pyrazol-4-yl]methyl}piperidine-1-carboxylate |
| I-163 | 1-(6-chloro-1H-1,3-benzodiazol-2-yl)-4-[2-(3-methyl-1H-pyrazol-1-yl)ethyl]-3- |
| | [4-(trifluoromethyl)phenyl]-1H-pyrazol-5-ol |
| I-164 | benzyl 4-[1-(1H-1,3-benzodiazol-2-yl)-5-hydroxy-3-[4- |
| | (trifluoromethyl)phenyl]-1H-pyrazol-4-yl]piperidine-1-carboxylate |
| I-165 | 1-(5-chloro-1H-1,3-benzodiazol-2-yl)-4-[2-(4-fluorophenyl)ethyl]-3-phenyl- |
| | 1H-pyrazol-5-ol |
| I-166 | benzyl 4-[1-(7-chloro-1H-1,3-benzodiazol-2-yl)-4-[2-(4-fluorophenyl)ethyl]-5- |
| | hydroxy-1H-pyrazol-3-yl]piperidine-1-carboxylate |
| I-167 | 1-(5-fluoro-1H-1,3-benzodiazol-2-yl)-4-[2-(4-fluorophenyl)ethyl]-3-[5- |
| | (trifluoromethyl)pyridin-2-yl]-1H-pyrazol-5-ol |
| I-168 | 1-(5-chloro-1H-1,3-benzodiazol-2-yl)-3-(4-methanesulfonylphenyl)-4-[2-(oxan- |
| | 4-yl)ethyl]-1H-pyrazol-5-ol |
| I-169 | 1-(5-fluoro-1H-1,3-benzodiazol-2-yl)-3-(4-methanesulfonylphenyl)-4-[2-(oxan- |
| | 4-yl)ethyl]-1H-pyrazol-5-ol |
| I-170 | 1-(5-chloro-1H-1,3-benzodiazol-2-yl)-3-cyclopropyl-4-[2-(4- |
| | fluorophenyl)ethyl]-1H-pyrazol-5-ol |
| I-171 | methyl 4-{2-[1-(5-chloro-1H-1,3-benzodiazol-2-yl)-5-hydroxy-3-phenyl-1H- |
| | pyrazol-4-yl]ethyl}benzoate |
| I-172 | 1-(5-chloro-1H-1,3-benzodiazol-2-yl)-3-(4-fluoropheny1)-4-[2-(4- |
| | fluorophenyl)ethyl]-1H-pyrazol-5-ol |
| I-179 | Benzyl 4-[1-(5-chloro-1H-1,3-benzodiazol-2-yl)-5-hydroxy-4-[2-(oxan-4- |
| | yl)ethy1]-1H-pyrazol-3-yl]piperidine-1-carboxylate |
| I-180 | 1-(1H-1,3-benzodiazol-2-yl)-3-tert-butyl-4-[(oxan-4-yl)methyl]-1H-pyrazol-5- |
| | ol |
| I-181 | 1-(1H-1,3-benzodiazol-2-yl)-3-(1-fluorocyclopropyl)-4-[2-(4- |
| | fluorophenyl)ethyl]-1H-pyrazol-5-ol |
| I-182 | 1-(1H-1,3-benzodiazol-2-yl)-3-(4-fluorophenyl)-4-[2-(oxan-4-yl)ethyl]-1H- |
| | pyrazol-5-ol |
| I-183 | 1-(1H-1,3-benzodiazol-2-yl)-4-[(oxan-4-yl)methyl]-3-phenyl-1H-pyrazol-5-ol |
| I-184 | 1-(1H-1,3-benzodiazol-2-yl)-3-(3-fluorophenyl)-4-[(oxan-4-yl)methyl]-1H- |
| | pyrazol-5-ol |
| I-185 | 1-(1H-1,3-benzodiazol-2-yl)-3-(3,4-difluorophenyl)-4-[(oxan-4-yl)methyl]-1H- |
| | pyrazol-5-ol |
| I-186 | 1-(1H-1,3-benzodiazol-2-yl)-3-(2-fluorophenyl)-4-[(oxan-4-yl)methyl]-1H- |
| | pyrazol-5-ol |
| I-187 | 1-(1H-1,3-benzodiazol-2-yl)-3-(2,4-difluorophenyl)-4-[(oxan-4-yl)methyl]-1H- |
| | pyrazol-5-ol |
| I-188 | 1-(1H-1,3-benzodiazol-2-yl)-3-(2,5-difluorophenyl)-4-[(oxan-4-yl)methyl]-1H- |
| | pyrazol-5-ol |
| I-189 | 1-(1H-1,3-benzodiazol-2-yl)-4-[(oxan-4-yl)methyl]-3-[3- |
| | (trifluoromethyl)phenyl]-1H-pyrazol-5-ol |
| I-190 | 1-(1H-1,3-benzodiazol-2-yl)-3-(3,5-difluorophenyl)-4-[(oxan-4-yl)methyl]-1H- |
| | pyrazol-5-ol |
| I-191 | 1-(5-chloro-1H-1,3-benzodiazol-2-yl)-4-[2-(pyridin-4-yl)ethyl]-3-[4- |
| | (trifluoromethyl)phenyl]-1H-pyrazol-5-ol |
| I-192 | ethyl 4-{2-[1-(5-chloro-1H-1,3-benzodiazol-2-yl)-5-hydroxy-3-phenyl-1H- |
| | pyrazol-4-yl]ethyl}benzoate |
| I-201 | tert-butyl 4-{2-[1-(1H-1,3-benzodiazol-2-yl)-5-hydroxy-3-(4- |
| | methanesulfonylphenyl)-1H-pyrazol-4-yl]ethyl}piperidine-1-carboxylate |
| I-202 | tert-butyl 4-[1-(1H-1,3-benzodiazol-2-yl)-4-[2-(4-fluorophenyl)ethyl]-5- |
| | hydroxy-1H-pyrazol-3-yl]piperidine-1-carboxylate |
| I-203 | tert-butyl 4-[1-(1H-1,3-benzodiazol-2-yl)-4-[(3,4-difluorophenyl)methyl]-5- |
| | hydroxy-1H-pyrazol-3-yl]piperidine-1-carboxylate |
| I-204 | tert-butyl 4-{2-[1-(1H-1,3-benzodiazol-2-yl)-5-hydroxy-3-[4- |
| | (trifluoromethyl)phenyl]-1H-pyrazol-4-yl]ethyl[piperidine-1-carboxylate |
| I-205 | tert-butyl 2-[1-(1H-1,3-benzodiazol-2-yl)-4-[2-(4-fluorophenyl)ethyl]-5- |
| | hydroxy-1H-pyrazol-3-yl]pyrrolidine-1-carboxylate |
| I-206 | tert-butyl 3-{[1-(1H-1,3-benzodiazol-2-yl)-4-[2-(4-fluorophenyl)ethyl]-5- |
| | hydroxy-1H-pyrazol-3-yl]methyl}pyrrolidine-1-carboxylate |
| I-207 | tert-butyl 2-{[1-(1H-1,3-benzodiazol-2-yl)-4-[2-(4-fluorophenyl)ethyl]-5- |
| | hydroxy-1H-pyrazol-3-yl]methyl}piperidine-1-carboxylate |
| I-208 | tert-butyl 4-[1-(1H-1,3-benzodiazol-2-yl)-4-[2-(4-fluorophenyl)ethyl]-5- |
| | hydroxy-1H-pyrazol-3-yl]benzoate |
| I-209 | tert-butyl 6-[1-(1H-1,3-benzodiazol-2-yl)-4-[2-(4-fluorophenyl)ethyl]-5- |
| | hydroxy-1H-pyrazol-3-yl]-3-azabicyclo[3.1.0]hexane-3-carboxylate |
| I-210 | tert-butyl 4-{2-[1-(1H-1,3-benzodiazol-2-yl)-5-hydroxy-3-(3- |
| | methanesulfonylphenyl)-1H-pyrazol-4-yl]ethyl}piperidine-1-carboxylate |
| I-211 | tert-butyl 4-{[1-(1H-1,3-benzodiazol-2-yl)-4-[2-(4-fluorophenyl)ethyl]-5- |
| | hydroxy-1H-pyrazol-3-yl]methyl}piperazine-1-carboxylate |
| I-212 | tert-butyl N-{[1-(1H-1,3-benzodiazol-2-yl)-4-[2-(4-fluorophenyl)ethyl]-5- |
| | hydroxy-1H-pyrazol-3-yl]methyl}carbamate |
| I-213 | tert-butyl N-{4-[1-(1H-l,3-benzodiazol-2-yl)-4-[2-(4-fluorophenyl)ethyl]-5- |
| | hydroxy-1H-pyrazol-3-yl]oxan-4-yl}carbamate |
| I-214 | tert-butyl 3-{[1-(1H-1,3-benzodiazol-2-yl)-4-[2-(4-fluorophenyl)ethyl]-5- |
| | hydroxy-1H-pyrazol-3-yl]methyl}piperidine-1-carboxylate |
| I-215 | tert-butyl 4-{2-[1-(5-chloro-1H-1,3-benzodiazol-2-yl)-5-hydroxy-3-(4- |
| | methanesulfonylphenyl)-1H-pyrazol-4-yl]ethyl}piperidine-1-carboxylate |
| I-216 | tert-butyl 4-[1-(5-chloro-1H-1,3-benzodiazol-2-yl)-4-[2-(4-fluorophenyl)ethyl]- |
| | 5-hydroxy-1H-pyrazol-3-yl]piperidine-1-carboxylate |
| I-217 | tert-butyl 4-[1-(7-chloro-1H-1,3-benzodiazol-2-yl)-4-[2-(4-fluorophenyl)ethyl]- |
| | 5-hydroxy-1H-pyrazol-3-yl]piperidine-1-carboxylate |
| I-218 | tert-butyl 4-{2-[1-(5-chloro-1H-1,3-benzodiazol-2-yl)-3-cyclopropyl-5- |
| | hydroxy-1H-pyrazol-4-yl]ethyl}piperidine-1-carboxylate |
| I-219 | tert-butyl 4-[1-(5,6-difluoro-1H-1,3-benzodiazol-2-yl)-4-[2-(4- |
| | fluorophenyl)ethyl]-5-hydroxy-1H-pyrazol-3-yl]piperidine-1-carboxylate |
| I-220 | tert-butyl 4-{4-[2-(4-fluorophenyl)ethyl]-5-hydroxy-1-{3H-imidazo[4,5- |
| | b]pyridin-2-yl}-1H-pyrazol-3-yl}piperidine-1-carboxylate |
| I-221 | tert-butyl 4-{4-[2-(4-fluorophenyl)ethyl]-5-hydroxy-1-{3H-imidazo[4,5- |
| | c]pyridin-2-yl}-1H-pyrazol-3-yl}piperidine-1-carboxylate |
| I-301 | 4-[2-(4-fluorophenyl)ethyl]-1-{1H-imidazo[4,5-b]pyridin-2-yl}-3-[4- |
| | (trifluoromethyl)phenyl]-1H-pyrazol-5-ol |
| I-302 | 4-[2-(4-fluorophenyl)ethyl]-1-{3H-imidazo[4,5-b]pyridin-2-yl}-3-[5- |
| | (trifluoromethyl)pyridin-2-yl]-1H-pyrazol-5-ol |
| I-303 | 1-{3H-imidazo[4,5-b]pyridin-2-yl}-4-[2-(3-methyl-1H-pyrazol-1-yl)ethyl]-3- |
| | [4-(trifluoromethyl)phenyl]-1H-pyrazol-5-ol |
| I-304 | 3-(3-fluorophenyl)-1-{3H-imidazo[4,5-b]pyridin-2-yl}-4-[2-(oxan-4-yl)ethyl]- |
| | 1H-pyrazol-5-ol |
| I-305 | 1-{3H-imidazo[4,5-b]pyridin-2-yl}-3-(3-methanesulfonylphenyl)-4-[2-(oxan-4- |
| | yl)ethyl]-1H-pyrazol-5-ol |
| I-306 | benzyl 4-{4-[2-(4-fluorophenyl)ethyl]-5-hydroxy-1-{3H-imidazo[4,5-b]pyridin- |
| | 2-yl}-1H-pyrazol-3-yl}piperidine-1-carboxylate |
| I-307 | 4-[2-(4-fluorophenyl)ethyl]-3-[3-(4-fluorophenyl)propyl]-1-{3H-imidazo[4,5- |
| | b]pyridin-2-yl}-1H-pyrazol-5-ol |
| I-308 | 3-cyclopropyl-4-[2-(4-fluorophenyl)ethyl]-1-{3H-imidazo[4,5-b]pyridin-2-yl}- |
| | 1H-pyrazol-5-ol |
| I-309 | 4-[2-(4-fluorophenyl)ethyl]-1-{3H-imidazo[4,5-c]pyridin-2-yl}-3-[4- |
| | (trifluoromethyl)phenyl]-1H-pyrazol-5-ol |
| I-310 | benzyl 4-{4-[2-(4-fluorophenyl)ethyl]-5-hydroxy-1-{3H-imidazo[4,5-c]pyridin- |
| | 2-yl}-1H-pyrazol-3-yl}piperidine-1-carboxylate |
| I-401 | 4-[2-(4-fluorophenyl)ethyl]-1-(1-methyl-1H-1,3-benzodiazol-2-yl)-3-[4- |
| | (trifluoromethyl)phenyl]-1H-pyrazol-5-ol |
| I-402 | 3-cyclopropyl-4-[2-(4-fluorophenyl)ethyl]-1-(1-methyl-1H-1,3-benzodiazo1-2- |
| | yl)-1H-pyrazol-5-ol |
| I-403 | 4-[2-(4-fluorophenyl)ethyl]-3-(4-methanesulfonylphenyl)-1-(1-methyl-1H-1,3- |
| | benzodiazol-2-yl)-1H-pyrazol-5-ol |
| I-404 | ethyl 1-(1H-1,3-benzodiazol-2-yl)-4-[2-(4-fluorophenyl)ethyl]-5-hydroxy-1H- |
| | pyrazole-3-carboxylate |
| I-405 | 4-[2-(4-fluorophenyl)ethyl]-3-(3-methanesulfonylphenyl)-1-(1-methyl-1H-1,3- |
| | benzodiazol-2-yl)-1H-pyrazol-5-ol |
| I-406 | 1-(1H-1,3-benzodiazol-2-yl)-4-[2-(4-fluorophenyl)ethyl]-3-(piperidine-1- |
| | carbonyl)-1H-pyrazol-5-ol |
| I-502 | 1-(1H-1,3-benzodiazol-2-yl)-4-[2-(4-fluorophenyl)ethyl]-3-(piperidin-3-yl)-1H- |
| | pyrazol-5-ol |
| I-503 | 1-(5-chloro-1H-1,3-benzodiazol-2-yl)-4-[2-(4-fluorophenyl)ethyl]-3-(piperidin- |
| | 4-yl)-1H-pyrazol-5-ol |
| I-504 | 1-(1H-1,3-benzodiazol-2-yl)-4-[(piperidin-4-yl)methyl]-3-[4- |
| | (trifluoromethyl)phenyl]-1H-pyrazol-5-ol |
| I-550 | 1-(5-chloro-1H-1,3-benzodiazol-2-yl)-3-(4-methanesulfonylphenyl)-4-[2- |
| | (piperidin-4-yl)ethyl]-1H-pyrazol-5-ol |
| I-551 | 1-(1H-1,3-benzodiazol-2-yl)-4-[2-(4-fluorophenyl)ethyl]-3-(piperidin-4-yl)-1H- |
| | pyrazol-5-ol |
| I-552 | 1-(1H-1,3-benzodiazol-2-yl)-4-[2-(piperidin-4-yl)ethyl]-3-[4- |
| | (trifluoromethyl)phenyl]-1H-pyrazol-5-ol |
| I-553 | 1-(1H-1,3-benzodiazol-2-yl)-4-[2-(4-fluorophenyl)ethyl]-3-[(piperidin-4- |
| | yl)methyl]-1H-pyrazol-5-ol |
| I-554 | 1-(5-chloro-1H-1,3-benzodiazol-2-yl)-3-cyclopropyl-4-[2-(piperidin-4- |
| | yl)ethyl]-1H-pyrazol-5-ol |
| I-555 | 1-(5-bromo-1H-1,3-benzodiazol-2-y1)-4-[2-(piperidin-4-yl)ethyl]-3-[5- |
| | (trifluoromethyl)pyridin-2-yl]-1H-pyrazol-5-ol |
| I-556 | 1-(1H-1,3-benzodiazol-2-yl)-4-[2-(4-fluorophenyl)ethyl]-3-[(piperidin-3- |
| | yl)methyl]-1H-pyrazol-5-ol |
| I-701 | propan-2-yl4-[1-(5-chloro-1H-1,3-benzodiazol-2-yl)-5-hydroxy-4-[2-(oxan-4- |
| | yl)ethy1]-1H-pyrazol-3-yl]piperidine-1-carboxylate |
| I-702 | 1-{4-[1-(5-chloro-1H-1,3-benzodiazol-2-yl)-4-[2-(4-fluorophenyl)ethyl]-5- |
| | hydroxy-1H-pyrazol-3-yl]piperidin-1-yl}ethan-1-one |
| I-703 | 1-{4-[1-(5-chloro-1H-1,3-benzodiazol-2-yl)-5-hydroxy-4-[2-(oxan-4-yl)ethyl]- |
| | 1H-pyrazol-3-yl]piperidin-1-yl}ethan-1-one |
| I-704 | 1-{4-[1-(5-chloro-1H-1,3-benzodiazol-2-yl)-5-hydroxy-4-[2-(2H-1,2,3-triazol- |
| | 2-yl)ethyl]-1H-pyrazol-3-yl]piperidin-1-yl}ethan-1-one |
| I-705 | methyl 4-[1-(5-chloro-1H-1,3-benzodiazol-2-yl)-5-hydroxy-4-[2-(2H-1,2,3- |
| | triazol-2-yl)ethyl]-1H-pyrazol-3-yl]piperidine-1-carboxylate |
| I-706 | 1-(5-chloro-1H-1,3-benzodiazol-2-yl)-3-(1-methanesulfonylpiperidin-4-yl)-4- |
| | [2-(oxan-4-yl)ethyl]-1H-pyrazol-5-ol |
| I-707 | 1-(5-chloro-1H-1,3-benzodiazol-2-yl)-3-(1-methanesulfonylpiperidin-4-yl)-4- |
| | [2-(2H-1,2,3-triazol-2-yl)ethyl]-1H-pyrazol-5-ol |
| I-708 | methyl 4-{2-[1-(5-bromo-1H-1,3-benzodiazol-2-yl)-5-hydroxy-3-[5- |
| | (trifluoromethyl)pyridin-2-yl]-1H-pyrazol-4-yl]ethyl}piperidine-1-carboxylate |
| I-709 | 1-(4-{2-[1-(5-bromo-1H-1,3-benzodiazol-2-yl)-5-hydroxy-3-[5- |
| | (trifluoromethyl)pyridin-2-yl]-1H-pyrazol-4-yl]ethyl}piperidin-1-yl)ethan-1- |
| | one |
| I-710 | 1-(5-chloro-1H-1,3-benzodiazol-2-yl)-3-cyclopropyl-4-{2-[1-(4- |
| | fluorobenzoyl)piperidin-4-yl]ethyl}-1H-pyrazol-5-ol |
| I-711 | 1-(5-chloro-1H-1,3-benzodiazol-2-yl)-4-[2-(1-cyclopropanecarbonylpiperidin- |
| | 4-yl)ethyl]-3-cyclopropyl-1H-pyrazol-5-ol |
| I-712 | 1-(4-{2-[1-(5-chloro-1H-1,3-benzodiazol-2-yl)-3-cyclopropyl-5-hydroxy-1H- |
| | pyrazol-4-yl]ethyl}piperidin-1-yl)-2-hydroxy-2-methylpropan-1-one |
| I-713 | 1-{4-[1-(1H-1,3-benzodiazol-2-yl)-5-hydroxy-3-[4-(trifluoromethyl)phenyl]- |
| | 1H-pyrazol-4-yl]piperidin-1-yl}ethan-1-one |
| I-714 | 1-(4-{2-[1-(1H-1,3-benzodiazol-2-yl)-5-hydroxy-3-[5-(trifluoromethyl)pyridin- |
| | 2-yl]-1H-pyrazol-4-yl]ethyl}piperidin-1-yl)ethan-1-one |
| I-715 | methyl 4-{2-[1-(1H-1,3-benzodiazol-2-yl)-5-hydroxy-3-[5- |
| | (trifluoromethyl)pyridin-2-yl]-1H-pyrazol-4-yl]ethyl}piperidine-1-carboxylate |
| I-716 | 1-(1H-1,3-benzodiazol-2-yl)-4-[2-(1-methanesulfonylpiperidin-4-yl)ethyl]-3-[5- |
| | (trifluoromethyl)pyridin-2-yl]-1H-pyrazol-5-ol |
| I-717 | propan-2-yl4-{2-[1-(1H-1,3-benzodiazol-2-yl)-3-(5-bromopyridin-2-yl)-5- |
| | hydroxy-1H-pyrazol-4-yl]ethyl}piperidine-1-carboxylate |
| I-718 | 1-(1H-1,3-benzodiazol-2-y1)-4-[(1-methanesulfonylpiperidin-4-yl)methyl]-3-[4- |
| | (trifluoromethyl)phenyl]-1H-pyrazol-5-ol |
| I-719 | 1-(4-{2-[1-(1H-1,3-benzodiazol-2-yl)-5-hydroxy-3-[4-(trifluoromethyl)phenyl]- |
| | 1H-pyrazol-4-yl]ethyl}piperidin-1-yl)ethan-1-one |
| I-720 | 1-(4-{[1-(1H-1,3-benzodiazol-2-yl)-5-hydroxy-3-[4-(trifluoromethyl)phenyl]- |
| | 1H-pyrazol-4-yl]methyl}piperidin-1-yl)ethan-1-one |
| I-601 | 4-[2-(4-fluorophenyl)ethyl]-5′-(trifluoromethyl)-3-[4-(trifluoromethyl)phenyl]- |
| | 2′H-[1,3′-bipyrazol]-5-ol |
| I-602 | 4-[2-(4-fluorophenyl)ethyl]-5′-(trifluoromethyl)-3-[5-(trifluoromethyl)pyridin- |
| | 2-yl]-2′H-[1,3′-bipyrazol]-5-ol |
| I-603 | 4-[2-(4-fluorophenyl)ethyl]-3-(4-methanesulfonylphenyl)-5′-(trifluoromethyl)- |
| | 2′H-[1,3′-bipyrazol]-5-ol |
| I-604 | 4-[2-(4-fluorophenyl)ethyl]-3-(3-methanesulfonylphenyl)-5′-(trifluoromethyl)- |
| | 2′H-[1,3′-bipyrazol]-5-ol |
| I-605 | propan-2-yl 4-{4-[2-(4-fluorophenyl)ethyl]-5-hydroxy-5′-(trifluoromethyl)-2′H- |
| | [1,3′-bipyrazol]-3-yl}piperidine-1-carboxylate |
| I-606 | 3-cyclopropyl-4-[2-(4-fluorophenyl)ethyl]-5′-(trifluoromethyl)-2′H-[1,3′- |
| | bipyrazol]-5-ol |
| I-607 | 3-(4-fluorophenyl)-4-[2-(oxan-4-yl)methyl]-5′-(trifluoromethyl)-2′H-[1,3′- |
| | bipyrazol]-5-amine |
| I-608 | 3-(3-methanesulfonylphenyl)-4-[2-(oxan-4-yl)ethyl]-5′-(trifluoromethyl)-2′H- |
| | [1,3′-bipyrazol]-5-ol |
| I-609 | 3-(4-fluorophenyl)-4-[2-(oxan-4-yl)ethyl]-5′-(trifluoromethyl)-2′H-[1,3′- |
| | bipyrazol]-5-ol |
| I-610 | 3-(3-fluorophenyl)-4-[2-(oxan-4-yl)ethyl]-5′-(trifluoromethyl)-2′H-[1,3′- |
| | bipyrazol]-5-ol |
| I-611 | 3-(4-methanesulfonylphenyl)-4-[2-(oxan-4-yl)ethyl]-5′-(trifluoromethyl)-2′H- |
| | [1,3′-bipyrazol]-5-ol |
| I-612 | tert-butyl 4-{2-[5-hydroxy-3-(3-methanesulfonylphenyl)-5′-(trifluoromethyl)- |
| | 2′H-[1,3′-bipyrazol]-4-yl]ethyl}piperidine-1-carboxylate |
| I-613 | 2-hydroxy-1-(4-{2-[5-hydroxy-3-(3-methanesulfonylphenyl)-5′- |
| | (trifluoromethyl)-2′H-[1,3′-bipyrazol]-4-yl]ethyl}piperidin-1-yl)-2- |
| | methylpropan-1-one |
| I-614 | 3-(4-methanesulfonylphenyl)-4-{2-[1-(2,2,2-trifluoroethyl)piperidin-4- |
| | yl]ethyl}-5′-(trifluoromethyl)-2′H-[1,3′-bipyrazol]-5-ol |
| I-615 | 3-(3-fluorophenyl)-4-[2-(pyridin-4-yl)ethyl]-5′-(trifluoromethyl)-2′H-[1,3′- |
| | bipyrazol]-5-ol |
| I-616 | 3-(4-fluorophenyl)-4-[2-(pyridin-4-yl)ethyl]-5′-(trifluoromethyl)-2′H-[1,3′- |
| | bipyrazol]-5-ol |
| I-617 | ethyl 4-{2-[5-hydroxy-3-phenyl-5′-(trifluoromethyl)-2′H-[1,3′-bipyrazol]-4- |
| | yl]ethyl}benzoate |
| I-618 | 4-{2-[5-hydroxy-3-phenyl-5′-(trifluoromethyl)-2′H-[1,3′-bipyrazol]-4- |
| | yl]ethyl}benzoic acid |
| I-619 | 5′-cyclopropyl-4-[2-(4-fluorophenyl)ethyl]-3-[4-(trifluoromethyl)phenyl]-2′H- |
| | [1,3′-bipyrazol]-5-ol |
| I-620 | 5′-cyclopropyl-3-(4-methanesulfonylphenyl)-4-[2-(oxan-4-yl)ethyl]-2′H-[1,3′- |
| | bipyrazol]-5-ol |
| I-721 | 2-methylpropyl 4-[1-(1H-1,3-benzodiazol-2-yl)-4-[2-(4-fluorophenyl)ethyl]-5- |
| | hydroxy-1H-pyrazol-3-yl]piperidine-1-carboxylate |
| I-722 | propan-2-yl 4-[1-(1H-1,3-benzodiazol-2-yl)-5-hydroxy-4-[2-(oxan-4-yl)ethyl]- |
| | 1H-pyrazol-3-yl]piperidine-1-carboxylate |
| I-723 | ethyl 4-[1-(1H-1,3-benzodiazol-2-yl)-5-hydroxy-4-[2-(oxan-4-yl)ethyl]-1H- |
| | pyrazol-3-yl]piperidine-1-carboxylate |
| I-724 | 4-[1-(1H-1,3-benzodiazol-2-yl)-5-hydroxy-4-[2-(oxan-4-yl)ethyl]-1H-pyrazol- |
| | 3-yl]-N,N-dimethylpiperidine-1-sulfonamide |
| I-725 | propan-2-yl 4-[1-(1H-1,3-benzodiazol-2-yl)-5-hydroxy-4-[(oxan-4-yl)methyl]- |
| | 1H-pyrazol-3-yl]piperidine-1-carboxylate |
| I-726 | 1-{4-[1-(1H-1,3-benzodiazol-2-yl)-4-[2-(4-fluorophenyl)ethyl]-5-hydroxy-1H- |
| | pyrazol-3-yl]piperidin-1-yl}-2-(4-methylpiperazin-1-yl)ethan-1-one |
| I-727 | (2R)-1-{4-[1-(1H-1,3-benzodiazol-2-yl)-4-[2-(4-fluorophenyl)ethyl]-5- |
| | hydroxy-1H-pyrazol-3-yl]piperidin-1-yl}-2-methoxypropan-1-one |
| I-728 | 1-(1H-1,3-benzodiazol-2-yl)-4-[2-(4-fluorophenyl)ethyl]-3-[1-(pyrazine-2- |
| | carbonyl)piperidin-4-yl]-1H-pyrazol-5-ol |
| I-729 | 2-{4-[1-(1H-1,3-benzodiazol-2-yl)-4-[2-(4-fluorophenyl)ethyl]-5-hydroxy-1H- |
| | pyrazol-3-yl]piperidine-1-carbonyl}benzene-1,3,5-triol |
| I-730 | 1-(1H-1,3-benzodiazol-2-yl)-4-[2-(4-fluorophenyl)ethyl]-3-[1-(pyridine-2- |
| | carbonyl)piperidin-4-yl]-1H-pyrazol-5-ol |
| I-731 | 6-{4-[1-(1H-1,3-benzodiazol-2-yl)-4-[2-(4-fluorophenyl)ethyl]-5-hydroxy-1H- |
| | pyrazol-3-yl]piperidine-1-carbonyl}pyridin-2-ol |
| I-732 | (2S,3S)-1-{4-[1-(1H-1,3-benzodiazol-2-yl)-4-[2-(4-fluorophenyl)ethyl]-5- |
| | hydroxy-1H-pyrazol-3-yl]piperidin-1-yl}-2-hydroxy-3-methylpentan-1-one |
| I-733 | (2S)-1-{4-[1-(1H-1,3-benzodiazol-2-yl)-4-[2-(4-fluorophenyl)ethyl]-5-hydroxy- |
| | 1H-pyrazol-3-yl]piperidin-1-yl}-2-hydroxybutan-1-one |
| I-734 | 1-{4-[1-(1H-1,3-benzodiazol-2-yl)-4-[2-(4-fluorophenyl)ethyl]-5-hydroxy-1H- |
| | pyrazol-3-yl]piperidin-1-yl}-2-(pyridin-4-yl)ethan-1-one |
| I-735 | 1-(1H-1,3-benzodiazol-2-yl)-4-[2-(4-fluorophenyl)ethyl]-3-[1-(4- |
| | hydroxycyclohexanecarbonyl)piperidin-4-yl]-1H-pyrazol-5-ol |
| I-736 | 1-(1H-1,3-benzodiazol-2-yl)-4-[2-(4-fluorophenyl)ethyl]-3-[1-(oxolane-3- |
| | carbonyl)piperidin-4-yl]-1H-pyrazol-5-ol |
| I-737 | 1-(1H-1,3-benzodiazol-2-yl)-4-[2-(4-fluorophenyl)ethyl]-3-[1-(furan-3- |
| | carbonyl)piperidin-4-yl]-1H-pyrazol-5-ol |
| I-738 | 5-(2-{4-[1-(1H-1,3-benzodiazol-2-yl)-4-[2-(4-fluorophenyl)ethyl]-5-hydroxy- |
| | 1H-pyrazol-3-yl]piperidin-1-yl}-2-oxoethyl)imidazolidine-2,4-dione |
| I-739 | 4-[1-(1H-1,3-benzodiazol-2-yl)-5-hydroxy-4-[2-(oxan-4-yl)ethyl]-1H-pyrazol- |
| | 3-yl]-N,N-dimethylpiperidine-1-carboxamide |
| I-740 | methyl 4-[1-(1H-1,3-benzodiazol-2-yl)-4-[2-(4-fluorophenyl)ethyl]-5-hydroxy- |
| | 1H-pyrazol-3-yl]piperidine-1-carboxylate |
| I-741 | 1-{4-[1-(1H-1,3-benzodiazol-2-yl)-4-[2-(4-fluorophenyl)ethyl]-5-hydroxy-1H- |
| | pyrazol-3-yl]piperidin-1-yl}ethan-1-one |
| I-742 | 1-(1H-1,3-benzodiazol-2-yl)-4-[2-(4-fluorophenyl)ethyl]-3-[1-(pyridine-3- |
| | sulfonyl)piperidin-4-yl]-1H-pyrazol-5-ol |
| I-743 | 1-(1H-1,3-benzodiazol-2-yl)-4-[2-(4-fluorophenyl)ethyl]-3-(1- |
| | methanesulfonylpiperidin-4-yl)-1H-pyrazol-5-ol |
| I-744 | 1-{4-[1-(1H-1,3-benzodiazol-2-yl)-4-[(3,4-difluorophenyl)methyl]-5-hydroxy- |
| | 1H-pyrazol-3-yl]piperidin-1-yl}ethan-1-one |
| I-745 | 1-{4-[1-(1H-1,3-benzodiazol-2-yl)-4-[2-(4-fluorophenyl)ethyl]-5-hydroxy-1H- |
| | pyrazol-3-yl]piperidin-1-yl}-2-hydroxy-2-methylpropan-1-one |
| I-746 | 4-[1-(1H-1,3-benzodiazo1-2-y1)-4-[2-(4-fluorophenyl)ethyl]-5-hydroxy-1H- |
| | pyrazol-3-yl]-N,N-dimethylpiperidine-1-sulfonamide |
| I-747 | 1-(1H-1,3-benzodiazo1-2-y1)-3-[1-(cyclohexanesulfonyl)piperidin-4-yl]-4-[2-(4- |
| | fluorophenyl)ethyl]-1H-pyrazol-5-ol |
| I-748 | 1-(1H-1,3-benzodiazol-2-yl)-3-[1-(cyclopropanesulfonyl)piperidin-4-yl]-4-[2- |
| | (4-fluorophenyl)ethyl]-1H-pyrazol-5-ol |
| I-749 | 3-[1-(4-aminobenzenesulfonyl)piperidin-4-yl]-1-(1H-1,3-benzodiazol-2-yl)-4- |
| | [2-(4-fluorophenyl)ethyl]-1H-pyrazol-5-ol |
| I-750 | 1-(1H-1,3-benzodiazol-2-yl)-4-[2-(4-fluorophenyl)ethyl]-3-[1-(2- |
| | methylpropanesulfonyl)piperidin-4-yl]-1H-pyrazol-5-ol |
| I-751 | 1-(1H-1,3-benzodiazol-2-yl)-4-[2-(4-fluorophenyl)ethyl]-3-[1-(propane-2- |
| | sulfonyl)piperidin-4-yl]-1H-pyrazol-5-ol |
| I-752 | 1-(1H-1,3-benzodiazol-2-yl)-3-[1-({6-chloroimidazo[2,1-b][1,3]thiazol-5- |
| | yl}sulfonyl)piperidin-4-yl]-4-[2-(4-fluorophenyl)ethyl]-1H-pyrazol-5-ol |
| I-753 | 1-(1H-1,3-benzodiazol-2-yl)-3-[1-(cyclopentanesulfonyl)piperidin-4-yl]-4-[2- |
| | (4-fluorophenyl)ethyl]-1H-pyrazol-5-ol |
| I-754 | 1-{3-[1-(1H-1,3-benzodiazol-2-yl)-4-[2-(4-fluorophenyl)ethyl]-5-hydroxy-1H- |
| | pyrazol-3-yl]piperidin-1-yl}ethan-1-one |
| I-755 | tert-butyl 3-[1-(1H-1,3-benzodiazol-2-yl)-4-[2-(4-fluorophenyl)ethyl]-5- |
| | hydroxy-1H-pyrazol-3-yl]piperidine-1-carboxylate |
| I-756 | 1-(1H-1,3-benzodiazol-2-yl)-4-[2-(4-fluorophenyl)ethyl]-3-[1-(pyrazine-2- |
| | carbonyl)piperidin-3-yl]-1H-pyrazol-5-ol |
| I-757 | 1-{3-[1-(1H-1,3-benzodiazol-2-yl)-4-[2-(4-fluorophenyl)ethyl]-5-hydroxy-1H- |
| | pyrazol-3-yl]piperidin-1-yl}-2-(pyridin-4-yl)ethan-1-one |
| I-758 | 1-(1H-1,3-benzodiazol-2-yl)-4-[2-(4-fluorophenyl)ethyl]-3-[1-(pyridine-3- |
| | sulfonyl)piperidin-3-yl]-1H-pyrazol-5-ol |
| I-759 | 1-(1H-1,3-benzodiazol-2-yl)-4-[2-(4-fluorophenyl)ethyl]-3-[1-(pyrazine-2- |
| | carbonyl)pyrrolidin-2-yl]-1H-pyrazol-5-ol |
| I-760 | 1-{2-[1-(1H-1,3-benzodiazol-2-yl)-4-[2-(4-fluorophenyl)ethyl]-5-hydroxy-1H- |
| | pyrazol-3-yl]pyrrolidin-1-yl}-2-(pyridin-4-yl)ethan-1-one |
| I-761 | 1-{2-[1-(1H-1,3-benzodiazol-2-yl)-4-[2-(4-fluorophenyl)ethyl]-5-hydroxy-1H- |
| | pyrazol-3-yl]pyrrolidin-1-yl}-2-hydroxy-2-methylpropan-1-one |
| I-762 | 2-{2-[1-(1H-1,3-benzodiazol-2-yl)-4-[2-(4-fluorophenyl)ethyl]-5-hydroxy-1H- |
| | pyrazol-3-yl]pyrrolidine-1-carbonyl}benzene-1,3,5-triol |
| I-763 | 1-{2-[1-(1H-1,3-benzodiazol-2-yl)-4-[2-(4-fluorophenyl)ethyl]-5-hydroxy-1H- |
| | pyrazol-3-yl]pyrrolidin-1-yl}-2-(morpholin-4-yl)ethan-1-one |
| I-764 | benzyl4-{[1-(1H-1,3-benzodiazol-2-yl)-4-[2-(4-fluorophenyl)ethyl]-5-hydroxy- |
| | 1H-pyrazol-3-yl]methyl}piperidine-1-carboxylate |
| I-765 | 1-(1H-1,3-benzodiazol-2-yl)-4-[2-(4-fluorophenyl)ethyl]-3-{[1-(oxolane-3- |
| | carbonyl)piperidin-4-yl]methyl}-1H-pyrazol-5-ol |
| I-766 | 6-(4-{[1-(1H-1,3-benzodiazol-2-yl)-4-[2-(4-fluorophenyl)ethyl]-5-hydroxy-1H- |
| | pyrazol-3-yl]methyl}piperidine-1-carbonyl)pyridin-2-ol |
| I-767 | 1-(1H-1,3-benzodiazol-2-yl)-4-[2-(4-fluorophenyl)ethyl]-3-{[1-(pyrazine-2- |
| | carbonyl)piperidin-4-yl]methyl}-1H-pyrazol-5-ol |
| I-768 | 1-(4-{[1-(1H-1,3-benzodiazol-2-yl)-4-[2-(4-fluorophenyl)ethyl]-5-hydroxy-1H- |
| | pyrazol-3-yl]methyl}piperidin-1-yl)-2-(4-methylpiperazin-1-yl)ethan-1-one |
| I-769 | 1-(4-{[1-(1H-1,3-benzodiazol-2-yl)-4-[2-(4-fluorophenyl)ethyl]-5-hydroxy-1H- |
| | pyrazol-3-yl]methyl}piperidin-1-yl)-2-[(pyridin-4-yl)methoxy]ethan-1-one |
| I-770 | 1-(4-{[1-(1H-1,3-benzodiazol-2-yl)-4-[2-(4-fluorophenyl)ethyl]-5-hydroxy-1H- |
| | pyrazol-3-yl]methyl}piperidin-1-yl)-3,3-dimethylbutan-1-one |
| I-771 | 1-(1H-1,3-benzodiazol-2-yl)-4-[2-(4-fluorophenyl)ethyl]-3-[(1- |
| | methanesulfonylpiperidin-4-yl)methyl]-1H-pyrazol-5-ol |
| I-772 | 1-(4-{[1-(1H-1,3-benzodiazol-2-yl)-4-[2-(4-fluorophenyl)ethyl]-5-hydroxy-1H- |
| | pyrazol-3-yl]methyl}piperidin-1-yl)ethan-1-one |
| I-773 | benzyl 3-{[1-(1H-1,3-benzodiazol-2-yl)-4-[2-(4-fluorophenyl)ethyl]-5-hydroxy- |
| | 1H-pyrazol-3-yl]methyl}piperidine-1-carboxylate |
| I-774 | N-{[1-(1H-1,3-benzodiazol-2-yl)-4-[2-(4-fluorophenyl)ethyl]-5-hydroxy-1H- |
| | pyrazo1-3-y1]methyl}pyridine-3-sulfonamide |
| I-775 | 1-(5-bromo-1H-1,3-benzodiazol-2-yl)-4-[2-(1-methanesulfonylpiperidin-4- |
| | yl)ethyl]-3-[5-(trifluoromethyl)pyridin-2-yl]-1H-pyrazol-5-ol |
| I-776 | 1-(4-{2-[1-(1H-1,3-benzodiazol-2-yl)-5-hydroxy-3-(3-methanesulfonylphenyl)- |
| | 1H-pyrazol-4-yl]ethyl}piperidin-1-yl)-2-hydroxy-2-methylpropan-1-one |
| I-777 | 1-(4-{[1-(1H-1,3-benzodiazol-2-yl)-4-[2-(4-fluorophenyl)ethyl]-5-hydroxy-1H- |
| | pyrazol-3-yl]methyl}piperidin-1-yl)-2-hydroxy-2-methylpropan-1-one |
| I-787 | 1-(4-{2-[1-(1H-1,3-benzodiazol-2-yl)-3-(4-fluorophenyl)-5-hydroxy-1H- |
| | pyrazol-4-yl]ethyl}piperidin-1-yl)ethan-1-one |
| I-788 | (2R)-1-(4-{2-[1-(1H-1,3-benzodiazol-2-yl)-3-(4-fluorophenyl)-5-hydroxy-1H- |
| | pyrazol-4-yl]ethyl}piperidin-1-yl)-3,3,3-trifluoro-2-hydroxy-2-methylpropan-1- |
| | one |
| I-789 | (2S)-1-(4-{2-[1-(1H-1,3-benzodiazol-2-yl)-3-(4-fluorophenyl)-5-hydroxy-1H- |
| | pyrazol-4-yl]ethyl}piperidin-1-yl)-3,3,3-trifluoro-2-hydroxy-2-methylpropan-1- |
| | one |
| I-790 | 1-(4-{2-[1-(1H-1,3-benzodiazol-2-yl)-3-(4-fluorophenyl)-5-hydroxy-1H- |
| | pyrazol-4-yl]ethyl}piperidin-1-yl)-2-hydroxy-2-methylpropan-1-one |
| I-801 | 1-(1H-1,3-benzodiazol-2-yl)-4-[2-(4-fluorophenyl)ethyl]-3-(1-methylpiperidin- |
| | 4-yl)-1H-pyrazol-5-ol |
| I-802 | 1-(1H-1,3-benzodiazol-2-yl)-4-[2-(4-fluorophenyl)ethyl]-3-{1-[(pyridin-2- |
| | yl)methyl]piperidin-4-yl}-1H-pyrazol-5-ol |
| I-803 | 1-(1H-1,3-benzodiazol-2-yl)-4-[2-(4-fluorophenyl)ethyl]-3-{1-[(pyridin-3- |
| | yl)methyl]piperidin-4-yl}-1H-pyrazol-5-ol |
| I-804 | 1-(1H-1,3-benzodiazol-2-yl)-4-[2-(4-fluorophenyl)ethyl]-3-{1-[(pyridin-4- |
| | yl)methyl]piperidin-4-yl}-1H-pyrazol-5-ol |
| I-805 | 1-(1H-1,3-benzodiazol-2-yl)-4-[2-(4-fluorophenyl)ethyl]-3-(1-{[2-(1H-pyrrol- |
| | 1-yl)phenyl]methyl}piperidin-4-yl)-1H-pyrazol-5-ol |
| I-806 | 1-(1H-1,3-benzodiazol-2-yl)-4-[2-(4-fluorophenyl)ethyl]-3-{1-[(oxan-4- |
| | yl)methyl]piperidin-4-yl}-1H-pyrazol-5-ol |
| I-807 | 1-(1H-1,3-benzodiazol-2-yl)-4-[2-(4-fluorophenyl)ethyl]-3-({1-[(pyridin-2- |
| | yl)methyl]piperidin-4-yl}methyl)-1H-pyrazol-5-ol |
| I-808 | 1-(1H-1,3-benzodiazol-2-yl)-4-[2-(4-fluorophenyl)ethyl]-3-({1-[(pyridin-3- |
| | yl)methyl]piperidin-4-yl}methyl)-1H-pyrazol-5-ol |
| I-809 | 1-(1H-1,3-benzodiazol-2-yl)-4-[2-(4-fluorophenyl)ethyl]-3-({1-[(pyridin-4- |
| | yl)methyl]piperidin-4-yl}methyl)-1H-pyrazol-5-ol |
| I-810 | 1-(1H-1,3-benzodiazol-2-yl)-4-[2-(4-fluorophenyl)ethyl]-3-({[(pyridin-4- |
| | yl)methyl]amino}methyl)-1H-pyrazol-5-ol |
| I-811 | 1-(1H-1,3-benzodiazol-2-yl)-4-[2-(4-fluorophenyl)ethyl]-3-({[(pyridin-3- |
| | yl)methyl]amino}methyl)-1H-pyrazol-5-ol |
| I-901 | 1-(5-chloro-1H-1,3-benzodiazol-2-yl)-3-cyclopropyl-4-{2-[1-(5- |
| | fluoropyrimidin-2-yl)piperidin-4-yl]ethyl}-1H-pyrazol-5-ol |
| I-902 | 1-(5-chloro-1H-1,3-benzodiazol-2-yl)-3-cyclopropyl-4-{2-[1-(2,6- |
| | dimethoxypyrimidin-4-yl)piperidin-4-yl]ethyl}-1H-pyrazol-5-ol |
| I-903 | 1-(1H-1,3-benzodiazol-2-yl)-4-[2-(4-fluorophenyl)ethyl]-3-[1-(pyrimidin-2- |
| | yl)piperidin-4-yl]-1H-pyrazol-5-ol |
| I-1001 | 1-(1H-1,3-benzodiazol-2-yl)-4-[(3,4-difluorophenyl)methyl]-3-[1-(2,2,2- |
| | trifluoroethyl)piperidin-4-yl]-1H-pyrazol-5-ol |
| I-1002 | 1-(1H-1,3-benzodiazol-2-yl)-4-[2-(4-fluorophenyl)ethyl]-3-[1-(2,2,2- |
| | trifluoroethyl)piperidin-3-yl]-1H-pyrazol-5-ol |
| I-1003 | 1-(1H-1,3-benzodiazol-2-yl)-4-[2-(4-fluorophenyl)ethyl]-3-[1-(2,2,2- |
| | trifluoroethyl)piperidin-4-yl]-1H-pyrazol-5-ol |
| I-1004 | 1-(1H-1,3-benzodiazol-2-yl)-4-[2-(4-fluorophenyl)ethyl]-3-{[1-(2,2,2- |
| | trifluoroethyl)piperidin-4-yl]methyl}-1H-pyrazol-5-ol |
| I-1005 | 1-(1H-1,3-benzodiazol-2-yl)-4-{[1-(2,2,2-trifluoroethyl)piperidin-4-yl]methyl}- |
| | 3-[4-(trifluoromethyl)phenyl]-1H-pyrazol-5-ol |
| I-1006 | 1-(1H-1,3-benzodiazol-2-yl)-4-{2-[1-(2,2,2-trifluoroethyl)piperidin-4-yl]ethyl}- |
| | 3-[4-(trifluoromethyl)phenyl]-1H-pyrazol-5-ol |
| I-1101 | 4-[1-(1H-1,3-benzodiazo1-2-y1)-4-[2-(4-fluorophenyl)ethyl]-5-hydroxy-1H- |
| | pyrazol-3-yl]benzoic acid |
| I-1201 | 4-[1-(1H-1,3-benzodiazol-2-y1)-4-[2-(4-fluorophenyl)ethyl]-5-hydroxy-1H- |
| | pyrazol-3-yl]-N-(cyclopropylmethyl)benzamide |
| I-1301 | 2-{4-[2-(4-fluorophenyl)ethyl]-5-methyl-3-phenyl-1H-pyrazol-1-yl}-1H-1,3- |
| | benzodiazole |
| I-1401 | 1-(1H-1,3-benzodiazol-2-yl)-3-(4-chlorophenyl)-4-[2-(4-fluorophenyl)ethyl]- |
| | 1H-pyrazol-5-amine |
| I-1501 | 3-(4-fluorophenyl)-4-(2-phenylethyl)-5′-(trifluoromethyl)-2′H-1,3′-bipyrazol |
| I-1502 | 2-[3-(4-fluorophenyl)-4-(2-phenylethyl)-1H-pyrazol-1-yl]-1H-1,3-benzodiazole |
| I-173 | 1-(5-fluoro-1H-1,3-benzodiazol-2-yl)-3-(4-fluorophenyl)-4-[(oxan-4-yl)methyl]- |
| | 1H-pyrazol-5-ol |
| I-174 | 1-(1H-1,3-benzodiazol-2-yl)-3-cyclopropyl-4-[(oxan-4-yl)methyl]-1H-pyrazol-5-ol |
| I-175 | 1-(1H-1,3-benzodiazol-2-yl)-3-(4-fluorophenyl)-4-[(oxan-3-yl)methyl]-1H- |
| | pyrazol-5-ol |
| I-176 | 1-(1H-1,3-benzodiazol-2-yl)-3-(3,3-difluorocyclobutyl)-4-[(oxan-4-yl)methyl]-1H- |
| | pyrazol-5-ol |
| I-177 | 1-(1H-1,3-benzodiazol-2-yl)-3-(oxan-4-yl)-4-[(oxan-4-yl)methyl]-1H-pyrazol-5-ol |
| I-178 | 1-(1H-1,3-benzodiazol-2-yl)-3-[2-(oxan-4-yl)ethyl]-4-[(oxan-4-yl)methyl]-1H- |
| | pyrazol-5-ol |
| I-311 | 4-[2-(4-fluorophenyl)ethyl]-1-{3H-imidazo[4,5-b]pyridin-2-yl}-3-(3- |
| | methanesulfonylphenyl)-1H-pyrazol-5-ol |
| I-312 | 1-{3H-imidazo[4,5-b]pyridin-2-yl}-3-(oxan-4-yl)-4-[(oxan-4-yl)methyl]-1H- |
| | pyrazol-5-ol |
| I-313 | 3-(4-fluorophenyl)-1-{3H-imidazo[4,5-b]pyridin-2-yl}-4-[(oxan-4-yl)methyl]-1H- |
| | pyrazol-5-ol |
| I-314 | 4-[2-(4-fluorophenyl)ethyl]-1-{3H-imidazo[4,5-b]pyridin-2-yl}-3-(oxan-4-yl)-1H- |
| | pyrazol-5-ol |
| I-315 | 3-cyclopropyl-1-{3H-imidazo[4,5-b]pyridin-2-yl}-4-[(oxan-4-yl)methyl]-1H- |
| | pyrazol-5-ol |
| I-316 | 3-(3,3-difluorocyclobutyl)-1-{3H-imidazo[4,5-b]pyridin-2-yl}-4-[(oxan-4- |
| | yl)methyl]-1H-pyrazol-5-ol |
| I-621 | 3-(oxan-4-yl)-4-[(oxan-4-yl)methyl]-5′-(trifluoromethyl)-2′H-[1,3′-bipyrazol]-5-ol |
| I-622 | 3-cyclopropyl-4-[(oxan-4-yl)methyl]-5′-(trifluoromethyl)-2′H-[1,3′-bipyrazol]-5-ol |
| I-623 | 4-[2-(4-fluorophenyl)ethyl]-3-(oxan-4-yl)-5′-(trifluoromethyl)-2′H-[1,3′- |
| | bipyrazol]-5-ol |
| I-778 | 1-(4-{2-[1-(1H-1,3-benzodiazol-2-yl)-3-cyclopropyl-5-hydroxy-1H-pyrazol-4- |
| | yl]ethyl}piperidin-1-yl)-2-hydroxy-2-methylpropan-1-one |
| I-779 | 1-(3-{[1-(1H-1,3-benzodiazol-2-yl)-3-cyclopropyl-5-hydroxy-1H-pyrazol-4- |
| | yl]methyl}piperidin-1-yl)-2-hydroxy-2-methylpropan-1-one |
| I-780 | 1-(3-{[1-(1H-1,3-benzodiazol-2-yl)-3-cyclopropyl-5-hydroxy-1H-pyrazol-4- |
| | yl]methyl}piperidin-1-yl)ethan-1-one |
| I-781 | 1-(1H-1,3-benzodiazol-2-yl)-3-cyclopropyl-4-[(1-methanesulfonylpiperidin-3- |
| | yl)methyl]-1H-pyrazol-5-ol |
| I-782 | 3-{[1-(1H-1,3-benzodiazol-2-yl)-3-cyclopropyl-5-hydroxy-1H-pyrazol-4- |
| | yl]methyl}-N,N-dimethylpiperidine-1-carboxamide |
| I-783 | (2R)-1-(4-{2-[1-(1H-1,3-benzodiazol-2-yl)-3-cyclopropyl-5-hydroxy-1H-pyrazol- |
| | 4-yl]ethyl}piperidin-1-yl)-3,3,3-trifluoro-2-hydroxy-2-methylpropan-1-one |
| I-784 | 1-(3-{[1-(1H-1,3-benzodiazol-2-yl)-3-(4-fluorophenyl)-5-hydroxy-1H-pyrazol-4- |
| | yl]methyl}piperidin-1-yl)ethan-1-one |
| I-785 | 1-(3-{[1-(1H-1,3-benzodiazol-2-yl)-3-(4-fluorophenyl)-5-hydroxy-1H-pyrazol-4- |
| | yl]methyl}piperidin-1-yl)-2-hydroxy-2-methylpropan-1-one |
| I-786 | (2S)-1-(4-{2-[1-(1H-1,3-benzodiazol-2-yl)-3-cyclopropyl-5-hydroxy-1H-pyrazol- |
| | 4-yl]ethyl}piperidin-1-yl)-3,3,3-trifluoro-2-hydroxy-2-methylpropan-1-one |

The following abbreviations have the indicated meanings:

| Abbreviation | Meaning |
|---|---|
| aq | aqueous |
| Boc | tert-butoxy carbonyl or t-butoxy carbonyl |
| (Boc)₂O | di-tert-butyl dicarbonate |
| cat. qty. | catalytic quantity |
| Cbz | Benzyloxycarbonyl |
| CbzCl | Benzyl chloroformate |
| c-Bu | cyclobutyl |
| c-Pr | cyclopropyl |
| DMAP | 4-(dimethylamino)pyridine |
| DMF | N,N-dimethylformamide |
| EDC•HCl, | 1-[3-(dimethylamino)propyl]-3-ethylcarbodiimide |
| EDCI | hydrochloride |
| Equiv | equivalents |
| EtOAc | ethyl acetate |
| h, hr | hour(s) |
| HOAc | Acetic acid |
| HOBt | 1-hydroxybenzotriazole |
| HATU | 2-(7-Aza-1H-benzotriazole-1-yl)-1,1,3,3- |
| | tetramethyluronium hexafluorophosphate |
| LC-MS | liquid chromatography-mass spectroscopy |
| m-CPBA | meta-chloroperoxybenzoic acid |
| min | minute |
| MS | mass spectrum |
| Prep HPLC | Preparative HPLC on a Gilson 215 system using a C18 |
| | reverse phase column, eluted with an acetonitrile/water |
| | gradient buffered with 0.1% TFA. |
| quant | quantitative yield |
| rt | room temperature |
| Satd, sat'd | saturated |
| Tf | trifluoromethanesulfonate |
| TFA | trifluoroacetic acid |
| t_{R} | retention time |
| TsOH | p-toluenesulfonic acid |

### General Description of Synthesis

A compound of Formula I, wherein X⁴ is OH, is prepared by reaction of a β-ketoester of Formula II, wherein R is alkyl, with a hydrazine of Formula III using an acid catalyst such as HOAc, HCl or TsOH in a suitable solvent such as ethanol, at a temperature between room temperature and 150° C. Heat is applied by means including an oil bath, heated metal block or microwave oven.

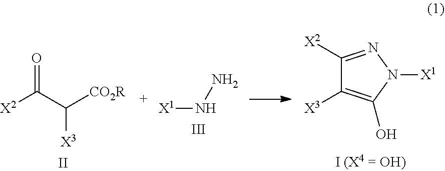

A compound of Formula I, wherein X⁴ is NH₂, is prepared by reaction of a ketonitrile of Formula IV with a hydrazine of Formula III, using an acid catalyst such as HOAc or TsOH in a suitable solvent such as ethanol, at a temperature between room temperature and 150° C.

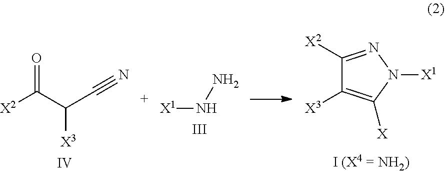

A compound of Formula I, wherein X⁴ is H or alkyl, is prepared by reaction of a 1,3-dicarbonyl compound of Formula V with a hydrazine of Formula III.

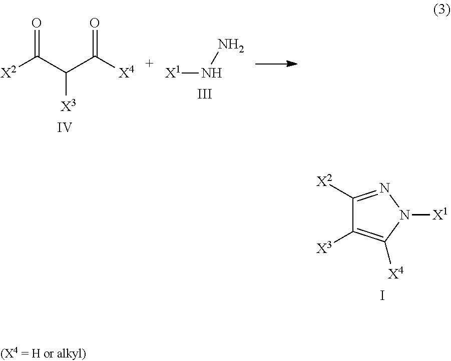

A β-ketoester of Formula II, wherein R is alkyl, is prepared by alkylation of a β-ketoester of Formula VI with a halide of Formula VII, wherein Hal is bromo, iodo, chloro or another leaving group such as methanesulfonate, tosylate of triflate, using a base such as K₂CO₃, NaH or NaOEt, in a solvent such as EtOH or DMF. In some cases, an iodide source such as KI is added to the reaction mixture.

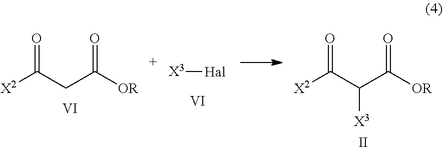

Alternatively, a β-ketoester of Formula II, wherein R is alkyl, is prepared by Claisen condensation of an ester of Formula VIII with another ester of Formula IX, using a strong base such as NaH in an inert solvent such as THF or toluene. Preferably R in VIII and IX are identical. Preferably in this process, VIII is a non enolizable ester, such as wherein X² is aryl or heteroaryl, both optionally substituted. Alternatively, esters VIII and IX are identical.

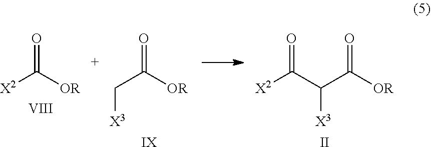

A hydrazine of Formula III, wherein X¹ is 4- or 5-azabenzimidazolyl, is prepared by direct reaction of a cyclic thiourea Formula X with hydrazine hydrate at temperatures from 50° C. to 150° C. Alternatively, X is alkylated on sulfur using a base, such as K₂CO₃, and an alkylating agent, such as iodomethane, to give an S-alkylisothiourea of Formula XI, wherein R is an alkyl group and XI is reacted with hydrazine hydrate, at temperatures from 50° C. to 150° C., to give hydrazine III.

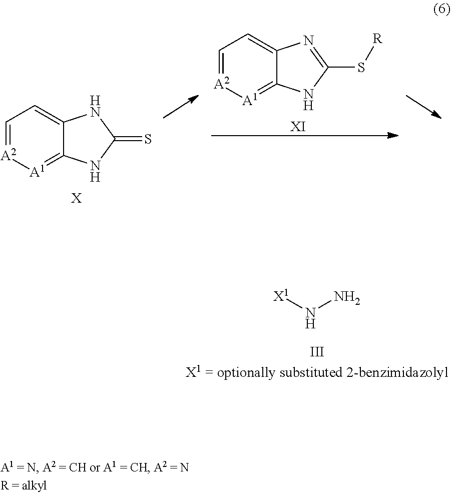

A hydrazine of Formula III, wherein X¹ is an optionally substituted benzimidazole-2-yl, is prepared by reaction of hydrazine hydrate with a 2-chlorobenzimidazole of Formula XIII, which is obtained by treatment of a cyclic urea of Formula XII with a chlorinating agent such as POCl₃ at elevated temperature.

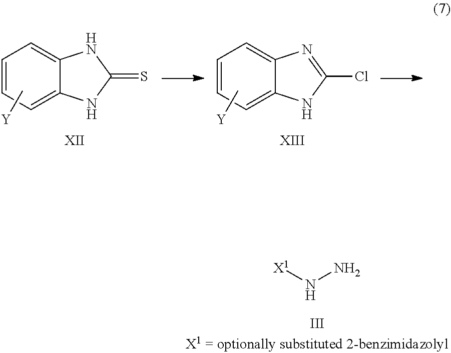

A compound of Formula I is also prepared from another compound of Formula I by transformation of groups on the molecule including, but not limited to, those listed below:
(a) An amine is converted to an amide by treatment with an acid chloride or reaction with a carboxylic acid mediated by a peptide coupling reagent such as EDCI or HATU.
(b) An amine is converted to a sulfonamide by reaction with a sulfonyl chloride.
(c) An amine is converted to a carbamate by reaction with a chloroformate.
(d) An amine is converted to a urea by reaction with an isocyanate or with a carbamyl chloride.
(e) An amine is alkylated by reaction with an alkylating agent, such as an alkyl halide or alkyl triflate.
(f) An amine is alkylated by reductive amination with an aldehyde or ketone using NaCNBH₃ or NaBH(OAc)₃.
(g) A carboxylic acid is reacted with an amine, using a peptide coupling reagent such as EDCI or HATU to give an amide.
(h) A t-butoxycarbonylamine is converted to an amine by treatment with an acid such as HCl or TFA.
(i) A benzyloxycarbonylamine is converted to an amine by treatment with an acid such as HBr or by catalytic hydrogenation.

### LC-MS Method

Column: Phenomenex Luna 3u C18(2) 100 A, 75×4.6 mm; Mobil phase: A: 0.1% TFA/water, B: 0.1% TFA/CH₃CN; Flow rate: 1.5 mL/min; Gradient 5% B to 95% B.

### Preparation of Synthetic Precursors

### Preparation 1

### Methyl 4-(4-fluorophenyl)-2-(4-(trifluoromethyl)benzoyl)butanoate (P1-001)

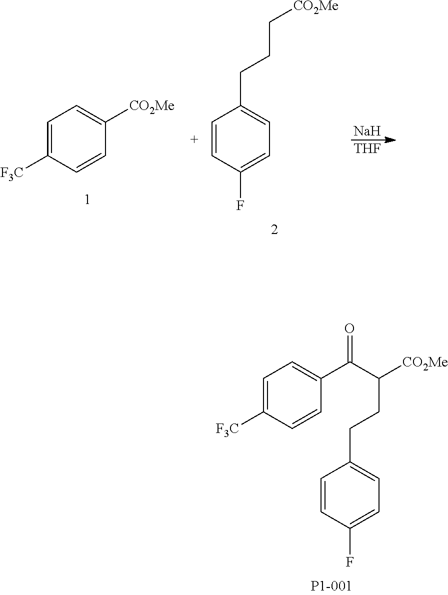

A mixture of ester 1 (2.41 g, 11.8 mmol), ester 2 (1.93 g, 9.8 mmol) and 60% NaH in oil (0.75 g, 18.7 mmol) was diluted with dry THF (10 mL) and stirred at reflux under N2 for 3 h. The mixture was cooled, diluted with EtOAc (90 mL), washed with 5% aq HCl (2×10 mL), sat′d aq NaHCO₃(10 mL) and brine (10 mL), and dried over Na₂SO₄. Removal of the solvent left an oil (5.34 g) which was chromatographed on a 40 g silica cartridge, eluted with a 0-25% EtOAc in hexanes gradient to give β-ketoester P1-001 (1.37 g, 38% yield). ¹H NMR CDCl₃ δ 7.97-8.00 (d, J=8.2 Hz, 2H), 7.70-7.73 (d, J=8.2 Hz, 2H), 7.08-7.13 (m, 2H), 6.93-6.99 (m, 2H), 4.28 (t, 1H), 3.69 (s, 3H), 2.63-2.69 (m, 2H), 2.30-2.37 (m, 2H). Additional less pure 3 (1.88 g) was recovered from mixed fractions.

The following β-ketoesters are prepared using analogous procedures:

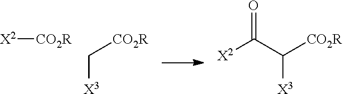

| Intermed. No. | X² | X³ | R |
|---|---|---|---|
| P1-002^{a} | 4-CF₃—C₆H₄— | (pyridin-4-yl)-CH₂CH₂— | Me |
| P1-003 | 4-CF₃—C₆H₄— | cyclopentylCH₂CH₂— | Me |
| P1-004 | 4-CF₃—C₆H₄— | (3-methyl-1-pyrazolyl)CH₂CH₂— | Me |
| P1-005^{a} | 4-CF₃—C₆H₄— | (Morpholin-4-yl)CH₂CH₂— | Me |
| P1-006 | 4-CF₃—C₆H₄— | (1-Boc-piperidin-4-yl)CH₂CH₂— | Me |
| P1-007^{a} | 4-CF₃—C₆H₄— | (2-methylimidazol-1-yl)CH₂CH₂— | Me |
| P1-008 | 4-CF₃—C₆H₄— | (2-oxopyrrolidin-1-yl)CH₂CH₂— | Me |
| P1-009 | 4-CF₃—C₆H₄— | 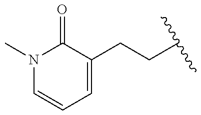 [SMILES: *CCc1cccn(C)c1=O] | Me |
| P1-010 | 4-CF3—C₆H₄— | 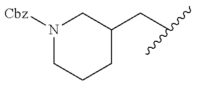 [SMILES: *CC1CCCN(C(=O)OCc2ccccc2)C1] | Me |
| P1-011 | 4-CF₃—C₆H₄— | 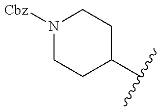 [SMILES: *C1CCN(C(=O)OCc2ccccc2)CC1] | Me |
| P1-012 | 4-t-BuO₂C—C₆H₄— | 4-F—C₆H₄CH₂CH₂— | Me |
| P1-013 | 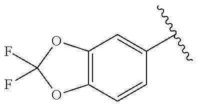 [SMILES: *c1ccc2c(c1)OC(F)(F)O2] | 4-F—C₆H₄CH₂CH₂— | Me |
| P1-014 | 5-CF₃-pyridin-2-yl | 4-F—C₆H₄CH₂CH₂— | Me |
| P1-015 | 6-MeO-pyridin-2-yl | 4-F—C₆H₄CH₂CH₂— | Me |
| P1-016^{a} | Pyridine-3-yl | 4-F—C₆H₄CH₂CH₂— | Me |
| P1-017 | 5-Br-pyridin-3-yl | 4-F—C₆H4CH₂CH₂— | Me |
| P1-018 | 6-CF3-pyridin-3-yl | 4-F—C₆H₄CH₂CH₂— | Me |
| P1-019^{a} | 6-Me-pyridin-3-yl | 4-F—C₆H₄CH₂CH₂— | Me |
| P1-020 | 2-CF3-pyrimidin-5-yl | 4-F—C₆H₄CH₂CH₂— | Me |
| P1-021 | 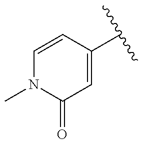 [SMILES: *c1ccn(C)c(=O)c1] | 4-F—C₆H₄CH₂CH₂— | Me |
| P1-022 | 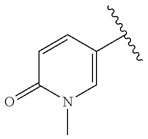 [SMILES: *c1ccc(=O)n(C)c1] | 4-F—C₆H₄CH₂CH₂— | Me |
| P1-023 | 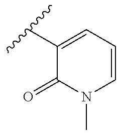 [SMILES: *c1cccn(C)c1=O] | 4-F—C₆H₄CH₂CH₂— | Me |
| P1-024 | 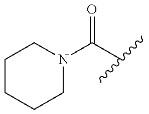 [SMILES: *C(=O)N1CCCCC1] | 4-F—C₆H₄CH₂CH₂— | Me |
| P1-025^{a} | 3-F—C₆H₄— | (pyridin-4-yl)CH₂CH₂— | |
| P1-026 | 5-CF₃-pyridin-2-yl | 4-Br—C6H4CH2CH2— | Me |
| P1-027 | 5-CF₃-pyridin-2-yl | (3-methyl-1-pyrazolyl)CH₂CH₂— | Me |
| P1-028^{a} | 5-CF₃-pyridin-2-yl | (pyridin-2-yl)CH₂CH₂— | Me |
| P1-029 | 5-CF3-pyridin-2-yl | (1-Boc-piperidin-4-yl)CH₂CH₂— | Me |
| P1-030 | 5-Br-pyridin-3-yl | (1-Boc-piperidin-4-yl)CH₂CH₂— | Me |
| P1-031 | 4-tetrahydropyranyl- | 4-tetrahydropyranyl-CH₂— | Me |
| | CH₂CH₂— | | |
| P1-032 | 3-CF₃—C₆H₄— | 4-tetrahydropyranyl-CH₂— | Me |

^{a}Water was used in place of 5% aq HCl during the workup.

### Preparation 2

### Ethyl 4-(4-fluorophenyl)-2-(3-(methylsulfonyl)benzoyl)butanoate (P2-001)

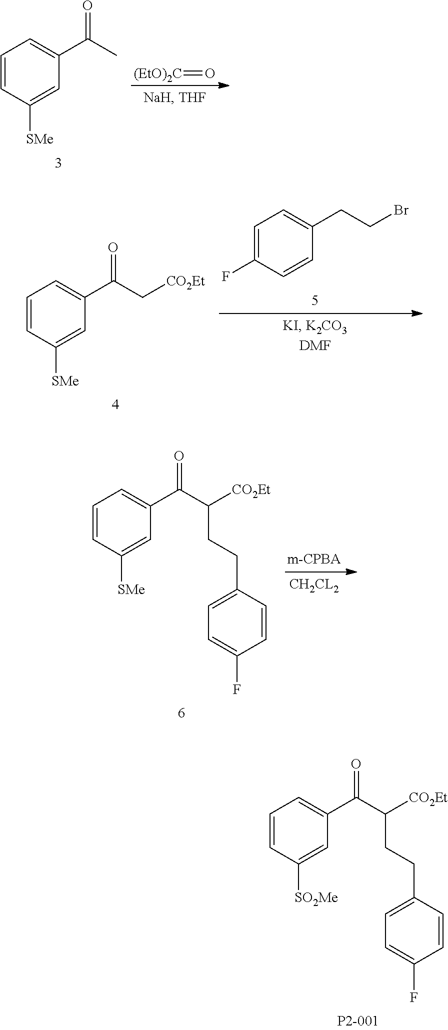

### Step 1

A stirred mixture of ketone 3 (1.97 g, 11.9 mmol), diethyl carbonate (2.9 mL, 23.8 mmol) and 60% NaH in oil (950 mg, 23.8 mmol) was diluted with dry THF (20 mL) and heated at reflux under N2 for 5 h. The mixture was concentrated, diluted with EtOAc (90 mL), washed with 5% aq HCl (10 mL) and 3:1 brine/sat′d aq NaHCO₃ (10 mL), and dried over Na₂SO₄. Removal of the solvent left a yellow oil (3.02 g) which was chromatographed on a 40 g silica cartridge, eluted with a 0-60% EtOAc in hexanes gradient, to give 4 (2.38 g, 84% yield) as a pale yellow oil. LC-MS t_{R} 4.75 min, m/z 239.

### Step 2

A mixture of 4 (560 mg, 2.4 mmol), bromide 5 (500 mg, 2.5 mmol), K₂CO₃ (360 mg, 2.6 mmol), KI (430 mg, 2.6 mmol) and dry DMF (4 mL) was heated at 70° C. for 4 h. The mixture was diluted with EtOAc (90 mL), washed with 5% aq HCl (15 mL), sat′d aq NaHCO₃ (15 mL) and brine (15 mL), and dried over Na₂SO₄. Removal of the solvent left crude 6 as an oil which was used directly in Step 3.

### Step 3

To a stirred, ice-cold solution of crude 6 (≤2.4 mmol) in CH₂Cl₂ (20 mL) was added ≤77% m-CPBA (1.16 g, ≤4.7 mmol). The mixture was stirred in the ice bath for 3 h, diluted with CH₂Cl₂ (70 mL), washed with satd aq NaHCO₃ (15 mL) and brine (15 mL), and dried over Na₂SO₄—Removal of the solvent left an oil (1.27 g) which was chromatographed on a 40 g silica cartridge, eluted with a 0-100% EtOAc in hexanes gradient, to afford P2-001 (720 mg, 78% yield over two steps). LC-MS t_{R} 5.11 min, m/z 393.

The following β-ketoesters are prepared using analogous procedures.

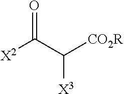

| Intermed. | | | |
|---|---|---|---|
| No. | X² | X³ | R |
| P2-002 | 3-MeSO₂—C₆H₄— | (1-Boc-piperidin-4-yl)CH₂CH₂— | Et |
| P2-003 | 3-MeSO₂—C₆H₄— | (tetrahydropyran-4-yl)CH₂CH₂— | Et |
| P2-004 | 4-MeSO₂—C₆H₄— | 4-F—C₆H₄CH₂CH₂— | Et |
| P2-005 | 4-MeSO₂—C₆H₄— | (1-Boc-piperidin-4-yl)CH₂CH₂— | Et |
| P2-006 | 4-MeSO₂—C₆H₄— | (tetrahydropyran-4-yl)CH₂CH₂— | Et |
| P2-007^{a} | 4-MeSO₂—C₆H₄— | (1-CF₃CH₂-piperidin-4- | Et |
| | | yl)CH₂CH₂— | |

^{a}3.5 equivs of m-CPBA were employed in Step 3 and, a 4^{th} step, bis(pinacolato)diboron was employed to reduce the piperidine N-oxide to the desired piperidine.

### Preparation 3

### Benzyl 4-(3-ethoxy-2-[2-(oxan-4-yl)ethyl]-3-oxopropanoyl)piperidine-1-carboxylate (P3-001)

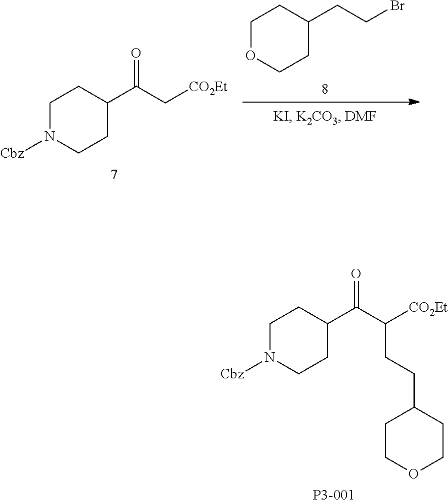 [SMILES: CCOC(=O)CC(=O)C1CCN(C(=O)OCc2ccccc2)CC1 ; BrCCC1CCOCC1 ; CCOC(=O)C(CCC1CCOCC1)C(=O)C1CCN(C(=O)OCc2ccccc2)CC1]

A stirred mixture β-ketoester 7 (550 mg, 1.65 mmol), bromide 8 (335 mg, 1.73 mmol), KI (302 mg, 1.8 mmol), K₂CO₃ (251 mg, 1.8 mmol) and dry DMF (5 mL) was heated at 70° C. for 3 h. The mixture was diluted with EtOAc (90 mL), washed with 1% aq HCl (15 mL) and brine (15 mL), and dried over Na₂SO₄. Removal of the solvent left a yellow oil (1.34 g) which was chromatographed on a 12 g silica cartridge, eluted with a 0-70% EtOAc in hexanes gradient, to give P3-001 (495 mg, 67% yield). LC-MS t_{R} 5.41 min, m/z 446.

The following β-ketoesters are prepared using analogous procedures.

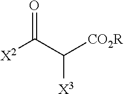

| Inter- | | | |
|---|---|---|---|
| med. | | | |
| No. | X² | X³ | R |
| P3-002 | 1-Boc-piperidin-4-yl | 4-F—C₆H₄—CH₂CH₂— | Et |
| P3-003 | 4-CF₃—C₆H₄— | Tetrahydropyran-2-yl-CH₂— | Et |
| P3-004 | 4-CF₃—C₆H₄— | 1-Cbz-piperidin-4-yl-CH₂— | Et |
| P3-005 | Me | 4-Cl-2-F—C₆H₃—CH₂— | Et |
| P3-006 | 4-CF₃—C₆H₄— | Tetrahydropyran- | Et |
| | | 4-yl-CH₂CH₂— | |
| P3-007 | 4-CF₃—C₆H₄— | l,2,3-triazol-2-yl-CH₂CH₂— | Et |
| P3-008 | 4-CF₃—C₆H₄— | 4-chloropyrazol- | Et |
| | | 1-yl-CH₂CH₂— | |
| P3-009 | l -Cbz-piperidin-4-yl | Tetrahydropyran-4- | Et |
| | | yl-CH₂CH₂— | |
| P3-010 | 1 -Cbz-piperidin-4-yl | l,2,3-triazol-2-yl-CH₂CH₂— | Et |
| P3-011 | Ph | 4- | Et |
| | | MeO2C—C6H4CH2CH2— | |
| P3-012 | Ph | 4-F—C₆H₄—CH₂CH₂— | Et |
| P3-013 | c-Pr | 4-F—C₆H₄—CH₂CH₂— | Et |
| P3-014 | 4-F—C₆H₄— | 4-F—C₆H₄—CH₂CH₂— | Me |
| P3-015 | c-Pr | 1-Boc-piperidin- | Et |
| | | 4-yl-CH₂CH₂— | |
| P3-016^{a} | 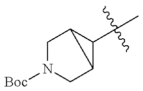 [SMILES: *C1C2CN(C(=O)OC(C)(C)C)CC12] | 4-F—C₆H₄—CH₂CH₂— | Et |
| P3-017 | 3-F—C₆H₄— | tetrahydropyran- | Me |
| | | 4-yl-CH₂CH₂— | |
| P3-018 | 4-F—C₆H₄— | tetrahydropyran- | Me |
| | | 4-yl-CH₂CH₂— | |
| P3-019 | 4-F—C₆H₄— | tetrahydropyran-4-yl-CH₂— | Me |
| P3-020 | 1-Cbz-piperidin-4-yl | tetrahydropyran-4-yl-CH₂— | Et |
| P3-021 | pyrazin-2-yl | 4-F—C₆H₄—CH₂CH₂— | Et |
| P3-022 | 4-Me—CH— | 4-F—C₆H₄—CH₂CH₂— | Et |
| P3-023 | 4-Cl—C₆H₄— | 4-F—C₆H₄—CH₂CH₂— | Et |
| P3-024 | pyridin-2-yl | 4-F—C₆H₄—CH₂CH₂— | Et |
| P3-025 | pyrazin-2-yl | 3,4-diF-C₆H₃—CH₂CH₂— | Et |
| P3-026 | pyrazin-2-yl | 3-Cl-4—F—C₆H₃-CH₂CH₂— | Et |
| P3-027 | pyridin-4-yl | 4-F—C₆H₄—CH₂CH₂— | Et |
| P3-029^{b} | 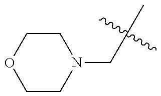 [SMILES: *CN1CCOCC1] | 4-F—C₆H₄—CH₂CH₂— | Et |
| P3-030^{b} | BocNHCH₂— | 4-F—C₆H₄—CH₂CH₂— | Et |
| P3-073 | tetrahydro-4-pyranyl | tetrahydropyran-4-yl-CH₂— | Et |
| P3-074 | 4-F—C₆H₄— | tetrahydropyran-4-yl-CH₂— | Me |
| P3-075 | c-Pr | tetrahydropyran-4-yl-CH₂— | Me |
| P3-076 | c-Pr | 1-Boc-piperidin- | Et |
| | | 4-yl-CH₂CH₂— | |
| P3-077 | c-Pr | l-Cbz-piperidin-3-yl-CH₂— | Et |
| P3-078 | 4-F—C₆H₄— | tetrahydropyran-3-yl-CH₂— | Me |
| P3-079 | 3,3-diF-c-Bu | tetrahydropyran-4-yl-CH₂— | Me |
| P3-080 | 4-F—C₆H₄— | l-Cbz-piperidin-3-yl-CH₂— | Me |
| P3-081 | t-Bu | tetrahydropyran-4-yl-CH₂— | Me |
| P3-082 | Ph | tetrahydropyran-4-yl-CH₂— | Et |
| P3-083 | 3-F—Ph | tetrahydropyran-4-yl-CH₂— | Et |
| P3-084 | 3,4-diF-Ph | tetrahydropyran-4-yl-CH₂— | Et |
| P3-085 | 2-F-Ph | tetrahydropyran-4-yl-CH₂— | Et |
| P3-086 | 2,4-diF-Ph | tetrahydropyran-4-yl-CH₂— | Et |
| P3-087 | 2,5-diF-Ph | tetrahydropyran-4-yl-CH₂— | Et |
| P3-088 | 3,5-diF-Ph | tetrahydropyran-4-yl-CH₂— | Et |
| P3-089 | 4-F—Ph | 1-Boc-piperidin- | Me |
| | | 4-yl-CH₂CH₂— | |

^{a}E/Z isomer mixture;
^{b}KI was omitted and DMF was replaced with acetone.

The following β-ketoesters are prepared by a similar procedure using NaOEt in place of K₂CO₃, omitting KI and using EtOH in place of DMF.

| | | | |
|---|---|---|---|
| P3-041 | 2-Cl—C₆H₄— | 4-F—C₆H₄—CH₂CH₂— | Et |
| P3-042 | 3-Cl—C₆H₄— | 4-F—C₆H₄—CH₂CH₂— | Et |
| P3-043 | 4-CF₃—C₆H₄ | allyl | Et |
| P3-044 | N-Cbz-piperidin-4-yl | 3,4-diF-benzyl | Et |
| P3-045 | N-Cbz-piperidin-3-yl | 4-F—C₆H₄—CH₂CH₂— | Et |

The following β-ketoesters are prepared by a similar procedure using NaH in place of K₂CO₃, and omitting KI.

| | | | |
|---|---|---|---|
| P3-028 | tetrahydropyran-4-yl | 4- | Et |
| | | F—C₆H₄—CH₂CH₂— | |
| P3-061 | MeOCH₂— | 4- | Me |
| | | F—C₆H₄—CH₂CH₂— | |
| P3-062 | MeOCH₂CH₂— | 4- | Me |
| | | F—C₆H₄—CH₂CH₂— | |
| P3-063 | t-Bu | 4- | Me |
| | | F—C₆H₄—CH₂CH₂— | |
| P3-064 | c-Bu | 4- | Et |
| | | F—C₆H₄—CH₂CH₂— | |
| P3-065 | Tetrahydrofuran-2-yl | 4- | Et |
| | | F—C₆H₄—CH₂CH₂— | |
| P3-066 | 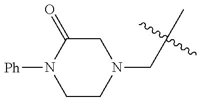 [SMILES: *CN1CCN(c2ccccc2)C(=O)C1] | 4- F—C₆H₄—CH₂CH₂— | Et |
| P3-067 | N-Boc-pyrrolidin-2-yl | 4- | Et |
| | | F—C₆H₄—CH₂CH₂— | |
| P3-068 | N-Boc-pyrrolidin-3-yl-CH₂— | 4- | Et |
| | | F—C₆H₄—CH₂CH₂— | |
| P3-069 | N-Boc-piperidin-2-yl-CH₂— | 4- | Et |
| | | F—C₆H₄—CH₂CH₂— | |
| P3-070 | 1-Boc-piperazin-1-yl-CH₂— | 4- | Et |
| | | F—C₆H₄—CH₂CH₂— | |
| P3-071 | 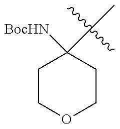 [SMILES: *C1(NC(=O)OC(C)(C)C)CCOCC1] | 4- F—C₆H₄—CH₂CH₂— | Et |
| P3-072 | N-Boc-piperidin-3-yl-CH₂— | 4- | Et |
| | | F—C₆H₄—CH₂CH₂— | |

### Preparation 4

### Methyl 2-(4-fluorobenzoyl)-4-(4-pyridyl)butanoate (P4-001)

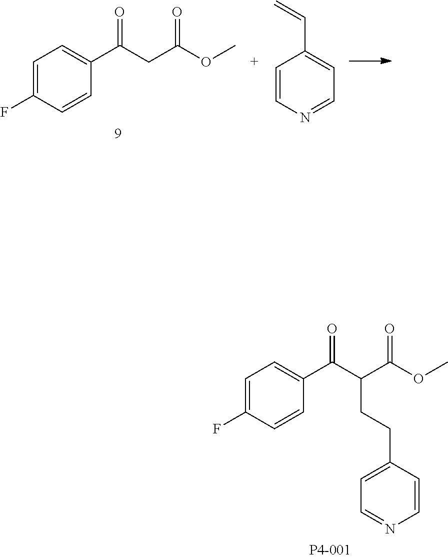

A mixture of β-ketoester 9 (490 mg, 2.5 mmol), 4-vinylpyridine (183 mg, 1.7 mmol), K₂CO₃ (41 mg, 0.3 mmol), and dry DMF (4 mL) was stirred at 70° C. for 2 d. The mixture was diluted with EtOAc (75 mL) and extracted with 5% aq HCl (2×20 mL). The combined aq HCl layer was neutralized by addition of NaHCO₃ powder and back extracted with EtOAc (2×50 mL). These EtOAc extracts were combined, washed with brine (10 mL), dried over Na₂SO₄ and concentrated to give P4-001 (290 mg) as a red oil, which was used without further purification. LC-MS t_{R} 3.28 min, m/z 302.

### Preparation 5

### 2-(4-fluorobenzoyl)-4-(4-fluorophenyl)butyronitrile (P5-001)

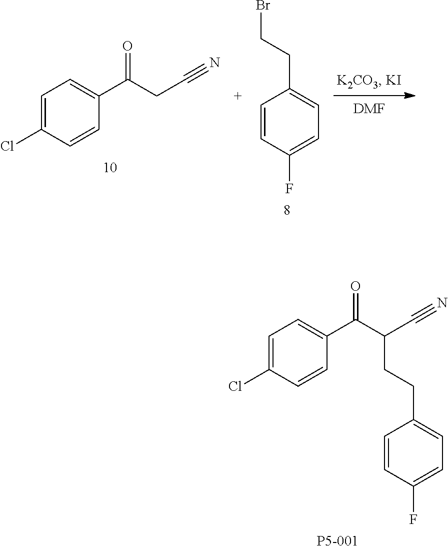

A procedure analogous to that described in Preparation 3 was adopted, starting with ketonitrile 10 (696 mg, 3.9 mmol), bromide 8 (830 mg, 4.1 mmol), K₂CO₃ (590 mg, 4.3 mmol), KI (710 mg, 4.3 mmol) and dry DMF (8 mL). Workup gave a red oil (1.58 g). Chromatography on a 40 g silica cartridge, eluted with a 0-40% EtOAc in hexanes gradient, gave a mixed fraction (652 mg) containing some of the desired P5-001 which was used without further purification.

The following compound is prepared using an analogous procedure:

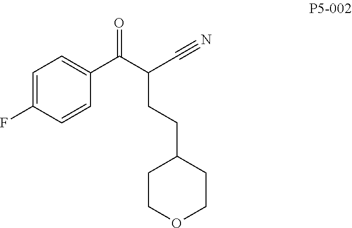 [SMILES: N#CC(CCC1CCOCC1)C(=O)c1ccc(F)cc1]

### Preparation 6

### 4-aza-2-hydrazinobenzimidazole (P6-001)

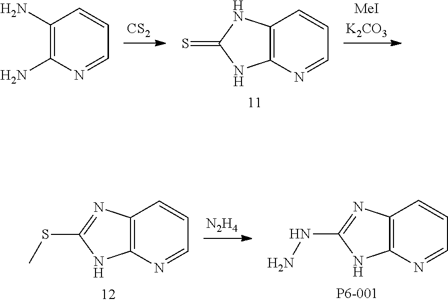

### Step 1

A mixture of 2,3-diaminopyridine (1.19 g, 10.9 mmol), CS₂ (5.3 mL, 87.2 mmol) and EtOH (15 mL) was heated at 40-45° C. for 2 d. The mixture was concentrated to leave crude 11 as a brown solid. LC-MS t_{R} 2.03 min, m/z 152.

### Step 2

The thiourea 11 prepared in Step 1 (≤10.9 mmol) was suspended in acetone (30 mL) and K₂CO₃ (4.52 g, 32.7 mmol) was added followed by MeI (0.75 ml, 12.0 mmol). The mixture was stirred at rt for 3 d and concentrated. The residue was diluted with water (40 mL) and extracted with EtOAc (3×-50 mL). The combined EtOAc layer was washed with brine (10 mL), dried over Na₂SO₄ and rotovaped to leave a brown solid (1.15 g). Chromatography on a 12 g silica cartridge, eluted with a 0-100% EtOAc in hexanes gradient, gave 5-methylisothiourea 12 (0.66 g, 38% over two steps). LC-MS t_{R} 1.91 min, m/z 166.

### Step 3

A mixture of 12 (0.66 g, 4.0 mmol) and hydrazine hydrate (2 mL) was heated at 100° C. for 5 d. The mixture was cooled, diluted with water (4 mL) and filtered. The solid collected was air dried to give P6-001 (0.31 g, 52%) as a tan solid. LC-MS t_{R} 1.33 min, m/z 150.

5-aza-2-hydrazinobenzimidazole (P6-002) is prepared following analogous procedures, except that Step 2 was omitted.
4-aza-2-hydrazinobenzimidazole (P6-001) was also prepared following the procedure in DE 3,340,932.

### Preparation 7

### 4-chloro-2-hydrazinobenzimidazole (P7-001)

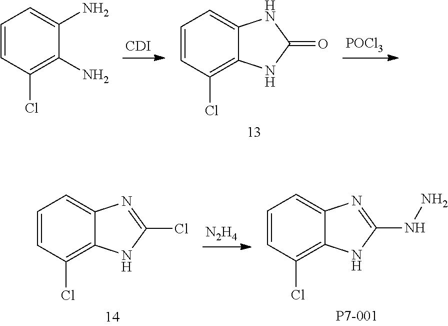

### Step 1

To a stirred solution of 3-chloro-1,2-diaminobenzene (1.60 g, 11.2 mmol) in CH₂Cl₂ (40 mL) was added carbonyl diimidazole (2.80 g, 17.0 mmol) in portions. The mixture was stirred at rt overnight and concentrated. The residue was suspended in EtOAc (80 mL) and 5% aq HCl (30 mL). The mixture was filtered and the solid collected was dried in vacuo to give 13 (1.70 g, 90% yield). LC-MS tR 3.11 min, m/z 210 (M+MeCN+H⁺).

### Step 2

A mixture of 13 (1.66 g, 9.8 mmol) and POCl₃ (20 mL) was heated at 90 C for 3 d and concentrated. The residue was taken up in EtOAc (90 mL), washed with sat′d aq NaHCO₃ (20 mL) and brine (20 mL). The combined aqueous washes were back extracted with EtOAc (40 mL). The combined EtOAc layer was dried over Na₂SO₄ and concentrated to leave crude 14 (1.79 g) as a brown solid. LC-MS t_{R} 3.68 min, m/z 187.

### Step 3

Crude 14 (1.79 g) was heated with hydrazine hydrate (15 mL) at 100° C. for 2 d. The mixture was diluted with water (5 mL) and concentrated. The brown residue was taken up in water (10 mL) and concentrated to leave P7-001 as a brown solid. LC-MS t_{R} 2.59 min, m/z 183.

### Preparation 8

### 3-cyclopropyl-5-hydrazinyl-1H-pyrazole (P8-001)

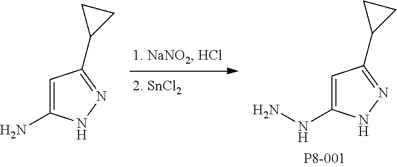 [SMILES: Nc1cc(C2CC2)n[nH]1 ; NNc1cc(C2CC2)n[nH]1]

To a stirred, ice-cold solution of 3-cyclopropyl-1H-pyrazol-5-amine (540 mg, 4.4 mmol) in conc HCl (6 mL) was added dropwise a solution of NaNO₂ (310 mg, 4.4 mmol) in water (4.5 mL). The mixture was stirred in the ice bath for 0.5 h and a solution of SnCl₂ (1.66 g, 8.8 mmol) in conc HCl (10 mL) was added. The mixture was stirred at rt for 2 h and concentrated. The residue was taken up in EtOH and concentrated to leave crude hydrazine (P9-001) as a white solid. LC-MS t_{R} 0.90 min, m/z 139.

### Preparation 9

### 1-(4-fluorophenyl)-4-phenylbutan-1-one (P9-001)

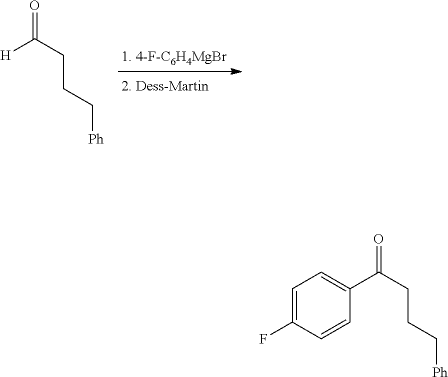 [SMILES: O=CCCCc1ccccc1 ; O=C(CCCc1ccccc1)c1ccc(F)cc1]

### Step 1

To a stirred solution of 4-phenylbutyraldehyde (2.00 g, 13.5 mmol) in dry CH₂Cl₂ (25 mL) at −70° C. was added 2 M 4-fluoromagnesium bromide in Et₂O (10 mL, 20 mmol). The cooling bath was allowed to expire and the mixture warmed to rt over 2 h. The mixture was diluted with CH₂Cl₂ (60 mL), washed with 5% aq HCl (2×10 mL), satd aq NaHCO₃ (10 mL) and brine (10 mL), and dried over Na₂SO₄. Removal of the solvent left crude 1-(4-fluorophenyl)-4-phenylbutan-1-ol (3.83 g) as an oil. ¹H NMR (CHLOROFORM-d) δ: 7.24-7.36 (m, 4H), 7.12-7.23 (m, 3H), 7.02 (t, J=8.7 Hz, 2H), 4.51-4.74 (m, 1H), 2.63 (t, J=7.2 Hz, 2H), 1.40-2.09 (m, 4H)

### Step 2

To a stirred, ice-cold solution of 1-(4-fluorophenyl)-4-phenylbutan-1-ol (3.83 g, 13.5 mmol) in CH₂Cl₂ (50 mL) was added solid Dess-Martin periodinane (9.98 g, 23.5 mmol). The mixture was stirred in the ice bath for 2 h. Satd aq NaHCO₃ (20 mL) was added, followed after 15 min by 10% aq Na₂S₂O₃ (20 mL). The mixture was stirred for 15 min and extracted with CH₂Cl₂ (3×50 mL). The combined CH₂Cl₂ layer was washed with satd aq NaHCO₃ (50 mL) and brine (50 mL), and dried over Na₂SO₄. Removal of the solvent left an oil (3.52 g) which was chromatographed on an 80-g silica cartridge, eluted with a 0-80% EtOAc in hexanes gradient, to afford P9-001 (2.43 g, 74% yield over 2 steps). ¹H NMR (CHLOROFORM-d) δ: 7.85-8.04 (m, 2H), 7.26-7.35 (m, 2H), 7.16-7.25 (m, 3H), 7.06-7.16 (m, 2H), 2.95 (t, J=7.3 Hz, 2H), 2.67-2.77 (m, 2H), 2.01-2.16 (m, 2H).

### Preparation 10

### Methyl 2-(1-fluorocyclopropanecarbonyl)-4-(4-fluorophenyl)butanoate (P10-001)

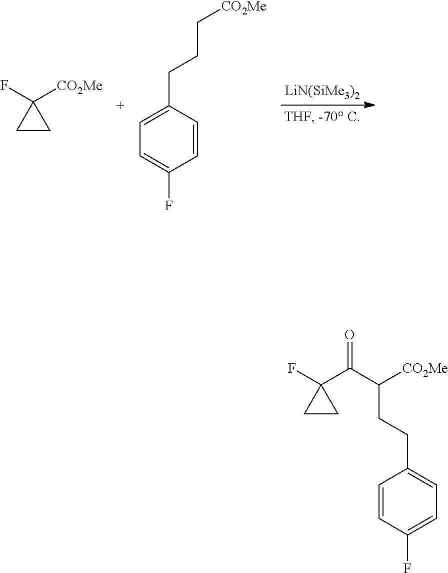

A stirred solution of methyl 1-fluorocyclopropane-1-carboxylate (591 mg, 5.0 mmol) and methyl 4-(4-fluorophenyl)butanoate (351 mg, 2.0 mmol) in dry THF (5 mL) was cooled to −70° C. and 1 M LiN(SiMe₃)₂ in THF (4.8 mL, 4.8 mmol) was added dropwise over 10 min. The mixture was stirred at −70° C. for 1 h and quenched by addition of sat′d aq NH₄Cl (5 mL). The mixture was allowed to warm to rt, diluted with EtOAc (90 mL), washed with water (10 mL) and brine (10 mL), and dried over Na₂SO₄. Removal of the solvent left an oil (760 mg) which was chromatographed on a 40 g silica cartridge, eluted with a 0-20% EtOAc in hexanes gradient, to give P10-001 (275 mg, 39%) as an oil. ¹H NMR (CHLOROFORM-d) δ: 7.09-7.22 (m, 2H), 6.86-7.06 (m, 2H), 3.97 (td, J=7.0, 2.7 Hz, 1H), 3.74 (s, 3H), 2.60-2.69 (m, 2H), 2.09-2.32 (m, 2H), 1.12-1.58 (m, 4H)

### Preparation 11

### 2-hydrazinyl-1-[2-(morpholin-4-yl)ethyl]-1H-1,3-benzodiazole

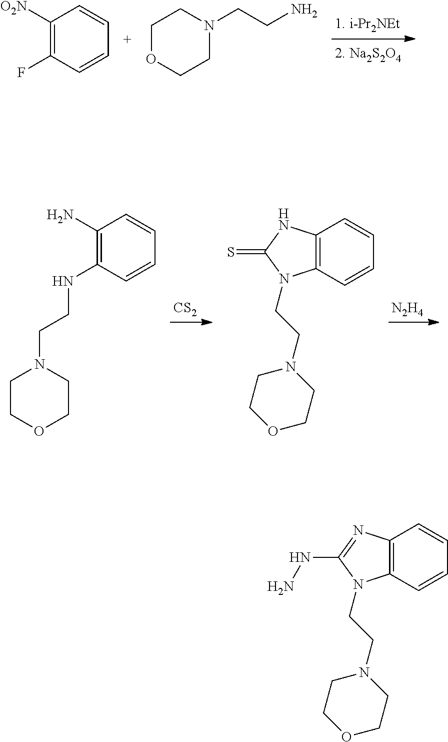

### Step 1

A mixture of 2-fluoronitrobenzene (2.10 g, 14.9 mmol), 2-(morpholin-4-yl)ethan-1-amine (1.94 g, 14.9 mmol), i-Pr₂NEt (2.12 g, 16.4 mmol) and i-PrOH (10 mL) was stirred at 60° C. for 2 h. The mixture was diluted with EtOAc (90 mL), washed with satd aq NaHCO₃ (15 mL), 10% aq K₂CO₃ (15 mL) and brine (15 mL), and dried over Na₂SO₄. Removal of the solvent left an orange oil (3.58 g). Chromatography on a 40 g silica cartridge, eluted with a 0-100% EtOAc in hexanes gradient, gave N-[2-(morpholin-4-yl)ethyl]-2-nitroaniline (2.75 g, 74%) as an orange oil. LC-MS t_{R} 2.55 min, m/z 252.

### Step 2

To a stirred solution of N-[2-(morpholin-4-yl)ethyl]-2-nitroaniline (2.75 g, 10.9 mmol) in 1:1 EtOH/H₂O (50 mL) was added sodium dithionite (10 g, 55 mmol). The mixture was stirred at rt for 0.5 h. Additional sodium dithionite was added until the color of the mixture changed from orange to colorless. The mixture was concentrated under reduced pressure to remove the EtOH. The aqueous residue was diluted with 1M aq NaOH (100 mL) and extracted with CH₂Cl₂ (3×60 mL). The combined CH₂Cl₂ layer was washed with brine (30 mL), dried over Na₂SO₄ and concentrated to leave N1-[2-(morpholin-4-yl)ethyl]benzene-1,2-diamine (0.97 g, 40%) as an oil. LC-MS t_{R} 1.33 min, m/z 222.

### Step 3

A mixture N1-[2-(morpholin-4-yl)ethyl]benzene-1,2-diamine (970 mg, 4.4 mmol), CS₂ (4 mL) and EtOH (10 mL) was stirred at 50 C for 1 d in a sealed vial. The mixture was concentrated to leave 1-[2-(morpholin-4-yl)ethyl]-2,3-dihydro-1H-1,3-benzodiazole-2-thione (1.10 g, 95%) as a yellow solid. LC-MS t_{R} 2.15 min, m/z 264.

### Step 4

A mixture of 1-[2-(morpholin-4-yl)ethyl]-2,3-dihydro-1H-1,3-benzodiazole-2-thione (1.10 g, 4.2 mmol) and hydrazine hydrate (10 mL) was heated at reflux for 6 d. The mixture was concentrated under reduced pressure. The residue was diluted with water and concentrated again. The residue was purified by prep HPLC to give 2-hydrazinyl-1-[2-(morpholin-4-yl)ethyl]-1H-1,3-benzodiazole bis TFA salt (670 mg, 33%). LC-MS t_{R} 1.97 min, m/z 262.

### Synthesis of Compounds of the Invention

### Example 1

### 1-(1H-1,3-benzodiazol-2-yl)-4-[2-(pyridin-4-yl)ethyl]-3-[4-(trifluoromethyl)phenyl]-1H-pyrazol-5-ol (I-101)

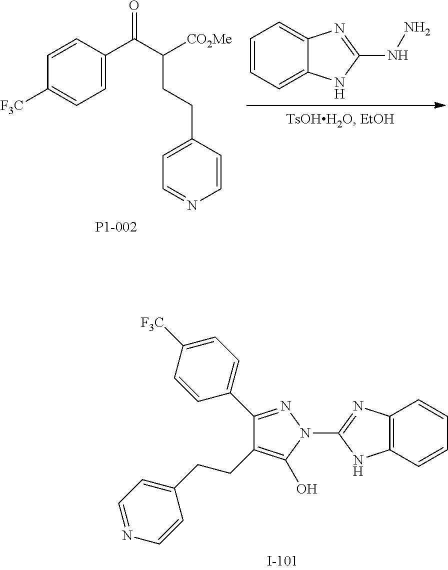

A mixture of β-ketoester P1-002 (158 mg, 0.45 mmol), 2-hydrazinobenzimidazole (134 mg, 0.90 mmol), TsOH.H₂O (cat. qty.) and EtOH (3 mL) was heated in the microwave at 130° C. for 3 h. Preparative HPLC, followed by lyophilization from aq HCl/MeCN, afforded the bis HCl salt of the I-101 (36 mg) as a tan solid. ¹H NMR CD₃OD δ: 8.61 (d, J=5.9 Hz, 2H), 7.79-7.88 (m, 6H), 7.73-7.76 (m, 2H), 7.52-7.55 (m, 2H), 3.14-3.17 (m, 4H); LC-MS t_{R} 3.66 min, m/z 450.

The following compounds are prepared using an analogous procedure.

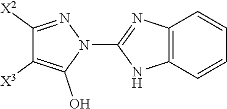

| | | | t_{R} | |
|---|---|---|---|---|
| Cpd No. | X² | X³ | (min) | Mass observed |
| I-102 | c-Pr | 4-F—C₆H₄—CH₂CH₂— | 4.57 | 363 |
| I-103 | pyrazin-2-yl | 4-F—C₆H₄—CH₂CH₂— | 4.98 | 401 |
| I-104 | 4-Me—C₆H₄— | 4-F—C₆H₄—CH₂CH₂— | 5.39 | 413 |
| I-105 | 4-Cl—C₆H₄— | 4-F—C₆H₄—CH₂CH₂— | 5.68 | 435, 433 |
| I-106 | 4-CF₃—C₆H₄— | 4-F—C₆H₄—CH₂CH₂— | 5.55 | 467 |
| I-107 | 4-MeO—C₆H₄— | 4-F—C₆H₄—CH₂CH₂— | 4.99 | 429 |
| I-108 | Ph | 4-F—C₆H₄—CH₂CH₂— | 5.13 | 399 |
| I-109 | pyridin-2-yl | 4-F—CH₄—CH₂CH₂— | 3.54 | 400 |
| I-110 | 3-F—C₆H₄— | 4-F—C₆H₄—CH₂CH₂— | 5.07 | 417 |
| I-111 | pyrazin-2-yl | 3,4-diF-C₆H₃—CH₂CH₂— | 4.81 | 419 |
| I-112 | pyrazin-2-yl | 3-Cl-4-F—C₆H₃—CH₂CH₂— | 5.11 | 437, 435 |
| I-113 | pyridin-4-yl | 4-F—C₆H₄—CH₂CH₂— | 3.23 | 400 |
| I-114 | tetrahydropyran-4-yl | 4-F—C₆H₄—CH₂CH₂— | 4.03 | 407 |
| I-115 | 4-CF₃—C₆H₄— | 4-F—C₆H₄—CH₂CH₂— | 5.10 | 385 |
| I-116 | 3-Cl—C₆H₄— | 4-F—C₆H₄—CH₂CH₂— | 5.39 | 433 |
| I-117 | 2-Cl—C₆H₄— | 4-F—C₆H₄—CH₂CH₂— | 5.28 | 435, 433 |
| I-118 | 5-Br-pyridin-3-yl | 4-F—C₆H₄—CH₂CH₂— | 4.91 | 480, 478 |
| I-119 | 5-CF₃-pyridin-2-yl | 4-F—C₆H₄—CH₂CH₂— | 5.67 | 468 |
| I-120 | 4-F—C₆H₄—CH₂CH₂CH₂— | 4-F—CH₄—CH₂CH₂— | 5.28 | 459 |
| I-121 | pyridin-3-yl | 4-F—C₆H₄—CH₂CH₂— | 3.62 | 400 |
| I-122 | 4-CF₃—C₆H₄— | tetrahydropyran-2-yl-CH₂— | 5.07 | 443 |
| I-123 | 4-CF₃—C₆H₄— | cyclopentylmethyl | 5.93 | 427 |
| I-124 | MeOCH₂— | 4-F—C₆H₄—CH₂CH₂— | 4.58 | 367 |
| I-125 | MeOCH₂CH₂— | 4-F—C₆H₄—CH₂CH₂— | 4.43 | 381 |
| I-126 | 2-CF₃-pyrimidin-5- | 4-F—C₆H₄—CH₂CH₂— | 5.09 | 469 |
| I-127 | 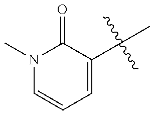 [SMILES: *c1cccn(C)c1=O] | 4-F—C₆H₄—CH₂CH₂— | 3.88 | 430 |
| I-128 | 4-CF₃—C₆H₄— | 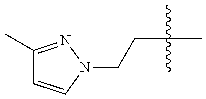 [SMILES: *CCn1ccc(C)n1] | 4.37 | 453 |
| I-129 | c-Bu | 4-F—C₆H₄—CH₂CH₂— | 4.84 | 377 |
| I-130 | t-Bu | 4-F—C₆H₄—CH₂CH₂— | 4.77 | 379 |
| I-131 | 4-CF₃—C₆H₄— | morpholin-4-yl-CH₂CH₂— | 3.89 | 458 |
| I-132 | 4-CF₃—C₆H₄— | 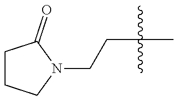 [SMILES: *CCN1CCCC1=O] | 4.19 | 456 |
| I-133 | 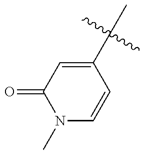 [SMILES: *c1ccn(C)c(=O)c1] | 4-F—C₆H₄—CH₂CH₂— | 4.01 | 430 |
| I-134 | 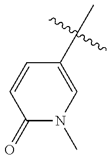 [SMILES: *c1ccc(=O)n(C)c1] | 4-F—C₆H₄—CH₂CH₂— | 3.78 | 430 |
| I-135 | 6-CF₃-pyridin-3-yl | 4-F—C₆H₄—CH₂CH₂— | 5.19 | 468 |
| I-136 | tetrahydrofuran-2-yl | 4-F—C₆H₄—CH₂CH₂— | 4.74 | 393 |
| I-137 | 4-CF₃—C₆H₄— | 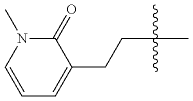 [SMILES: *CCc1cccn(C)c1=O] | 4.28 | 480 |
| I-138 | 6-MeO-pyridin-2-yl | 4-F—C₆H₄—CH₂CH₂— | 5.18 | 430 |
| I-139 | 4-CF₃—C₆H₄— | tetrahydropyran-4-yl-CH₂CH₂— | 5.09 | 457 |
| I-140 | 6-Me-pyridin-3-yl | 4-F—C₆H₄—CH₂CH₂— | 3.52 | 414 |
| I-141 | 4-CF₃—C₆H₄— | 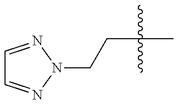 [SMILES: *CCn1nccn1] | 4.56 | 440 |
| I-142 | 4-CF₃—C₆H₄— | 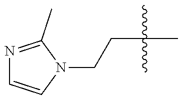 [SMILES: *CCn1ccnc1C] | 3.53 | 453 |
| I-143 | 4-CF₃—C₆H₄— | 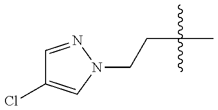 [SMILES: *CCn1cc(Cl)cn1] | 4.90 | 475, 473 |
| I-144 | 5-CF₃-pyridin-2-yl | 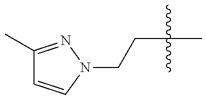 [SMILES: *CCn1ccc(C)n1] | 4.07 | 454 |
| I-145 | 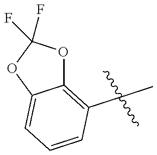 [SMILES: *c1cccc2c1OC(F)(F)O2] | 4-F—C₆H₄—CH₂CH₂— | 5.32 | 479 |
| I-146 | 5-CF₃-pyridin-2-yl | 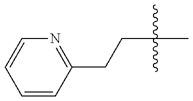 [SMILES: *CCc1ccccn1] | 3.58 | 451 |
| I-147 | 5-CF₃-pyridin-2-yl | 4-Br—C₆H₄—CH₂CH₂— | 6.08 | 530, 528 |
| I-148 | 4-MeSO₂—C₆H₄— | 4-F—C₆H₄—CH₂CH₂— | 4.40 | 477 |
| I-149 | 4-MeSO₂—CH₄— | tetrahydropyran-4-yl-CH₂CH₂— | 3.67 | 467 |
| I-150 | 3-F—C₆H₄— | tetrahydropyran-4-yl-CH₂CH₂— | 4.47 | 407 |
| I-151 | 4-F—C₆H— | tetrahydropyran-4-yl-CH₂— | 4.13 | 393 |
| I-152 | 4-F—C₆H₄— | 4-F—CH₆ H₄—CH₂CH₂— | 5.28 | 417 |
| I-153^{a} | 4-CF₃—C₆H₄— | HOCH₂CH₂— | 4.08 | 389 |
| I-154 | 4-MeSO₂—CH₄— | N—CF₃CH₂-piperidin-4-yl- | 3.58 | 548 |
| | | CH₂CH₂— | | |
| I-155 | 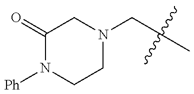 [SMILES: *CN1CCN(c2ccccc2)C(=O)C1] | 4-F—C₆H₄—CH₂CH₂— | 3.93 | 511 |
| I-156 | Me | 4-Cl-2-F—CH₃—CH₂— | 4.43 | 359, 357 |
| I-157 | morpholin-4-yl-CH₂— | 4-F—C₆H₄—CH₂CH₂— | 3.28 | 422 |
| I-158 | N-Cbz-piperidin-4-yl | 4-F—C₆H₄—CH₂CH₂— | 5.28 | 540 |
| I-159 | N-Cbz-piperidin-4-yl | 3,4-diF—C₆H₃—CH₂— | w | 544 |
| I-160 | N-Cbz-piperidin-3-yl | 4-F—C₆H₄—CH₂CH₂— | 5.13 | 540 |
| I-162 | 4-CF₃—C₆H₄— | N-Cbz-piperidin-4-yl-CH₂— | 5.64 | 576 |
| I-164 | 4-CF₃—C₆H₄— | N-Cbz-piperidin-4-yl- | 5.73 | 562 |
| I-174 | c-Pr | tetrahydropyran-4-yl-CH₂— | 3.40 | 339 |
| I-175 | 4-F—C₆H₄— | tetrahydropyran-3-yl-CH₂— | 4.28 | 393 |
| I-176 | 3,3-diF-c-Bu | tetrahydropyran-4-yl-CH₂— | 3.98 | 389 |
| I-177 | tetrahydropyran-4-yl | tetrahydropyran-4-yl-CH₂— | 3.23 | 383 |
| I-178 | tetrahydropyran-4- | tetrahydropyran-4-yl-CH₂— | 3.60 | 411 |
| | yl-CH₂CH₂— | | | |
| I-180 | t-Bu— | tetrahydropyran-4-yl-CH₂— | 3.88 | 355 |
| I-181 | 1-F-c-Pr— | tetrahydropyran-4-yl-CH₂— | 4.87 | 381 |
| I-182 | 4-F—C₆H₄— | tetrahydropyran-4-yl-CH₂CH₂— | 4.30 | 407 |
| I-183 | Ph— | tetrahydropyran-4-yl-CH₂— | 4.07 | 375 |
| I-184 | 3-F-C₆H₄— | tetrahydropyran-4-yl-CH₂— | 4.26 | 393 |
| I-185 | 3,4-diF-C₆H₃— | tetrahydropyran-4-yl-CH₂— | 4.41 | 411 |
| I-186 | 2-F—C₆H₄— | tetrahydropyran-4-yl-CH₂— | 4.14 | 393 |
| I-187 | 2,4-diF-C₆H₃— | tetrahydropyran-4-yl-CH₂— | 4.27 | 411 |
| I-188 | 2,5-diF-C₆H₃— | tetrahydropyran-4-yl-CH₂— | 4.42 | 411 |
| I-189 | 3-CF₃—C₆H₄— | tetrahydropyran-4-yl-CH₂— | 4.88 | 443 |
| I-190 | 3,5-diF-C₆H₃— | tetrahydropyran-4-yl-CH₂— | 4.67 | 411 |

^{a}The β-ketoester employed was 3-(4-(trifluoromethyl)benzoyl)-γ-butyrolactone.

### Benzyl 4-[1-(5-chloro-1H-1,3-benzodiazol-2-yl)-5-hydroxy-4-[2-(oxan-4-yl)ethyl]-1H-pyrazol-3-yl]piperidine-1-carboxylate (I-179)

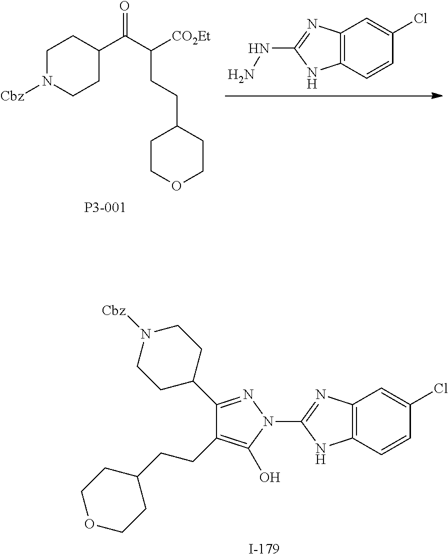 [SMILES: CCOC(=O)C(CCC1CCOCC1)C(=O)C1CCN(C(=O)OCc2ccccc2)CC1 ; NNc1nc2cc(Cl)ccc2[nH]1 ; O=C(OCc1ccccc1)N1CCC(c2nn(-c3nc4cc(Cl)ccc4[nH]3)c(O)c2CCC2CCOCC2)CC1]

A mixture of 11 (245 mg, 0.55 mmol), 5-chloro-2-hydrazinobenzimidazole (150 mg, 0.82 mmol), TsOH.H₂O (cat. qty.) and EtOH (4 mL) was heated in the microwave at 130° C. for 3 h. The mixture was diluted with EtOAc (90 mL), washed with 5% aq HCl (15 mL), sat′d aq NaHCO₃ (15 mL) and brine (15 mL), and dried over Na₂SO₄. Removal of the solvent left crude I-179 (350 mg) as a dark oil. LC-MS t_{R} 5.19 min, m/z 518, 516.

The following compounds are prepared using an analogous procedure.

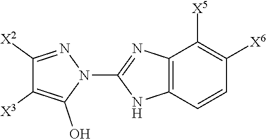

| | | | | t_{R} | Mass |
|---|---|---|---|---|---|
| Cpd No. | X² | X³ | X⁵/X⁶ | (min) | observed |
| I-161 | 4-CF₃—C₆H₄— | 4-F—C₆H₄—CH₂CH₂— | H/Cl | 6.51 | 503, 501 |
| I-163 | 4-CF₃—C₆H₄— | 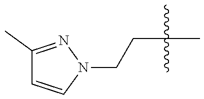 [SMILES: *CCn1ccc(C)n1] | H/Cl | 4.93 | 489, 487 |
| I-165 | Ph | 4-F—C₆H₄—CH₂CH₂— | H/Cl | 5.87 | 435, 433 |
| I-166 | N-Cbz-piperidin-4- | 4-F—C₆H₄—CH₂CH₂— | Cl/H | 6.28 | 576, 574 |
| | yl | | | | |
| I-167 | 5-CF₃-pyridin-2-yl | 4-F—C₆H₄—CH₂CH₂— | H/F | 6.29 | 486 |
| I-168 | 4-MeSO₂—C₆H₄— | tetrahydropyran-4-yl-CH₂CH₂— | H/Cl | 4.91 | 503, 501 |
| I-169 | 4-MeSO₂—C₆H₄— | tetrahydropyran-4-yl-CH₂CH₂— | H/F | 4.32 | 485 |
| I-170 | c-Pr | 4-F—CH₄—CH₂CH₂— | H/Cl | 5.18 | 399, 397 |
| I-171 | Ph | 4-MeO₂C—C₆H₄—CH₂CH₂— | H/Cl | 5.68 | 473 |
| I-172 | 4-F—C₆H₄— | 4-F—C₆H₄—CH₂CH₂— | H/Cl | 5.95 | 453, 451 |
| I-173 | 4-F—C₆H₄— | tetrahydropyran-4-yl-CH₂— | H/F | 4.76 | 411 |
| I-191 | 4-CF₃—C₆H₄— | 4-pyridinyl-CH₂CH₂— | H/Cl | 4.83 | 486, 484, |
| | | | | | 393, 391 |
| I-192^{a} | Ph | 4-EtO₂C—C₆H₄—CH₂CH₂— | H/Cl | 5.63 | 489, 487 |

^{a}Byproduct when reaction of P3-011 and 5-chloro-2-hydrazinobenzimidazole is run in ethanol.

### Example 2

### tert-butyl 4-{2-[1-(1H-1,3-benzodiazol-2-yl)-5-hydroxy-3-(4-methanesulfonylphenyl)-1H-pyrazol-4-yl]ethyl}piperidine-1-carboxylate (I-201)

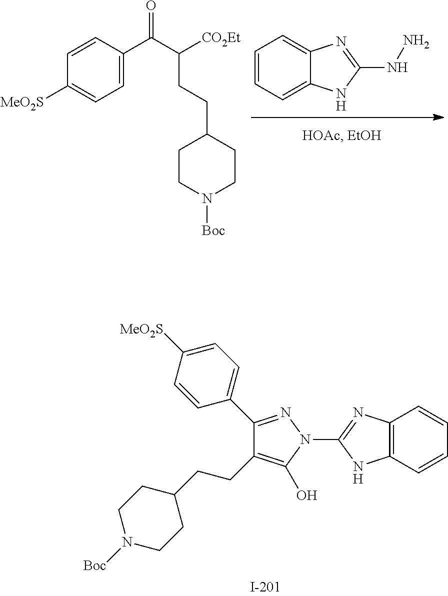 [SMILES: CCOC(=O)C(CCC1CCN(C(=O)OC(C)(C)C)CC1)C(=O)c1ccc(S(C)(=O)=O)cc1 ; NNc1nc2ccccc2[nH]1 ; CC(C)(C)OC(=O)N1CCC(CCc2c(-c3ccc(S(C)(=O)=O)cc3)nn(-c3nc4ccccc4[nH]3)c2O)CC1]

A mixture of β-ketoester (111 mg, 0.25 mmol), 2-hydrazinobenzimidazole (110 mg, 0.75 mmol), HOAc (0.5 mL) and EtOH (1.5 mL) was heated in the microwave at 130° C. for 1.5 h. Preparative HPLC gave the TFA salt of I-201 (35 mg) as a white solid. 1H NMR (CD3OD) δ: 8.09 (d, J=8.2 Hz, 2H), 8.01 (d, J=8.2 Hz, 2H), 7.65-7.70 (m, 2H), 7.42-7.47 (m, 2H), 3.99 (m, 2H), 3.19 (s, 3H), 2.60-2.71 (m, 4H), 1.64 (m, 2H), 1.38-1.50 (m, 12H), 1.03 (m, 2H).

LC-MS t_{R} 4.84 min, m/z 566, 510, 466.

The following compounds are prepared using analogous procedures

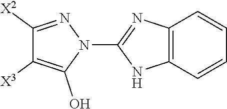

| | | | t_{R} | |
|---|---|---|---|---|
| Cpd no. | X² | X³ | (min) | Mass observed |
| I-202 | N-Boc-piperidin-4-yl | 4-F—C₆H₄—CH₂CH₂— | 5.07 | 506 |
| I-203 | N-Boc-piperidin-4-yl | 3,4-diF—C₆H₃—CH₂— | 5.11 | 510 |
| I-204 | 4-CF₃—C₆H₄— | N-Boc-piperidin-4-yl-CH₂CH₂— | 5.85 | 557 |
| I-205 | N-Boc-pyrrolidin-2-yl | 4-F—C₆H₄—CH₂CH₂— | 5.11 | 492 |
| I-206 | N-Boc-pyrrolidin-3-yl- | 4-F—C₆H₄—CH₂CH₂— | 5.01 | 506 |
| | CH₂— | | | |
| I-207 | N-Boc-piperidin-4-yl- | 4-F—CH₄—CH₂CH₂— | 5.43 | 520, 420 |
| | CH₂— | | | |
| I-208 | 4-(t-BuO₂C)—C6H₄— | 4-F—C₆H₄—CH₂CH₂— | 5.72 | 499 |
| I-209^{a} | 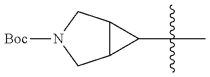 [SMILES: *C1C2CN(C(=O)OC(C)(C)C)CC12] | 4-F—C₆H₄—CH₂CH₂— | 4.82 | 504 |
| I-210 | 3-MeSO₂—C₆H₄— | N-Boc-piperidin-4-yl-CH₂CH₂— | 4.91 | 566, 510, 466 |
| I-211 | 4-Boc-piperazin-1-yl-CH₂— | 4-F—C₆H₄—CH₂CH₂— | 4.02 | 521 |
| I-212 | BocNHCH₂— | 4-F—C₆H₄—CH₂CH₂— | 4.86 | 452, 396 |
| I-213 | 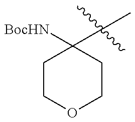 [SMILES: *C1(NC(=O)OC(C)(C)C)CCOCC1] | 4-F—C₆H₄—CH₂CH₂— | 5.72 | 460 |
| I-214 | N-Boc-piperidin-3-yl- | 4-F—C₆H₄—CH₂CH₂— | 5.22 | 520, 420 |
| | CH₂— | | | |

^{a}X² is a mixture of cis and trans isomers.

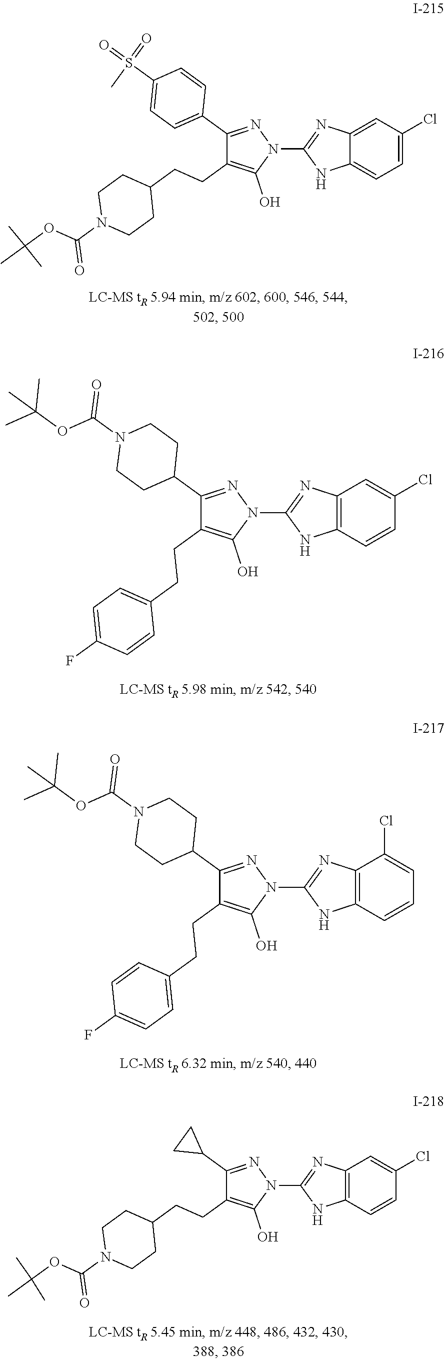 [SMILES: CC(C)(C)OC(=O)N1CCC(CCc2c(-c3ccc(S(C)(=O)=O)cc3)nn(-c3nc4cc(Cl)ccc4[nH]3)c2O)CC1 ; CC(C)(C)OC(=O)N1CCC(c2nn(-c3nc4cc(Cl)ccc4[nH]3)c(O)c2CCc2ccc(F)cc2)CC1 ; CC(C)(C)OC(=O)N1CCC(c2nn(-c3nc4c(Cl)cccc4[nH]3)c(O)c2CCc2ccc(F)cc2)CC1 ; CC(C)(C)OC(=O)N1CCC(CCc2c(C3CC3)nn(-c3nc4cc(Cl)ccc4[nH]3)c2O)CC1]

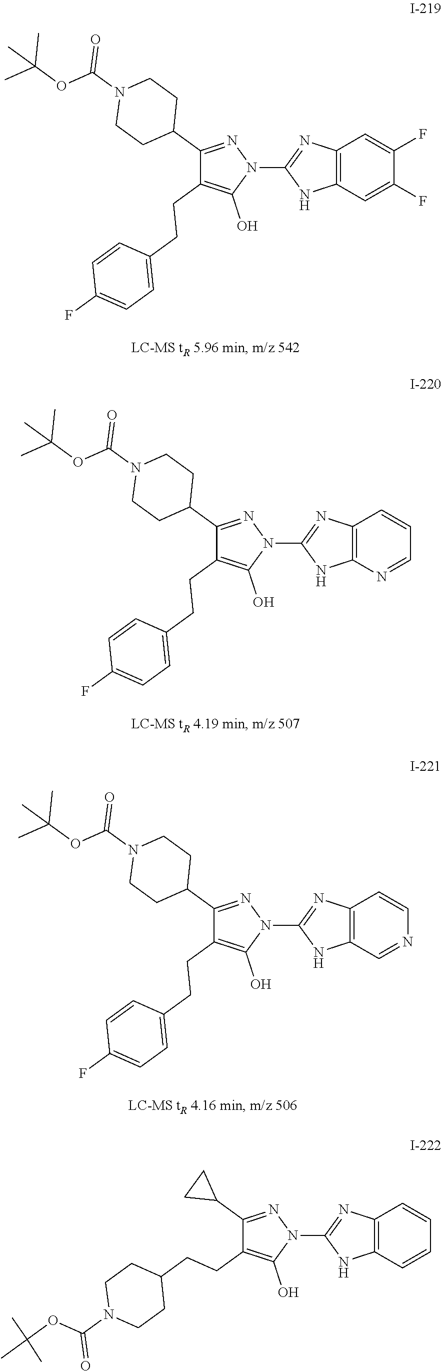 [SMILES: CC(C)(C)OC(=O)N1CCC(c2nn(-c3nc4cc(F)c(F)cc4[nH]3)c(O)c2CCc2ccc(F)cc2)CC1 ; CC(C)(C)OC(=O)N1CCC(c2nn(-c3nc4cccnc4[nH]3)c(O)c2CCc2ccc(F)cc2)CC1 ; CC(C)(C)OC(=O)N1CCC(c2nn(-c3nc4ccncc4[nH]3)c(O)c2CCc2ccc(F)cc2)CC1 ; CC(C)(C)OC(=O)N1CCC(CCc2c(C3CC3)nn(-c3nc4ccccc4[nH]3)c2O)CC1]

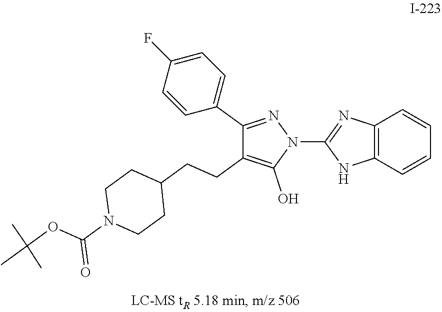 [SMILES: CC(C)(C)OC(=O)N1CCC(CCc2c(-c3ccc(F)cc3)nn(-c3nc4ccccc4[nH]3)c2O)CC1]

### Example 3

### 4-[2-(4-fluorophenyl)ethyl]-1-{1H-imidazo[4,5-b]pyridin-2-yl}-3-[4-(trifluoromethyl)phenyl]-1H-pyrazol-

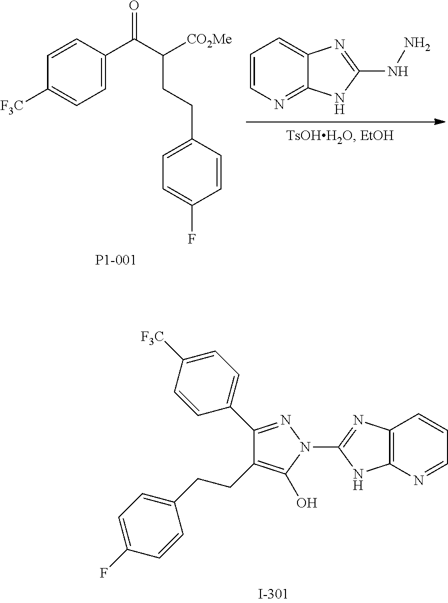 [SMILES: COC(=O)C(CCc1ccc(F)cc1)C(=O)c1ccc(C(F)(F)F)cc1 ; NNc1nc2cccnc2[nH]1 ; Oc1c(CCc2ccc(F)cc2)c(-c2ccc(C(F)(F)F)cc2)nn1-c1nc2cccnc2[nH]1]

A mixture of β-ketoester P1-001 (48 mg, 0.13 mmol), 2-hydrazino-4-azabenzimidazole (97 mg, 0.65 mmol), TsOH.H₂O (cat. qty.) and EtOH (2 mL) was heated in the microwave at 130° C. for 3 h. Prep HPLC afforded I-301 (16 mg, 26%) as a pale pink solid. ¹H NMR (DMSO-d₆) δ: 8.38 (dd, J=5.0, 1.5 Hz, 1H), 8.01 (dd, J=8.0, 1.5 Hz, 1H), 7.83-7.93 (m, 4H), 7.26-7.39 (m, 1H), 7.14-7.24 (m, 2H), 7.01-7.10 (m, 2H), 2.79-2.88 (m, 4H); LC-MS t_{R} 4.47 min, m/z 468.

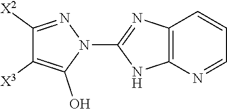

| Cpd | | | t_{R} | Mass |
|---|---|---|---|---|
| No. | X² | X³ | (min) | observed |
| I-302 | 5-CF₃-pyridin-2-yl | 4-F—C₆H₄—CH₂CH₂— | 4.62 | 469 |
| I-303 | 4-CF₃—C₆H₄— | 3-Me-pyrazol-1-yl- | 3.52 | 454 |
| | | CH₂CH₂— | | |
| I-304 | 3-F—C₆H₄— | tetrahydropyran-4-yl- | 3.47 | 408 |
| | | CH₂CH₂— | | |
| I-305 | 3-MeSO₂—C₆H₄— | tetrahydropyran-4-yl- | 3.10 | 468 |
| | | CH₂CH₂— | | |
| I-306 | N-Cbz-piperidin-4-yl | 4-F—C₆H₄—CH₂CH₂— | 4.32 | 541 |
| I-307 | 4-F—C₆H₄—CH₂CH₂— | 4-F—C₆H₄—CH₂CH₂— | 4.04 | 460 |
| I-308 | c-Pr | 4-F—C₆H₄—CH₂CH₂— | 3.62 | 364 |
| I-311 | 3-MeSO₂—C₆H₄— | 4-F—C₆H₄—CH₂CH₂— | 4.18 | 478 |
| I-312 | tetrahydropyran-4-yl | tetrahydropyran-4-yl-CH₂— | 2.58 | 384 |
| I-313 | 4-F—C₆H₄— | tetrahydropyran-4-yl-CH₂— | 3.22 | 394 |
| I-314 | tetrahydropyran-4-yl | 4-F—C₆H₄—CH₂CH₂— | 3.39 | 408 |
| I-315 | c-Pr | tetrahydropyran-4-yl-CH₂— | 2.68 | 340 |
| I-316 | 3,3-diF-c-Bu | tetrahydropyran-4-yl-CH₂— | 3.13 | 390 |

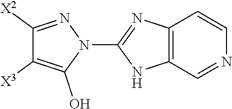

| Cpd | | | t_{R} | Mass |
|---|---|---|---|---|
| No. | X² | X³ | (min) | observed |
| I-309 | 4-CF₃—C₆H₄— | 4-F—C₆H₄—CH₂CH₂— | 4.31 | 468 |
| I-310 | N-Cbz-piperidin-4- | 4-F—C₆H₄—CH₂CH₂— | 4.23 | 541 |
| | yl | | | |

### Example 4

### 4-[2-(4-fluorophenyl)ethyl]-1-(1-methyl-1H-1,3-benzodiazol-2-yl)-3-[4-(trifluoromethyl)phenyl]-1H-pyrazol-5-ol (I-401)

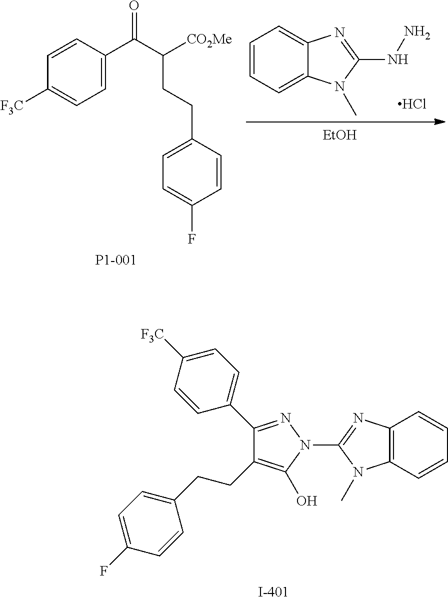 [SMILES: "EtOH" ; COC(=O)C(CCc1ccc(F)cc1)C(=O)c1ccc(C(F)(F)F)cc1 ; Cn1c(NN)nc2ccccc21 ; Cl ; Cn1c(-n2nc(-c3ccc(C(F)(F)F)cc3)c(CCc3ccc(F)cc3)c2O)nc2ccccc21]

A mixture of P1-001 (100 mg, 0.27 mmol), 1-methyl-2-hydrazinobenzimidazole HCl salt (60 mg, 0.30 mmol) and EtOH (1 mL) was stirred at 40° C. for 2 d. Preparative HPLC, followed by lyophilization from aq HCl/MeCN, afforded I-401 as its HCl salt as a white solid. ¹H NMR (CD₃OD) δ: 7.73 (d, J=3.3 Hz, 6H), 7.39-7.53 (m, 2H), 7.05 (dd, J=8.5, 5.5 Hz, 2H), 6.81-6.92 (m, 2H), 4.04 (s, 3H), 2.86-2.97 (m, 2H), 2.82 (d, J=7.0 Hz, 2H); LC-MS t_{R} 6.63 min, m/z 481.

The following compounds are prepared using analogous procedures:

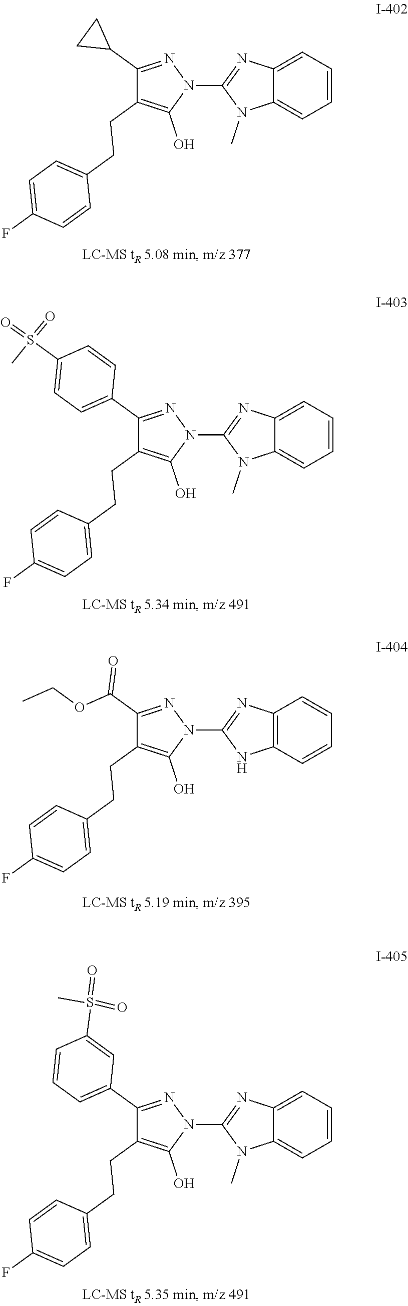 [SMILES: Cn1c(-n2nc(C3CC3)c(CCc3ccc(F)cc3)c2O)nc2ccccc21 ; Cn1c(-n2nc(-c3ccc(S(C)(=O)=O)cc3)c(CCc3ccc(F)cc3)c2O)nc2ccccc21 ; CCOC(=O)c1nn(-c2nc3ccccc3[nH]2)c(O)c1CCc1ccc(F)cc1 ; Cn1c(-n2nc(-c3cccc(S(C)(=O)=O)c3)c(CCc3ccc(F)cc3)c2O)nc2ccccc21]

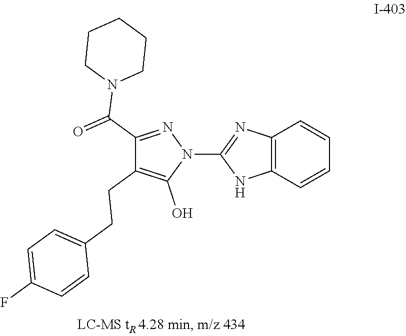 [SMILES: O=C(c1nn(-c2nc3ccccc3[nH]2)c(O)c1CCc1ccc(F)cc1)N1CCCCC1]

### Example 5

### 1-(5-chloro-1H-1,3-benzodiazol-2-yl)-3-(1-methanesulfonylpiperidin-4-yl)-4-[2-(2H-1,2,3-triazol-2-yl)ethyl]-1H-pyrazol-5-ol (I-501)

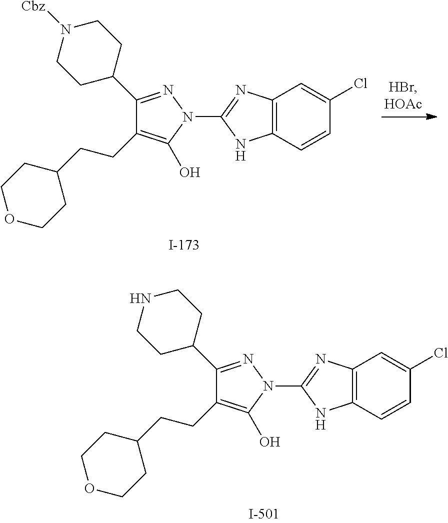 [SMILES: O=C(OCc1ccccc1)N1CCC(c2nn(-c3nc4cc(Cl)ccc4[nH]3)c(O)c2CCC2CCOCC2)CC1 ; Oc1c(CCC2CCOCC2)c(C2CCNCC2)nn1-c1nc2cc(Cl)ccc2[nH]1]

Crude I-179 (350 mg, ≤0.55 mmol) was dissolved in 30% HBr in HOAc (8 mL) and stirred at rt for 3 h. The mixture was concentrated. The residue was taken up in water (45 mL), washed with ether (10 mL) and lyophilized to give crude I-501 (350 mg) as a dark solid. LC-MS t_{R} 3.21 min, m/z 414.

The following compounds are prepared using analogous procedures.

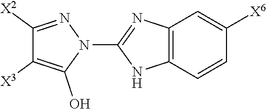

| Cpd | | | | t_{R} | Mass |
|---|---|---|---|---|---|
| No. | X² | X³ | X⁶ | (min) | observed |
| I- | piperidin-3-yl | 4-F—C₆H₄—CH₂CH₂— | H | 3.60 | 406 |
| 502 | | | | | |
| I- | piperidin-4-yl | 4-F—C₆H₄—CH₂CH₂— | Cl | 4.06 | 440 |
| 503 | | | | | |
| I- | 4- | piperidin-4-yl-CH₂— | H | 3.64 | 442 |
| 504 | CF₃—C₆H₄— | | | | |
| I- 505 | piperidin-4-yl | 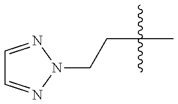 [SMILES: *CCn1nccn1] | Cl | 3.14 | 415, 413 |
| I- | piperidin-4-yl | tetrahydropyran-4-yl- | H | 2.93 | 396 |
| 506 | | CH₂CH₂— | | | |
| I- | piperidin-4-yl | tetrahydropyran-4-yl- | H | 2.65 | 382 |
| 507 | | CH₂— | | | |
| I- | 4- | piperidin-4-yl | H | 3.66 | 428 |
| 508 | CF₃—C₆H₄— | | | | |
| I- | piperidin-4-yl | 3,4-diF—C₆H₃—CH₂— | H | 3.42 | 410 |
| 509 | | | | | |

Alternatively, the Cbz group is replaced by a Boc group and 3:1 CH₂Cl₂/TFA or 4 M HCl in dioxane are employed in place of 30% HBr in HOAc.

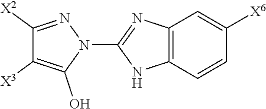

| Cpd | | | | t_{R} | Mass |
|---|---|---|---|---|---|
| No. | X² | X³ | X⁶ | (min) | observed |
| I-550 | 4-MeSO₂—C₆H₄— | piperidin-4-yl-CH₂CH₂— | Cl | 3.15 | 502, 500 |
| I-551 | piperidin-4-yl | 4-F—C₆H₄—CH₂CH₂— | H | 3.04 | 406 |
| I-552 | 4-CF₃—C₆H₄— | piperidin-4-yl-CH₂CH₂— | H | 3.75 | 456 |
| I-553 | piperidin-4-yl- | 4-F—C₆H₄—CH₂CH₂— | H | 3.15 | 420d=′ |
| | CH₂— | | | | |
| I-554 | c-Pr | piperidin-4-yl-CH₂CH₂— | Cl | 3.40 | 388, 386 |
| I-555 | 5-CF₃-pyrid-2-yl | piperidin-4-yl-CH₂CH₂— | Br | 4.27 | 536, 534 |
| I-556 | piperidin-3-yl- | 4-F—C₆H₄—CH₂CH₂— | H | 3.13 | 420 |
| | CH₂— | | | | |
| I-557 | 3-MeSO₂—C₆H₄— | piperidin-4-yl-CH₂CH₂— | H | 3.33 | 484 |
| I-558 | 5-CF₃-pyrid-2-yl | piperidin-4-yl-CH₂CH₂— | H | 3.59 | 457 |
| I-559 | 5-Br-pyrid-2-yl | piperidin-4-yl-CH₂CH₂— | H | 3.27 | 469, 467 |
| I-560 | pyrrolidin-2-yl | 4-F—C₆H₄—CH₂CH₂— | H | | |
| I-561 | H₂NCH₂— | 4-F—C₆H₄—CH₂CH₂— | H | | |
| I-562 | c-Pr | piperidin-3-yl-CH₂— | H | | |
| I-563 | 4-F—C₆H₄— | piperidin-3-yl-CH₂— | H | 3.22 | 392 |
| I-564 | 4-F—C₆H₄— | piperidin-4-yl-CH₂CH₂— | H | | |

### Example 6

### 4-[2-(4-fluorophenyl)ethyl]-5′-(trifluoromethyl)-3-[4-(trifluoromethyl)phenyl]-2′H-[1,3′-bipyrazol]-5-ol (I-601)

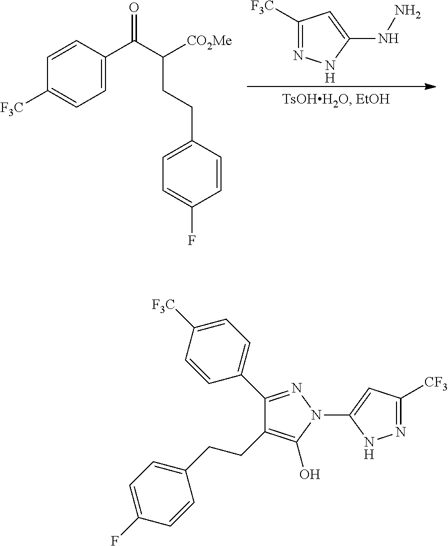 [SMILES: COC(=O)C(CCc1ccc(F)cc1)C(=O)c1ccc(C(F)(F)F)cc1 ; NNc1cc(C(F)(F)F)n[nH]1 ; Oc1c(CCc2ccc(F)cc2)c(-c2ccc(C(F)(F)F)cc2)nn1-c1cc(C(F)(F)F)n[nH]1]

A mixture of β-ketoester 3 (350 mg, 0.92 mmol), 3-hydrazino-5-(trifluoromethyl)pyrazole (148 mg, 0.89 mmol), TsOH.H₂O (cat. qty.) and EtOH (4 mL) was heated in the microwave at 130° C. for 1 h. Prep HPLC afforded the title compound (350 mg, % yield) as an off-white solid. ¹H NMR (CD₃OD) δ: 7.60-7.73 (m, 4H), 6.97-7.06 (m, 2H), 6.80-6.90 (m, 3H), 2.81-2.90 (m, 2H), 2.77 (d, J=6.6 Hz, 2H). LC-MS t_{R} 5.88 min, m/z 485.

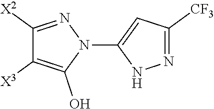

| Cpd | | | t_{R} | Mass |
|---|---|---|---|---|
| No. | X² | X³ | (min) | observed |
| I-602 | 5-CF₃-pyridin-2-yl | 4-F—C₆H₄—CH₂CH₂— | 6.0 | 486 |
| I-603 | 4-MeSO₂—C₆H₄— | 4-F—C₆H₄—CH₂CH₂— | 4.63 | 495 |
| I-604 | 3-MeSO₂—C₆H₄— | 4-F—C₆H₄—CH₂CH₂— | 4.95 | 495 |
| I-605 | N—i-PrO₂C-piperidin-4-yl | 4-F—C₆H₄—CH₂CH₂— | 4.7 | 500 |
| I-606 | c-Pr | 4-F—C₆H₄—CH₂CH₂— | 4.93 | 381 |
| I-607 | 4-F—C₆H₄— | tetrahydropyran-4-yl- | 4.63 | 411 |
| | | CH₂— | | |
| I-608 | 3-MeSO₂—C₆H₄— | tetrahydropyran-4-yl- | 4.35 | 485 |
| | | CH₂CH₂— | | |
| I-609 | 4-F—C₆H₄— | tetrahydropyran-4-yl- | 4.92 | 425 |
| | | CH₂CH₂— | | |
| I-610 | 3-F—C₆H₄— | tetrahydropyran-4-yl- | 5.02 | 425 |
| | | CH₂CH₂— | | |
| I-611 | 4-MeSO₂—C₆H₄— | tetrahydropyran-4-yl- | 4.28 | 485 |
| | | CH₂CH₂— | | |
| I-612^{a} | 3-MeSO₂—C₆H₄— | N—Boc-piperidin-4-yl- | 5.13 | 528, 484 |
| | | CH₂CH₂— | | |
| I-614 | 4-MeSO₂—C₆H₄— | N—CF₃CH₂-piperidin-4- | 3.6 | 566 |
| | | yl-CH₂CH₂— | | |
| I-615 | 3-F—C₆H₄— | pyridin-4-yl-CH₂CH₂— | 3.47 | 418 |
| I-616 | 4-F—C₆H₄— | pyridin-4-yl-CH₂CH₂— | 3.38 | 418 |
| I-617 | Ph | 4-EtO₂C—C₆H₄—CH₂CH₂— | 5.45 | 471 |
| I-618 | Ph | 4-HO₂C—C₆H₄—CH₂CH₂— | 4.47 | 443 |
| I-621 | tetrahydropyran-4-yl | tetrahydropyran-4-yl- | 3.64 | 401 |
| | | CH₂— | | |
| I-622 | c-Pr | tetrahydropyran-4-yl- | 3.98 | 357 |
| | | CH₂— | | |
| I-623 | tetrahydropyran-4-yl | 4-F—C₆H₄—CH₂CH₂— | 4.67 | 425 |

^{a}TsOH was omitted and 3:1 EtOH/HOAc was used in place of EtOH.

The following compounds are prepared using analogous procedures:

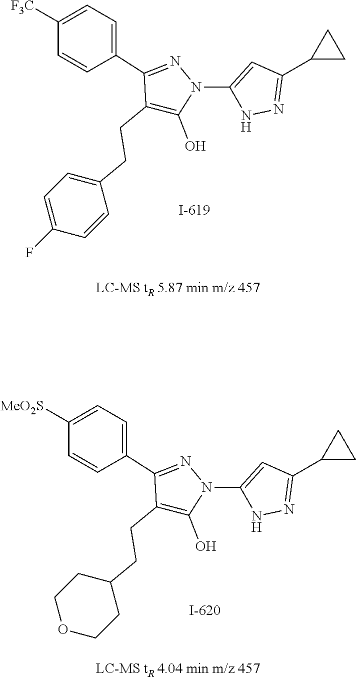 [SMILES: Oc1c(CCc2ccc(F)cc2)c(-c2ccc(C(F)(F)F)cc2)nn1-c1cc(C2CC2)n[nH]1 ; CS(=O)(=O)c1ccc(-c2nn(-c3cc(C4CC4)n[nH]3)c(O)c2CCC2CCOCC2)cc1]

Compound I-613 is prepared from I-612 following procedures analogous to those in Example 5 and Example 7, using AcOC(Me)₂COCl in place of i-PrOCOCl.

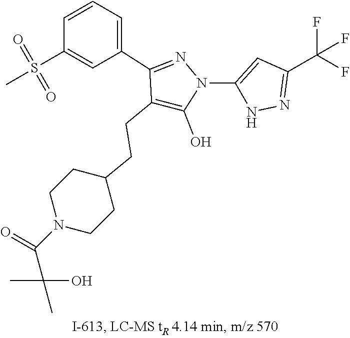 [SMILES: CC(C)(O)C(=O)N1CCC(CCc2c(-c3cccc(S(C)(=O)=O)c3)nn(-c3cc(C(F)(F)F)n[nH]3)c2O)CC1]

### Example 7

### Propan-2-yl 4-[1-(5-chloro-1H-1,3-benzodiazol-2-yl)-5-hydroxy-4-[2-(oxan-4-yl)ethyl]-1H-pyrazol-3-yl]piperidine-1-carboxylate (I-701)

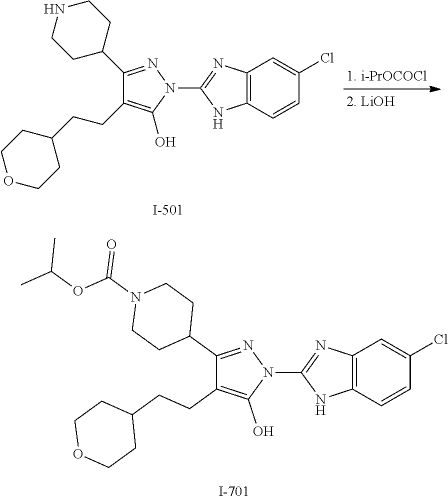 [SMILES: Oc1c(CCC2CCOCC2)c(C2CCNCC2)nn1-c1nc2cc(Cl)ccc2[nH]1 ; CC(C)OC(=O)N1CCC(c2nn(-c3nc4cc(Cl)ccc4[nH]3)c(O)c2CCC2CCOCC2)CC1]

To a stirred ice-cold solution of crude I-501 (200 mg, 0.39 mmol) and i-Pr₂NEt (0.42 mL, 2.35 mmol) in CH₂Cl₂ (20 mL) was added dropwise 2M i-PrOCOCl in toluene (0.60 mL, 1.2 mmol). The mixture was stirred in the ice bath for 2 h and concentrated. The residue was taken up in 3:1 H₂O/MeOH (12 mL) and treated with LiOH (100 mg, 4.2 mmol). The mixture was stirred at rt for 18 h and concentrated. The residue was diluted with 5% aq HCl (20 mL) and extracted with EtOAc (90 mL). The EtOAc layer was washed with brine (10 mL) and rotovaped to leave a dark oil. Prep HPLC gave I-701 (57 mg, % yield) as a white solid. 1H NMR (CD3OD) δ: 7.51-7.59 (m, 2H), 7.29 (dd, J=8.6, 1.9 Hz, 1H), 4.22 (br d, J=13.4 Hz, 2H), 3.92 (br dd, J=11.4, 4.3 Hz, 2H), 3.35-3.40 (m, 2H), 2.89-3.00 (m, 3H), 2.39-2.45 (m, 2H), 1.67-1.90 (m, 6H), 1.44-1.58 (m, 3H), 1.21-1.34 (m, 9H) LC-MS tR 5.19 min, m/z 518, 516.

The following compounds are prepared using analogous procedures. Depending on reagent availability, other acylating agents such as carboxylic acid/HATU or anhydrides were used in place of acid chlorides:

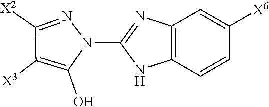

| Cpd | | | | t_{R} | Mass |
|---|---|---|---|---|---|
| No. | X² | X³ | X⁶ | (min) | observed |
| I-702 | N-acetylpiperidin-4-yl | 4-F—C₆H₄—CH₂CH₂— | Cl | 4.76 | 482 |
| I-703 | N-acetylpiperidin-4-yl | tetrahydropyran-4-yl- | Cl | 4.06 | 474, 472 |
| | | CH₂CH₂— | | | |
| I-704 | N-acetylpiperidin-4-yl | 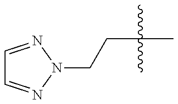 [SMILES: *CCn1nccn1] | Cl | 3.62 | 457, 455 |
| I-705 | N-(MeO₂C)-piperidin-4- yl | 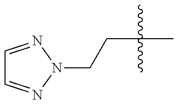 [SMILES: *CCn1nccn1] | Cl | 4.09 | 473, 471 |
| I-706 | N-(MeSO₂)-piperidin-4- | tetrahydropyran-4-yl- | Cl | 4.58 | 510, 508 |
| | yl | CH₂CH₂— | | | |
| I-707 | N-(MeSO₂)-piperidin-4- yl | 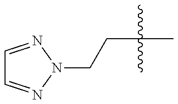 [SMILES: *CCn1nccn1] | Cl | 4.08 | 493, 491 |
| I-708 | 5-CF₃-pyridin-2-yl | N—(MEO₂C)-piperidin-4- | Br | 6.68 | 595, 593 |
| | | yl-CH₂CH₂— | | | |
| I-709 | 5-CF₃-pyridin-2-yl | N—Ac-piperidin-4-yl- | Br | 6.00 | 579, 577 |
| | | CH₂CH₂— | | | |
| I-710 | c-Pr | N-(4-F—C₆H₄—C(═O))- | Cl | 4.95 | 510, 508 |
| | | piperidin-4-yl-CH₂CH₂— | | | |
| I-711 | c-Pr | N-(c-PrC(═O)))- | Cl | 4.57 | 456, 454 |
| | | piperidin-4-yl-CH₂CH₂— | | | |
| I-712 | c-Pr | N—(HOC(Me)₂C(═O))- | Cl | 4.27 | 474, 472 |
| | | piperidin-4-yl-CH₂CH₂— | | | |

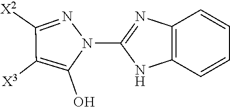

| Cpd | | | t_{R} | Mass |
|---|---|---|---|---|
| No. | X² | X³ | (min) | observed |
| I-713 | 4-CF₃—C₆H₄— | N—Ac-piperidin-4-yl- | 4.43 | 470 |
| I-714 | 5-CF₃-pyridin-2-yl | N—Ac-piperidin-4-yl-CH₂CH₂— | 4.53 | 499 |
| I-715 | 5-CF₃-pyridin-2-yl | N—(MeO₂C)-piperidin-4-yl-CH₂CH₂— | 5.19 | 515 |
| I-716 | 5-CF₃-pyridin-2-yl | N—MeSO₂-piperidin-4-yl-CH₂CH₂— | 4.91 | 535 |
| I-717 | 5-Br-pyridin-2-yl | N—(i-PrO₂C)-piperidin-4-yl-CH₂CH₂— | 5.49 | 555, 553 |
| I-718 | 4-CF₃—C₆H₄— | N—MeSO₂-piperidin-4-yl-CH₂— | 4.72 | 520 |
| I-719 | 4-CF₃—C₆H₄— | N—Ac-piperidin-4-yl-CH₂CH₂— | 4.04 | 498 |
| I-720 | 4-CF₃—C₆H₄— | N—Ac-piperidin-4-yl-CH₂— | 4.42 | 484 |

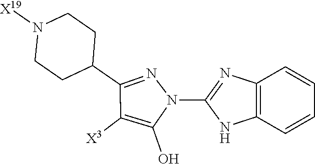

| Cpd | | | t_{R} | Mass |
|---|---|---|---|---|
| No. | X¹⁹ | X³ | (min) | observed |
| I-721 | i-BuO₂C— | 4-F—C₆H₄—CH₂CH₂— | 5.32 | 506 |
| I-722 | i-PrO₂C— | tetrahydropyran-4-yl-CH₂CH₂— | 4.21 | 482 |
| I-723 | EtO₂C— | tetrahydropyran-4-yl-CH₂CH₂— | 3.97 | 468 |
| I-724 | Me₂NSO₂— | tetrahydropyran-4-yl-CH₂CH₂— | 397 | 503 |
| I-725 | i-PrO₂c— | tetrahdyropyran-4-yl-CH₂— | 4.05 | 468 |
| I-726 | 1-Me-piperazin-4-yl- | 4-F—C₆H₄—CH₂CH₂— | 3.01 | 546 |
| | CH₂CO | | | |
| I-727 | (R)-2-MeO-propionyl | 4-F—C₆H₄—CH₂CH₂— | 4.20 | 492 |
| I-728 | pyrazin-2-carbonyl | 4-F—C₆H₄—CH₂CH₂— | 4.13 | 512 |
| I-729 | 2,4,6-trihydroxybenzoyl | 4-F—C₆H₄—CH₂CH₂— | 2.76 | 504 |
| I-730 | pyridin-2-yl-carbonyl | 4-F—C₆H₄—CH₂CH₂— | 4.10 | 511 |
| I-731 | 6-HO-pyridin-2-yl-carbonyl | 4-F—C₆H₄—CH₂CH₂— | 3.83 | 527 |
| I-732 | (2S,3S)-2-hydroxy-3-Me- | 4-F—C₆H₄—CH₂CH₂— | 4.72 | 520 |
| | butanoyl | | | |
| I-733 | (S)-2-hydroxy-propionyl | 4-F—C₆H₄—CH₂CH₂— | 4.28 | 492 |
| I-734 | pyridin-4-yl-CH₂CO— | 4-F—C₆H₄—CH₂CH₂— | 2.97 | 525 |
| I-735 | 4-HO-cyclohexyl-carbonyl | 4-F—C₆H₄—CH₂CH₂— | 4.03 | 532 |
| I-736 | tetrahydrofuran-3-yl- | 4-F—C₆H₄—CH₂CH₂— | 4.13 | 504 |
| | carbonyl | | | |
| I-737 | furyl-3-carbonyl | 4-F—C₆H₄—CH₂CH₂— | 4.37 | 500 |
| I-738 | 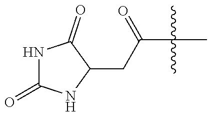 [SMILES: *C(=O)CC1NC(=O)NC1=O] | 4-F—C₆H₄—CH₂CH₂— | 3.58 | 546 |
| I-739 | Me₂NCO— | 4-F—C₆H₄—CH₂CH₂— | 3.57 | 467 |
| I-740 | MeO₂C— | 4-F—C₆H₄—CH₂CH₂— | 4.45 | 464 |
| I-741 | N-acetylpiperidin-4-yl | 4-F—C₆H₄—CH₂CH₂— | 3.72 | 448 |
| I-742 | pyridin-3-SO₂— | 4-F—C₆H₄—CH₂CH₂— | 4.38 | 547 |
| I-743 | MeSO₂— | 4-F—C₆H₄—CH₂CH₂— | 4.07 | 484 |
| I-744 | Acetyl | 3,4-diF-C₆H₃—CH₂— | 3.98 | 452 |
| I-745 | HOC(Me)₂CO— | 4-F—C₆H₄—CH₂CH₂— | 4.15 | 492 |
| I-746 | Me₂NSO₂— | 4-F—CH₄—CH₂CH₂— | 4.55 | 513 |
| I-747 | cyclohexyl-SO₂— | 4-F—C₄H₄—CH₂CH₂— | 5.07 | 552 |
| I-748 | c-Pr—SO₂— | 4-F—C₆H₄—CH₂CH₂— | 4.64 | 510 |
| I-749 | 4-H₂N—C₆H₄—SO₂— | 4-F—C₆H₄—CH₂CH₂— | 4.52 | 561 |
| I-750 | i-Bu—SO₂— | 4-F—C₆H₄—CH₂CH₂— | 4.87 | 526 |
| I-751 | i-Pr—SO₂— | 4-F—CH₄—CH₂CH₂— | 4.61 | 512 |
| I-752 | 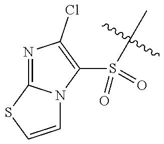 [SMILES: *S(=O)(=O)c1c(Cl)nc2sccn12] | 4-F—C₆H₄—CH₂CH₂— | 4.87 | 628, 626 |
| I-753 | cyclopentyl-SO₂— | 4-F—C₆H₄—CH₂CH₂— | 4.93 | 538 |

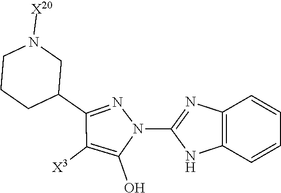

| Cpd | | | t_{R} | Mass |
|---|---|---|---|---|
| No. | X²⁰ | X³ | (min) | observed |
| I-754 | Acetyl | 4-F—C₆H₄—CH₂CH₂— | 4.07 | 448 |
| I-755 | Boc | 4-F—C₆H₄—CH₂CH₂— | 5.13 | 506 |
| I-756 | pyrazin-2-yl-CO— | 4-F—C₆H₄—CH₂CH₂— | 4.12 | 512 |
| I-757 | pyridin-4-yl-CH₂—CO— | 4-F—C₆H₄—CH₂CH₂— | 3.38 | 525 |
| I-758 | pyridin-3-yl-SO₂— | 4-F—C₆H₄—CH₂CH₂— | 4.67 | 547 |

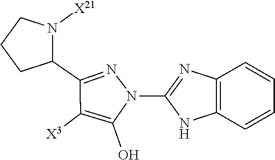

| Cpd | | | t_{R} | Mass |
|---|---|---|---|---|
| No. | X²¹ | X³ | (min) | observed |
| I-759 | pyrazin-2-yl-CO— | 4-F—C₆H₄—CH₂CH₂— | 4.02 | 498 |
| I-760 | pyridin-4-yl-CH₂—CO— | 4-F—C₆H₄—CH₂CH₂— | 3.43 | 511 |
| I-761 | HOC(Me)₂CO— | 4-F—C₆H₄—CH₂CH₂— | 4.18 | 478 |
| I-762 | 2,4,6-trihydroxybenzoyl- | 4-F—C₆H₄—CH₂CH₂— | 3.93 | 490 |
| I-763 | morpholin-4-yl-CH₂— | 4-F—C₆H₄—CH₂CH₂— | 3.47 | 519 |

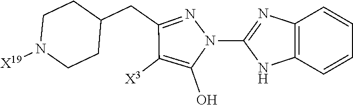

| Cpd | | | t_{R} | Mass |
|---|---|---|---|---|
| No. | X¹⁹ | X³ | (min) | observed |
| I-764 | Cbz | 4-F—C₆H₄—CH₂CH₂— | 5.19 | 554 |
| I-765 | tetrahydrofuran-3-yl-carbonyl | 4-F—C₆H₄—CH₂CH₂— | 4.09 | 518 |
| I-766 | 6-HO-pyridin-2-yl-carbonyl | 4-F—C₆H₄—CH₂CH₂— | 3.74 | 541 |
| I-767 | pyrazin-2-yl-CO— | 4-F—C₆H₄—CH₂CH₂— | 4.01 | 526 |
| I-768 | 1-Me-piperazin-4-yl-CH₂CO— | 4-F—C₆H₄—CH₂CH₂— | 2.99 | 560 |
| I-769 | pyridin-4-yl-CH₂OCH₂CO— | 4-F—C₆H₄—CH₂CH₂— | 3.29 | 569 |
| I-770 | Boc | 4-F—C₆H₄—CH₂CH₂— | 5.28 | 520, 420 |
| I-771 | MeSO₂— | 4-F—C₆H₄—CH₂CH₂— | 4.23 | 498 |
| I-772 | Acetyl | 4-F—C₆H₄—CH₂CH₂— | 4.09 | 462 |

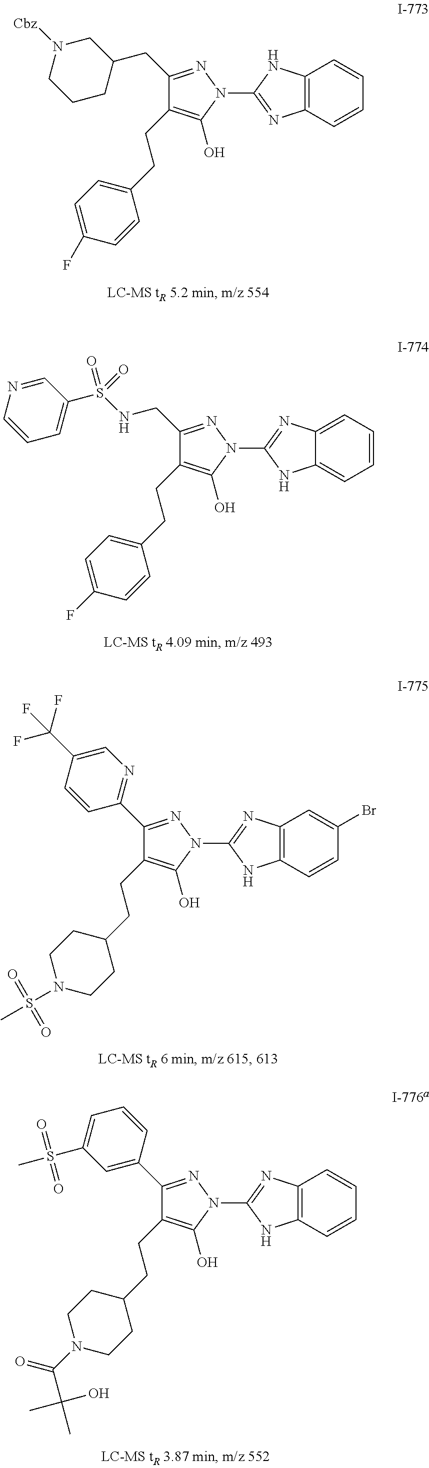 [SMILES: O=C(OCc1ccccc1)N1CCCC(Cc2nn(-c3nc4ccccc4[nH]3)c(O)c2CCc2ccc(F)cc2)C1 ; O=S(=O)(NCc1nn(-c2nc3ccccc3[nH]2)c(O)c1CCc1ccc(F)cc1)c1cccnc1 ; CS(=O)(=O)N1CCC(CCc2c(-c3ccc(C(F)(F)F)cn3)nn(-c3nc4cc(Br)ccc4[nH]3)c2O)CC1 ; CC(C)(O)C(=O)N1CCC(CCc2c(-c3cccc(S(C)(=O)=O)c3)nn(-c3nc4ccccc4[nH]3)c2O)CC1]

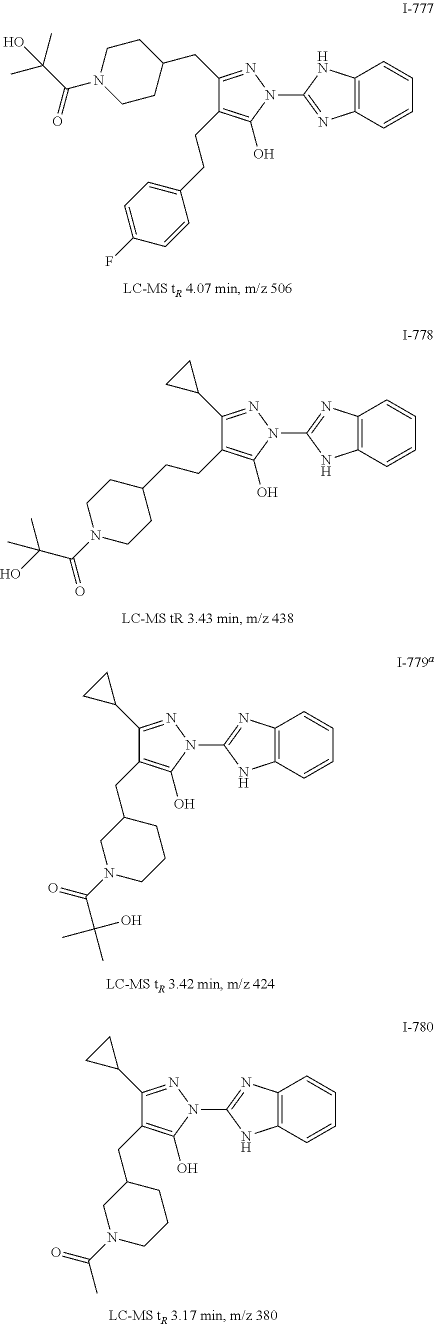 [SMILES: CC(C)(O)C(=O)N1CCC(Cc2nn(-c3nc4ccccc4[nH]3)c(O)c2CCc2ccc(F)cc2)CC1 ; CC(C)(O)C(=O)N1CCC(CCc2c(C3CC3)nn(-c3nc4ccccc4[nH]3)c2O)CC1 ; CC(C)(O)C(=O)N1CCCC(Cc2c(C3CC3)nn(-c3nc4ccccc4[nH]3)c2O)C1 ; CC(=O)N1CCCC(Cc2c(C3CC3)nn(-c3nc4ccccc4[nH]3)c2O)C1]

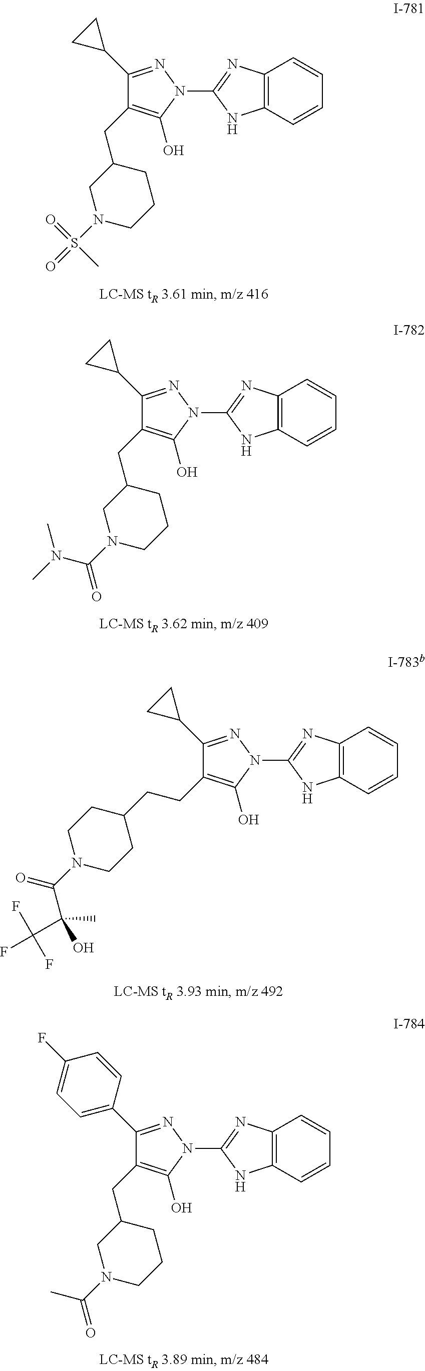 [SMILES: CS(=O)(=O)N1CCCC(Cc2c(C3CC3)nn(-c3nc4ccccc4[nH]3)c2O)C1 ; CN(C)C(=O)N1CCCC(Cc2c(C3CC3)nn(-c3nc4ccccc4[nH]3)c2O)C1 ; C[C@@](O)(C(=O)N1CCC(CCc2c(C3CC3)nn(-c3nc4ccccc4[nH]3)c2O)CC1)C(F)(F)F ; CC(=O)N1CCCC(Cc2c(-c3ccc(F)cc3)nn(-c3nc4ccccc4[nH]3)c2O)C1]

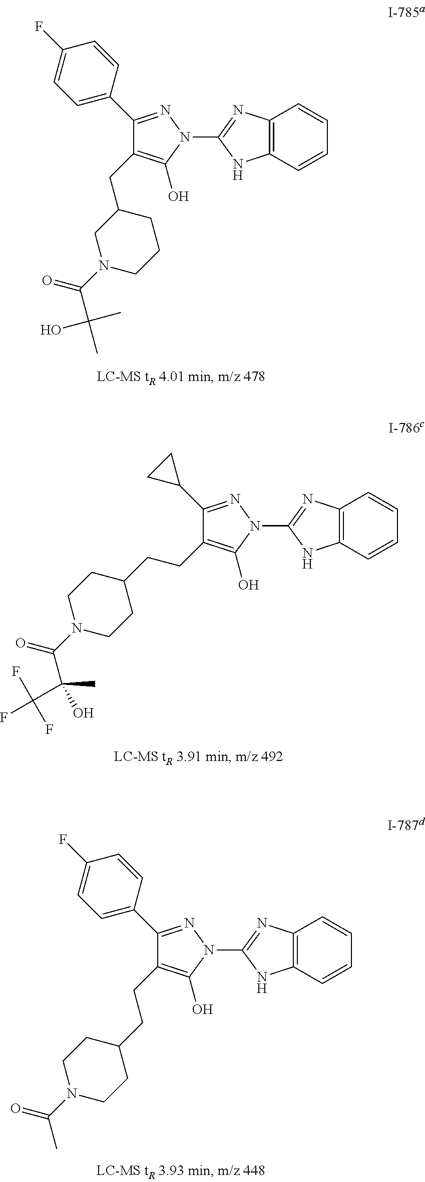 [SMILES: CC(C)(O)C(=O)N1CCCC(Cc2c(-c3ccc(F)cc3)nn(-c3nc4ccccc4[nH]3)c2O)C1 ; C[C@](O)(C(=O)N1CCC(CCc2c(C3CC3)nn(-c3nc4ccccc4[nH]3)c2O)CC1)C(F)(F)F ; CC(=O)N1CCC(CCc2c(-c3ccc(F)cc3)nn(-c3nc4ccccc4[nH]3)c2O)CC1]

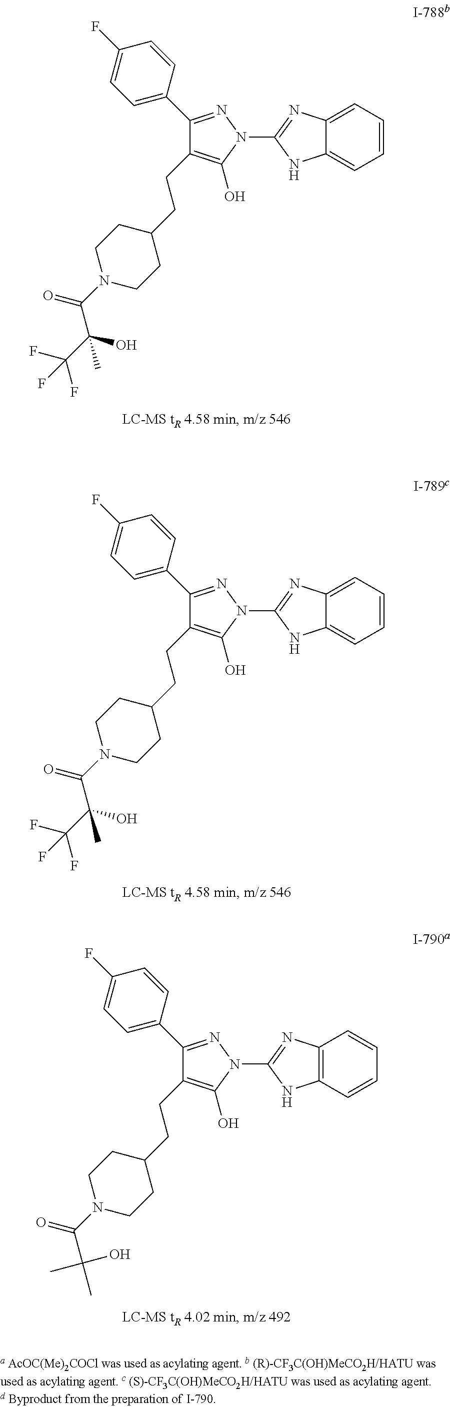 [SMILES: C[C@@](O)(C(=O)N1CCC(CCc2c(-c3ccc(F)cc3)nn(-c3nc4ccccc4[nH]3)c2O)CC1)C(F)(F)F ; C[C@](O)(C(=O)N1CCC(CCc2c(-c3ccc(F)cc3)nn(-c3nc4ccccc4[nH]3)c2O)CC1)C(F)(F)F ; CC(C)(O)C(=O)N1CCC(CCc2c(-c3ccc(F)cc3)nn(-c3nc4ccccc4[nH]3)c2O)CC1]

### Example 8

### 1-(1H-1,3-benzodiazol-2-yl)-4-[2-(4-fluorophenyl)ethyl]-3-(1-methylpiperidin-4-yl)-1H-pyrazol-5-ol (I-801)

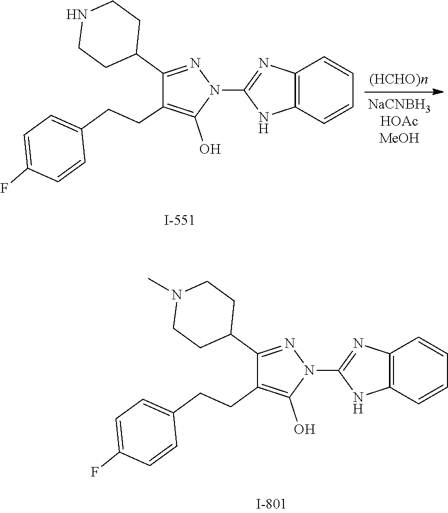 [SMILES: Oc1c(CCc2ccc(F)cc2)c(C2CCNCC2)nn1-c1nc2ccccc2[nH]1 ; CN1CCC(c2nn(-c3nc4ccccc4[nH]3)c(O)c2CCc2ccc(F)cc2)CC1]

To a stirred solution of I-551 HCl salt (26 mg, 60 μmol) in MeOH (1 mL) were added paraformaldehyde (9 mg, 0.3 mmol) and HOAc (100 μL). The mixture was stirred at rt for 1.5 h and NaCNBH3 (10 mg, 0.15 mmol) was added. After stirring overnight the mixture was concentrated and the residue was purified by prep HPLC to give (I-801 (19 mg, 59% yield). ¹H NMR (CD₃OD) δ: 7.63 (dd, J=6.0, 3.2 Hz, 1H), 7.39 (dd, J=6.0, 3.2 Hz, 2H), 7.18-7.24 (m, 2H), 6.96-7.03 (m, 2H), 3.52-3.59 (m, 2H), 3.01-3.06 (m, 2H), 2.84-2.94 (m, 6H), 2.63-2.74 (m, 2H), 1.85-2.02 (m, 4H); LC-MS t_{R} 3.97 min, m/z 420.

The following compounds are prepared using analogous procedures.

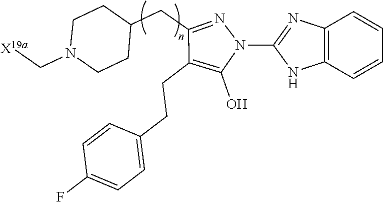

| Cpd. No. | X^{19a} | n | t_{R} | Mass observed |
|---|---|---|---|---|
| I-802 | pyridin-2-yl | 0 | 3.39 | 497 |
| I-803 | pyridin-3-yl | 0 | 3.39 | 497 |
| I-804 | pyridin-4-yl | 0 | 3.15 | 497 |
| I-805 | 2-(pyrazol-1-yl)phenyl | 0 | 3.92 | 562 |
| I-806 | tetrahydropyran-4-yl | 0 | 3.28 | 504 |
| I-807 | pyridin-2-yl | 1 | 3.42 | 511 |
| I-808 | pyridin-3-yl | 1 | 2.97 | 511 |
| I-809 | pyridin-4-yl | 1 | 2.90 | 511 |

The following compounds are made using analogous procedures.

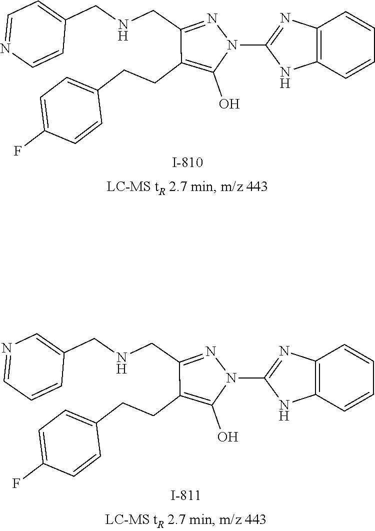 [SMILES: Oc1c(CCc2ccc(F)cc2)c(CNCc2ccncc2)nn1-c1nc2ccccc2[nH]1 ; Oc1c(CCc2ccc(F)cc2)c(CNCc2cccnc2)nn1-c1nc2ccccc2[nH]1]

### Example 9

### 1-(5-chloro-1H-1,3-benzodiazol-2-yl)-3-cyclopropyl-4-{2-[1-(5-fluoropyrimidin-2-yl)piperidin-4-yl]ethyl}-1H-pyrazol-5-ol (I-901)

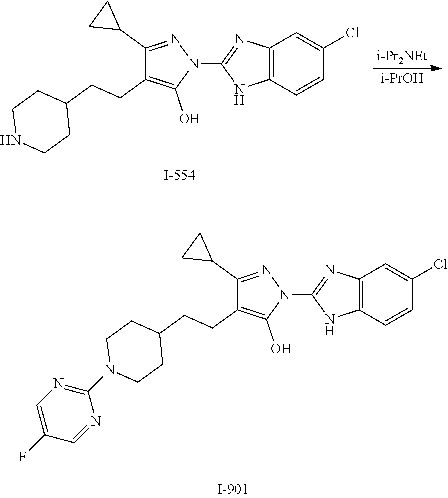 [SMILES: "i-Pr2NEt" ; "i-PrOH" ; Oc1c(CCC2CCNCC2)c(C2CC2)nn1-c1nc2cc(Cl)ccc2[nH]1 ; Oc1c(CCC2CCN(c3ncc(F)cn3)CC2)c(C2CC2)nn1-c1nc2cc(Cl)ccc2[nH]1]

A mixture of I-554.HCl salt (50 mg, 0.11 mmol), 2-chloro-5-fluoropyrimidine (14.5 mg, 0.11 mmol), i-Pr₂NEt (78 μL, 0.44 mmol) and i-PrOH (2 mL) was stirred at 80° C. for 1 d and at 100° C. for 1 d. Preparative HPLC gave I-901 (3.6 mg). ¹H NMR (CD₃OD) δ: 8.19-8.29 (m, 2H), 7.44-7.71 (m, 2H), 7.32 (dd, J=8.6, 1.9 Hz, 1H), 4.66 (dt, J=13.0, 2.7 Hz, 2H), 2.73-2.93 (m, 3H), 2.37-2.67 (m, 2H), 1.72-2.03 (m, 3H), 1.43-1.71 (m, 3H), 0.96-1.31 (m, 5H); LC-MS t_{R} 5.57 min, 484, 482.

The following compounds are prepared using analogous procedures.

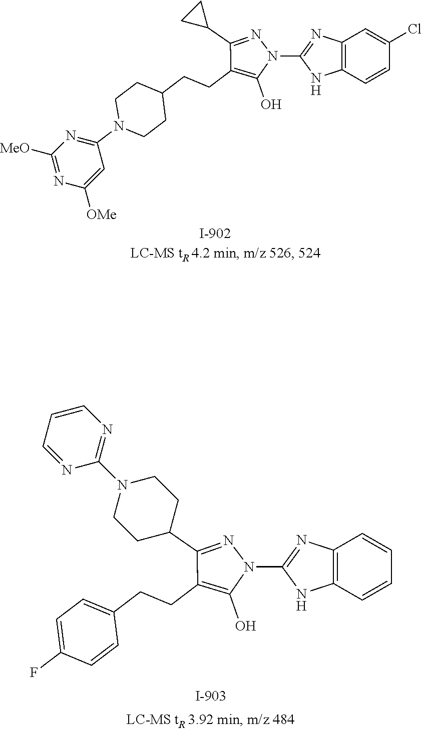 [SMILES: COc1cc(N2CCC(CCc3c(C4CC4)nn(-c4nc5cc(Cl)ccc5[nH]4)c3O)CC2)nc(OC)n1 ; Oc1c(CCc2ccc(F)cc2)c(C2CCN(c3ncccn3)CC2)nn1-c1nc2ccccc2[nH]1]

### Example 10

### 1-(1H-1,3-benzodiazol-2-yl)-4-[(3,4-difluorophenyl)methyl]-3-[1-(2,2,2-trifluoroethyl)piperidin-4-yl]-1H-pyrazol-5-ol (I-1001)

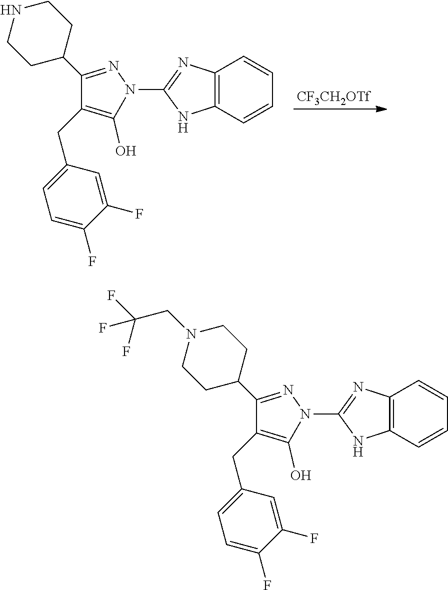

To a stirred solution of I-509.HBr salt (22 mg, 45 μmol) and i-Pr₂NEt (25 μL, 0.14 mmol) in EtOH (1 mL) was added CF₃CH₂OTf (7 μL, 49 μmol). The mixture was stirred at rt for 2 d and purified by prep HPLC to give I-1001 (7.5 mg). LC-MS t_{R} 3.84 min, m/z 492.

The following compounds are prepared using analogous procedures.

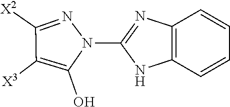

| Cpd. | | | t_{R} | Mass |
|---|---|---|---|---|
| No. | X² | X³ | (min) | observed |
| I-1002 | N—CF₃CH₂-piperidin-3-yl | 4-F—C₆H₄—CH₂CH₂— | 4.18 | 488 |
| I-1003 | N—CF₃CH₂-piperidin-4-yl | 4-F—C₆H₄—CH₂CH₂— | 3.92 | 488 |
| I-1004 | N—CF₃CH₂-piperidin-4-yl- | 4-F—C₆H₄—CH₂CH₂— | 3.57 | 502 |
| | CH₂— | | | |
| I-1005 | 4-CF₃—C₆H₄— | N—CF₃CH₂-piperidin-4-yl- | 4.02 | 524 |
| | | CH₂— | | |
| I-1006 | 4-CF₃—C₆H₄— | N—CF₃CH₂-piperidin-4-yl- | 4.03 | 538 |
| | | CH₂CH₂— | | |

### Example 11

### 4-[1-(1H-1,3-benzodiazol-2-yl)-4-[2-(4-fluorophenyl)ethyl]-5-hydroxy-1H-pyrazol-3-yl]benzoic acid (I-1101)

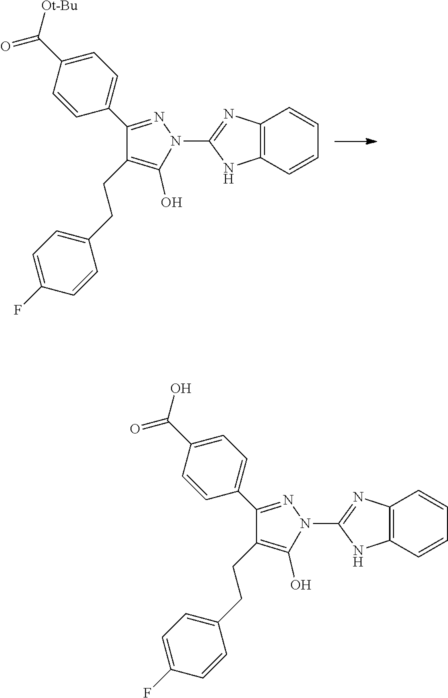

A solution of I-208 (34 mg, 68 μmol) in TFA (4 mL) was stirred at rt for 3 h and concentrated. The residue was purified by prep HPLC to give I-1101 (20 mg) as a solid. ¹H NMR (CD₃OD) δ: 8.09-8.14 (m, 2H), 7.69-7.75 (m, 4H), 7.48-7.54 (m, 2H), 7.03-7.08 (m, 2H), 6.85-6.92 (m, 2H), 2.87-2.97 (m, 2H), 2.75-2.85 (m, 2H); LC-MS t_{R} 3.95 min, m/z 443.

### Example 12

### 4-[1-(1H-1,3-benzodiazol-2-yl)-4-[2-(4-fluorophenyl)ethyl]-5-hydroxy-1H-pyrazol-3-yl]-N-(cyclopropylmethyl)benzamide (I-1201)

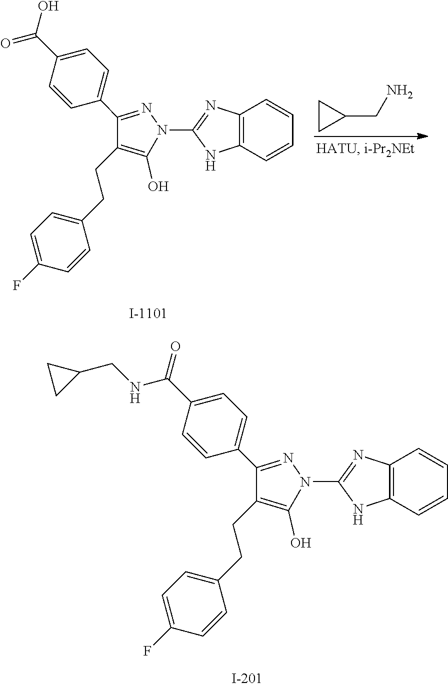

To a stirred solution of I-1101 (15 mg, 34 μmol), i-Pr₂NEt (50 μL, 0.27 μmmol) and c-PrNH₂.HCl (15 mg, 0.14 mmol) in CH₂Cl₂ (1 mL) was added solid HATU (20 mg, 51 μmol). After stirring overnight, the mixture was concentrated and the residue was purified by prep HPLC to give I-1201 (6 mg) as a solid. ¹H NMR (CD₃OD) δ: 7.91-7.97 (m, 2H), 7.69-7.77 (m, 4H), 7.51-7.59 (m, 2H), 7.06 (m, 2H), 6.90 (m, 2H), 2.95 (m, 2H), 2.77 (m, 2H), 2.20 (s, 1H), 1.94 (s, 1H), 1.28 (m, 1H), 0.55 (m, 1H), 0.31 (m, 1H), −0.02-0.03 (m, 3H); LC-MS t_{R} 4.46 min, m/z 496.

### Example 13

### 2-{4-[2-(4-fluorophenyl)ethyl]-5-methyl-3-phenyl-1H-pyrazol-1-yl}-1H-1,3-benzodiazole (I-1301)

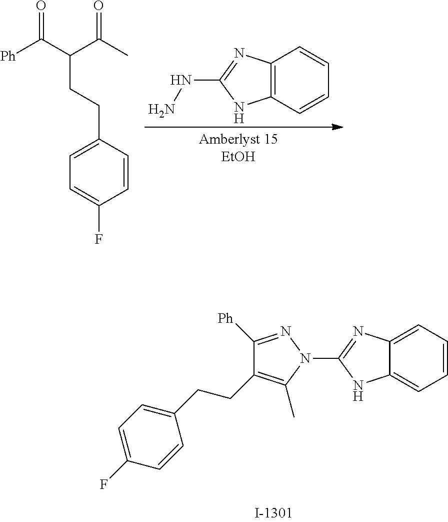 [SMILES: CC(=O)C(CCc1ccc(F)cc1)C(=O)c1ccccc1 ; NNc1nc2ccccc2[nH]1 ; Cc1c(CCc2ccc(F)cc2)c(-c2ccccc2)nn1-c1nc2ccccc2[nH]1]

A mixture of 2-oxo-3-benzoyl-5-(4-fluorophenyl)pentane (100 mg, 0.35 mmol), 2-hydrazinobenzimidazole (104 mg, 0.70 mmol), Amberlyst 15 (25 mg) and EtOH (1.75 mL) was heated at 60° C. for 2 d. The mixture was filtered and the filtrate was purified by prep HPLC to give I-1301 (138 mg, quant) as a white powder. ¹H NMR (DMSO-d₆) δ: 7.30-7.47 (m, 4H), 7.22-7.27 (m, 2H), 6.97-7.19 (m, 7H), 2.53-2.78 (m, 4H), 2.22 (s, 3H); LC-MS t_{R} 5.33 min, m/z 397.

### Example 14

### 1-(1H-1,3-benzodiazol-2-yl)-3-(4-chlorophenyl)-4-[2-(4-fluorophenyl)ethyl]-1H-pyrazol-5-amine (I-1401)

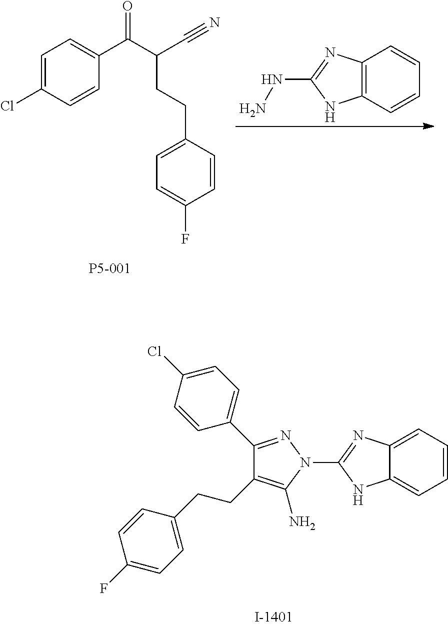 [SMILES: N#CC(CCc1ccc(F)cc1)C(=O)c1ccc(Cl)cc1 ; NNc1nc2ccccc2[nH]1 ; Nc1c(CCc2ccc(F)cc2)c(-c2ccc(Cl)cc2)nn1-c1nc2ccccc2[nH]1]

A mixture of P5-001 (99 mg, 0.33 mmol), 2-hydrazinobenzimidazole (94 mg, 0.65 mmol), TsOH.H₂O (cat. qty.) and EtOH (2 mL) was stirred at 80 C for 3 d. Prep HPLC gave I-1401 (15 mg) as an off white solid. ¹H NMR (CD₃OD) δ: 7.52-7.57 (m, 4H), 7.40-7.43 (m, 2H), 7.23-7.26 (m, 2H), 7.06-7.11 (m, 2H), 6.89 (t, J=8.2 Hz, 2H), 2.73-2.87 (m, 4H); LC-MS t_{R} 7.53 min, m/z 434, 432

The following compound is prepared by an analogous procedure.

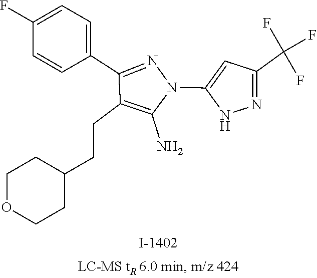 [SMILES: Nc1c(CCC2CCOCC2)c(-c2ccc(F)cc2)nn1-c1cc(C(F)(F)F)n[nH]1]

### Example 15

### 3-(4-fluorophenyl)-4-(2-phenylethyl)-5′-(trifluoromethyl)-2′H-1,3′-bipyrazol (I-1501)

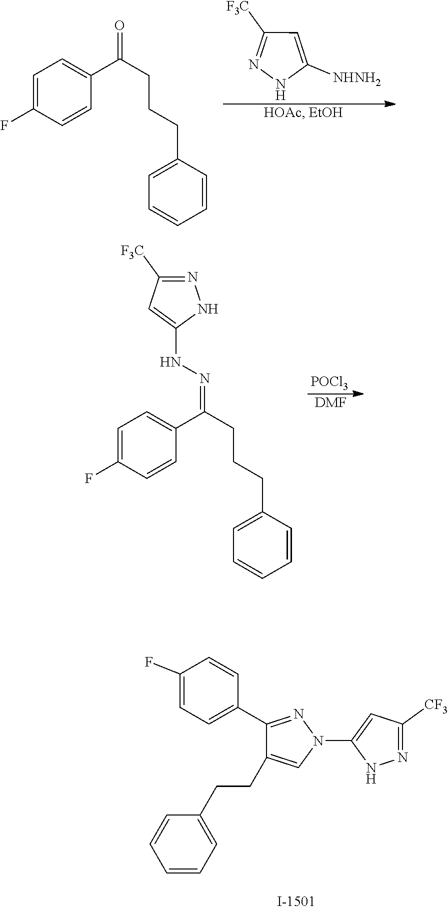

### Step 1

A mixture of 1-(4-fluorophenyl)-4-phenylbutan-1-one (P9-001, 146 mg, 0.60 mmol), 3-hydrazino-5-(trifluoromethyl)pyrazole (100 mg, 0.60 mmol), HOAc (0.5 mL) and EtOH (2 mL) was heated at 50° C. for 2 h and concentrated to leave crude hydrazone (249 mg) as an orange oil which was used without purification. LC-MS t_{R} 7.73 min, m/z 391.

### Step 2

To a stirred, ice-cold solution of crude hydrazine from Step 1 (34 mg, 87 μmol) in DMF (0.5 mL) was added POCl₃ (25 μL, 0.27 mmol). The mixture was heated at 90° C. for 3 h, diluted with EtOAc (45 mL), washed with satd aq NaHCO₃ (7 mL) and brine (7 mL), and dried over Na₂SO₄. Removal of the solvent left an oil (45 mg) which was applied to a 2-g silica SPE cartridge which was eluted sequentially with 0, 10, 25, 50 and 100% EtOAc in hexanes (10 mL of each) to give five fractions. Fractions 2 and 3 were combined and concentrated to leave an oil (8 mg). Prep HPLC gave I-1501 (1.9 mg) as a white solid. ¹H NMR (CD₃OD) δ 8.02 (s, 1H), 7.65 (dd, J=9.0, 5.5 Hz, 2H), 7.00-7.33 (m, 7H), 6.85 (s, 1H), 2.79-3.07 (m, 4H); LC-MS t_{R} 7.22 min, m/z 401.

The following compound is prepared by an analogous procedure.

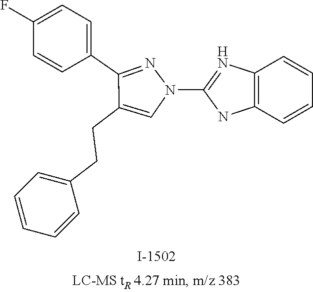

### Example 16

### 1-(4-{[1-(1H-1,3-benzodiazol-2-yl)-5-hydroxy-3-[4-(trifluoromethyl)phenyl]-1H-pyrazol-4-yl]methyl}piperidin-1-yl)ethan-1-one (I-720)

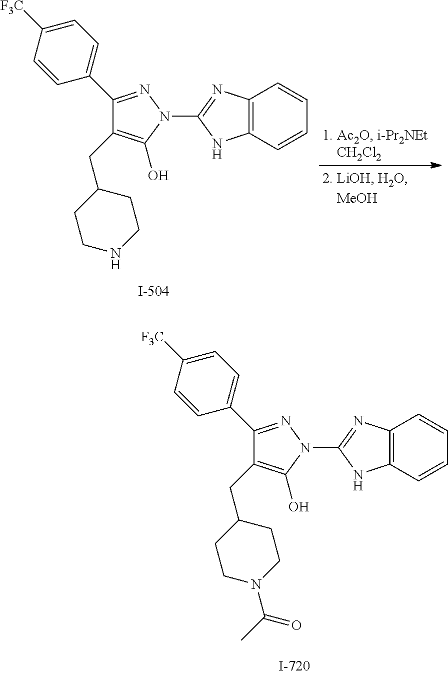 [SMILES: Oc1c(CC2CCNCC2)c(-c2ccc(C(F)(F)F)cc2)nn1-c1nc2ccccc2[nH]1 ; CC(=O)N1CCC(Cc2c(-c3ccc(C(F)(F)F)cc3)nn(-c3nc4ccccc4[nH]3)c2O)CC1]

To a stirred suspension of I-504 HBr salt (28 mg, 46 μmol) and i-Pr₂NEt (30 μL, 0.16 mmol) in CH₂Cl₂ (1 mL) was added acetic anhydride (6 μL, 63 μmol). The mixture was stirred at rt for 2 h and concentrated. The residue was taken up in 3:1 MeOH/H₂O (2 mL) and LiOH (˜25 mg) was added. After stirring overnight, the mixture was diluted with HOAc (1 mL) and purified by prep HPLC to give I-720 TFA salt (23 mg) as a white solid. ¹H NMR (CD₃OD) δ: 7.94-7.98 (m, J=8.1 Hz, 2H), 7.79-7.84 (m, J=8.2 Hz, 2H), 7.66-7.72 (m, 2H), 7.43-7.49 (m, 2H), 4.41 (br d, J=13.0 Hz, 1H), 3.82 (br d, J=13.6 Hz, 1H), 2.91-2.97 (m, 1H), 2.62 (d, J=6.7 Hz, 2H), 2.42-2.48 (m, 1H), 2.03 (s, 3H), 1.63-1.78 (m, 3H), 1.00-1.17 (m, 2H); LC-MS t_{R} 4.42 min, m/z 484.

### Example 17

### 3-cyclopropyl-4-[2-(4-fluorophenyl)ethyl]-1-{3H-imidazo[4,5-b]pyridin-2-yl}-1H-pyrazol-5-ol (I-308)

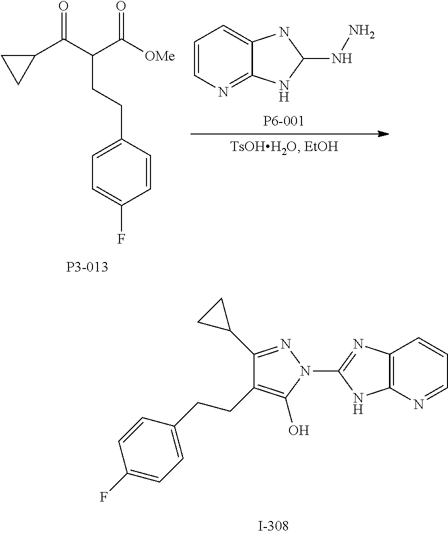

A mixture of P3-013 (40 mg, 0.14 mmol), P6-001 (42 mg, 0.29 mmol), TsOH.H₂O (cat qty) and EtOH (1 mL) was heated in the microwave at 130° C. for 3 h. Prep HPLC gave I-308 TFA salt (12 mg) as a white solid. ¹H NMR (CD₃OD) δ: 8.32 (d, J=5.1 Hz, 1H), 8.10 (dd, J=8.0, 1.4 Hz, 1H), 7.39 (dd, J=8.0, 5.4 Hz, 1H), 7.18-7.23 (m, 2H), 6.93-6.97 (m, 2H), 2.85-2.91 (m, 2H), 2.69-2.74 (m, 2H), 1.69-1.77 (m, 1H), 0.89-0.98 (m, 4H); LC-MS t_{R} 3.62 min, m/z 364

### Example 18

### 1-(4-{2-[1-(5-chloro-1H-1,3-benzodiazol-2-yl)-3-cyclopropyl-5-hydroxy-1H-pyrazol-4-yl]ethyl}piperidin-1-yl)-2-hydroxy-2-methylpropan-1-one (I-712)

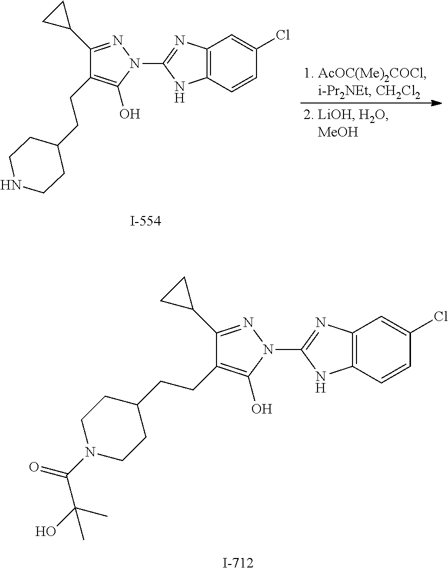 [SMILES: Oc1c(CCC2CCNCC2)c(C2CC2)nn1-c1nc2cc(Cl)ccc2[nH]1 ; CC(C)(O)C(=O)N1CCC(CCc2c(C3CC3)nn(-c3nc4cc(Cl)ccc4[nH]3)c2O)CC1]

To a stirred mixture of I-554 HCl salt (50 mg, 0.11 mmol), i-Pr₂NEt (0.1 mL, 0.55 mmol) and CH₂Cl₂ (2 mL) was added 1-chloro-2-methyl-1-oxopropan-2-yl acetate (32 μL, 0.22 mmol). The mixture was stirred at rt for 2 h and concentrated. The residue was taken up in 3:1 MeOH/H₂O (4 mL) and LiOH (˜75 mg) was added. After stirring overnight, the mixture was purified by prep HPLC to give I-712 TFA salt (10 mg) as a solid. ¹H NMR (CD₃OD) δ: 7.54-7.70 (m, 2H), 7.34-7.40 (m, 1H), 4.95-5.03 (m, 2H), 4.69-4.88 (m, 2H), 2.92-3.12 (m, 1H), 2.46-2.55 (m, 2H), 1.74-2.04 (m, 3H), 1.48-1.66 (m, 2H), 1.35 (s, 6H), 1.01-1.24 (m, 8H), 0.80-0.97 (m, 1H); LC-MS t_{R} 4.27 min, m/z 474, 472.

### Example 19

### 4-[2-(4-fluorophenyl)ethyl]-1-{1-[2-(morpholin-4-yl)ethyl]-1H-1,3-benzodiazol-2-yl}-3-[4-(trifluoromethyl)phenyl]-1H-pyrazol-5-ol (I-1901)

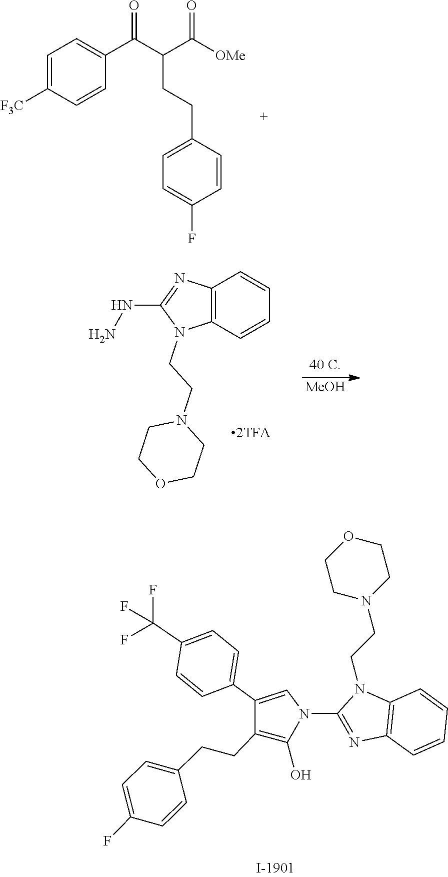

A mixture of methyl 4-(4-fluorophenyl)-2-[4-(trifluoromethyl)benzoyl]butanoate (254 mg, 0.70 mmol), -hydrazinyl-1-[2-(morpholin-4-yl)ethyl]-1H-1,3-benzodiazole bis TFA salt (135 mg, 0.28 mmol) and MeOH (2.5 mL) was stirred at 40° C. for 6 d. Prep HPLC gave the title compound bis TFA salt (49 mg, 22%) as a white solid. ¹H NMR (METHANOL-d₄) δ: 7.66-7.82 (m, 6H), 7.37-7.50 (m, 2H), 7.04 (dd, J=8.5, 5.5 Hz, 2H), 6.80-6.91 (m, 2H), 5.07 (t, J=6.6 Hz, 2H), 3.78 (t, J=6.6 Hz, 2H), 3.71 (br s, 4H), 3.35 (br s, 4H), 2.86 (dd, J=15.8, 6.1 Hz, 4H); LC-MS t_{R} 4.87 min, m/z 580.
¹H NMR data for selected compounds are provided below:

| | |
|---|---|
| I-148 | ¹H NMR (CD₃OD) δ: 8.02-8.05 (m, 2H), 7.83-7.86 (m, 2H), 7.73-7.76 (m, 2H), 7.52- |
| | 7.56 (m, 2H), 7.00-7.05 (m, 2H), 6.82-6.89 (m, 2H), 3.20 (s, 3H), 2.95-3.01 (m, 2H), |
| | 2.76-2.79 (m, 2H) |
| I-151 | ¹H NMR (CD₃OD) δ: 7.73-7.83 (m, 2H), 7.63-7.69 (m, 2H), 7.36-7.49 (m, 2H), 7.21- |
| | 7.32 (m, 2H), 3.80-3.88 (m, 2H), 3.16-3.28 (m, 2H), 2.56 (d, J = 7.2 Hz, 2H), 1.72 (td, |
| | J = 7.5, 3.7 Hz, 1H), 1.49-1.59 (m, 2H), 1.14-1.32 (m, 2H) |
| I-170 | ¹H NMR (CD₃OD) δ: 7.63-7.71 (m, 2H), 7.50 (d, J = 1.9 Hz, 1H), 7.18-7.23 (m, 2H), |
| | 6.93-7.00 (m, 3H), 2.85-2.91 (m, 2H), 2.75-2.81 (m, 2H), 1.72-1.80 (m, 1H), 0.95- |
| | 1.01 (m, 4H) |
| I-210 | ¹H NMR (CD₃OD) δ: 8.34 (t, J = 1.7 Hz, 1H), 8.04-8.13 (m, 2H), 7.79 (t, J = 7.6 Hz, |
| | 1H), 7.64-7.70 (m, 2H), 7.41-7.47 (m, 2H), 4.00 (br d, J = 13.2 Hz, 2H), 3.18 (s, 3H), |
| | 2.67 (m, 4H), 1.67 (br d, J = 13.0 Hz, 2H), 1.39-1.50 (m, 12H), 1.05 (br d, J = 11.8 Hz, |
| | 2H) |
| I-503 | ¹H NMR (CD₃OD) δ: 7.49-7.59 (m, 2H), 7.28 (dd, J = 8.6, 2.0 Hz, 1H), 7.17-7.23 (m, |
| | 2H), 6.95-7.03 (m, 2H), 3.41-3.49 (m, 2H), 2.96-3.07 (m, 2H), 2.83-2.90 (m, 2H), |
| | 2.63-2.74 (m, 3H), 1.76-1.98 (m, 4H). |
| I-604 | ¹H NMR (CD₃OD) δ: 8.12 (s, 1H), 7.96-7.98 (m, 1H), 7.84 (d, J = 7.4 Hz, 1H), 7.64- |
| | 7.67 (m, 1H), 7.06-7.11 (m, 2H), 6.86-6.89 (m, 2H), 6.83 (s, 1H), 3.16 (s, 3H), 2.84- |
| | 2.85-2.92 (m, 2H), 2.76-2.83 (m, 2H) |
| I-721 | ¹H NMR (CD₃OD) δ: 7.64 (dd, J = 6.1, 3.2 Hz, 2H), 7.40 (dd, J = 6.1, 3.2 Hz, 2H), 7.17- |
| | 7.22 (m, 2H), 6.99 (t, J = 8.2 Hz, 2H), 4.16 (br d, J = 13.5 Hz, 1H), 3.86 (d, J = 6.6 Hz, |
| | 2H), 2.77-2.89 (m, 4H), 2.72 (d, J = 6.7 Hz, 2H), 2.45-2.55 (m, 1H), 1.92 (d, J = 6.6 Hz, |
| | 2H), 1.54-1.68 (m, 4H), 0.95 (d, J = 6.8 Hz, 6H) |
| I-747 | ¹H NMR (CD₃OD) δ: 7.63-7.67 (m, 2H), 7.39-7.43 (m, 2H), 7.17-7.22 (m, 2H), 6.99 |
| | (t, J = 8.3 Hz, 2H), 3.77-3.83 (m, 2H), 3.03-3.05 (m, 2H), 2.82-2.95 (m, 4H), 2.68-2.74 |
| | (m, 2H), 2.41-2.49 (m, 1H), 2.06-2.13 (m, 2H), 1.85-1.92 (m, 2H), 1.68-1.79 (m, 3H), |
| | 1.05-1.59 (m, 6H) |
| I-746 | ¹H NMR (CD₃OD) δ: 7.61-7.65 (m, 2H), 7.37-7.40 (m, 2H), 7.17-7.22 (m, 2H), 6.96- |
| | 7.02 (m, 2H), 3.71 (br d, J = 12.5 Hz, 2H), 2.79-2.87 (m, 10H), 2.67-2.73 (m, 2H), |
| | 2.45-2.55 (m, 1H),1.65-1.80 (m, 2H), 1.55-1.64 (s, 2H) |
| I-110 | 1H NMR (CD₃OD) δ: 7.54-7.75 (m, 2H), 7.36-7.51 (m, 2H), 7.19 (dd, J = 8.5, 5.5 Hz, |
| | 2H), 6.88-7.09 (m, 2H), 4.10 (br d, J = 13.3 Hz, 2H), 2.61-2.94 (m, 6H), 2.43-2.55 (m, |
| | 1H), 1.52-1.81 (m, 4H), 1.46 (s, 9H) |
| I-161 | 1H NMR (CD₃OD) δ: 8.35 (dd, J = 5.3, 1.4 Hz, 1H), 8.05-8.12 (m, 1H), 7.65-7.80 (m, |
| | 4H), 7.38 (dd, J = 8.0, 5.3 Hz, 1H), 7.06 (dd, J = 8.6, 5.4 Hz, 2H), 6.79-6.91 (m, 2H), |
| | 2.78-2.94 (m, 5H) |
| I-119 | 1H NMR (CD₃OD) δ: 8.94 (d, J = 2.2 Hz, 1H), 8.19-8.25 (m, 1H), 8.14 (d, J = 2.3 Hz, |
| | 1H), 7.63-7.71 (m, 2H), 7.39-7.47 (m, 2H), 7.16 (dd, J = 8.4, 5.5 Hz, 2H), 6.87 (t, J = 8.8 |
| | Hz, 2H), 3.08-3.18 (m, 2H), 2.79-2.89 (m, 2H). |
| I-123 | 1H NMR (CD₃OD) δ: 7.90-8.01 (m, 2H), 7.80 (d, J = 8.0 Hz, 2H), 7.59-7.70 (m, 2H), |
| | 7.32-7.45 (m, 2H), 2.64 (d, J = 7.5 Hz, 2H), 1.92-2.14 (m, 1H), 1.37-1.74 (m, 6H), 1.06- |
| | 1.24 (m, 2H) |
| I-171 | 1H NMR (CD₃OD) δ: 7.81 (d, J = 7.8 Hz, 2H), 7.40-7.63 (m, 7H), 7.29 (dd, J = 8.6, 2.0 |
| | Hz, 1H), 7.18 (d, J = 7.9 Hz, 2H), 3.86 (s, 3H), 3.30 (br s, 2H), 2.89 (br s, 2H). |
| I-216 | 1H NMR (CD₃OD) δ: 7.52-7.63 (m, 2H), 7.31 (dd, J = 8.6, 2.0 Hz, 1H), 7.14-7.23 (m, |
| | 2H), 6.93-7.05 (m, 2H), 4.10 (br d, J = 13.3 Hz, 2H), 2.62-2.90 (m, 6H), 2.42-2.58 (m, |
| | 1H), 1.70-1.48 (br dd, J = 17.5, 4.3 Hz, 4H), 1.46 (s, 9H). |
| I-302 | 1H NMR (CD₃OD) δ: 7.58-7.71 (m, 2H), 7.41 (dd, J = 6.1, 3.2 Hz, 2H), 7.18 (dd, |
| | J = 8.5, 5.5 Hz, 2H), 6.90-7.04 (m, 2H), 3.39 (dd, J = 9.2, 8.2 Hz, 1H), 2.78 (d, J = 7.5 Hz, |
| | 2H), 2.55-2.66 (m, 2H), 1.82-2.40 (m, 6H) |

Illustrative preferred compounds are shown below:

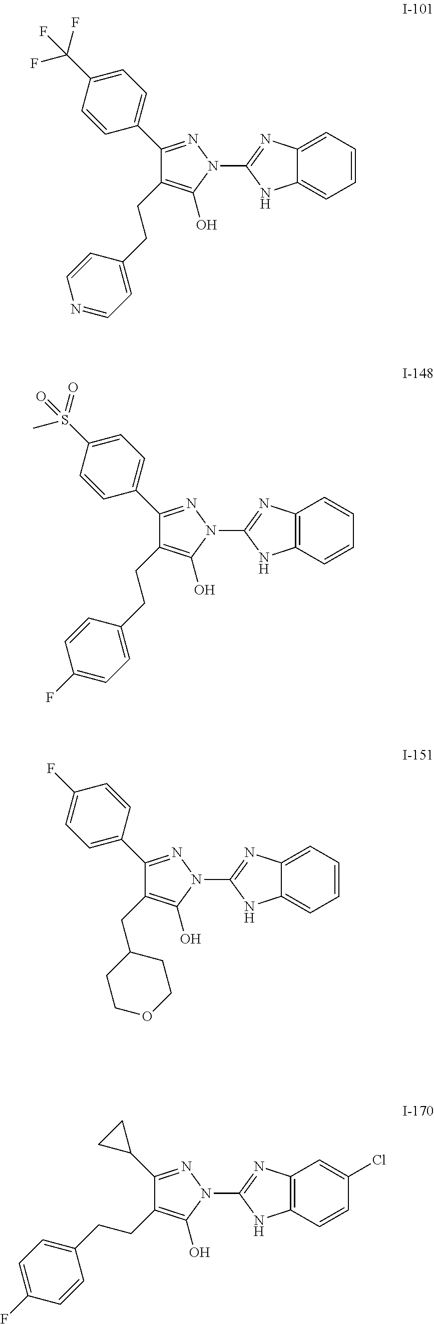 [SMILES: Oc1c(CCc2ccncc2)c(-c2ccc(C(F)(F)F)cc2)nn1-c1nc2ccccc2[nH]1 ; CS(=O)(=O)c1ccc(-c2nn(-c3nc4ccccc4[nH]3)c(O)c2CCc2ccc(F)cc2)cc1 ; Oc1c(CC2CCOCC2)c(-c2ccc(F)cc2)nn1-c1nc2ccccc2[nH]1 ; Oc1c(CCc2ccc(F)cc2)c(C2CC2)nn1-c1nc2cc(Cl)ccc2[nH]1]

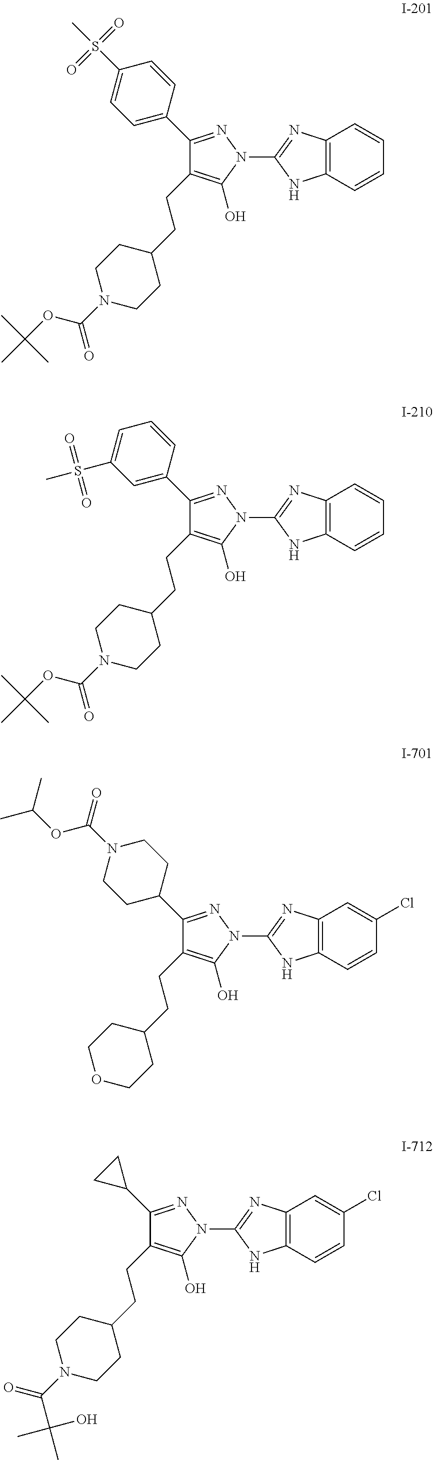 [SMILES: CC(C)(C)OC(=O)N1CCC(CCc2c(-c3ccc(S(C)(=O)=O)cc3)nn(-c3nc4ccccc4[nH]3)c2O)CC1 ; CC(C)(C)OC(=O)N1CCC(CCc2c(-c3cccc(S(C)(=O)=O)c3)nn(-c3nc4ccccc4[nH]3)c2O)CC1 ; CC(C)OC(=O)N1CCC(c2nn(-c3nc4cc(Cl)ccc4[nH]3)c(O)c2CCC2CCOCC2)CC1 ; CC(C)(O)C(=O)N1CCC(CCc2c(C3CC3)nn(-c3nc4cc(Cl)ccc4[nH]3)c2O)CC1]

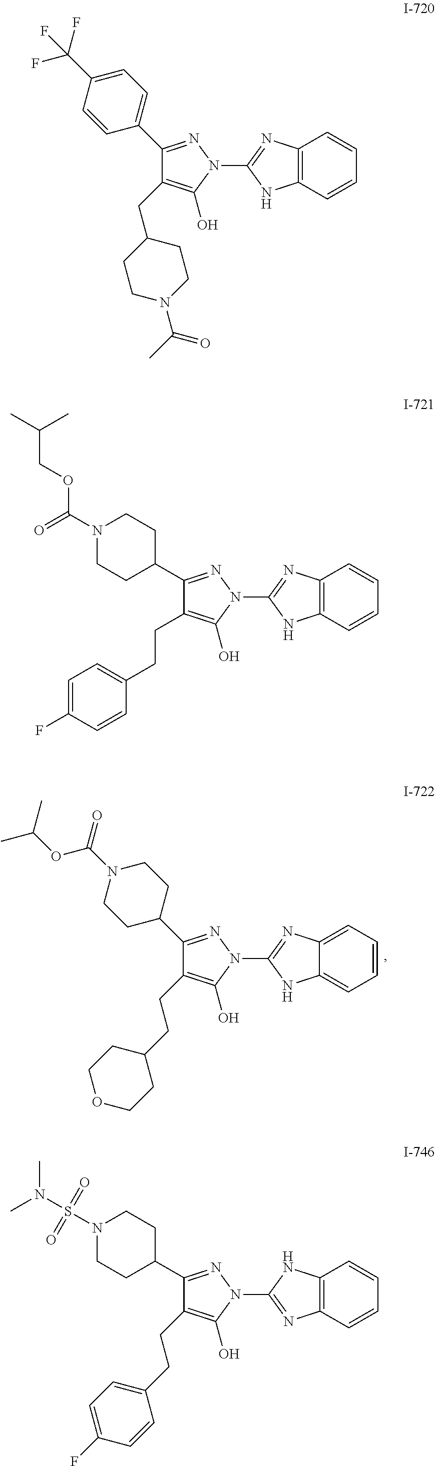 [SMILES: CC(=O)N1CCC(Cc2c(-c3ccc(C(F)(F)F)cc3)nn(-c3nc4ccccc4[nH]3)c2O)CC1 ; CC(C)COC(=O)N1CCC(c2nn(-c3nc4ccccc4[nH]3)c(O)c2CCc2ccc(F)cc2)CC1 ; CC(C)OC(=O)N1CCC(c2nn(-c3nc4ccccc4[nH]3)c(O)c2CCC2CCOCC2)CC1 ; CN(C)S(=O)(=O)N1CCC(c2nn(-c3nc4ccccc4[nH]3)c(O)c2CCc2ccc(F)cc2)CC1]

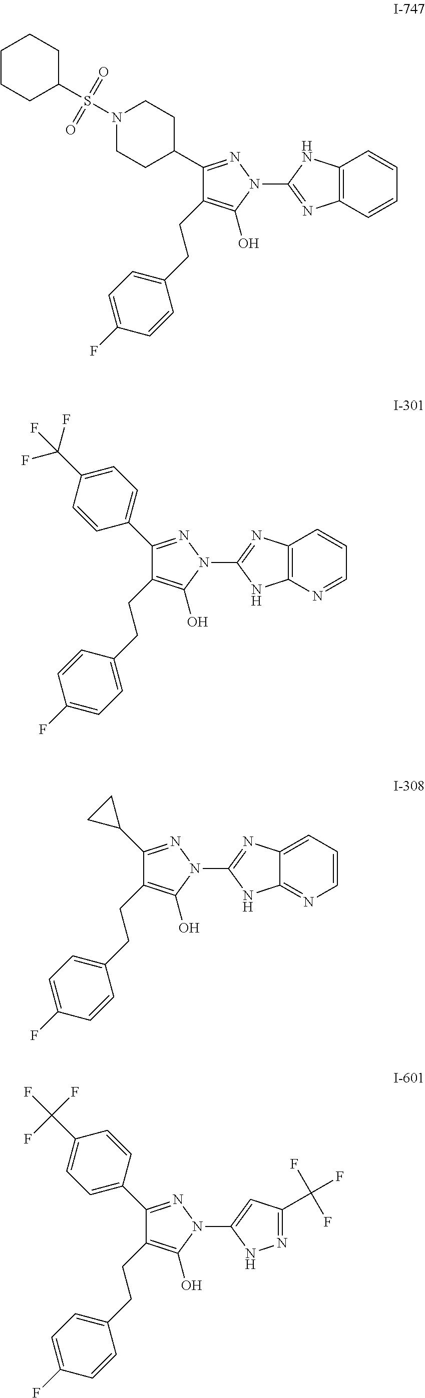 [SMILES: O=S(=O)(C1CCCCC1)N1CCC(c2nn(-c3nc4ccccc4[nH]3)c(O)c2CCc2ccc(F)cc2)CC1 ; Oc1c(CCc2ccc(F)cc2)c(-c2ccc(C(F)(F)F)cc2)nn1-c1nc2cccnc2[nH]1 ; Oc1c(CCc2ccc(F)cc2)c(C2CC2)nn1-c1nc2cccnc2[nH]1 ; Oc1c(CCc2ccc(F)cc2)c(-c2ccc(C(F)(F)F)cc2)nn1-c1cc(C(F)(F)F)n[nH]1]

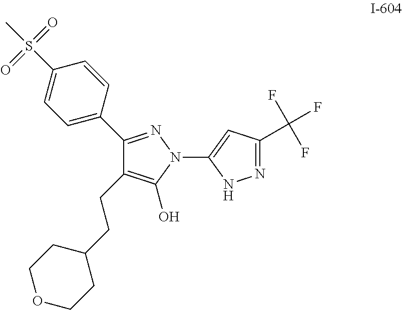 [SMILES: CS(=O)(=O)c1ccc(-c2nn(-c3cc(C(F)(F)F)n[nH]3)c(O)c2CCC2CCOCC2)cc1]

Additional illustrative compounds are shown below:

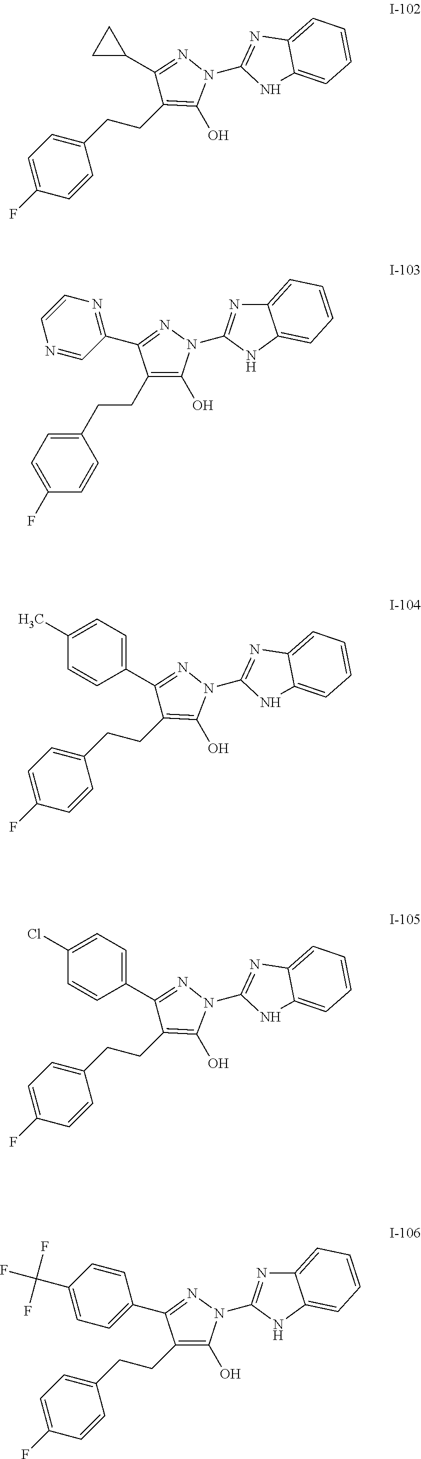 [SMILES: Oc1c(CCc2ccc(F)cc2)c(C2CC2)nn1-c1nc2ccccc2[nH]1 ; Oc1c(CCc2ccc(F)cc2)c(-c2cnccn2)nn1-c1nc2ccccc2[nH]1 ; Cc1ccc(-c2nn(-c3nc4ccccc4[nH]3)c(O)c2CCc2ccc(F)cc2)cc1 ; Oc1c(CCc2ccc(F)cc2)c(-c2ccc(Cl)cc2)nn1-c1nc2ccccc2[nH]1 ; Oc1c(CCc2ccc(F)cc2)c(-c2ccc(C(F)(F)F)cc2)nn1-c1nc2ccccc2[nH]1]

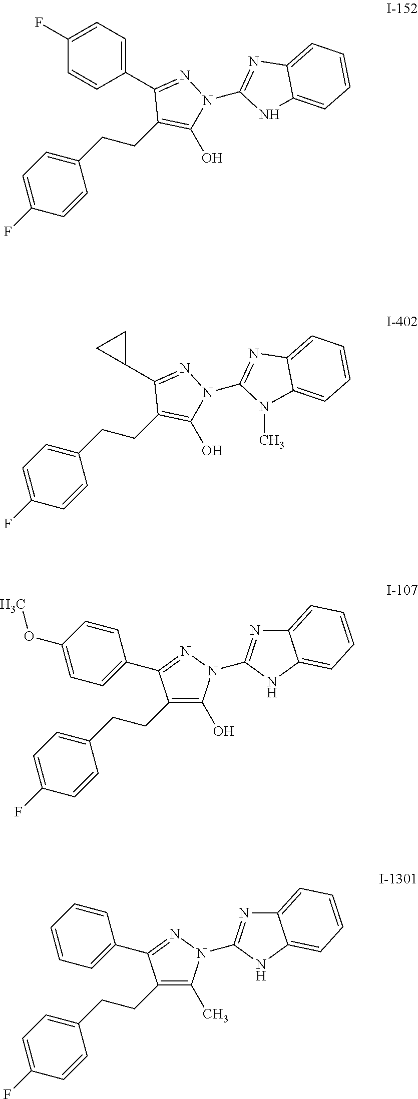 [SMILES: Oc1c(CCc2ccc(F)cc2)c(-c2ccc(F)cc2)nn1-c1nc2ccccc2[nH]1 ; Cn1c(-n2nc(C3CC3)c(CCc3ccc(F)cc3)c2O)nc2ccccc21 ; COc1ccc(-c2nn(-c3nc4ccccc4[nH]3)c(O)c2CCc2ccc(F)cc2)cc1 ; Cc1c(CCc2ccc(F)cc2)c(-c2ccccc2)nn1-c1nc2ccccc2[nH]1]

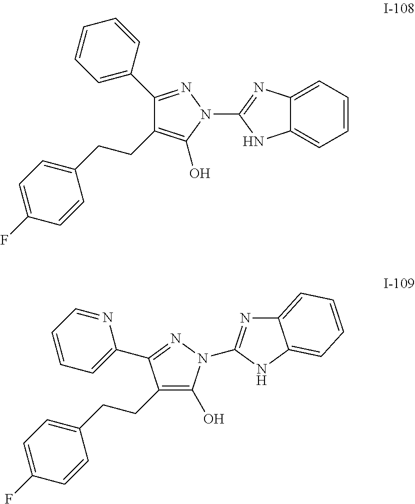 [SMILES: Oc1c(CCc2ccc(F)cc2)c(-c2ccccc2)nn1-c1nc2ccccc2[nH]1 ; Oc1c(CCc2ccc(F)cc2)c(-c2ccccn2)nn1-c1nc2ccccc2[nH]1]

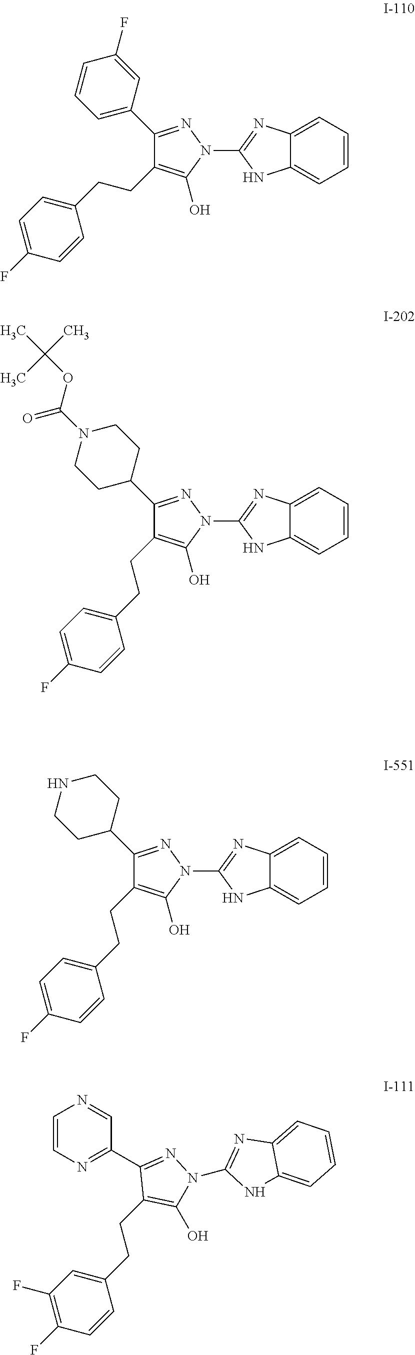 [SMILES: Oc1c(CCc2ccc(F)cc2)c(-c2cccc(F)c2)nn1-c1nc2ccccc2[nH]1 ; CC(C)(C)OC(=O)N1CCC(c2nn(-c3nc4ccccc4[nH]3)c(O)c2CCc2ccc(F)cc2)CC1 ; Oc1c(CCc2ccc(F)cc2)c(C2CCNCC2)nn1-c1nc2ccccc2[nH]1 ; Oc1c(CCc2ccc(F)c(F)c2)c(-c2cnccn2)nn1-c1nc2ccccc2[nH]1]

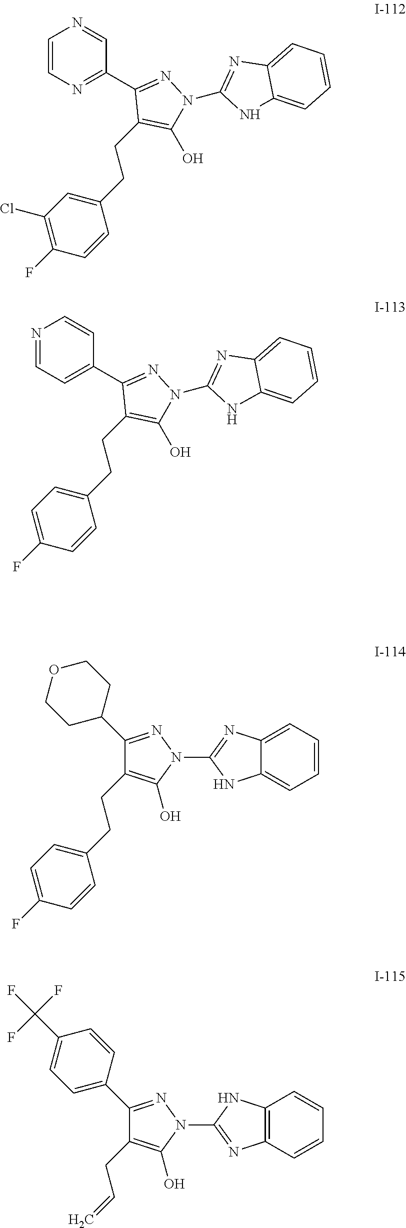 [SMILES: Oc1c(CCc2ccc(F)c(Cl)c2)c(-c2cnccn2)nn1-c1nc2ccccc2[nH]1 ; Oc1c(CCc2ccc(F)cc2)c(-c2ccncc2)nn1-c1nc2ccccc2[nH]1 ; Oc1c(CCc2ccc(F)cc2)c(C2CCOCC2)nn1-c1nc2ccccc2[nH]1 ; C=CCc1c(-c2ccc(C(F)(F)F)cc2)nn(-c2nc3ccccc3[nH]2)c1O]

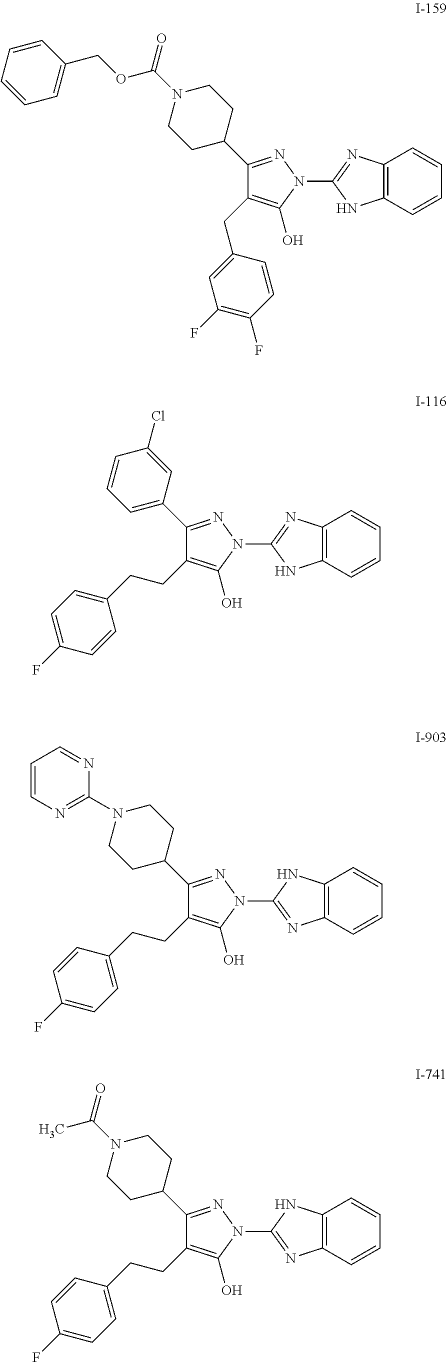 [SMILES: O=C(OCc1ccccc1)N1CCC(c2nn(-c3nc4ccccc4[nH]3)c(O)c2Cc2ccc(F)c(F)c2)CC1 ; Oc1c(CCc2ccc(F)cc2)c(-c2cccc(Cl)c2)nn1-c1nc2ccccc2[nH]1 ; Oc1c(CCc2ccc(F)cc2)c(C2CCN(c3ncccn3)CC2)nn1-c1nc2ccccc2[nH]1 ; CC(=O)N1CCC(c2nn(-c3nc4ccccc4[nH]3)c(O)c2CCc2ccc(F)cc2)CC1]

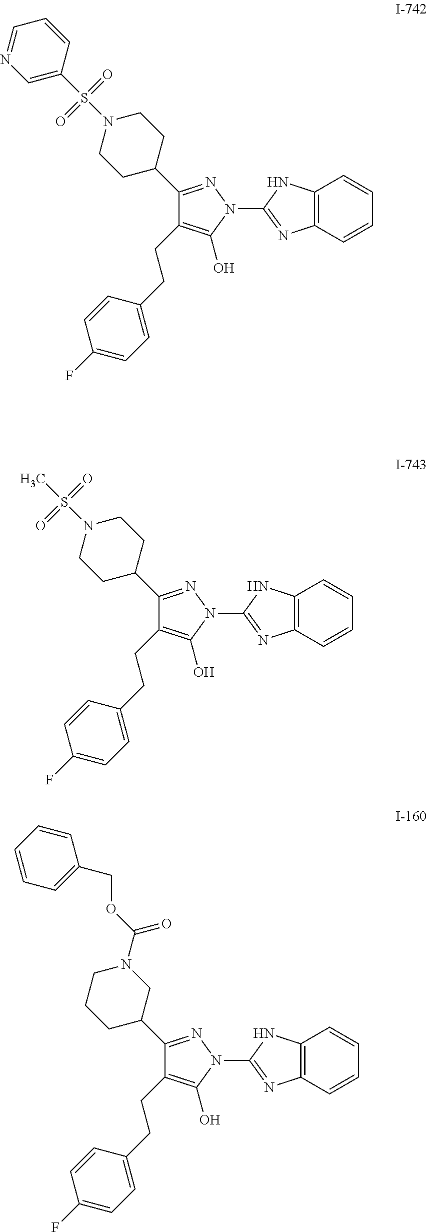 [SMILES: O=S(=O)(c1cccnc1)N1CCC(c2nn(-c3nc4ccccc4[nH]3)c(O)c2CCc2ccc(F)cc2)CC1 ; CS(=O)(=O)N1CCC(c2nn(-c3nc4ccccc4[nH]3)c(O)c2CCc2ccc(F)cc2)CC1 ; O=C(OCc1ccccc1)N1CCCC(c2nn(-c3nc4ccccc4[nH]3)c(O)c2CCc2ccc(F)cc2)C1]

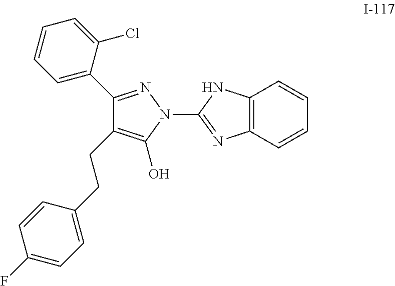 [SMILES: Oc1c(CCc2ccc(F)cc2)c(-c2ccccc2Cl)nn1-c1nc2ccccc2[nH]1]

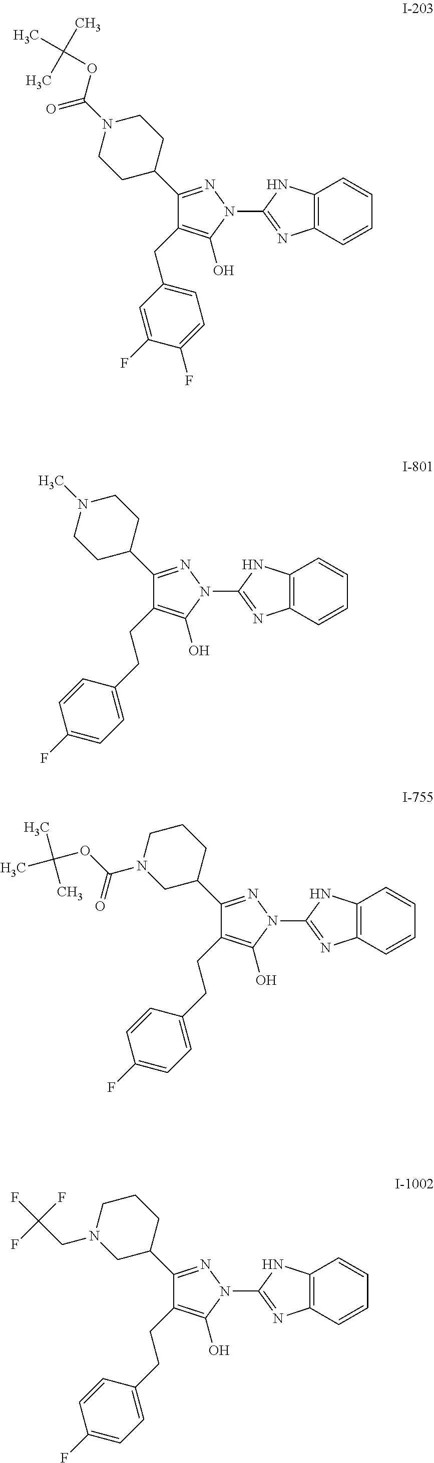 [SMILES: CC(C)(C)OC(=O)N1CCC(c2nn(-c3nc4ccccc4[nH]3)c(O)c2Cc2ccc(F)c(F)c2)CC1 ; CN1CCC(c2nn(-c3nc4ccccc4[nH]3)c(O)c2CCc2ccc(F)cc2)CC1 ; CC(C)(C)OC(=O)N1CCCC(c2nn(-c3nc4ccccc4[nH]3)c(O)c2CCc2ccc(F)cc2)C1 ; Oc1c(CCc2ccc(F)cc2)c(C2CCCN(CC(F)(F)F)C2)nn1-c1nc2ccccc2[nH]1]

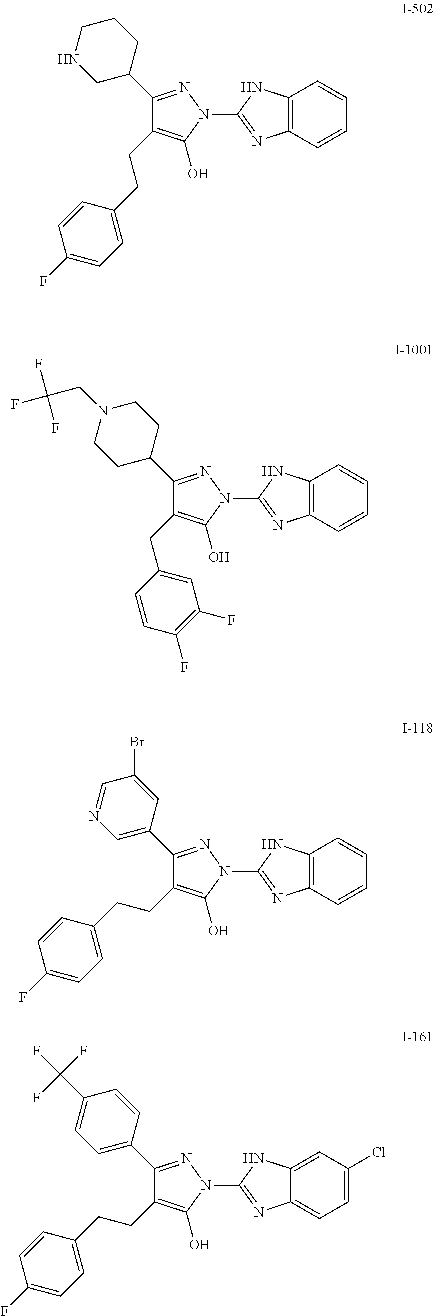 [SMILES: Oc1c(CCc2ccc(F)cc2)c(C2CCCNC2)nn1-c1nc2ccccc2[nH]1 ; Oc1c(Cc2ccc(F)c(F)c2)c(C2CCN(CC(F)(F)F)CC2)nn1-c1nc2ccccc2[nH]1 ; Oc1c(CCc2ccc(F)cc2)c(-c2cncc(Br)c2)nn1-c1nc2ccccc2[nH]1 ; Oc1c(CCc2ccc(F)cc2)c(-c2ccc(C(F)(F)F)cc2)nn1-c1nc2ccc(Cl)cc2[nH]1]

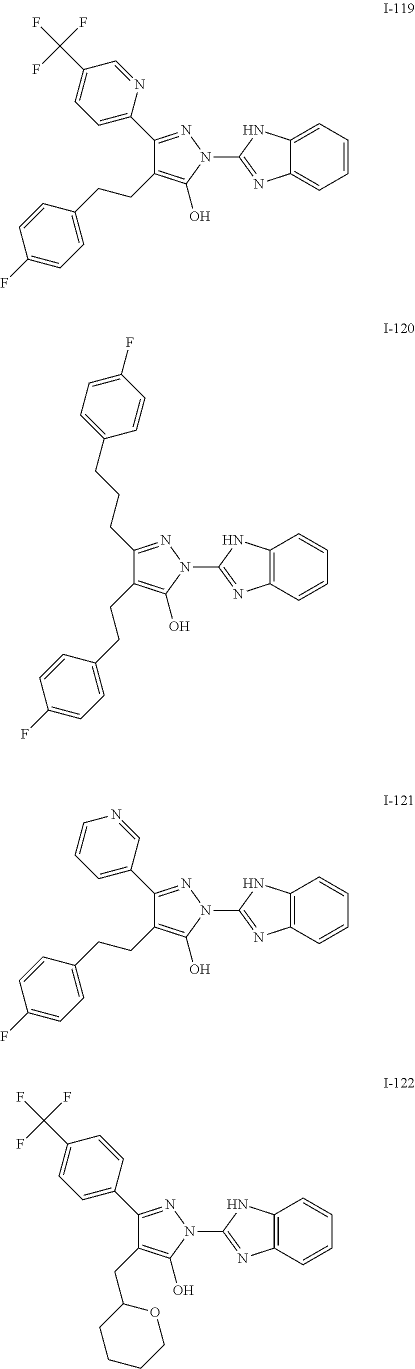 [SMILES: Oc1c(CCc2ccc(F)cc2)c(-c2ccc(C(F)(F)F)cn2)nn1-c1nc2ccccc2[nH]1 ; Oc1c(CCc2ccc(F)cc2)c(CCCc2ccc(F)cc2)nn1-c1nc2ccccc2[nH]1 ; Oc1c(CCc2ccc(F)cc2)c(-c2cccnc2)nn1-c1nc2ccccc2[nH]1 ; Oc1c(CC2CCCCO2)c(-c2ccc(C(F)(F)F)cc2)nn1-c1nc2ccccc2[nH]1]

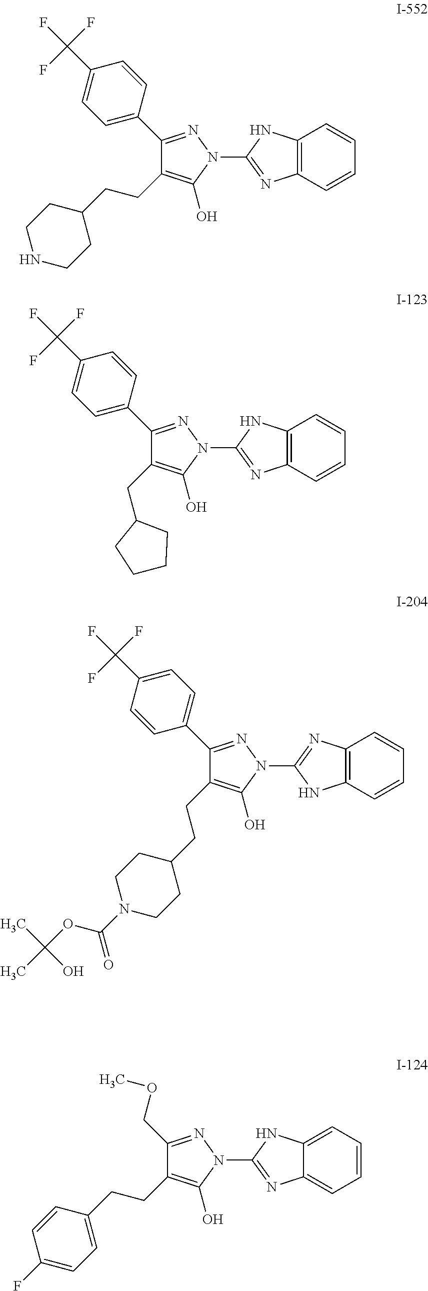 [SMILES: Oc1c(CCC2CCNCC2)c(-c2ccc(C(F)(F)F)cc2)nn1-c1nc2ccccc2[nH]1 ; Oc1c(CC2CCCC2)c(-c2ccc(C(F)(F)F)cc2)nn1-c1nc2ccccc2[nH]1 ; CC(C)(O)OC(=O)N1CCC(CCc2c(-c3ccc(C(F)(F)F)cc3)nn(-c3nc4ccccc4[nH]3)c2O)CC1 ; COCc1nn(-c2nc3ccccc3[nH]2)c(O)c1CCc1ccc(F)cc1]

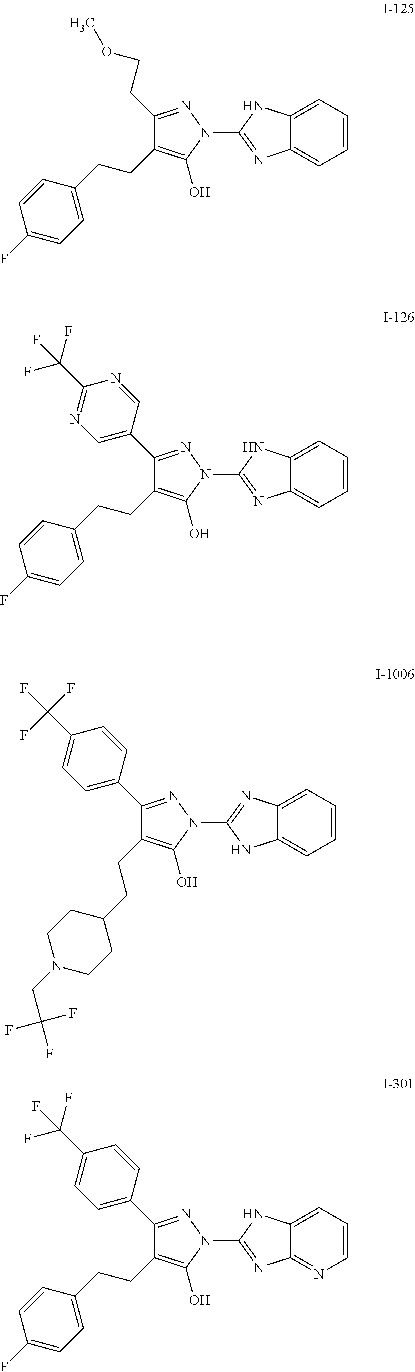 [SMILES: COCCc1nn(-c2nc3ccccc3[nH]2)c(O)c1CCc1ccc(F)cc1 ; Oc1c(CCc2ccc(F)cc2)c(-c2cnc(C(F)(F)F)nc2)nn1-c1nc2ccccc2[nH]1 ; Oc1c(CCC2CCN(CC(F)(F)F)CC2)c(-c2ccc(C(F)(F)F)cc2)nn1-c1nc2ccccc2[nH]1 ; Oc1c(CCc2ccc(F)cc2)c(-c2ccc(C(F)(F)F)cc2)nn1-c1nc2ncccc2[nH]1]

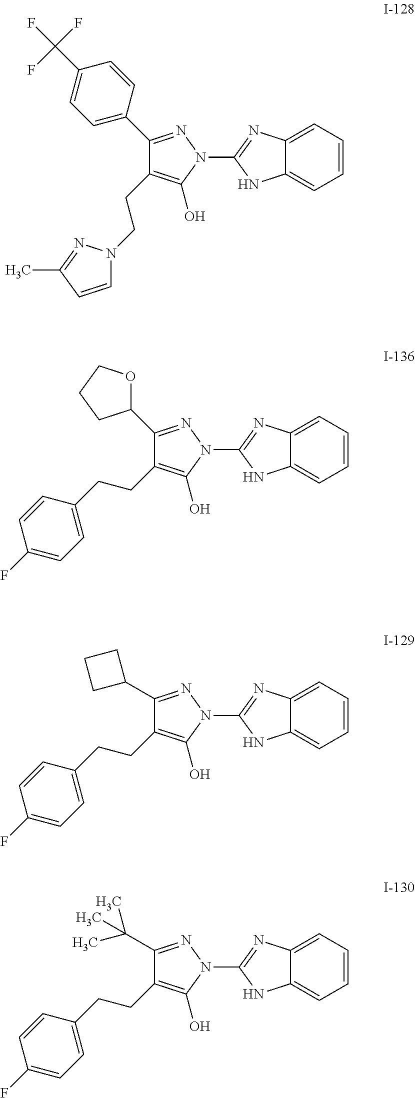 [SMILES: Cc1ccn(CCc2c(-c3ccc(C(F)(F)F)cc3)nn(-c3nc4ccccc4[nH]3)c2O)n1 ; Oc1c(CCc2ccc(F)cc2)c(C2CCCO2)nn1-c1nc2ccccc2[nH]1 ; Oc1c(CCc2ccc(F)cc2)c(C2CCC2)nn1-c1nc2ccccc2[nH]1 ; CC(C)(C)c1nn(-c2nc3ccccc3[nH]2)c(O)c1CCc1ccc(F)cc1]

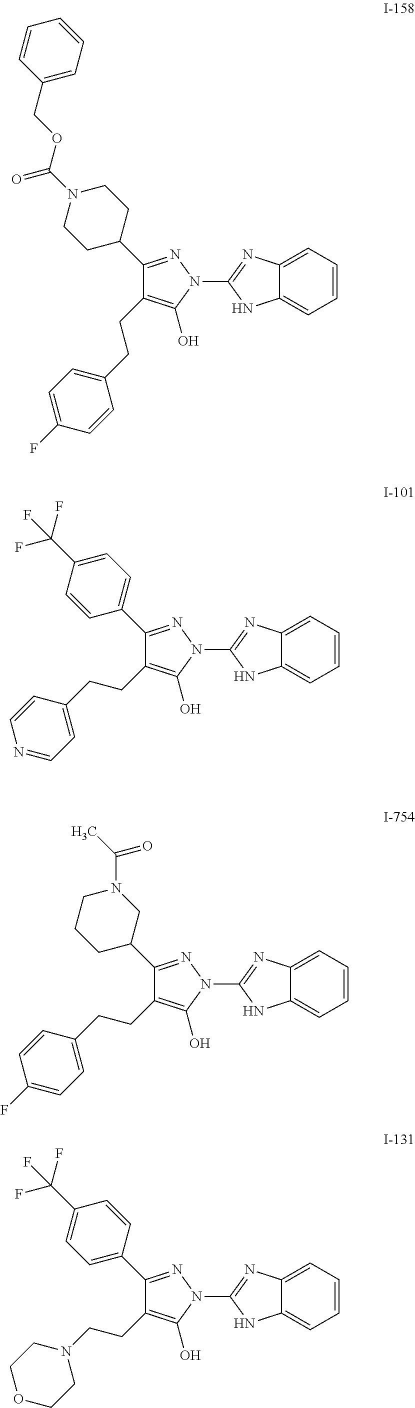 [SMILES: O=C(OCc1ccccc1)N1CCC(c2nn(-c3nc4ccccc4[nH]3)c(O)c2CCc2ccc(F)cc2)CC1 ; Oc1c(CCc2ccncc2)c(-c2ccc(C(F)(F)F)cc2)nn1-c1nc2ccccc2[nH]1 ; CC(=O)N1CCCC(c2nn(-c3nc4ccccc4[nH]3)c(O)c2CCc2ccc(F)cc2)C1 ; Oc1c(CCN2CCOCC2)c(-c2ccc(C(F)(F)F)cc2)nn1-c1nc2ccccc2[nH]1]

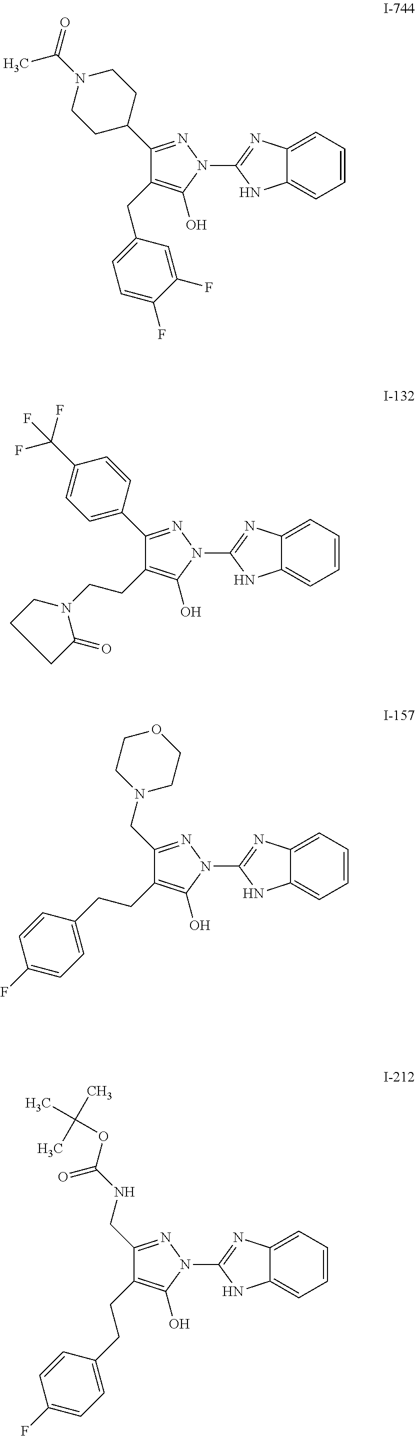 [SMILES: CC(=O)N1CCC(c2nn(-c3nc4ccccc4[nH]3)c(O)c2Cc2ccc(F)c(F)c2)CC1 ; O=C1CCCN1CCc1c(-c2ccc(C(F)(F)F)cc2)nn(-c2nc3ccccc3[nH]2)c1O ; Oc1c(CCc2ccc(F)cc2)c(CN2CCOCC2)nn1-c1nc2ccccc2[nH]1 ; CC(C)(C)OC(=O)NCc1nn(-c2nc3ccccc3[nH]2)c(O)c1CCc1ccc(F)cc1]

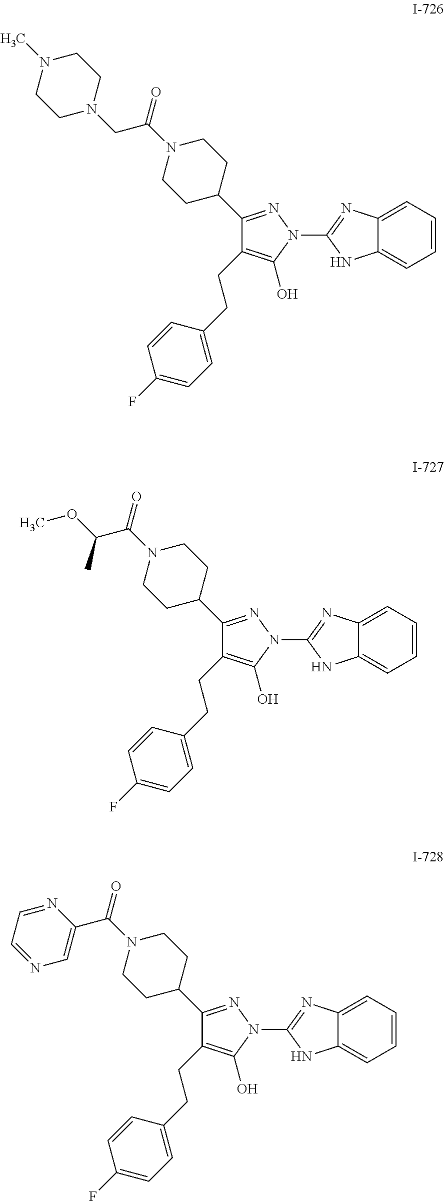 [SMILES: CN1CCN(CC(=O)N2CCC(c3nn(-c4nc5ccccc5[nH]4)c(O)c3CCc3ccc(F)cc3)CC2)CC1 ; CO[C@H](C)C(=O)N1CCC(c2nn(-c3nc4ccccc4[nH]3)c(O)c2CCc2ccc(F)cc2)CC1 ; O=C(c1cnccn1)N1CCC(c2nn(-c3nc4ccccc4[nH]3)c(O)c2CCc2ccc(F)cc2)CC1]

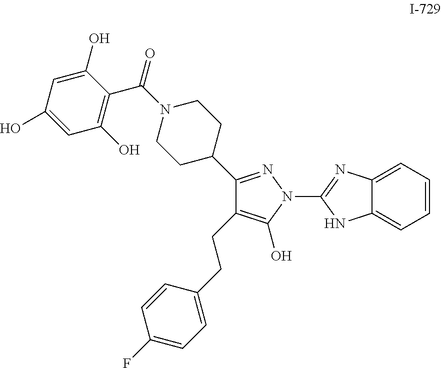 [SMILES: O=C(c1c(O)cc(O)cc1O)N1CCC(c2nn(-c3nc4ccccc4[nH]3)c(O)c2CCc2ccc(F)cc2)CC1]

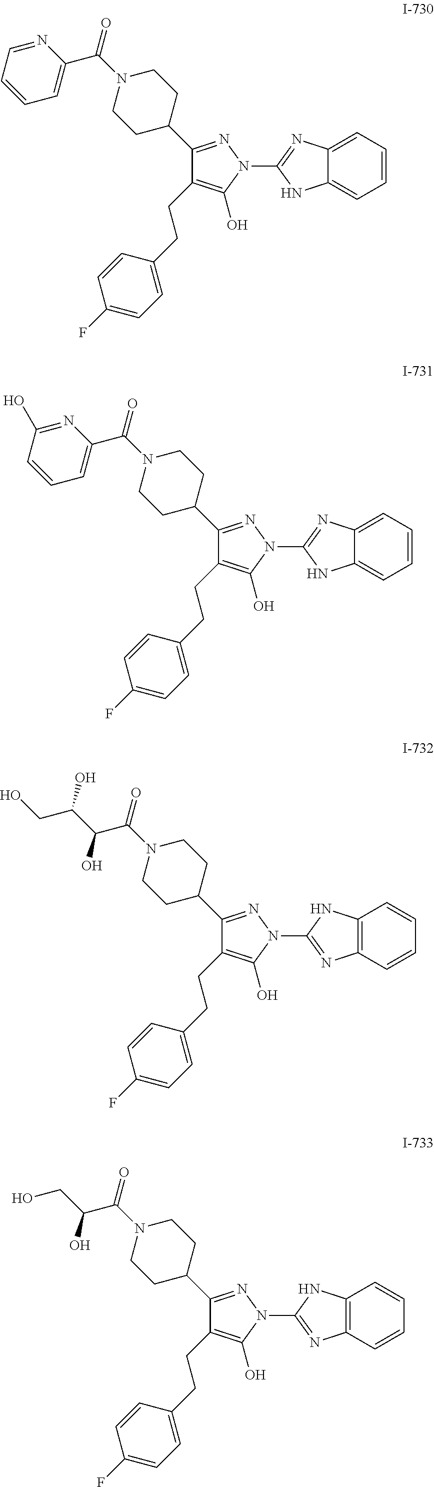 [SMILES: O=C(c1ccccn1)N1CCC(c2nn(-c3nc4ccccc4[nH]3)c(O)c2CCc2ccc(F)cc2)CC1 ; O=C(c1cccc(O)n1)N1CCC(c2nn(-c3nc4ccccc4[nH]3)c(O)c2CCc2ccc(F)cc2)CC1 ; O=C([C@@H](O)[C@@H](O)CO)N1CCC(c2nn(-c3nc4ccccc4[nH]3)c(O)c2CCc2ccc(F)cc2)CC1 ; O=C([C@@H](O)CO)N1CCC(c2nn(-c3nc4ccccc4[nH]3)c(O)c2CCc2ccc(F)cc2)CC1]

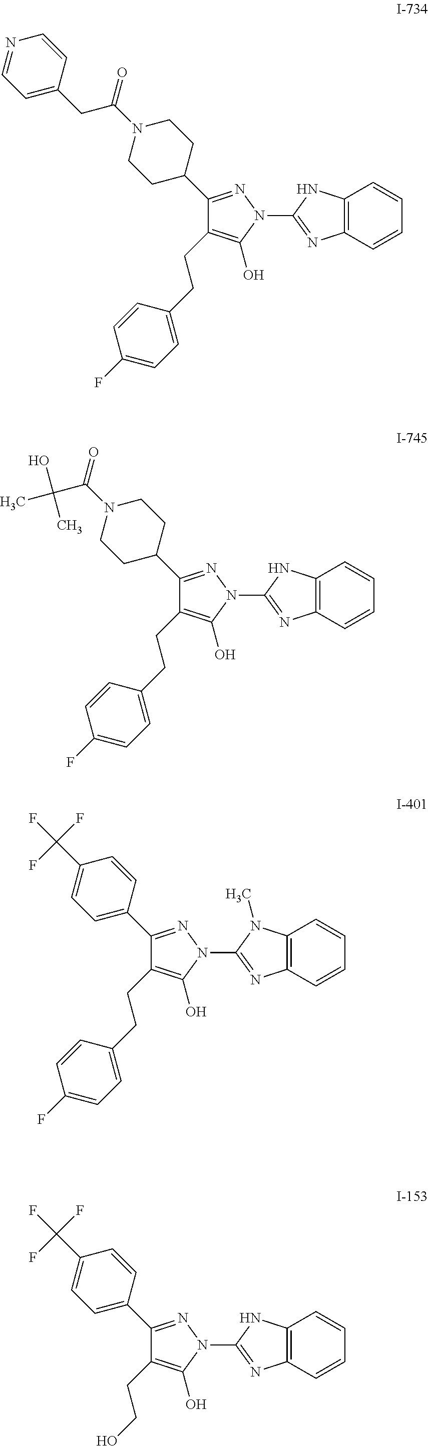 [SMILES: O=C(Cc1ccncc1)N1CCC(c2nn(-c3nc4ccccc4[nH]3)c(O)c2CCc2ccc(F)cc2)CC1 ; CC(C)(O)C(=O)N1CCC(c2nn(-c3nc4ccccc4[nH]3)c(O)c2CCc2ccc(F)cc2)CC1 ; Cn1c(-n2nc(-c3ccc(C(F)(F)F)cc3)c(CCc3ccc(F)cc3)c2O)nc2ccccc21 ; OCCc1c(-c2ccc(C(F)(F)F)cc2)nn(-c2nc3ccccc3[nH]2)c1O]

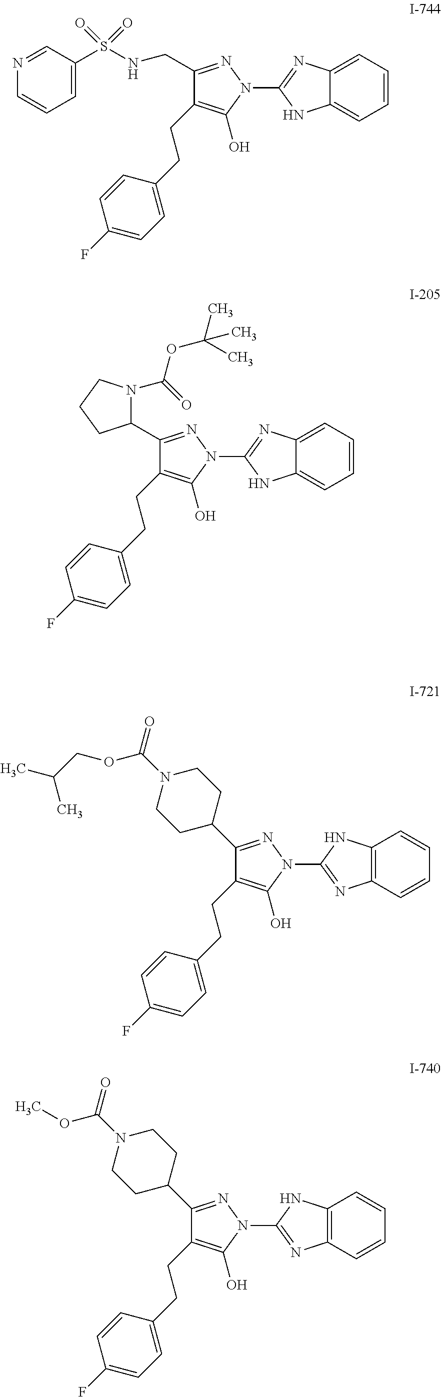 [SMILES: O=S(=O)(NCc1nn(-c2nc3ccccc3[nH]2)c(O)c1CCc1ccc(F)cc1)c1cccnc1 ; CC(C)(C)OC(=O)N1CCCC1c1nn(-c2nc3ccccc3[nH]2)c(O)c1CCc1ccc(F)cc1 ; CC(C)COC(=O)N1CCC(c2nn(-c3nc4ccccc4[nH]3)c(O)c2CCc2ccc(F)cc2)CC1 ; COC(=O)N1CCC(c2nn(-c3nc4ccccc4[nH]3)c(O)c2CCc2ccc(F)cc2)CC1]

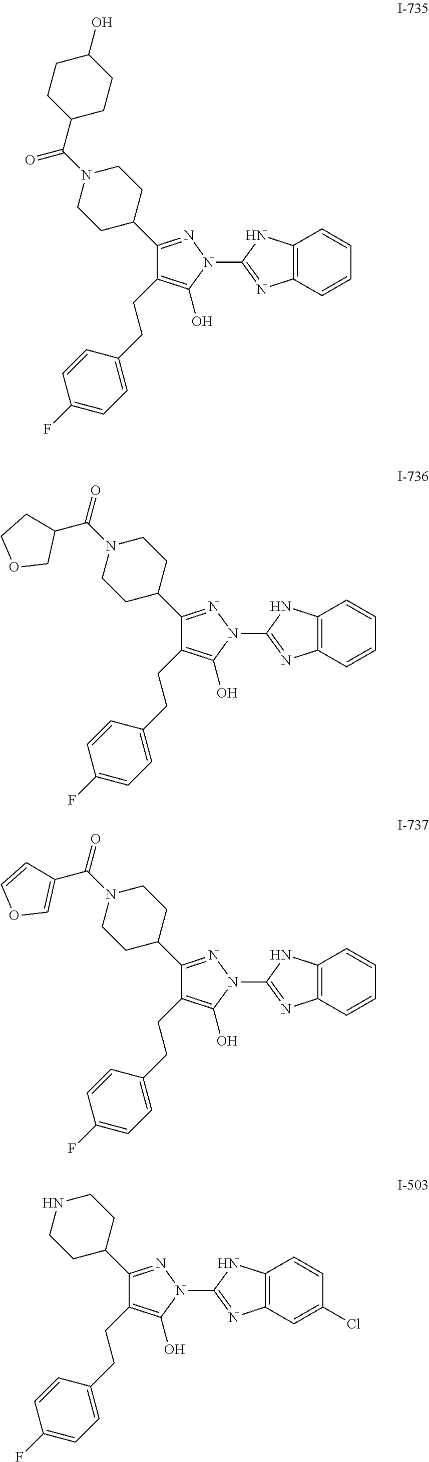

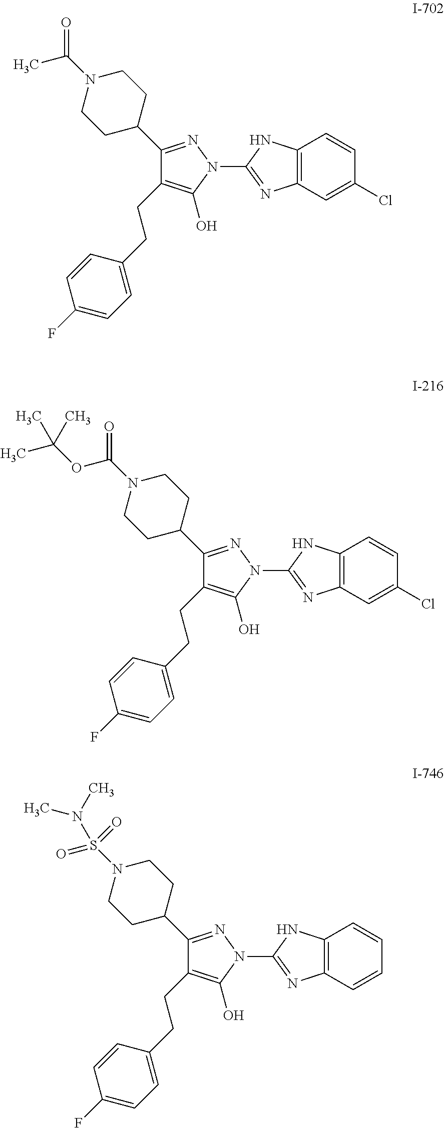 [SMILES: CC(=O)N1CCC(c2nn(-c3nc4cc(Cl)ccc4[nH]3)c(O)c2CCc2ccc(F)cc2)CC1 ; CC(C)(C)OC(=O)N1CCC(c2nn(-c3nc4cc(Cl)ccc4[nH]3)c(O)c2CCc2ccc(F)cc2)CC1 ; CN(C)S(=O)(=O)N1CCC(c2nn(-c3nc4ccccc4[nH]3)c(O)c2CCc2ccc(F)cc2)CC1]

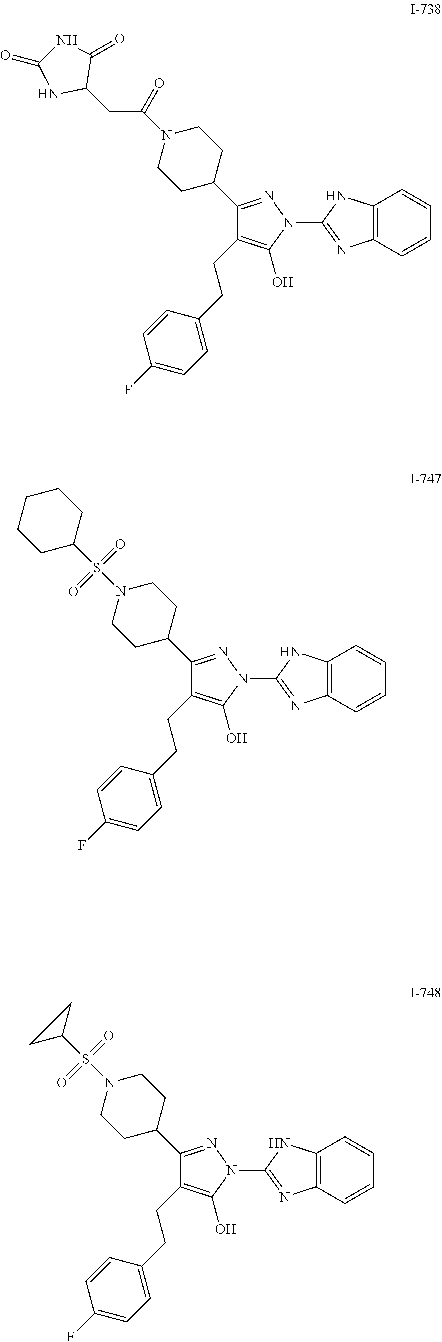 [SMILES: O=C1NC(=O)C(CC(=O)N2CCC(c3nn(-c4nc5ccccc5[nH]4)c(O)c3CCc3ccc(F)cc3)CC2)N1 ; O=S(=O)(C1CCCCC1)N1CCC(c2nn(-c3nc4ccccc4[nH]3)c(O)c2CCc2ccc(F)cc2)CC1 ; O=S(=O)(C1CC1)N1CCC(c2nn(-c3nc4ccccc4[nH]3)c(O)c2CCc2ccc(F)cc2)CC1]

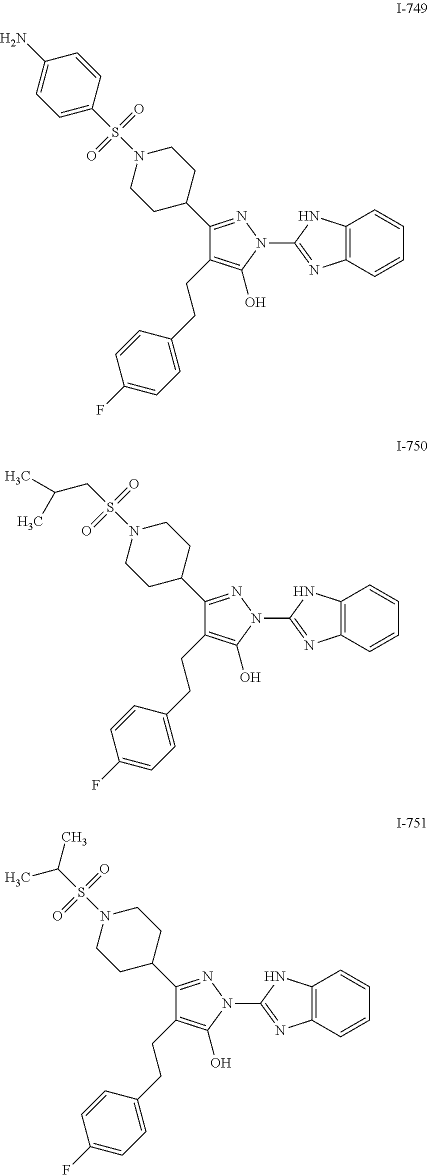 [SMILES: Nc1ccc(S(=O)(=O)N2CCC(c3nn(-c4nc5ccccc5[nH]4)c(O)c3CCc3ccc(F)cc3)CC2)cc1 ; CC(C)CS(=O)(=O)N1CCC(c2nn(-c3nc4ccccc4[nH]3)c(O)c2CCc2ccc(F)cc2)CC1 ; CC(C)S(=O)(=O)N1CCC(c2nn(-c3nc4ccccc4[nH]3)c(O)c2CCc2ccc(F)cc2)CC1]

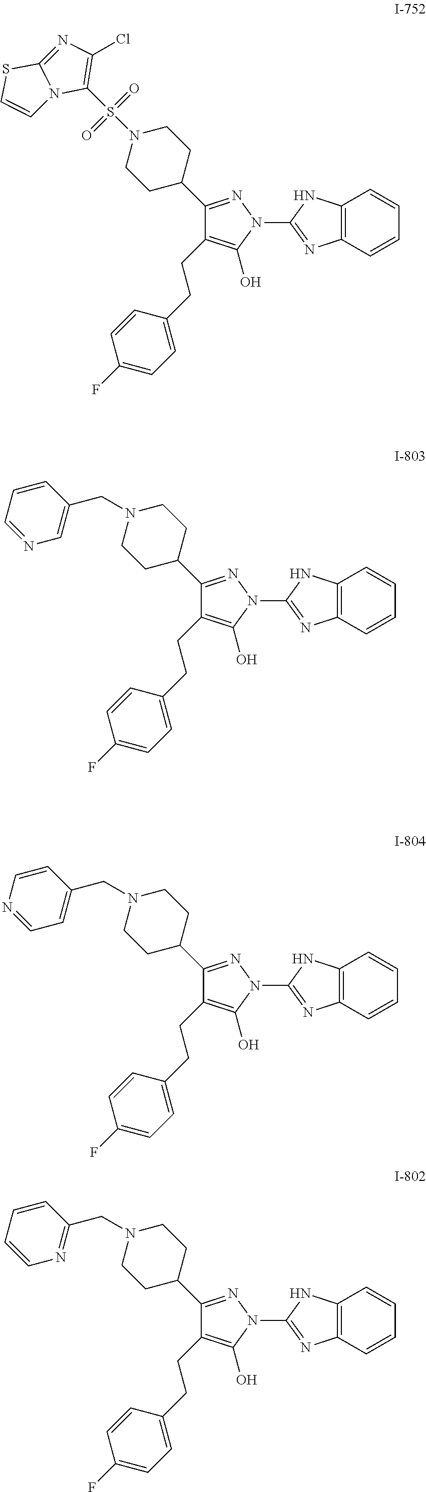 [SMILES: O=S(=O)(c1c(Cl)nc2sccn12)N1CCC(c2nn(-c3nc4ccccc4[nH]3)c(O)c2CCc2ccc(F)cc2)CC1 ; Oc1c(CCc2ccc(F)cc2)c(C2CCN(Cc3cccnc3)CC2)nn1-c1nc2ccccc2[nH]1 ; Oc1c(CCc2ccc(F)cc2)c(C2CCN(Cc3ccncc3)CC2)nn1-c1nc2ccccc2[nH]1 ; Oc1c(CCc2ccc(F)cc2)c(C2CCN(Cc3ccccn3)CC2)nn1-c1nc2ccccc2[nH]1]

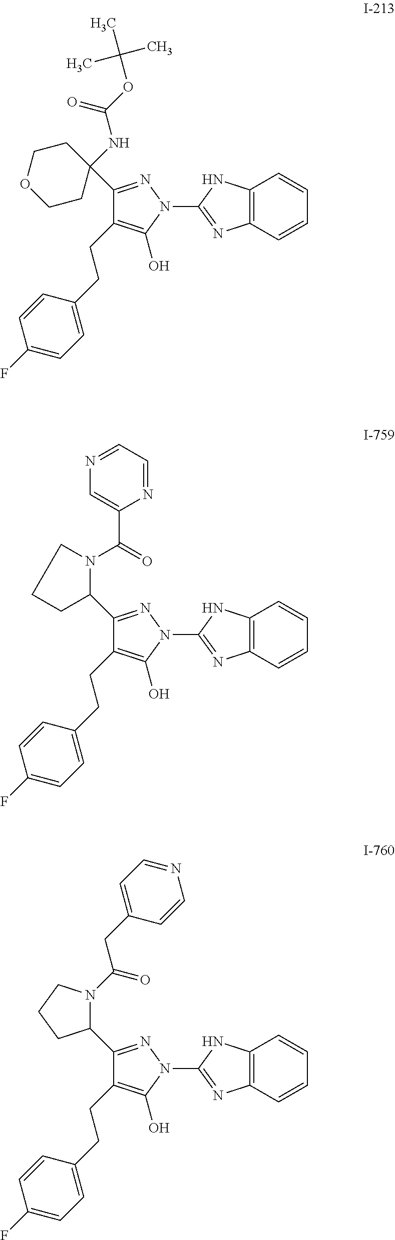 [SMILES: CC(C)(C)OC(=O)NC1(c2nn(-c3nc4ccccc4[nH]3)c(O)c2CCc2ccc(F)cc2)CCOCC1 ; O=C(c1cnccn1)N1CCCC1c1nn(-c2nc3ccccc3[nH]2)c(O)c1CCc1ccc(F)cc1 ; O=C(Cc1ccncc1)N1CCCC1c1nn(-c2nc3ccccc3[nH]2)c(O)c1CCc1ccc(F)cc1]

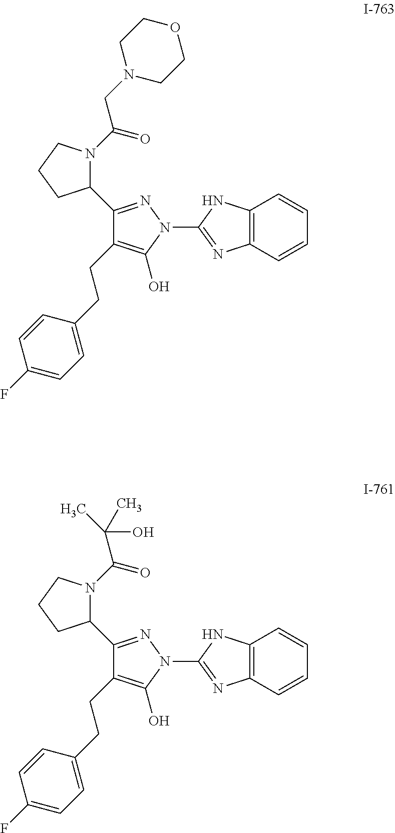 [SMILES: O=C(CN1CCOCC1)N1CCCC1c1nn(-c2nc3ccccc3[nH]2)c(O)c1CCc1ccc(F)cc1 ; CC(C)(O)C(=O)N1CCCC1c1nn(-c2nc3ccccc3[nH]2)c(O)c1CCc1ccc(F)cc1]

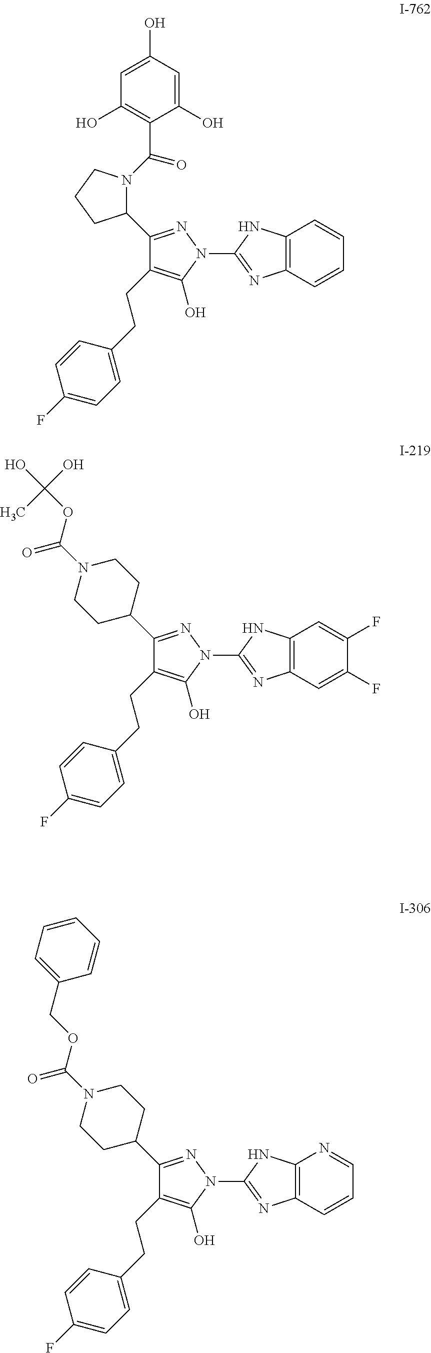 [SMILES: O=C(c1c(O)cc(O)cc1O)N1CCCC1c1nn(-c2nc3ccccc3[nH]2)c(O)c1CCc1ccc(F)cc1 ; CC(O)(O)OC(=O)N1CCC(c2nn(-c3nc4cc(F)c(F)cc4[nH]3)c(O)c2CCc2ccc(F)cc2)CC1 ; O=C(OCc1ccccc1)N1CCC(c2nn(-c3nc4cccnc4[nH]3)c(O)c2CCc2ccc(F)cc2)CC1]

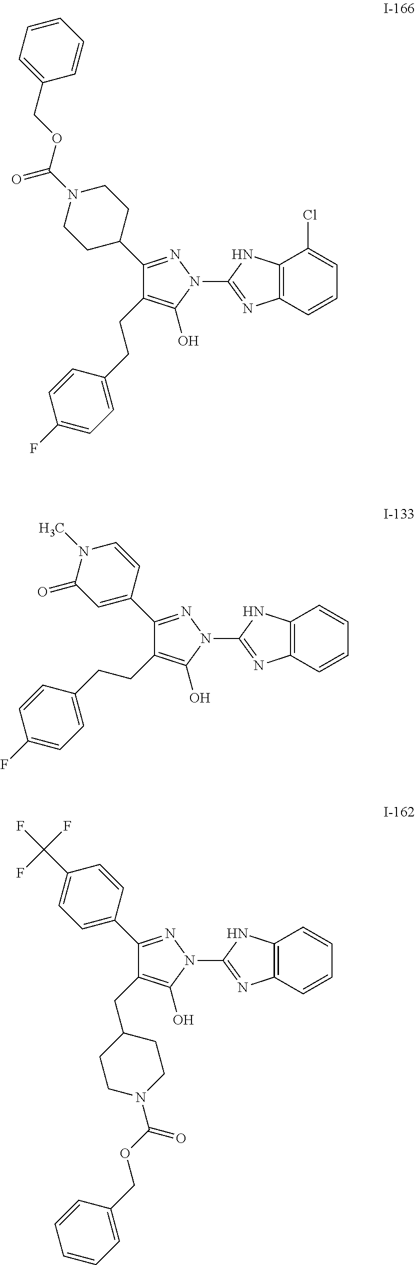 [SMILES: O=C(OCc1ccccc1)N1CCC(c2nn(-c3nc4cccc(Cl)c4[nH]3)c(O)c2CCc2ccc(F)cc2)CC1 ; Cn1ccc(-c2nn(-c3nc4ccccc4[nH]3)c(O)c2CCc2ccc(F)cc2)cc1=O ; O=C(OCc1ccccc1)N1CCC(Cc2c(-c3ccc(C(F)(F)F)cc3)nn(-c3nc4ccccc4[nH]3)c2O)CC1]

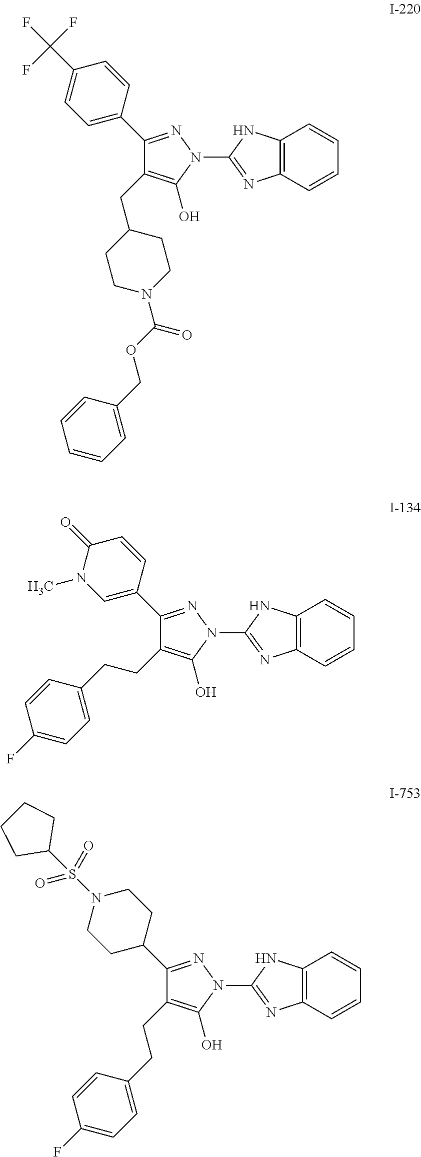

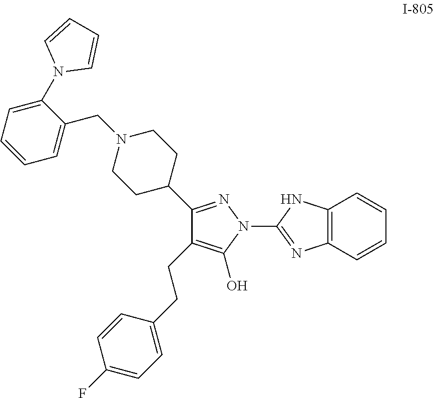 [SMILES: Oc1c(CCc2ccc(F)cc2)c(C2CCN(Cc3ccccc3-n3cccc3)CC2)nn1-c1nc2ccccc2[nH]1]

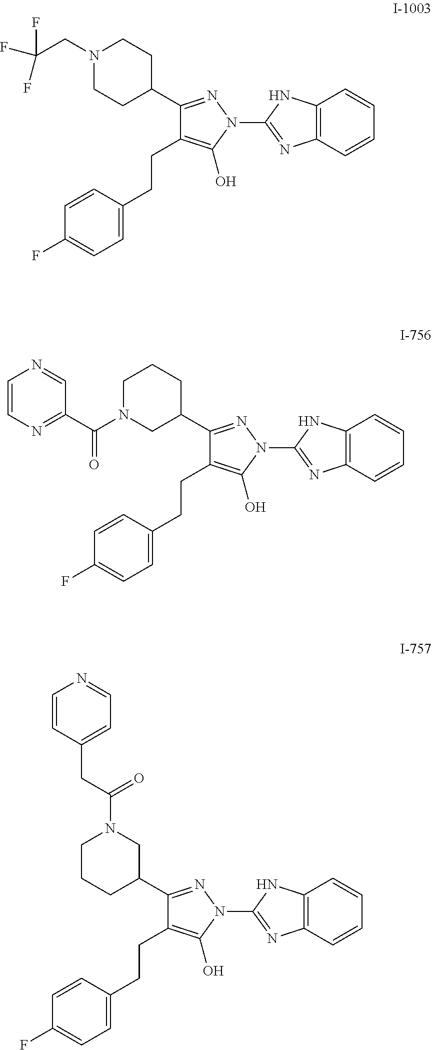 [SMILES: Oc1c(CCc2ccc(F)cc2)c(C2CCN(CC(F)(F)F)CC2)nn1-c1nc2ccccc2[nH]1 ; O=C(c1cnccn1)N1CCCC(c2nn(-c3nc4ccccc4[nH]3)c(O)c2CCc2ccc(F)cc2)C1 ; O=C(Cc1ccncc1)N1CCCC(c2nn(-c3nc4ccccc4[nH]3)c(O)c2CCc2ccc(F)cc2)C1]

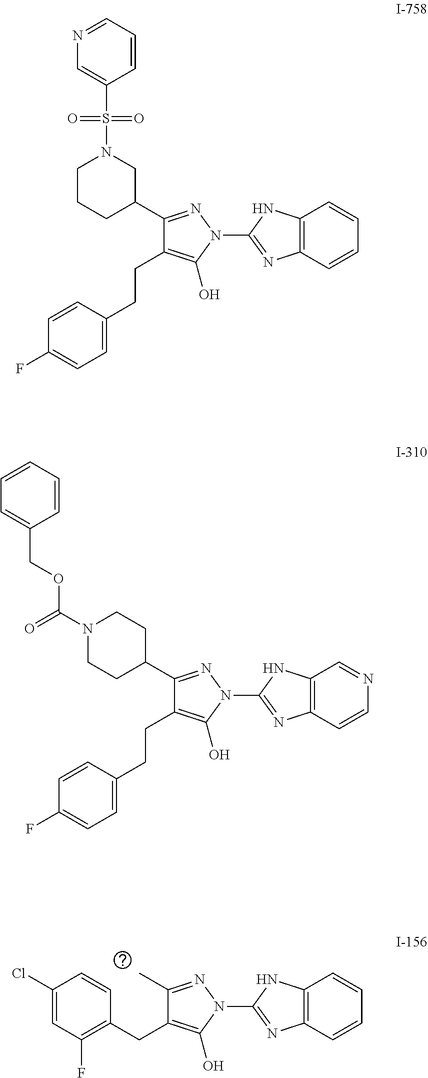 [SMILES: O=S(=O)(c1cccnc1)N1CCCC(c2nn(-c3nc4ccccc4[nH]3)c(O)c2CCc2ccc(F)cc2)C1 ; O=C(OCc1ccccc1)N1CCC(c2nn(-c3nc4ccncc4[nH]3)c(O)c2CCc2ccc(F)cc2)CC1 ; Cc1nn(-c2nc3ccccc3[nH]2)c(O)c1Cc1ccc(Cl)cc1F]

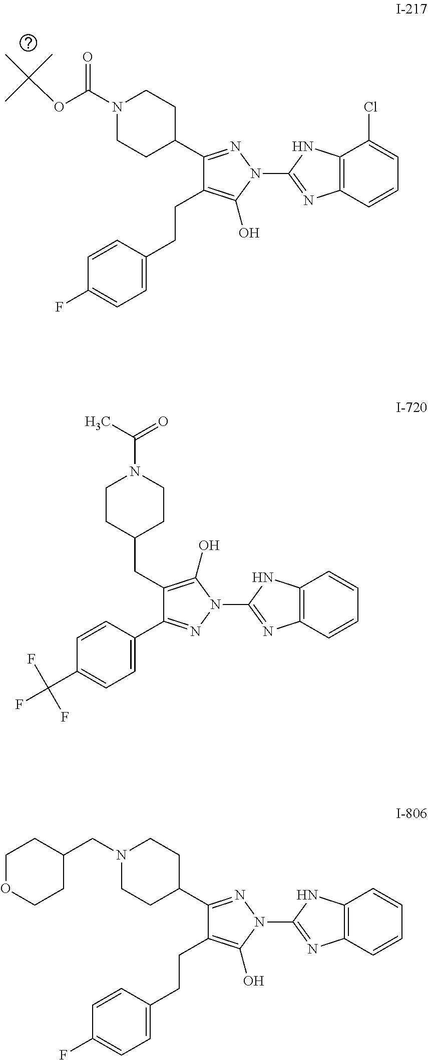 [SMILES: CC(C)(C)OC(=O)N1CCC(c2nn(-c3nc4cccc(Cl)c4[nH]3)c(O)c2CCc2ccc(F)cc2)CC1 ; CC(=O)N1CCC(Cc2c(-c3ccc(C(F)(F)F)cc3)nn(-c3nc4ccccc4[nH]3)c2O)CC1 ; Oc1c(CCc2ccc(F)cc2)c(C2CCN(CC3CCOCC3)CC2)nn1-c1nc2ccccc2[nH]1]

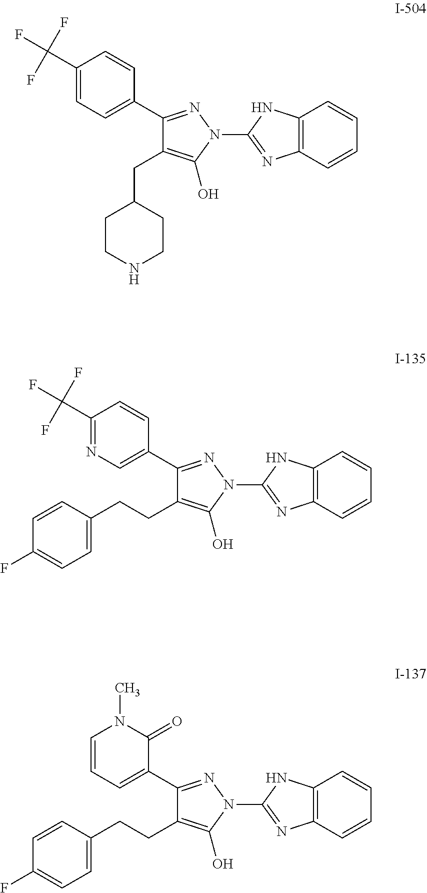 [SMILES: Oc1c(CC2CCNCC2)c(-c2ccc(C(F)(F)F)cc2)nn1-c1nc2ccccc2[nH]1 ; Oc1c(CCc2ccc(F)cc2)c(-c2ccc(C(F)(F)F)nc2)nn1-c1nc2ccccc2[nH]1 ; Cn1cccc(-c2nn(-c3nc4ccccc4[nH]3)c(O)c2CCc2ccc(F)cc2)c1=O]

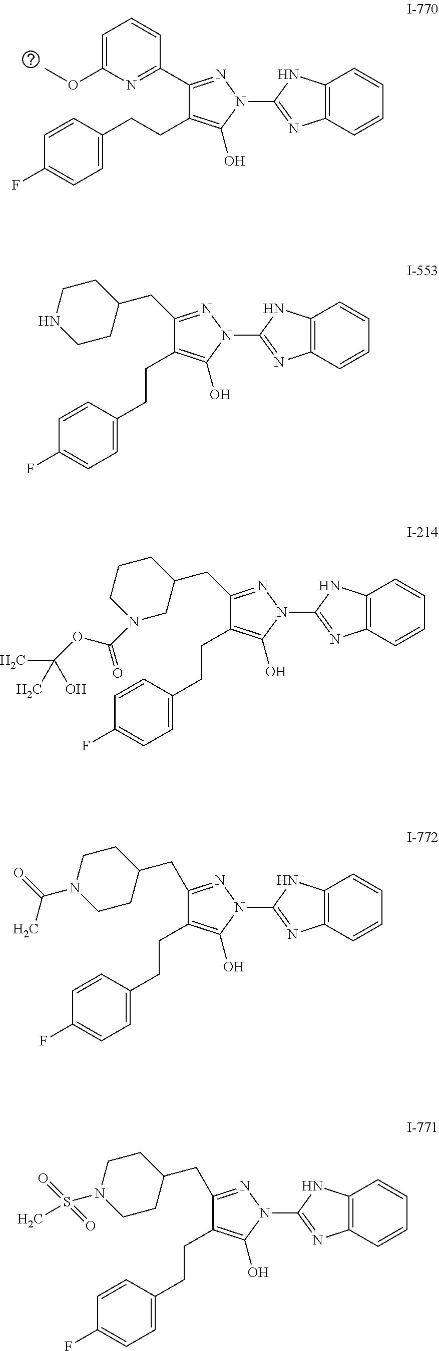 [SMILES: COc1cccc(-c2nn(-c3nc4ccccc4[nH]3)c(O)c2CCc2ccc(F)cc2)n1 ; Oc1c(CCc2ccc(F)cc2)c(CC2CCNCC2)nn1-c1nc2ccccc2[nH]1 ; CC(C)(O)OC(=O)N1CCCC(Cc2nn(-c3nc4ccccc4[nH]3)c(O)c2CCc2ccc(F)cc2)C1 ; CC(=O)N1CCC(Cc2nn(-c3nc4ccccc4[nH]3)c(O)c2CCc2ccc(F)cc2)CC1 ; CS(=O)(=O)N1CCC(Cc2nn(-c3nc4ccccc4[nH]3)c(O)c2CCc2ccc(F)cc2)CC1]

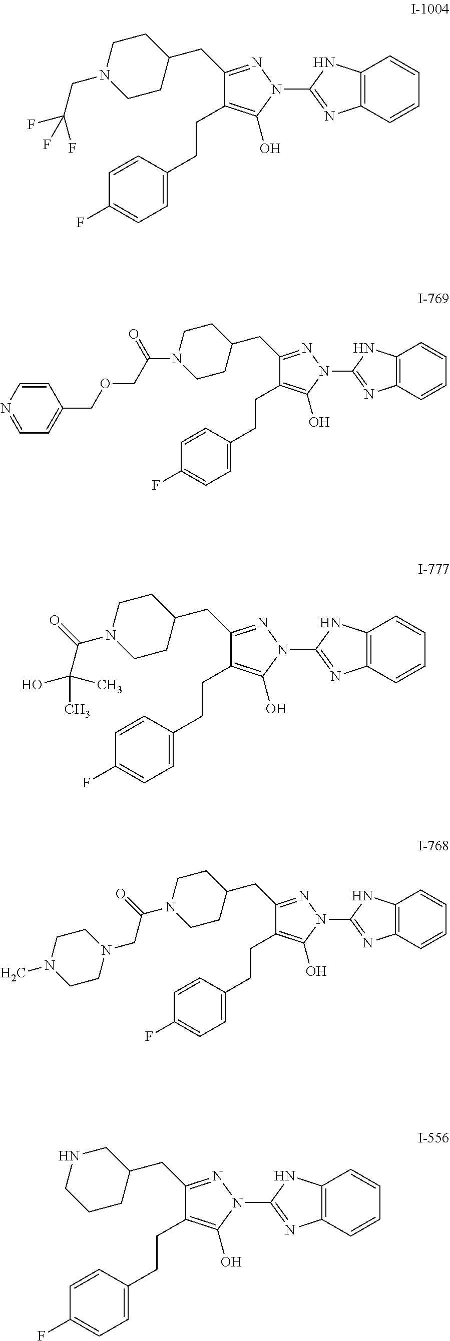 [SMILES: Oc1c(CCc2ccc(F)cc2)c(CC2CCN(CC(F)(F)F)CC2)nn1-c1nc2ccccc2[nH]1 ; O=C(COCc1ccncc1)N1CCC(Cc2nn(-c3nc4ccccc4[nH]3)c(O)c2CCc2ccc(F)cc2)CC1 ; CC(C)(O)C(=O)N1CCC(Cc2nn(-c3nc4ccccc4[nH]3)c(O)c2CCc2ccc(F)cc2)CC1 ; CN1CCN(CC(=O)N2CCC(Cc3nn(-c4nc5ccccc5[nH]4)c(O)c3CCc3ccc(F)cc3)CC2)CC1 ; Oc1c(CCc2ccc(F)cc2)c(CC2CCCNC2)nn1-c1nc2ccccc2[nH]1]

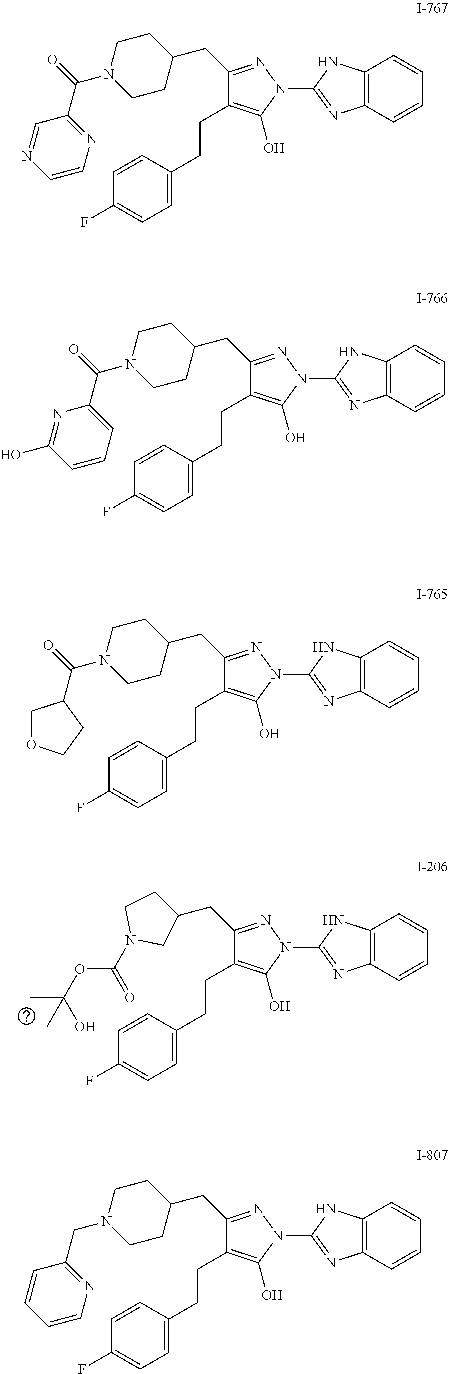 [SMILES: O=C(c1cnccn1)N1CCC(Cc2nn(-c3nc4ccccc4[nH]3)c(O)c2CCc2ccc(F)cc2)CC1 ; O=C(c1cccc(O)n1)N1CCC(Cc2nn(-c3nc4ccccc4[nH]3)c(O)c2CCc2ccc(F)cc2)CC1 ; O=C(C1CCOC1)N1CCC(Cc2nn(-c3nc4ccccc4[nH]3)c(O)c2CCc2ccc(F)cc2)CC1 ; CC(C)(O)OC(=O)N1CCC(Cc2nn(-c3nc4ccccc4[nH]3)c(O)c2CCc2ccc(F)cc2)C1 ; Oc1c(CCc2ccc(F)cc2)c(CC2CCN(Cc3ccccn3)CC2)nn1-c1nc2ccccc2[nH]1]

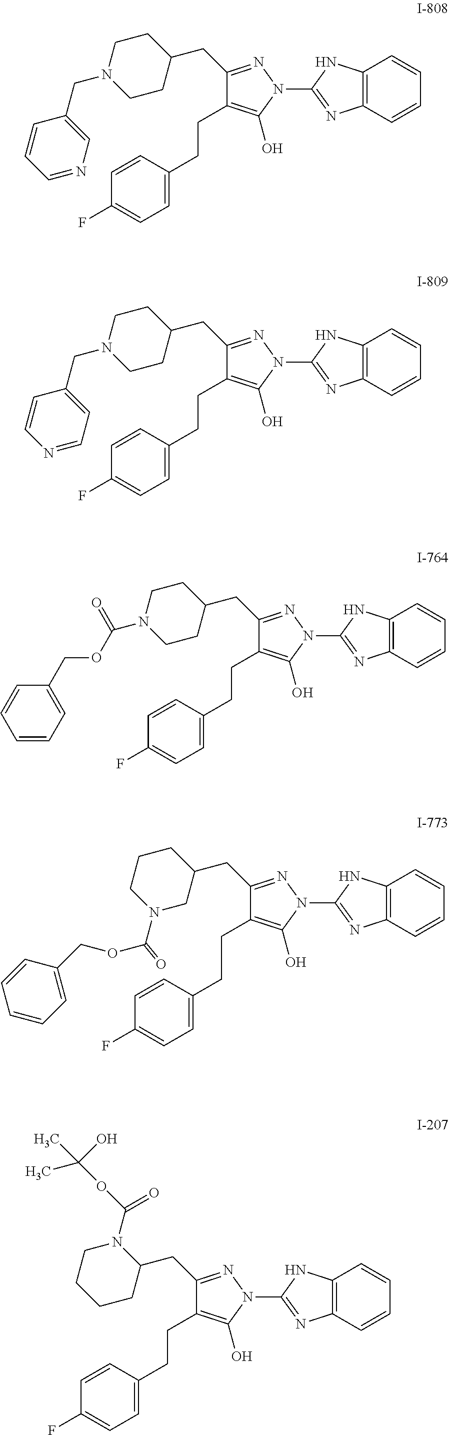 [SMILES: Oc1c(CCc2ccc(F)cc2)c(CC2CCN(Cc3cccnc3)CC2)nn1-c1nc2ccccc2[nH]1 ; Oc1c(CCc2ccc(F)cc2)c(CC2CCN(Cc3ccncc3)CC2)nn1-c1nc2ccccc2[nH]1 ; O=C(OCc1ccccc1)N1CCC(Cc2nn(-c3nc4ccccc4[nH]3)c(O)c2CCc2ccc(F)cc2)CC1 ; O=C(OCc1ccccc1)N1CCCC(Cc2nn(-c3nc4ccccc4[nH]3)c(O)c2CCc2ccc(F)cc2)C1 ; CC(C)(O)OC(=O)N1CCCCC1Cc1nn(-c2nc3ccccc3[nH]2)c(O)c1CCc1ccc(F)cc1]

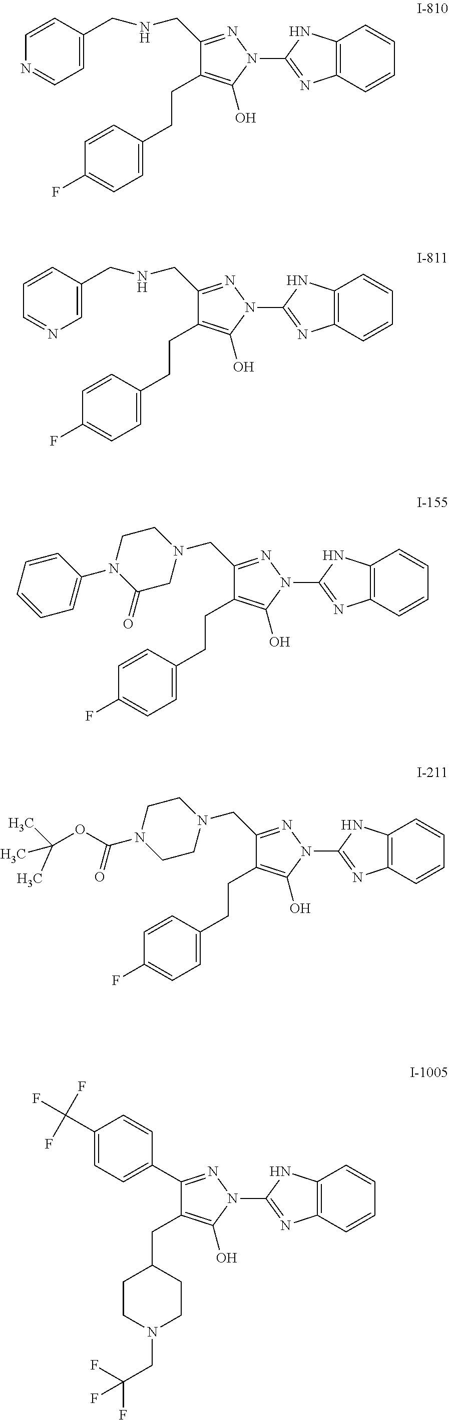 [SMILES: Oc1c(CCc2ccc(F)cc2)c(CNCc2ccncc2)nn1-c1nc2ccccc2[nH]1 ; Oc1c(CCc2ccc(F)cc2)c(CNCc2cccnc2)nn1-c1nc2ccccc2[nH]1 ; O=C1CN(Cc2nn(-c3nc4ccccc4[nH]3)c(O)c2CCc2ccc(F)cc2)CCN1c1ccccc1 ; CC(C)(C)OC(=O)N1CCN(Cc2nn(-c3nc4ccccc4[nH]3)c(O)c2CCc2ccc(F)cc2)CC1 ; Oc1c(CC2CCN(CC(F)(F)F)CC2)c(-c2ccc(C(F)(F)F)cc2)nn1-c1nc2ccccc2[nH]1]

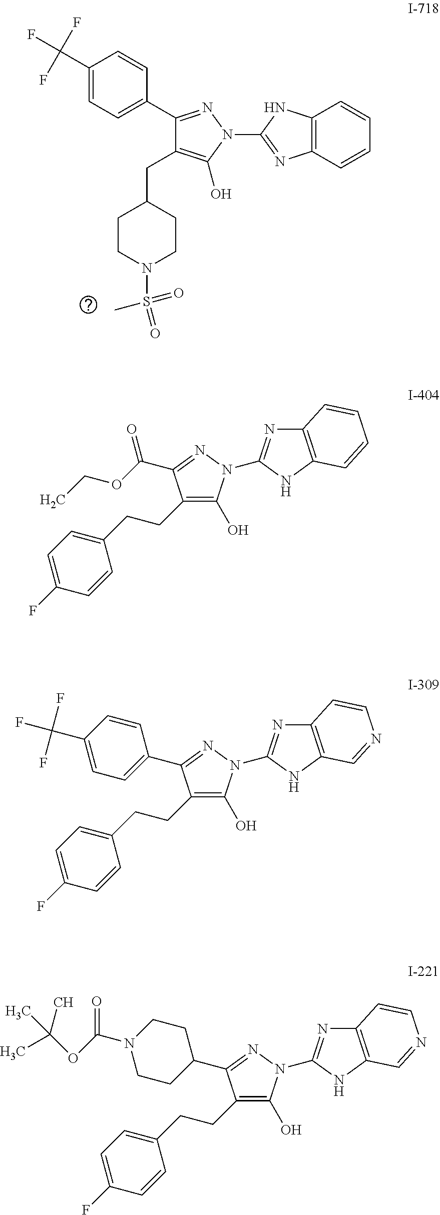 [SMILES: CS(=O)(=O)N1CCC(Cc2c(-c3ccc(C(F)(F)F)cc3)nn(-c3nc4ccccc4[nH]3)c2O)CC1 ; CCOC(=O)c1nn(-c2nc3ccccc3[nH]2)c(O)c1CCc1ccc(F)cc1 ; Oc1c(CCc2ccc(F)cc2)c(-c2ccc(C(F)(F)F)cc2)nn1-c1nc2ccncc2[nH]1 ; CC(C)(C)OC(=O)N1CCC(c2nn(-c3nc4ccncc4[nH]3)c(O)c2CCc2ccc(F)cc2)CC1]

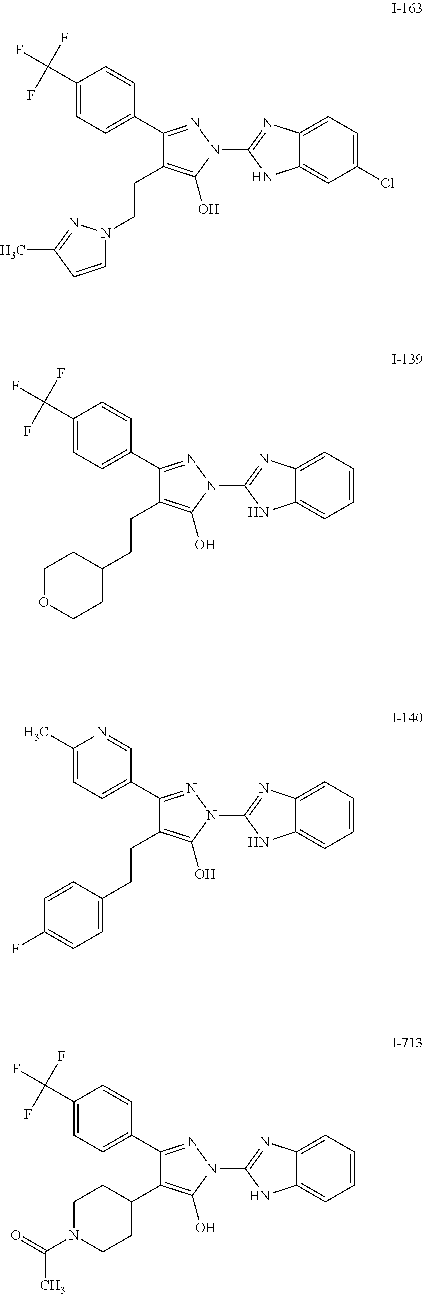 [SMILES: Cc1ccn(CCc2c(-c3ccc(C(F)(F)F)cc3)nn(-c3nc4ccc(Cl)cc4[nH]3)c2O)n1 ; Oc1c(CCC2CCOCC2)c(-c2ccc(C(F)(F)F)cc2)nn1-c1nc2ccccc2[nH]1 ; Cc1ccc(-c2nn(-c3nc4ccccc4[nH]3)c(O)c2CCc2ccc(F)cc2)cn1 ; CC(=O)N1CCC(c2c(-c3ccc(C(F)(F)F)cc3)nn(-c3nc4ccccc4[nH]3)c2O)CC1]

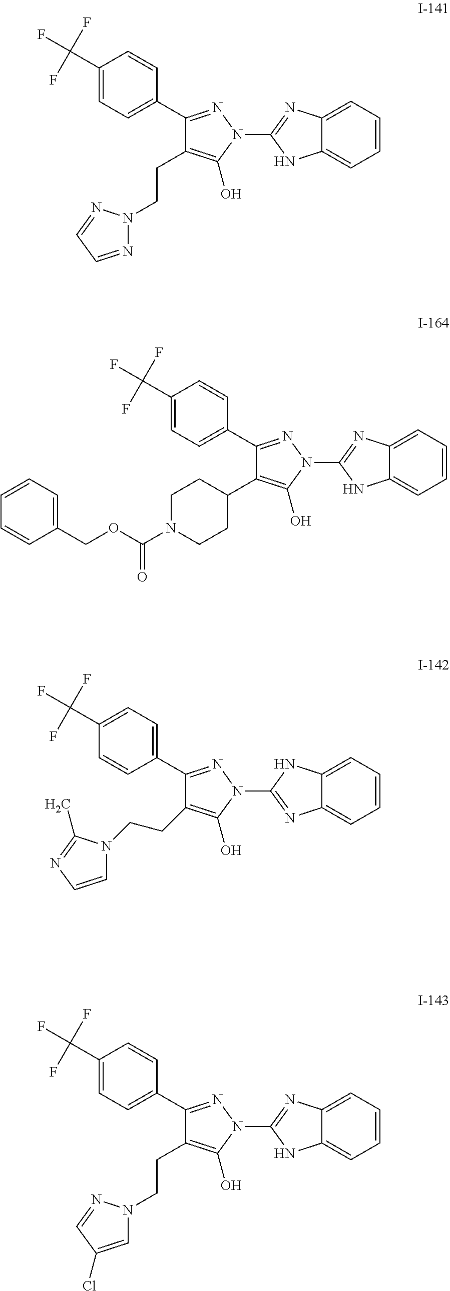 [SMILES: Oc1c(CCn2nccn2)c(-c2ccc(C(F)(F)F)cc2)nn1-c1nc2ccccc2[nH]1 ; O=C(OCc1ccccc1)N1CCC(c2c(-c3ccc(C(F)(F)F)cc3)nn(-c3nc4ccccc4[nH]3)c2O)CC1 ; Cc1nccn1CCc1c(-c2ccc(C(F)(F)F)cc2)nn(-c2nc3ccccc3[nH]2)c1O ; Oc1c(CCn2cc(Cl)cn2)c(-c2ccc(C(F)(F)F)cc2)nn1-c1nc2ccccc2[nH]1]

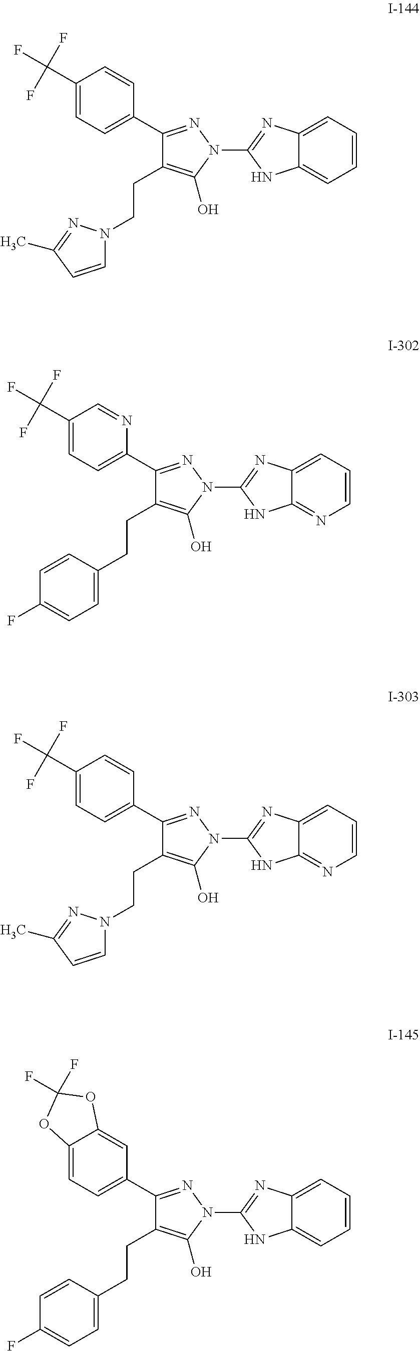 [SMILES: Cc1ccn(CCc2c(-c3ccc(C(F)(F)F)cc3)nn(-c3nc4ccccc4[nH]3)c2O)n1 ; Oc1c(CCc2ccc(F)cc2)c(-c2ccc(C(F)(F)F)cn2)nn1-c1nc2cccnc2[nH]1 ; Cc1ccn(CCc2c(-c3ccc(C(F)(F)F)cc3)nn(-c3nc4cccnc4[nH]3)c2O)n1 ; Oc1c(CCc2ccc(F)cc2)c(-c2ccc3c(c2)OC(F)(F)O3)nn1-c1nc2ccccc2[nH]1]

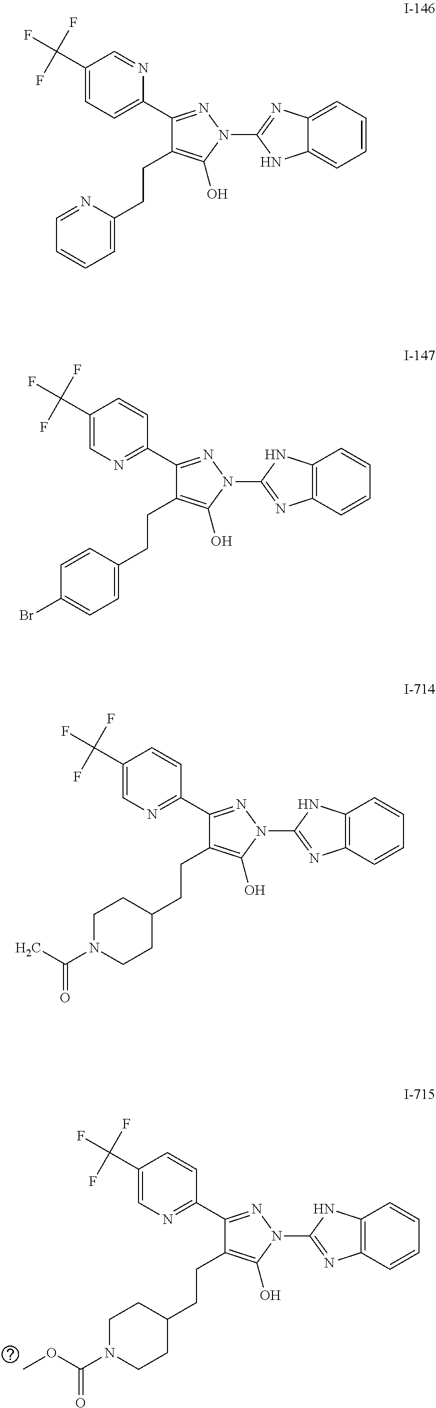 [SMILES: Oc1c(CCc2ccccn2)c(-c2ccc(C(F)(F)F)cn2)nn1-c1nc2ccccc2[nH]1 ; Oc1c(CCc2ccc(Br)cc2)c(-c2ccc(C(F)(F)F)cn2)nn1-c1nc2ccccc2[nH]1 ; CC(=O)N1CCC(CCc2c(-c3ccc(C(F)(F)F)cn3)nn(-c3nc4ccccc4[nH]3)c2O)CC1 ; COC(=O)N1CCC(CCc2c(-c3ccc(C(F)(F)F)cn3)nn(-c3nc4ccccc4[nH]3)c2O)CC1]

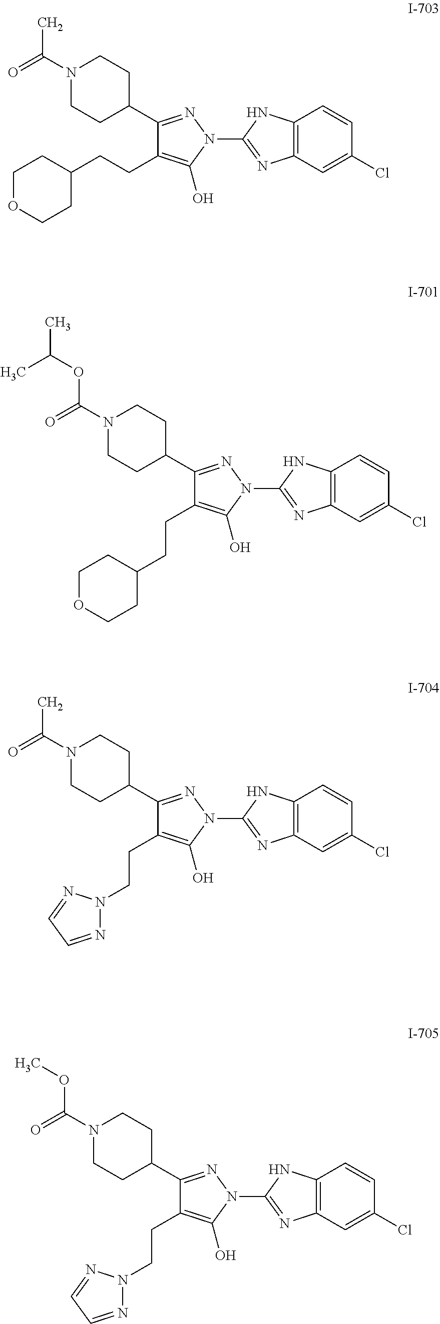 [SMILES: CC(=O)N1CCC(c2nn(-c3nc4cc(Cl)ccc4[nH]3)c(O)c2CCC2CCOCC2)CC1 ; CC(C)OC(=O)N1CCC(c2nn(-c3nc4cc(Cl)ccc4[nH]3)c(O)c2CCC2CCOCC2)CC1 ; CC(=O)N1CCC(c2nn(-c3nc4cc(Cl)ccc4[nH]3)c(O)c2CCn2nccn2)CC1 ; COC(=O)N1CCC(c2nn(-c3nc4cc(Cl)ccc4[nH]3)c(O)c2CCn2nccn2)CC1]

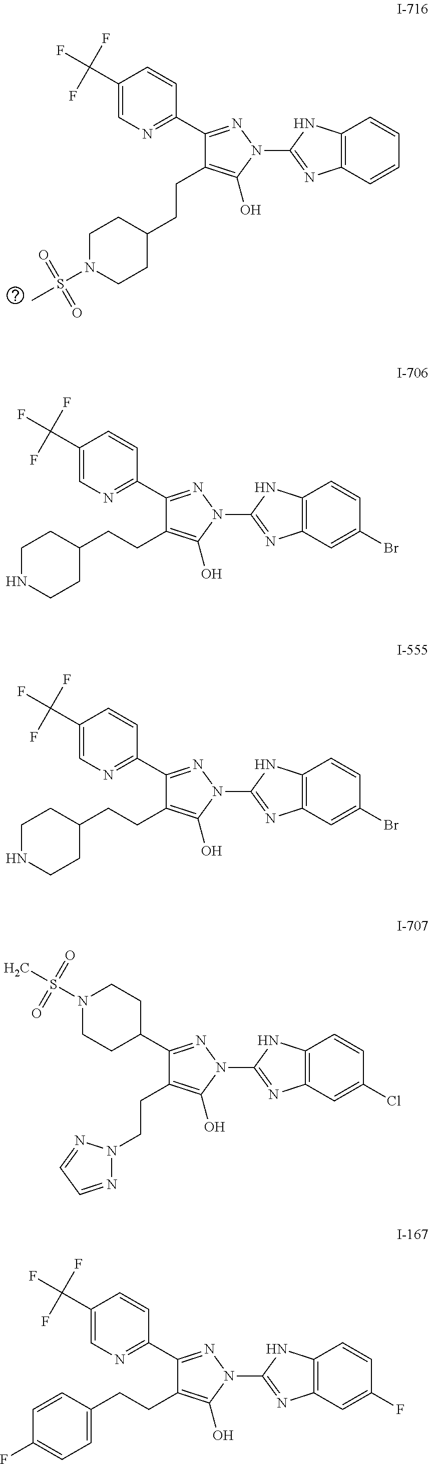 [SMILES: CS(=O)(=O)N1CCC(CCc2c(-c3ccc(C(F)(F)F)cn3)nn(-c3nc4ccccc4[nH]3)c2O)CC1 ; Oc1c(CCC2CCNCC2)c(-c2ccc(C(F)(F)F)cn2)nn1-c1nc2cc(Br)ccc2[nH]1 ; Oc1c(CCC2CCNCC2)c(-c2ccc(C(F)(F)F)cn2)nn1-c1nc2cc(Br)ccc2[nH]1 ; CS(=O)(=O)N1CCC(c2nn(-c3nc4cc(Cl)ccc4[nH]3)c(O)c2CCn2nccn2)CC1 ; Oc1c(CCc2ccc(F)cc2)c(-c2ccc(C(F)(F)F)cn2)nn1-c1nc2cc(F)ccc2[nH]1]

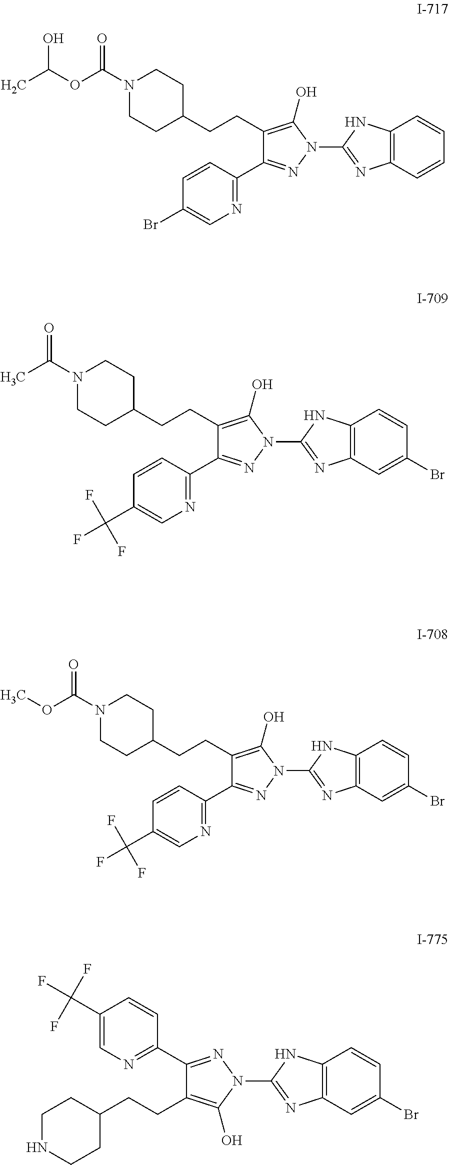 [SMILES: CC(O)OC(=O)N1CCC(CCc2c(-c3ccc(Br)cn3)nn(-c3nc4ccccc4[nH]3)c2O)CC1 ; CC(=O)N1CCC(CCc2c(-c3ccc(C(F)(F)F)cn3)nn(-c3nc4cc(Br)ccc4[nH]3)c2O)CC1 ; COC(=O)N1CCC(CCc2c(-c3ccc(C(F)(F)F)cn3)nn(-c3nc4cc(Br)ccc4[nH]3)c2O)CC1 ; Oc1c(CCC2CCNCC2)c(-c2ccc(C(F)(F)F)cn2)nn1-c1nc2cc(Br)ccc2[nH]1]

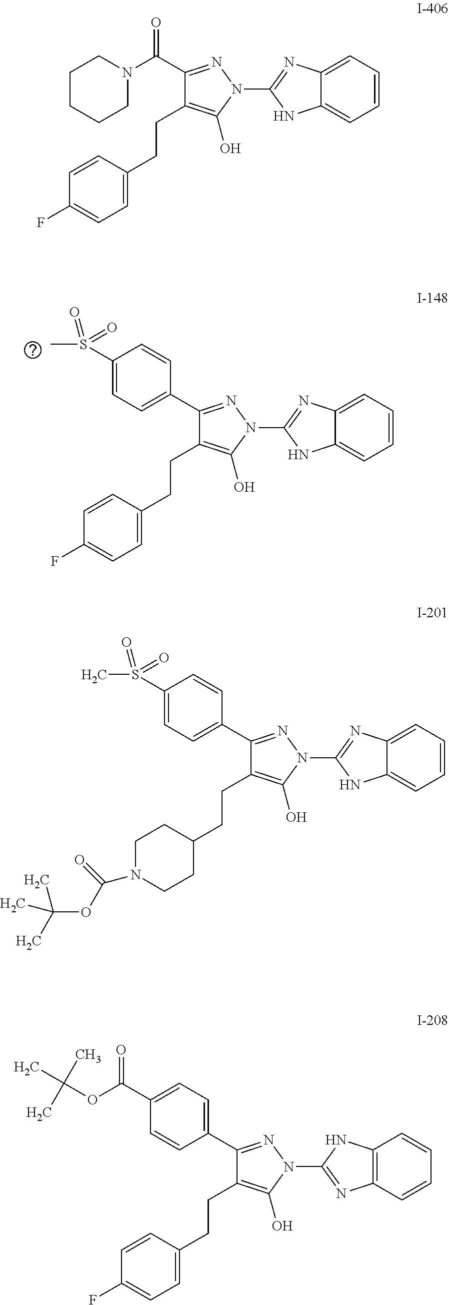 [SMILES: O=C(c1nn(-c2nc3ccccc3[nH]2)c(O)c1CCc1ccc(F)cc1)N1CCCCC1 ; CS(=O)(=O)c1ccc(-c2nn(-c3nc4ccccc4[nH]3)c(O)c2CCc2ccc(F)cc2)cc1 ; CC(C)(C)OC(=O)N1CCC(CCc2c(-c3ccc(S(C)(=O)=O)cc3)nn(-c3nc4ccccc4[nH]3)c2O)CC1 ; CC(C)(C)OC(=O)c1ccc(-c2nn(-c3nc4ccccc4[nH]3)c(O)c2CCc2ccc(F)cc2)cc1]

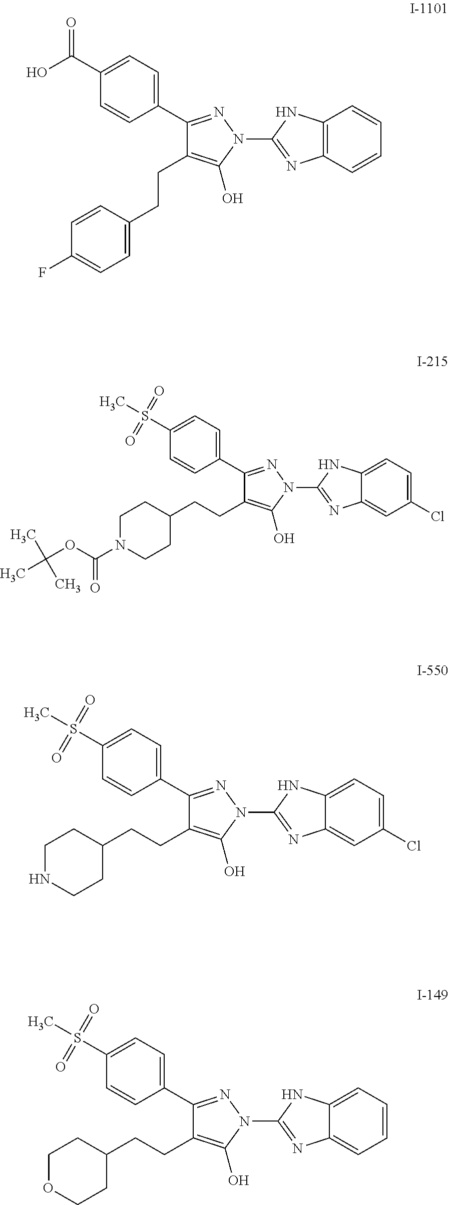 [SMILES: O=C(O)c1ccc(-c2nn(-c3nc4ccccc4[nH]3)c(O)c2CCc2ccc(F)cc2)cc1 ; CC(C)(C)OC(=O)N1CCC(CCc2c(-c3ccc(S(C)(=O)=O)cc3)nn(-c3nc4cc(Cl)ccc4[nH]3)c2O)CC1 ; CS(=O)(=O)c1ccc(-c2nn(-c3nc4cc(Cl)ccc4[nH]3)c(O)c2CCC2CCNCC2)cc1 ; CS(=O)(=O)c1ccc(-c2nn(-c3nc4ccccc4[nH]3)c(O)c2CCC2CCOCC2)cc1]

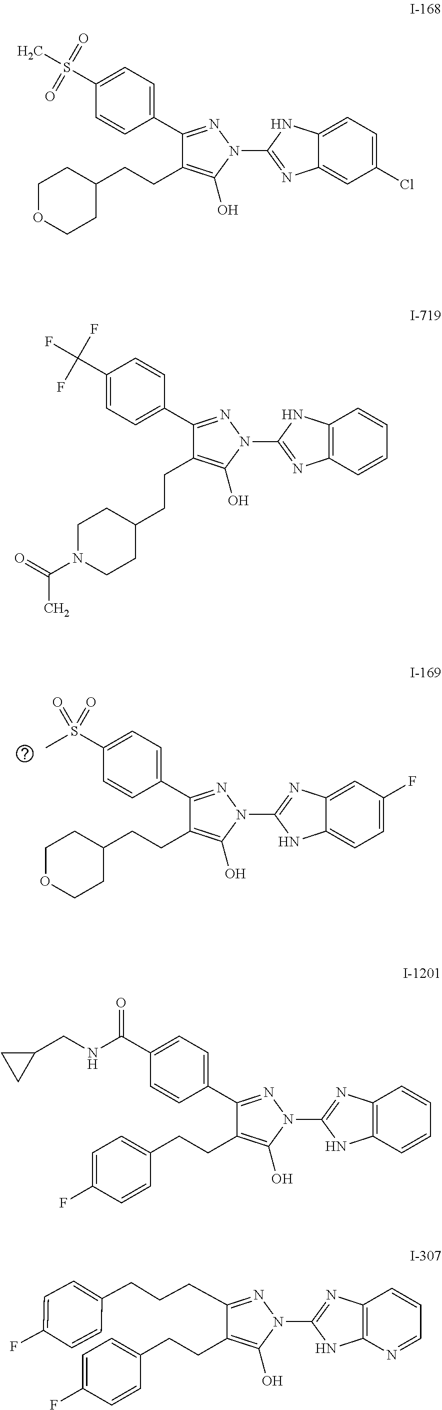 [SMILES: CS(=O)(=O)c1ccc(-c2nn(-c3nc4cc(Cl)ccc4[nH]3)c(O)c2CCC2CCOCC2)cc1 ; CC(=O)N1CCC(CCc2c(-c3ccc(C(F)(F)F)cc3)nn(-c3nc4ccccc4[nH]3)c2O)CC1 ; CS(=O)(=O)c1ccc(-c2nn(-c3nc4cc(F)ccc4[nH]3)c(O)c2CCC2CCOCC2)cc1 ; O=C(NCC1CC1)c1ccc(-c2nn(-c3nc4ccccc4[nH]3)c(O)c2CCc2ccc(F)cc2)cc1 ; Oc1c(CCc2ccc(F)cc2)c(CCCc2ccc(F)cc2)nn1-c1nc2cccnc2[nH]1]

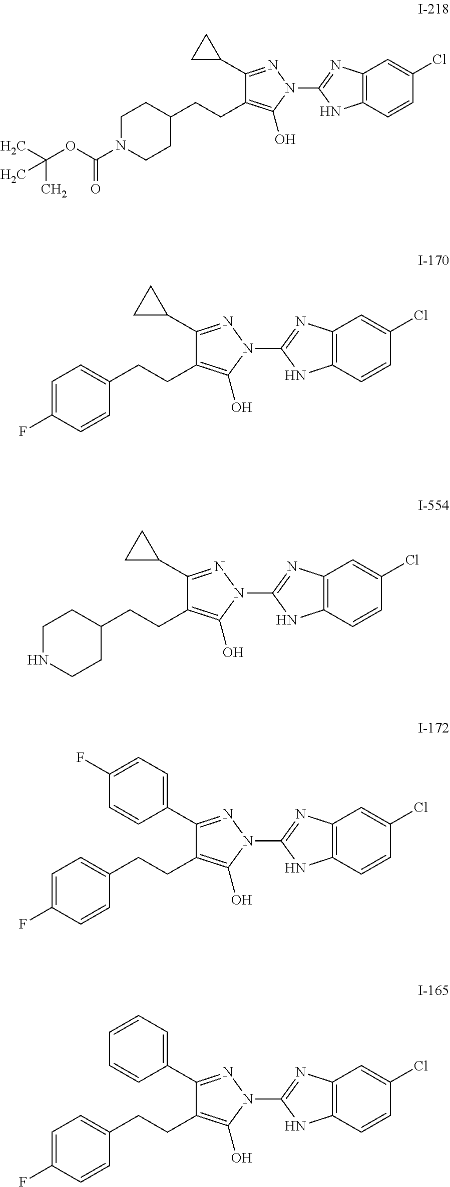 [SMILES: CC(C)(C)OC(=O)N1CCC(CCc2c(C3CC3)nn(-c3nc4cc(Cl)ccc4[nH]3)c2O)CC1 ; Oc1c(CCc2ccc(F)cc2)c(C2CC2)nn1-c1nc2cc(Cl)ccc2[nH]1 ; Oc1c(CCC2CCNCC2)c(C2CC2)nn1-c1nc2cc(Cl)ccc2[nH]1 ; Oc1c(CCc2ccc(F)cc2)c(-c2ccc(F)cc2)nn1-c1nc2cc(Cl)ccc2[nH]1 ; Oc1c(CCc2ccc(F)cc2)c(-c2ccccc2)nn1-c1nc2cc(Cl)ccc2[nH]1]

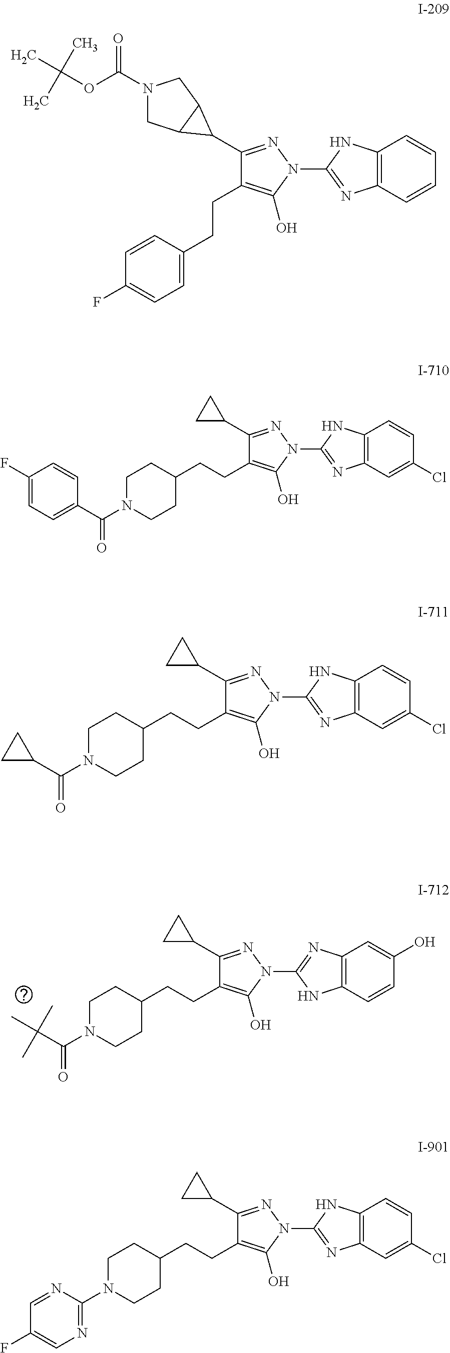 [SMILES: CC(C)(C)OC(=O)N1CC2C(C1)C2c1nn(-c2nc3ccccc3[nH]2)c(O)c1CCc1ccc(F)cc1 ; O=C(c1ccc(F)cc1)N1CCC(CCc2c(C3CC3)nn(-c3nc4cc(Cl)ccc4[nH]3)c2O)CC1 ; O=C(C1CC1)N1CCC(CCc2c(C3CC3)nn(-c3nc4cc(Cl)ccc4[nH]3)c2O)CC1 ; CC(C)(C)C(=O)N1CCC(CCc2c(C3CC3)nn(-c3nc4cc(O)ccc4[nH]3)c2O)CC1 ; Oc1c(CCC2CCN(c3ncc(F)cn3)CC2)c(C2CC2)nn1-c1nc2cc(Cl)ccc2[nH]1]

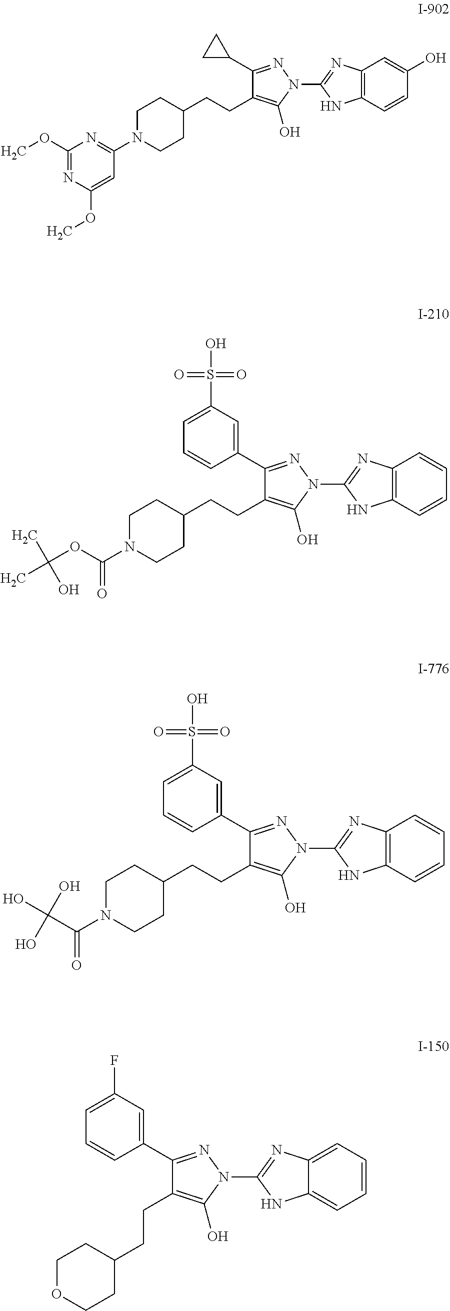 [SMILES: COc1cc(N2CCC(CCc3c(C4CC4)nn(-c4nc5cc(O)ccc5[nH]4)c3O)CC2)nc(OC)n1 ; CC(C)(O)OC(=O)N1CCC(CCc2c(-c3cccc(S(=O)(=O)O)c3)nn(-c3nc4ccccc4[nH]3)c2O)CC1 ; O=C(N1CCC(CCc2c(-c3cccc(S(=O)(=O)O)c3)nn(-c3nc4ccccc4[nH]3)c2O)CC1)C(O)(O)O ; Oc1c(CCC2CCOCC2)c(-c2cccc(F)c2)nn1-c1nc2ccccc2[nH]1]

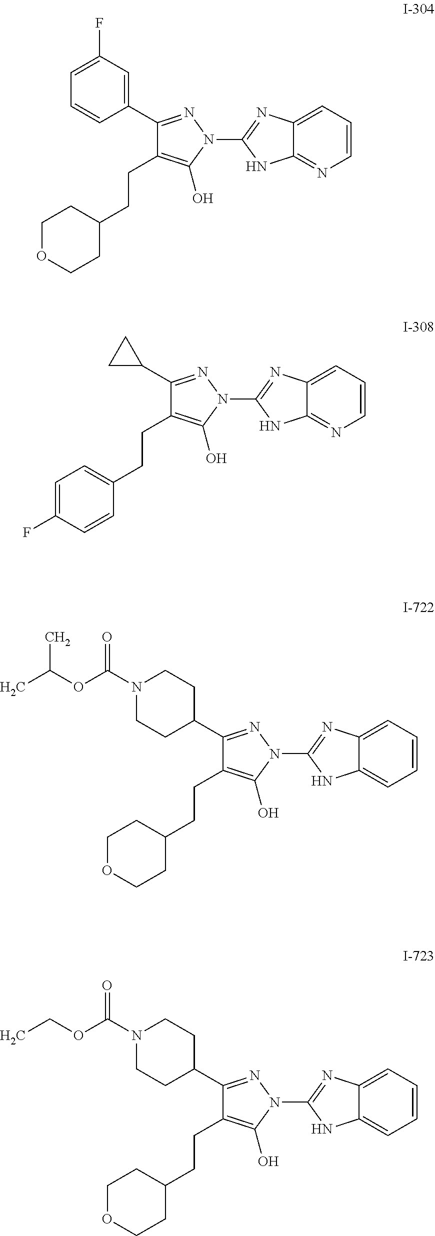 [SMILES: Oc1c(CCC2CCOCC2)c(-c2cccc(F)c2)nn1-c1nc2cccnc2[nH]1 ; Oc1c(CCc2ccc(F)cc2)c(C2CC2)nn1-c1nc2cccnc2[nH]1 ; CC(C)OC(=O)N1CCC(c2nn(-c3nc4ccccc4[nH]3)c(O)c2CCC2CCOCC2)CC1 ; CCOC(=O)N1CCC(c2nn(-c3nc4ccccc4[nH]3)c(O)c2CCC2CCOCC2)CC1]

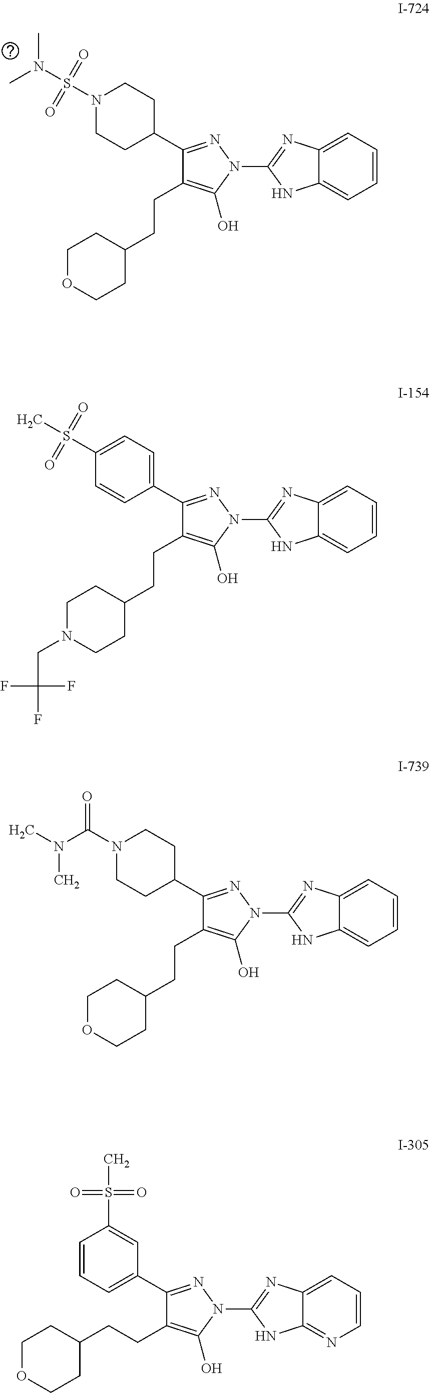 [SMILES: CN(C)S(=O)(=O)N1CCC(c2nn(-c3nc4ccccc4[nH]3)c(O)c2CCC2CCOCC2)CC1 ; CS(=O)(=O)c1ccc(-c2nn(-c3nc4ccccc4[nH]3)c(O)c2CCC2CCN(CC(F)(F)F)CC2)cc1 ; CN(C)C(=O)N1CCC(c2nn(-c3nc4ccccc4[nH]3)c(O)c2CCC2CCOCC2)CC1 ; CS(=O)(=O)c1cccc(-c2nn(-c3nc4cccnc4[nH]3)c(O)c2CCC2CCOCC2)c1]

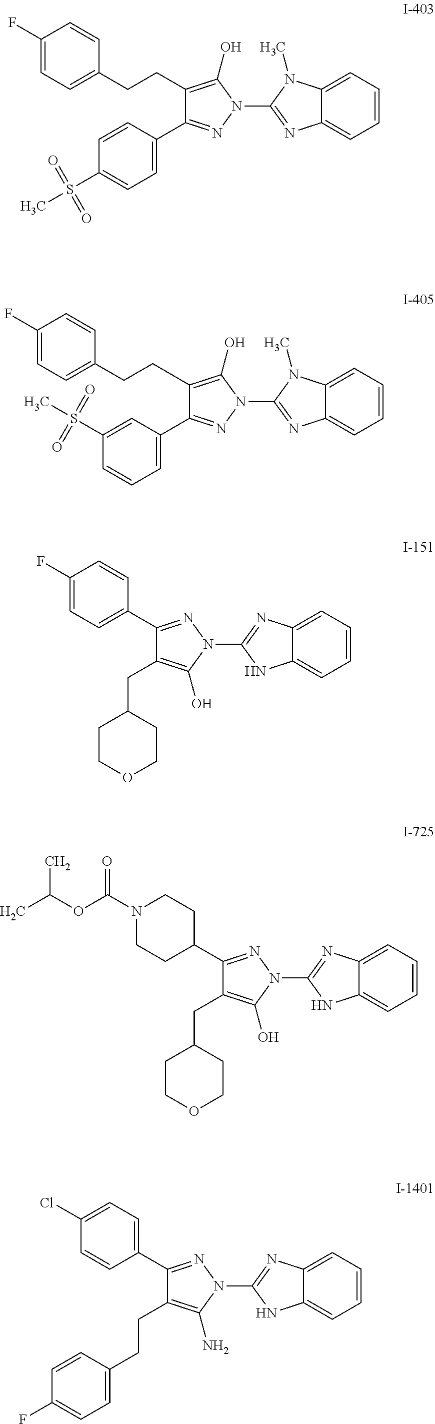 [SMILES: Cn1c(-n2nc(-c3ccc(S(C)(=O)=O)cc3)c(CCc3ccc(F)cc3)c2O)nc2ccccc21 ; Cn1c(-n2nc(-c3cccc(S(C)(=O)=O)c3)c(CCc3ccc(F)cc3)c2O)nc2ccccc21 ; Oc1c(CC2CCOCC2)c(-c2ccc(F)cc2)nn1-c1nc2ccccc2[nH]1 ; CC(C)OC(=O)N1CCC(c2nn(-c3nc4ccccc4[nH]3)c(O)c2CC2CCOCC2)CC1 ; Nc1c(CCc2ccc(F)cc2)c(-c2ccc(Cl)cc2)nn1-c1nc2ccccc2[nH]1]

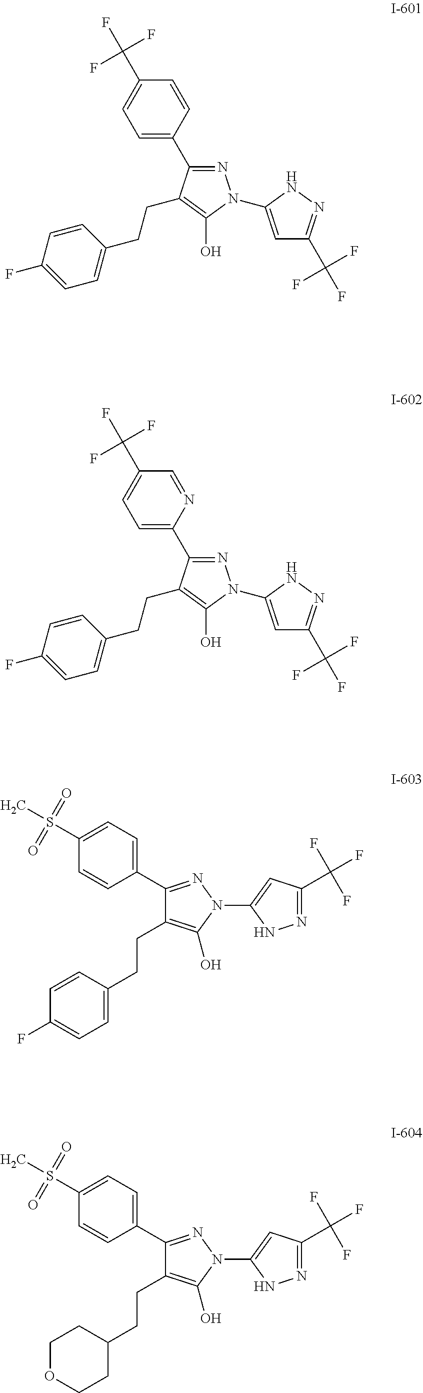 [SMILES: Oc1c(CCc2ccc(F)cc2)c(-c2ccc(C(F)(F)F)cc2)nn1-c1cc(C(F)(F)F)n[nH]1 ; Oc1c(CCc2ccc(F)cc2)c(-c2ccc(C(F)(F)F)cn2)nn1-c1cc(C(F)(F)F)n[nH]1 ; CS(=O)(=O)c1ccc(-c2nn(-c3cc(C(F)(F)F)n[nH]3)c(O)c2CCc2ccc(F)cc2)cc1 ; CS(=O)(=O)c1ccc(-c2nn(-c3cc(C(F)(F)F)n[nH]3)c(O)c2CCC2CCOCC2)cc1]

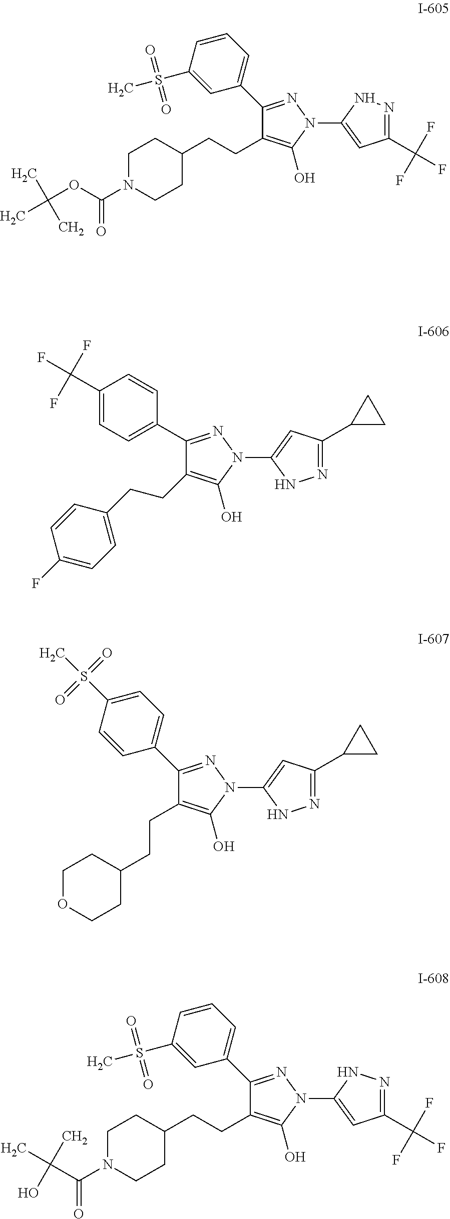 [SMILES: CC(C)(C)OC(=O)N1CCC(CCc2c(-c3cccc(S(C)(=O)=O)c3)nn(-c3cc(C(F)(F)F)n[nH]3)c2O)CC1 ; Oc1c(CCc2ccc(F)cc2)c(-c2ccc(C(F)(F)F)cc2)nn1-c1cc(C2CC2)n[nH]1 ; CS(=O)(=O)c1ccc(-c2nn(-c3cc(C4CC4)n[nH]3)c(O)c2CCC2CCOCC2)cc1 ; CC(C)(O)C(=O)N1CCC(CCc2c(-c3cccc(S(C)(=O)=O)c3)nn(-c3cc(C(F)(F)F)n[nH]3)c2O)CC1]

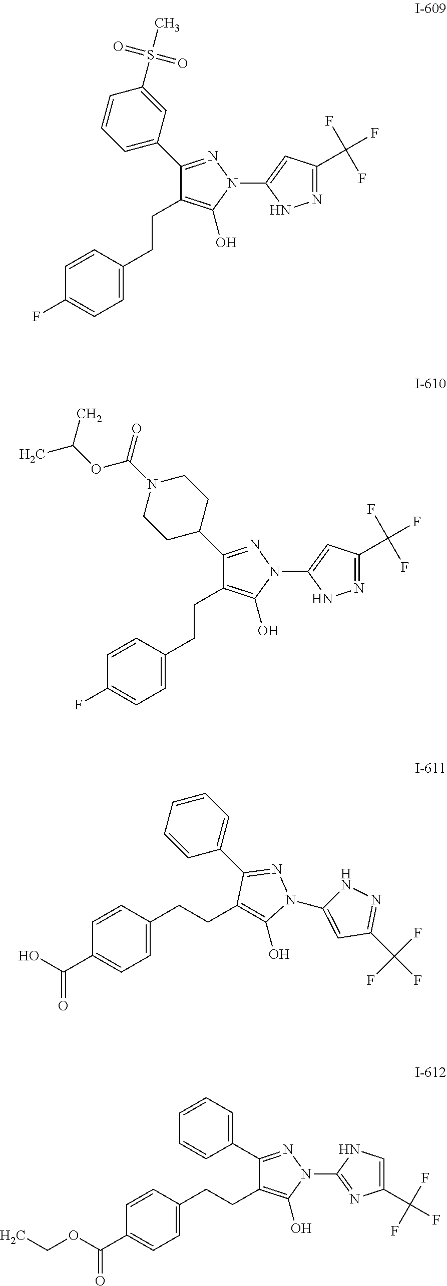 [SMILES: CS(=O)(=O)c1cccc(-c2nn(-c3cc(C(F)(F)F)n[nH]3)c(O)c2CCc2ccc(F)cc2)c1 ; CC(C)OC(=O)N1CCC(c2nn(-c3cc(C(F)(F)F)n[nH]3)c(O)c2CCc2ccc(F)cc2)CC1 ; O=C(O)c1ccc(CCc2c(-c3ccccc3)nn(-c3cc(C(F)(F)F)n[nH]3)c2O)cc1 ; CCOC(=O)c1ccc(CCc2c(-c3ccccc3)nn(-c3nc(C(F)(F)F)c[nH]3)c2O)cc1]

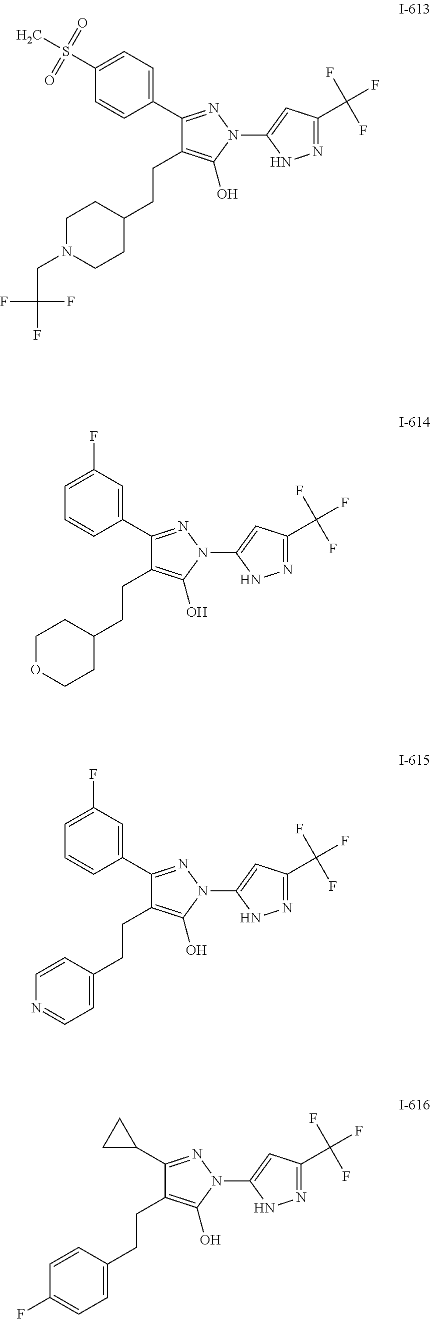 [SMILES: CS(=O)(=O)c1ccc(-c2nn(-c3cc(C(F)(F)F)n[nH]3)c(O)c2CCC2CCN(CC(F)(F)F)CC2)cc1 ; Oc1c(CCC2CCOCC2)c(-c2cccc(F)c2)nn1-c1cc(C(F)(F)F)n[nH]1 ; Oc1c(CCc2ccncc2)c(-c2cccc(F)c2)nn1-c1cc(C(F)(F)F)n[nH]1 ; Oc1c(CCc2ccc(F)cc2)c(C2CC2)nn1-c1cc(C(F)(F)F)n[nH]1]

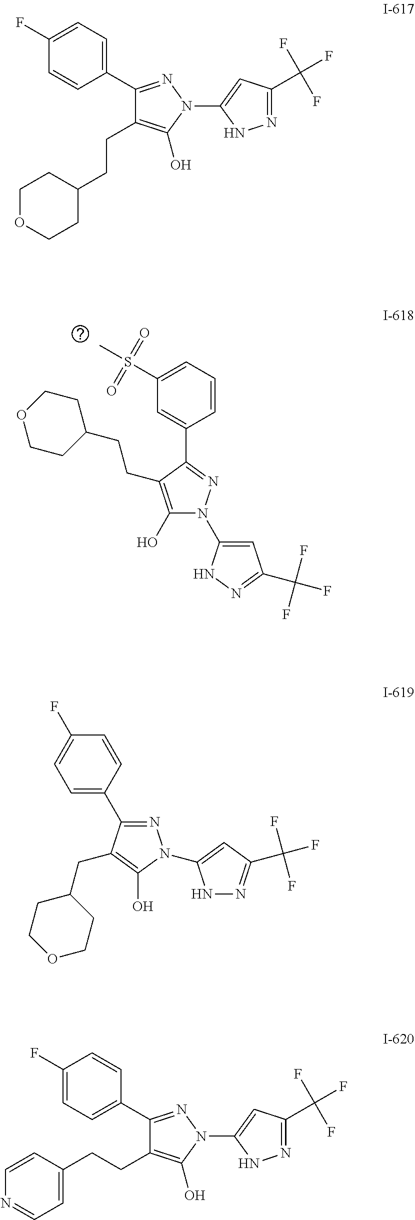 [SMILES: Oc1c(CCC2CCOCC2)c(-c2ccc(F)cc2)nn1-c1cc(C(F)(F)F)n[nH]1 ; CS(=O)(=O)c1cccc(-c2nn(-c3cc(C(F)(F)F)n[nH]3)c(O)c2CCC2CCOCC2)c1 ; Oc1c(CC2CCOCC2)c(-c2ccc(F)cc2)nn1-c1cc(C(F)(F)F)n[nH]1 ; Oc1c(CCc2ccncc2)c(-c2ccc(F)cc2)nn1-c1cc(C(F)(F)F)n[nH]1]

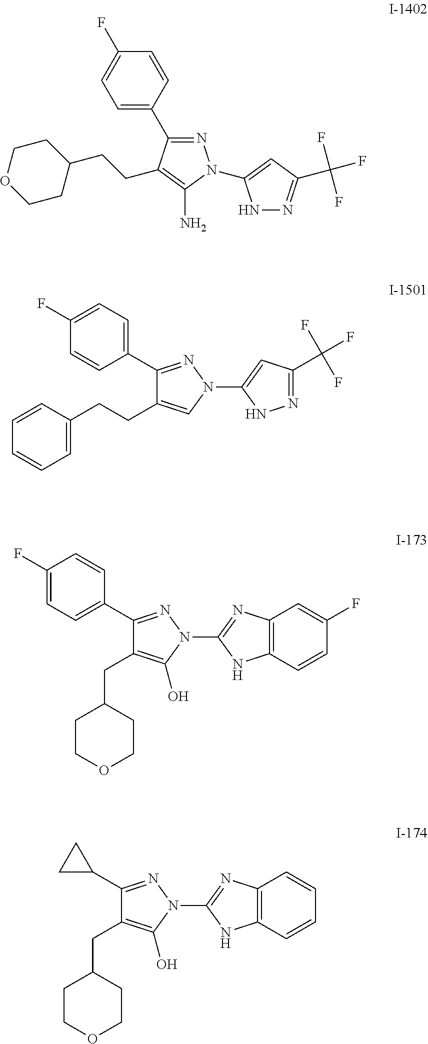 [SMILES: Nc1c(CCC2CCOCC2)c(-c2ccc(F)cc2)nn1-c1cc(C(F)(F)F)n[nH]1 ; Fc1ccc(-c2nn(-c3cc(C(F)(F)F)n[nH]3)cc2CCc2ccccc2)cc1 ; Oc1c(CC2CCOCC2)c(-c2ccc(F)cc2)nn1-c1nc2cc(F)ccc2[nH]1 ; Oc1c(CC2CCOCC2)c(C2CC2)nn1-c1nc2ccccc2[nH]1]

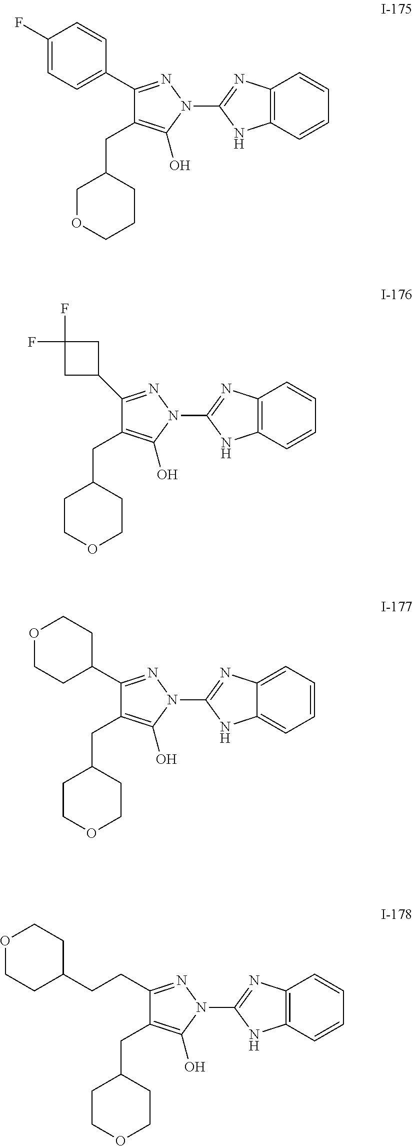 [SMILES: Oc1c(CC2CCCOC2)c(-c2ccc(F)cc2)nn1-c1nc2ccccc2[nH]1 ; Oc1c(CC2CCOCC2)c(C2CC(F)(F)C2)nn1-c1nc2ccccc2[nH]1 ; Oc1c(CC2CCOCC2)c(C2CCOCC2)nn1-c1nc2ccccc2[nH]1 ; Oc1c(CC2CCOCC2)c(CCC2CCOCC2)nn1-c1nc2ccccc2[nH]1]

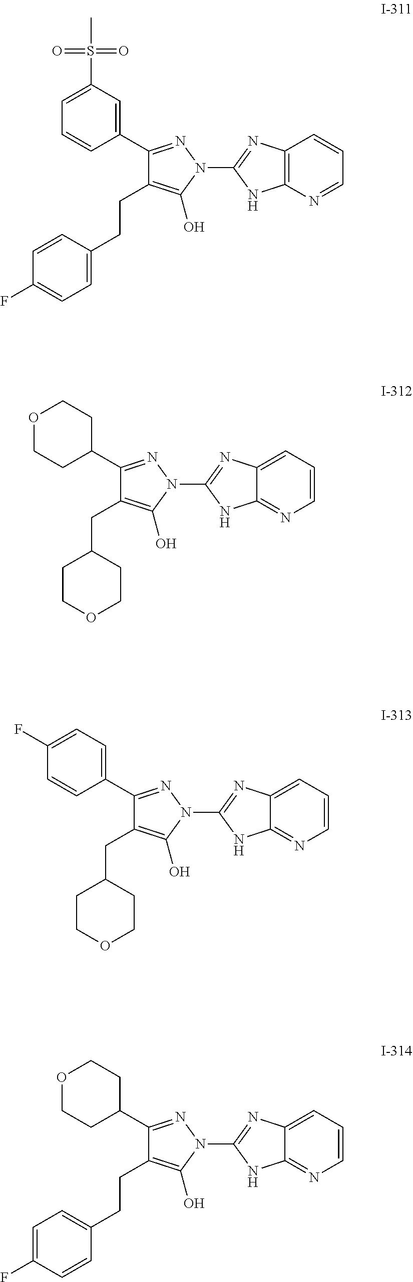 [SMILES: CS(=O)(=O)c1cccc(-c2nn(-c3nc4cccnc4[nH]3)c(O)c2CCc2ccc(F)cc2)c1 ; Oc1c(CC2CCOCC2)c(C2CCOCC2)nn1-c1nc2cccnc2[nH]1 ; Oc1c(CC2CCOCC2)c(-c2ccc(F)cc2)nn1-c1nc2cccnc2[nH]1 ; Oc1c(CCc2ccc(F)cc2)c(C2CCOCC2)nn1-c1nc2cccnc2[nH]1]

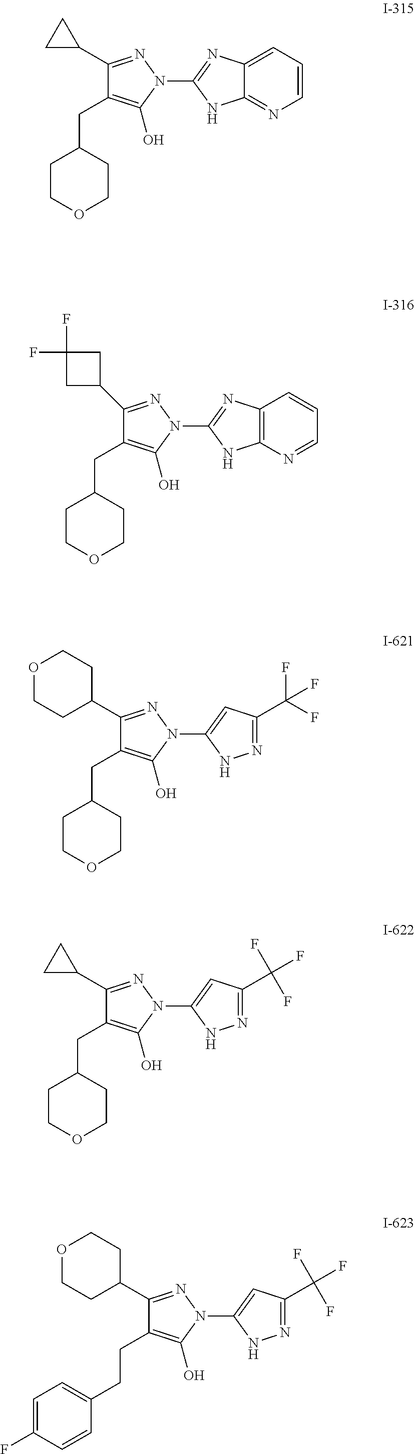 [SMILES: Oc1c(CC2CCOCC2)c(C2CC2)nn1-c1nc2cccnc2[nH]1 ; Oc1c(CC2CCOCC2)c(C2CC(F)(F)C2)nn1-c1nc2cccnc2[nH]1 ; Oc1c(CC2CCOCC2)c(C2CCOCC2)nn1-c1cc(C(F)(F)F)n[nH]1 ; Oc1c(CC2CCOCC2)c(C2CC2)nn1-c1cc(C(F)(F)F)n[nH]1 ; Oc1c(CCc2ccc(F)cc2)c(C2CCOCC2)nn1-c1cc(C(F)(F)F)n[nH]1]

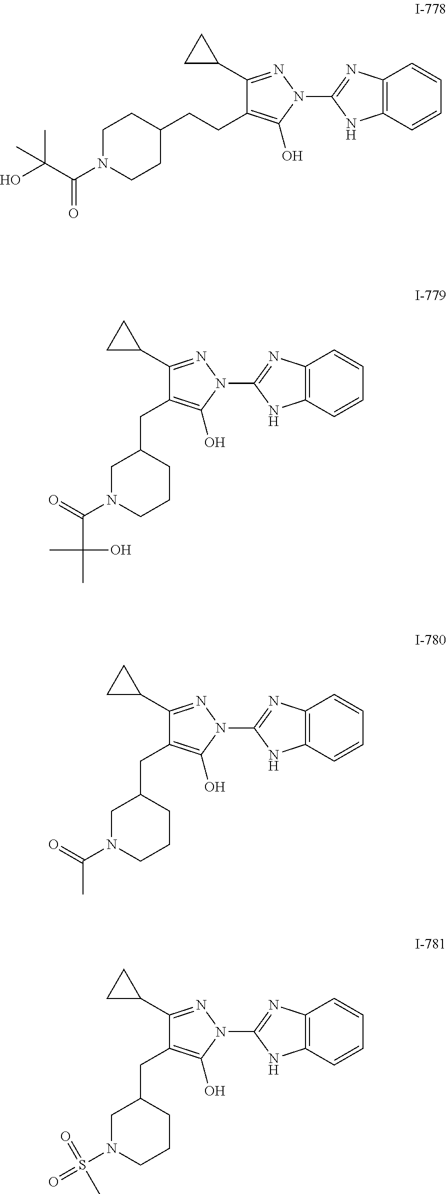 [SMILES: CC(C)(O)C(=O)N1CCC(CCc2c(C3CC3)nn(-c3nc4ccccc4[nH]3)c2O)CC1 ; CC(C)(O)C(=O)N1CCCC(Cc2c(C3CC3)nn(-c3nc4ccccc4[nH]3)c2O)C1 ; CC(=O)N1CCCC(Cc2c(C3CC3)nn(-c3nc4ccccc4[nH]3)c2O)C1 ; CS(=O)(=O)N1CCCC(Cc2c(C3CC3)nn(-c3nc4ccccc4[nH]3)c2O)C1]

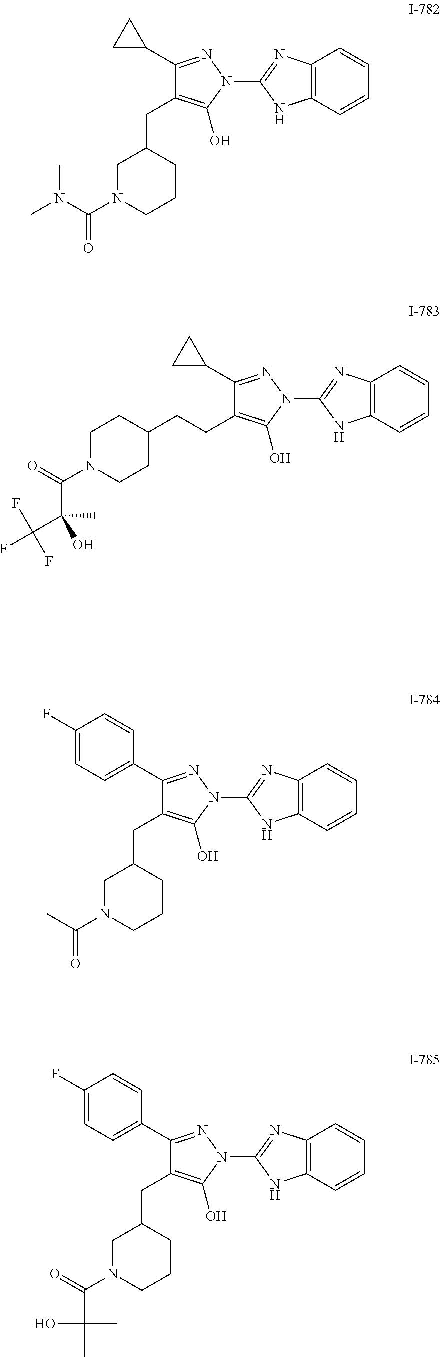 [SMILES: CN(C)C(=O)N1CCCC(Cc2c(C3CC3)nn(-c3nc4ccccc4[nH]3)c2O)C1 ; C[C@@](O)(C(=O)N1CCC(CCc2c(C3CC3)nn(-c3nc4ccccc4[nH]3)c2O)CC1)C(F)(F)F ; CC(=O)N1CCCC(Cc2c(-c3ccc(F)cc3)nn(-c3nc4ccccc4[nH]3)c2O)C1 ; CC(C)(O)C(=O)N1CCCC(Cc2c(-c3ccc(F)cc3)nn(-c3nc4ccccc4[nH]3)c2O)C1]

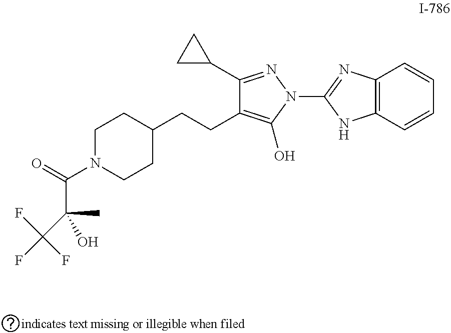 [SMILES: C[C@](O)(C(=O)N1CCC(CCc2c(C3CC3)nn(-c3nc4ccccc4[nH]3)c2O)CC1)C(F)(F)F]

### Pharmaceutical Compositions and Methods of Use

Also disclosed herein is a pharmaceutical formulation comprising a compound as disclosed herein, or a pharmaceutically acceptable salt thereof, and at least one pharmaceutically acceptable carrier, excipient, or combination thereof. The pharmaceutical formulation may further comprise a pharmacologically active agent other than the compound. In particular disclosed embodiments, the pharmacologically active agent is an antiretroviral drug. The antiretroviral drug may be selected from an entry inhibitor, a CCR5 receptor antagonist, a nucleoside reverse transcriptase inhibitor, a non-nucleoside reverse transcriptase inhibitor, a protease inhibitor, an integrase inhibitor, a maturation inhibitor, or combinations thereof. In particular disclosed embodiments, the antiretroviral drug is selected from maraviroc, enfuvirtide, aplaviroc, vicriviroc, zidovudine, didanosine, zalcitabine, stavudine, lamivudine, abacavir, emtricitabine, entecavir, apricitabine, tenofovir, adefovir, efavirenz, nevirapine, delavirdine, etravirine, rilpivirine, saquinavir, ritonavir, indinavir, nelfinavir, amprenavir, lopinavir, atazanavir, fosamprenavir, tipranavir, darunavir, MK-2048, elvitegravir, bevirimat, MPC-9055, or a combination thereof.

Also disclosed herein is a method for inhibiting a biological function of Nef, comprising contacting Nef with an effective amount of a compound disclosed herein. The biological function of Nef may be selected from HIV infectivity, HIV replication, Nef-mediated downregulation of infected cell surface MHC-1/HIV antigen complexes, and AIDS progression.

Further disclosed herein is a method of inhibiting an activity of a Nef-dependent kinase comprising contacting the Nef-dependent kinase with an effective amount of a compound as disclosed herein. In particular disclosed embodiments, the Nef-dependent kinase is coupled with Nef.

Also disclosed is a method of treating a Nef-mediated disease, comprising administering to a subject an effective amount of a compound disclosed herein. Further embodiments concern a method of treating HIV, comprising administering to a subject an effective amount of a compound disclosed herein.

Particular disclosed embodiments concern a method of treating an HIV-related condition comprising administering to a subject an effective amount of a compound disclosed herein. The HIV-related condition may be selected from HIV replication, HIV-associated CD₄+ T-cell loss and immunodeficiency, HIV-induced infection, Kaposi's sarcoma, HIV-associated nephropathy, AIDS dementia complex, and combinations thereof. The subject may be suffering from the HIV-related condition. Also, the subject may be administered the compound prophylactically. In other embodiments, the subject may be administered the compound post-exposure prophylactically.

The compound may also be administered as a formulation. The formulation may comprise the compound and a pharmaceutically acceptable carrier. The formulation also may further comprise at least one antiretroviral drug, as disclosed herein. The subject may be an animal or human, and any one of the disclosed embodiments of the method may be performed in vitro or in vivo.

Embodiments of the disclosed method may be used when the subject is suffering from the HIV-related condition, or the method may be practiced prophylactically or post-exposure prophylactically. The HIV-related condition may be selected from HIV replication, HIV-associated CD₄+ T-cell loss and immunodeficiency, HIV-induced infection, Kaposi's sarcoma, HIV-associated nephropathy, AIDS dementia complex, and combinations thereof.

The effective amount used in the disclosed method may be that which is best suited for treating the subject. The effective amount may range from greater than zero to about 1000 mg/kg/day. In particular disclosed embodiments, the effective amount ranges from 1 mg/kg/day to about 100 mg/kg/day. The subject of the disclosed method may be human or an animal and the method may be performed in vitro or in vivo.

The compound disclosed herein may be used in therapy for a Nef-dependent disorder. As disclosed herein, the compound may be used to treat and/or inhibit a biological pathway that is activated by Nef. Such pathways include, but are not limited to, pathways involving a Src-family kinase, such as Hck, as well as Tec-family kinases including Itk and Btk. In particular disclosed embodiments, the compound may be used to treat or inhibit Nef-dependent HIV-1 replication both in vitro and in vivo. The disclosed compound also may be used to treat or inhibit Nef-dependent HIV-1 infectivity.

In other disclosed embodiments, the compound may be used to treat or inhibit SIV infectivity or replication.

Particular disclosed embodiments of the compound disclosed herein are potent and selective inhibitors of Nef-dependent Hck activity and therefore may be used in in vitro, in vivo, and ex vivo contexts to regulate or inhibit this activity, prevent any Nef-dependent HIV-1 replication, and downregulate MHC-1, as well as the biological responses that result from such activity. In particular disclosed embodiments, the compound may be used to inhibit HIV-1 infectivity and replication in cell types selected from, but not limited to, U87MG astroglioma cells, CEM-T4 lymphoblasts, TZM-bl reporter cell line, and CEM-174. Particular disclosed embodiments of the compound disclosed herein may be used to inhibit Nef-dependent HIV replication in the submicromolar range. Embodiments of the disclosed compound may exhibit IC₅₀ values for Nef-induced Hck activation, as well as Nef-induced Itk activation, in vitro of less than about 3.0 μM; more typically less that about 2.5 μM; even more typically less than about 2.0 μM.

In particular disclosed embodiments, the compound is capable of preventing and/or inhibiting Nef-dependent enhancement of HIV-1 infectivity and replication. The compound is not limited to being active against any particular Nef allele. For instance, embodiments of the disclosed compound are active against a variety of Nef alleles, particularly those that comprise the HIV-1 M-group clades. Exemplary embodiments of the compound may inhibit the replication of HIV-1 in donor PBMCs with an IC₅₀ value of 1 nM to about 100 nM; more typically from about 200 nM to about 350 nM; even more typically from about 250 nM to about 300 nM.

In particular disclosed embodiments, the compound may be used to block Nef-dependent HIV replication and infectivity.

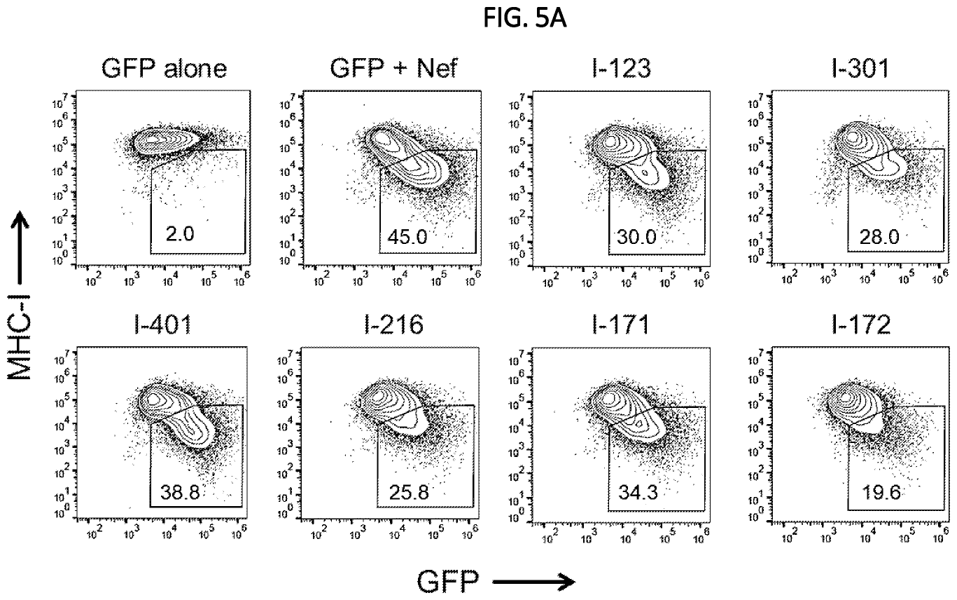

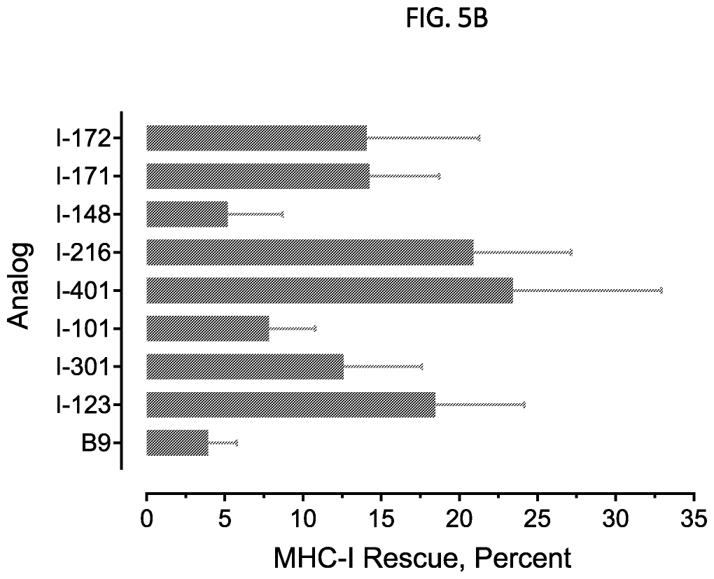

Nef is well-known to prevent cell-surface display of MHC-I in complex with HIV-1 antigenic peptides on infected cells, promoting escape from detection by cytotoxic T lymphocytes. As a consequence, this effect of Nef prevents clearance of the virus from the infected host, and may contribute to establishment and maintenance of the persistent viral reservoir. However, compounds disclosed herein restore MHC-I to the surface of HIV-infected CD₄+ T cells (see FIGS. 5A and 5B). Moreover, when inhibitor-treated cells were co-cultured with autologous CD₈ T cells expanded in the presence of HIV-1 antigenic peptides, the CD₈ T cells were activated and displayed CTL responses against the infected target cells despite the presence of Nef. This result suggests that Nef inhibitors, including those described herein, have the potential to enhance CTL-mediated responses against HIV+ cells in vivo as part of a strategy to clear the latent viral reservoir.

The compounds may be administered orally, parenterally (including subcutaneous injections (SC or depo-SC), intravenous (IV), intramuscular (IM or depo-IM), intrasternal injection or infusion techniques), sublingually, intranasally (inhalation), intrathecally, topically, ophthalmically, or rectally. The pharmaceutical composition may be administered in dosage unit formulations containing conventional non-toxic pharmaceutically acceptable carriers, adjuvants, and/or vehicles. The compounds are preferably formulated into suitable pharmaceutical preparations such as tablets, capsules, or elixirs for oral administration or in sterile solutions or suspensions for parenteral administration. Typically, the compounds described above are formulated into pharmaceutical compositions using techniques and procedures well known in the art.

In some embodiments, one or more of the disclosed compounds (including compounds linked to a detectable label or cargo moiety) are mixed or combined with a suitable pharmaceutically acceptable carrier to prepare a pharmaceutical composition. Pharmaceutical carriers or vehicles suitable for administration of the compounds provided herein include any such carriers known to be suitable for the particular mode of administration. Remington: The Science and Practice of Pharmacy, The University of the Sciences in Philadelphia, Editor, Lippincott, Williams, & Wilkins, Philadelphia, Pa., 21^{st} Edition (2005), describes exemplary compositions and formulations suitable for pharmaceutical delivery of the compounds disclosed herein. In addition, the compounds may be formulated as the sole pharmaceutically active ingredient in the composition or may be combined with other active ingredients.

Upon mixing or addition of the compound(s) to a pharmaceutically acceptable carrier, the resulting mixture may be a solution, suspension, emulsion, or the like. Liposomal suspensions may also be suitable as pharmaceutically acceptable carriers. These may be prepared according to methods known to those skilled in the art. The form of the resulting mixture depends upon a number of factors, including the intended mode of administration and the solubility of the compound in the selected carrier or vehicle. Where the compounds exhibit insufficient solubility, methods for solubilizing may be used. Such methods are known and include, but are not limited to, using cosolvents such as dimethylsulfoxide (DMSO), using surfactants such as Tween®, and dissolution in aqueous sodium bicarbonate. Derivatives of the compounds, such as salts or prodrugs may also be used in formulating effective pharmaceutical compositions. The disclosed compounds may also be prepared with carriers that protect them against rapid elimination from the body, such as time-release formulations or coatings. Such carriers include controlled release formulations, such as, but not limited to, microencapsulated delivery systems.

The disclosed compounds and/or compositions can be enclosed in multiple or single dose containers. The compounds and/or compositions can also be provided in kits, for example, including component parts that can be assembled for use. For example, one or more of the disclosed compounds may be provided in a lyophilized form and a suitable diluent may be provided as separated components for combination prior to use. In some examples, a kit may include a disclosed compound and a second therapeutic agent (such as an anti-retroviral agent) for co-administration. The compound and second therapeutic agent may be provided as separate component parts. A kit may include a plurality of containers, each container holding one or more unit dose of the compound. The containers are preferably adapted for the desired mode of administration, including, but not limited to tablets, gel capsules, sustained-release capsules, and the like for oral administration; depot products, pre-filled syringes, ampoules, vials, and the like for parenteral administration; and patches, medipads, creams, and the like for topical administration.

The active compound is included in the pharmaceutically acceptable carrier in an amount sufficient to exert a therapeutically useful effect in the absence of undesirable side effects on the subject treated. A therapeutically effective concentration may be determined empirically by testing the compounds in known in vitro and in vivo model systems for the treated disorder. In some examples, a therapeutically effective amount of the compound is an amount that lessens or ameliorates at least one symptom of the disorder for which the compound is administered. Typically, the compositions are formulated for single dosage administration. The concentration of active compound in the drug composition will depend on absorption, inactivation, and excretion rates of the active compound, the dosage schedule, and amount administered as well as other factors known to those of skill in the art.

In some examples, about 0.1 mg to 1000 mg of a disclosed compound, a mixture of such compounds, or a physiologically acceptable salt or ester thereof, is compounded with a physiologically acceptable vehicle, carrier, excipient, binder, preservative, stabilizer, flavor, etc., in a unit dosage form. The amount of active substance in those compositions or preparations is such that a suitable dosage in the range indicated is obtained. The term “unit dosage form” refers to physically discrete units suitable as unitary dosages for human subjects and other mammals, each unit containing a predetermined quantity of active material calculated to produce the desired therapeutic effect, in association with a suitable pharmaceutical excipient. In some examples, the compositions are formulated in a unit dosage form, each dosage containing from about 1 mg to about 1000 mg (for example, about 2 mg to about 500 mg, about 5 mg to 50 mg, about 10 mg to 100 mg, or about 25 mg to 75 mg) of the one or more compounds. In other examples, the unit dosage form includes about 0.1 mg, about 1 mg, about 5 mg, about 10 mg, about 20 mg, about 30 mg, about 40 mg, about 50 mg, about 60 mg, about 70 mg, about 80 mg, about 90 mg, about 100 mg, about 150 mg, about 200 mg, about 250 mg, about 300 mg, about 400 mg, about 500 mg, about 600 mg, about 700 mg, about 800 mg, about 900 mg, about 1000 mg, or more of the disclosed compound(s).

The disclosed compounds or compositions may be administered as a single dose, or may be divided into a number of smaller doses to be administered at intervals of time. The therapeutic compositions can be administered in a single dose delivery, by continuous delivery over an extended time period, in a repeated administration protocol (for example, by a multi-daily, daily, weekly, or monthly repeated administration protocol). It is understood that the precise dosage, timing, and duration of treatment is a function of the disease being treated and may be determined empirically using known testing protocols or by extrapolation from in vivo or in vitro test data (such as testing in an animal model of HIV infection). It is to be noted that concentrations and dosage values may also vary with the severity of the condition to be alleviated. In addition, it is understood that for a specific subject, dosage regimens may be adjusted over time according to the individual need and the professional judgment of the person administering or supervising the administration of the compositions, and that the concentration ranges set forth herein are exemplary only.

When administered orally as a suspension, these compositions are prepared according to techniques well known in the art of pharmaceutical formulation and may contain microcrystalline cellulose for imparting bulk, alginic acid or sodium alginate as a suspending agent, methylcellulose as a viscosity enhancer, and sweeteners/flavoring agents. As immediate release tablets, these compositions may contain microcrystalline cellulose, dicalcium phosphate, starch, magnesium stearate and lactose and/or other excipients, binders, extenders, disintegrants, diluents and lubricants. If oral administration is desired, the compound is typically provided in a composition that protects it from the acidic environment of the stomach. For example, the composition can be formulated in an enteric coating that maintains its integrity in the stomach and releases the active compound in the intestine. The composition may also be formulated in combination with an antacid or other such ingredient.

Oral compositions will generally include an inert diluent or an edible carrier and may be compressed into tablets or enclosed in gelatin capsules. For the purpose of oral therapeutic administration, the active compound or compounds can be incorporated with excipients and used in the form of tablets, capsules, or troches. Pharmaceutically compatible binding agents and adjuvant materials can be included as part of the composition. The tablets, pills, capsules, troches, and the like can contain any of the following ingredients or compounds of a similar nature: a binder such as, but not limited to, gum tragacanth, acacia, corn starch, or gelatin; an excipient such as microcrystalline cellulose, starch, or lactose; a disintegrating agent such as, but not limited to, alginic acid and corn starch; a lubricant such as, but not limited to, magnesium stearate; a gildant, such as, but not limited to, colloidal silicon dioxide; a sweetening agent such as sucrose or saccharin; and a flavoring agent such as peppermint, methyl salicylate, or fruit flavoring.

When the dosage unit form is a capsule, it can contain, in addition to material of the above type, a liquid carrier such as a fatty oil. In addition, dosage unit forms can contain various other materials, which modify the physical form of the dosage unit, for example, coatings of sugar and other enteric agents. The compounds can also be administered as a component of an elixir, suspension, syrup, wafer, chewing gum or the like. A syrup may contain, in addition to the active compounds, sucrose as a sweetening agent and certain preservatives, dyes and colorings, and flavors.

When administered orally, the compounds can be administered in usual dosage forms for oral administration. These dosage forms include the usual solid unit dosage forms of tablets and capsules as well as liquid dosage forms such as solutions, suspensions, and elixirs. When the solid dosage forms are used, it is preferred that they be of the sustained release type so that the compounds need to be administered only once or twice daily. In some examples, an oral dosage form is administered to the subject 1, 2, 3, 4, or more times daily. In additional examples, the compounds can be administered orally to humans in a dosage range of 1 to 1000 mg/kg body weight in single or divided doses. One illustrative dosage range is 0.1 to 200 mg/kg body weight orally (such as 0.5 to 100 mg/kg body weight orally) in single or divided doses. For oral administration, the compositions may be provided in the form of tablets containing about 1 to 1000 milligrams of the active ingredient, particularly 1, 5, 10, 15, 20, 25, 50, 75, 100, 150, 200, 250, 300, 400, 500, 600, 750, 800, 900, or 1000 milligrams of the active ingredient. It will be understood, however, that the specific dose level and frequency of dosage for any particular patient may be varied and will depend upon a variety of factors including the activity of the specific compound employed, the metabolic stability and length of action of that compound, the age, body weight, general health, sex, diet, mode and time of administration, rate of excretion, drug combination, the severity of the particular condition, and the host undergoing therapy.

Injectable solutions or suspensions may also be formulated, using suitable non-toxic, parenterally-acceptable diluents or solvents, such as mannitol, 1,3-butanediol, water, Ringer's solution or isotonic sodium chloride solution, or suitable dispersing or wetting and suspending agents, such as sterile, bland, fixed oils, including synthetic mono- or diglycerides, and fatty acids, including oleic acid. Solutions or suspensions used for parenteral, intradermal, subcutaneous, or topical application can include any of the following components: a sterile diluent such as water for injection, saline solution, fixed oil, a naturally occurring vegetable oil such as sesame oil, coconut oil, peanut oil, cottonseed oil, and the like, or a synthetic fatty vehicle such as ethyl oleate, and the like, polyethylene glycol, glycerine, propylene glycol, or other synthetic solvent; antimicrobial agents such as benzyl alcohol and methyl parabens; antioxidants such as ascorbic acid and sodium bisulfite; chelating agents such as ethylenediaminetetraacetic acid (EDTA); buffers such as acetates, citrates, and phosphates; and agents for the adjustment of tonicity such as sodium chloride and dextrose. Parenteral preparations can be enclosed in ampoules, disposable syringes, or multiple dose vials made of glass, plastic, or other suitable material. Buffers, preservatives, antioxidants, and the like can be incorporated as required.

Where administered intravenously, suitable carriers include physiological saline, phosphate buffered saline (PBS), and solutions containing thickening and solubilizing agents such as glucose, polyethylene glycol, polypropyleneglycol, and mixtures thereof. Liposomal suspensions including tissue-targeted liposomes may also be suitable as pharmaceutically acceptable carriers.

The compounds can be administered parenterally, for example, by IV, IM, depo-IM, SC, or depo-SC. When administered parenterally, a therapeutically effective amount of about 0.1 to about 500 mg/day (such as about 1 mg/day to about 100 mg/day, or about 5 mg/day to about 50 mg/day) may be delivered. When a depot formulation is used for injection once a month or once every two weeks, the dose may be about 0.1 mg/day to about 100 mg/day, or a monthly dose of from about 3 mg to about 3000 mg.

The compounds can also be administered sublingually. When given sublingually, the compounds should be given one to four times daily in the amounts described above for IM administration.

The compounds can also be administered intranasally. When given by this route, the appropriate dosage forms are a nasal spray or dry powder. The dosage of the compounds for intranasal administration is the amount described above for IM administration. When administered by nasal aerosol or inhalation, these compositions may be prepared according to techniques well known in the art of pharmaceutical formulation and may be prepared as solutions in saline, employing benzyl alcohol or other suitable preservatives, absorption promoters to enhance bioavailability, fluorocarbons, and/or other solubilizing or dispersing agents.

The compounds can be administered intrathecally. When given by this route, the appropriate dosage form can be a parenteral dosage form. The dosage of the compounds for intrathecal administration is the amount described above for IM administration.

The compounds can be administered topically. When given by this route, the appropriate dosage form is a cream, ointment, or patch. When administered topically, an illustrative dosage is from about 0.5 mg/day to about 200 mg/day. Because the amount that can be delivered by a patch is limited, two or more patches may be used.

The compounds can be administered rectally by suppository. When administered by suppository, an illustrative therapeutically effective amount may range from about 0.5 mg to about 500 mg. When rectally administered in the form of suppositories, these compositions may be prepared by mixing the drug with a suitable non-irritating excipient, such as cocoa butter, synthetic glyceride esters of polyethylene glycols, which are solid at ordinary temperatures, but liquefy and/or dissolve in the rectal cavity to release the drug.

It should be apparent to one skilled in the art that the exact dosage and frequency of administration will depend on the particular compounds administered, the particular condition being treated, the severity of the condition being treated, the age, weight, general physical condition of the particular subject, and other medication the individual may be taking as is well known to administering physicians or other clinicians who are skilled in therapy of retroviral infections, diseases, and associated disorders.

### Examples

The tables below show the in vitro antiretroviral activity data for illustrative compounds along with in vitro binding data for recombinant HIV-1 Nef as obtained by surface plasmon resonance.

In particular, the tables show antiretroviral activity as percent inhibition of HIV-1 infectivity at 1 μM. In brief, this assay uses the TZM-bl reporter cell line, which carries a stable copy of the HIV-1 LTR coupled to luciferase. Cells are infected with HIV-1_{NL4-3} in the presence and absence of each compound, and luciferase activity is measured 48 h later. This assay measures early events in the HIV-1 life cycle, including viral entry, reverse transcription, nuclear entry and integration, and viral transcription. All assays include control cells infected with Nef-defective HIV-1, which replicates at 30-50% of wild-type in this system. Results are normalized to percent inhibition relative to DMSO-treated control cells infected with wild-type HIV-1 (0% inhibition) and DMSO-treated cells infected with Nef-defective HIV-1 (100% of the Nef-dependent effect)—see column 3. In addition, cell viability is measured in the TZM-bl cell line in the presence of each test compound and in the absence of HIV-1 infection, using the Cell Titer Blue cell viability assay (Promega; column 4).

Also shown are SPR data recorded on a Reichert 4-channel SPR instrument. These data were generated using recombinant full-length Nef. The Nef alleles used for recombinant protein expression are SF2 and NL4-3, which matches the strain used in the infectivity assays; both SF2 and NL4-3 are derived from HIV-1 M-group, subtype B. In addition, Nef from SIV is used (from SIV strain mac239). Recombinant Nef is immobilized on the biosensor surface, and each compound is injected and association/dissociation kinetics are recorded. The resulting data for the 3 isoforms are each best-fit using both a two-site model and a two-step, induced-fit model. Dissociation constants (K_{D}), calculated from the kinetic association and dissociation constants from all three isoforms, are shown in columns 6-7. Data were fit using three regimes and the resulting K_{D}: from each model are shown in the Table: 1:1 Langmuir fit; 2-site fit, which yields low and high affinity components; and a 2-step induced fit model.

In general, the most promising compounds show >50% inhibition of HIV-1 infectivity at 1.0 μM without cytotoxicity (cell viability >80%), and show K_{D} values of at least 1 μM (10⁻⁶ M) in at least one of the fitting models with an RUmax≥15 at 30 μM.

The tables also show data for HIV-1 Replication in normal donor peripheral blood mononuclear cells (PBMCs), which is assay is conducted as follows:

Non-HLA-typed buffy coats (Source: Pittsburgh Central Blood Bank, #E3755) are received and processed within 24 hours of blood donation. PBMCs are isolated via rocking for RBC separation following 1:1 dilution with PBS, followed by Ficoll gradient centrifugation. The lymphocyte fraction is removed and washed several times for a final concentration of ˜150-300×10⁶ cells per donor. Cells are activated for 3 days with 1-5 μg/mL PHA (Sigma) and 50 U/mL IL-2. Cells are plated at 750,000 cells/ml and viability is checked at day 3 using the Cell Titer Blue assay (Promega). If cells are >90% viable, then they are infected with HIV-1 NL4-3, wild-type or Δ-Nef mutant (input typically 30-500 pg/mL p24 capsid equivalents) in the presence or absence of Nef inhibitor analogs. IL-2 is included in the media at the time of infection. After 4 days, cells are lysed with 1% Triton X-100 for 1.5 h, checked for viability via Cell Titer Blue, and frozen/stored at −20° C. HIV-1 replication is assessed with a high-sensitivity HIV-1 p24 AlphaLISA assay (Perkin Elmer) in ½-volume 96-well plates, using one-half of the recommended assay volumes for all reagents (including samples). Assays are read on the BioTek Cytation5 instrument; readout is in AlphaUnits and replication in p24 pg/mL is determined using an HIV-1 p24 capsid protein standard included in the Alpha kit.

TABLE 1

| | HIV | | HIV-1 | Percent | | | |
|---|---|---|---|---|---|---|---|
| | Replication | | Infectivity % | Viability | Nef NL4-3 Binding, SPR | | |
| | PBMC IC50 | | Inhibition | @ 1 μM | 1:1 or 2-site | | 2-state |
| Compound | vs. HIV | | Norm. to Δ- | (TZM-bl | fitting | | fitting |
| No. | NL4-3 | | Nef @ 1 μM | cells) | KD1, M | KD2, M | KD, M |
| I-102 | | | | | | | |
| I-103 | | | | | 2.30 × 10⁻¹ | 2.54 × 10⁻⁵ | N/A |
| I-104 | | | | | 2.37 × 10⁻⁶ | 2.26 × 10⁻⁶ | N/A |
| I-105 | | | | | | | |
| I-106 | | | 98.2 | 84.5 | 2.02 × 10⁻⁵ | 3.26 × 10⁻⁶ | N/A |
| I-152 | | | | | | | |
| I-402 | | | | | | | |
| I-107 | | | | | | | |
| I-1301 | | | | | | | |
| I-108 | | | | | | | |
| I-109 | | | | | 3.62 × 10⁻⁸ | 1 to 1 | N/A |
| | | | | | | fitting | |
| I-110 | Not active | | | | 6.40 × 10⁻⁴ | 6.26 × 10⁻¹⁴ | N/A |
| I-110 | | | 84.9 | 88.4 | 2.92 × 10⁻¹⁰ | 2.14 × 10⁻⁶ | 3.02 × 10⁻⁸ |
| I-202 | Not active | | | | 5.80 × 10⁻⁴ | 1.04 × 10⁻¹² | N/A |
| I-202 | | | 105.0 | 79.2 | 7.37 × 10⁻⁷ | 2.32 × 10⁻⁵ | 2.22 × 10⁻⁶ |
| I-551 | | | | | No Interaction | | |
| I-111 | | | | | No Interaction | | |
| I-112 | | | | | No Interaction | | |
| I-113 | | | | | 3.35 × 10⁻⁶ | 1 to 1 | N/A |
| | | | | | | fitting | |
| I-114 | | | | | 1.77 × 10⁻⁶ | 1 to 1 | N/A |
| | | | | | | fitting | |
| I-114 | | | 78.2 | 99.5 | 1.17 × 10⁻⁶ | 4.40 × 10⁻⁶ | 5.56 × 10⁻⁶ |
| I-115 | | | | | 6.87 × 10⁻⁷ | 5.70 × 10⁻⁶ | N/A |
| I-159 | | | | | 3.84 × 10⁻⁷ | 1 to 1 | N/A |
| | | | | | | fitting | |
| I-116 | | | | | 2.01 × 10⁻⁶ | 1 to 1 | N/A |
| | | | | | | fitting | |
| I-903 | | | | | 9.98 × 10⁻⁶ | 1 to 1 | N/A |
| | | | | | | fitting | |
| I-741 | | | | | 3.26 × 10⁻⁶ | 1.06 × 10⁻⁵ | N/A |
| I-742 | | | 124.6 | 77.3 | 2.46 × 10⁻¹³ | 5.62 × 10⁻⁵ | 1.43 × 10⁻¹² |
| I-743 | | | | | No Interaction | | |
| I-160 | | | 33.4 | 93.7 | 6.30 × 10⁻¹² | 2.62 × 10⁻⁶ | N/A |
| I-117 | | | | | 1.71 × 10⁻⁶ | 2.47 × 10⁻⁵ | N/A |
| I-203 | | | | | 8.21 × 10⁻⁷ | 4.54 × 10⁻⁵ | N/A |
| I-801 | | | | | No Interaction | | |
| I-755 | | | | | 5.27 × 10⁻⁸ | 1 to 1 | 2.96 × 10⁻⁷ |
| | | | | | | fitting | |
| I-1002 | | | 22.7 | 96.4 | 8.04 × 10⁻¹³ | 1.71 × 10⁻⁶ | 1.12 × 10⁻¹⁴ |
| I-502 | | | | | 2.45 × 10⁻⁶ | 1 to 1 | 4.51 × 10⁻⁶ |
| | | | | | | fitting | |
| I-1001 | | | | | 2.57 × 10⁻⁷ | 5.00 × 10⁻⁷ | 1.57 × 10⁻¹³ |
| I-118 | | | 100.8 | 81.6 | 9.56 × 10⁻⁸ | 1.71 × 10⁻⁵ | 2.16 × 10⁻¹⁰ |
| I-161 | Not active | | 38.7 | 89.9 | 4.16 × 10⁻¹⁴ | 5.08 × 10⁻¹⁴ | 1.59 × 10⁻¹³ |
| I-119 | Not active | | 80.7 | 77.1 | 1.89 × 10⁻¹¹ | 6.83 × 10⁻⁶ | 8.08 × 10⁻¹³ |
| I-120 | | | 70.1 | 89.1 | 9.32 × 10⁻¹⁵ | 9.66 × 10⁻¹⁴ | 7.42 × 10⁻¹⁴ |
| I-121 | | | 29.6 | 97.8 | 3.83 × 10⁻¹¹ | 2.61 × 10⁻⁶ | 4.74 × 10⁻⁷ |
| I-122 | | | 95.4 | 75.2 | 1.24 × 10⁻⁶ | 1.16 × 10⁻⁵ | 1.86 × 10⁻⁶ |
| I-552 | | | 50.5 | 91.4 | 5.54 × 10⁻⁷ | 1.65 × 10⁻⁵ | 7.49 × 10⁻⁷ |
| I-123 | Not active | | 96.1 | 85.3 | 8.88 × 10⁻⁸ | 8.78 × 10⁻⁵ | 9.36 × 10⁻¹¹ |
| I-204 | ≤1,000 | nM | 5.8 | 95.1 | 1.52 × 10⁻¹³ | 6.35 × 10⁻¹¹ | 2.29 × 10⁻¹¹ |
| I-124 | | | 47.9 | 101.3 | 1.74 × 10⁻⁶ | 1 to 1 | 4.71 × 10⁻⁶ |
| | | | | | | fitting | |
| I-125 | | | 21.2 | 101.1 | 3.07 × 10⁻⁶ | 1 to 1 | 3.13 × 10⁻⁶ |
| | | | | | | fitting | |
| I-126 | | | −13.9 | 107.8 | No Interaction | | |
| I-1006 | | | 83.1 | 85.2 | 4.72 × 10⁻⁷ | 1.51 × 10⁻⁵ | 1.24 × 10⁻⁹ |
| I-301 | | | 81.2 | 90.7 | 3.70 × 10⁻⁷ | 2.31 × 10⁻⁵ | 6.00 × 10⁻¹¹ |
| I-301 | ≤1 | nM | 81.2 | 90.7 | 3.70 × 10⁻⁷ | 2.31 × 10⁻⁵ | 6.00 × 10⁻¹¹ |
| I-301 | ≤10 nM | | 8.4 | 108.4 | 4.21 × 10⁻⁵ | 1 to 1 | 2.93 × 10⁻⁵ |
| | Toxic @ 1 μM | | | | | fitting | |
| I-301 | | | 73.4 | 100.3 | 4.56 × 10⁻⁶ | 1 to 1 | N/A |
| | | | | | | fitting | |
| I-128 | | | 33.5 | 97.9 | 1.46 × 10⁻¹⁰ | 4.33 × 10⁻⁶ | 7.64 × 10⁻⁹ |
| I-136 | | | 26.5 | 99.9 | 1.60 × 10⁻⁷ | 4.77 × 10⁻⁶ | 2.46 × 10⁻⁶ |
| I-129 | ≤10 | nM | 60.9 | 99.3 | 2.38 × 10⁻⁹ | 1.68 × 10⁻⁵ | 3.71 × 10⁻¹¹ |
| I-130 | | | 43.6 | 98.7 | 7.02 × 10⁻⁷ | 1.30 × 10⁻⁵ | 3.80 × 10⁻⁶ |
| I-158 | | | 101.1 | 92.0 | No Interaction | | |
| I-101 | ≤1 nM | | 128.4 | 76.3 | 1.77 × 10⁻⁶ | 1.47 × 10⁻⁵ | 2.91 × 10⁻¹⁰ |
| | Toxic @ 1 μM | | | | | | |
| I-101 | | | 78.5 | 70.8 | No Interaction | | |
| I-754 | | | 37.4 | 97.2 | 4.77 × 10⁻⁷ | 7.32 × 10⁻⁶ | 2.33 × 10⁻¹¹ |
| I-131 | | | 14.2 | 96.4 | 1.36 × 10⁻⁶ | 1.78 × 10⁻⁵ | 4.93 × 10⁻⁶ |
| I-744 | | | 51.2 | 98.2 | 2.35 × 10⁻⁸ | 7.28 × 10⁻⁶ | 1.95 × 10⁻⁶ |
| I-132 | | | 15.7 | 96.5 | 6.18 × 10⁻⁸ | 8.01 × 10⁻⁶ | 1.70 × 10⁻⁶ |
| I-157 | | | 13.2 | 100.7 | 7.80 × 10⁻⁶ | 1 to 1 | N/A |
| | | | | | | fitting | |
| I-212 | | | 50.9 | 92.9 | 5.38 × 10⁻⁷ | 1.48 × 10⁻⁶ | 2.32 × 10⁻⁶ |
| I-726 | | | −4.6 | 102.4 | No Interaction | | |
| I-727 | | | 8.7 | 95.1 | No Interaction | | |
| I-728 | | | 53.7 | 99.1 | 1.42 × 10⁻⁷ | 1 to 1 | N/A |
| | | | | | | fitting | |
| I-729 | | | 57.2 | 102.4 | No Interaction | | |
| I-730 | | | 24.6 | 95.1 | No Interaction | | |
| I-731 | | | 11.5 | 98.9 | No Interaction | | |
| I-732 | | | 85.5 | 91.0 | 6.30 × 10⁻⁷ | 1.77 × 10⁻⁶ | 4.21 × 10⁻⁶ |
| I-733 | | | 56.5 | 98.8 | 2.69 × 10⁻⁵ | 1 to 1 | N/A |
| | | | | | | fitting | |
| I-734 | | | −8.2 | 96.0 | 5.76 × 10⁻⁷ | 1 to 1 | N/A |
| | | | | | | fitting | |
| I-745 | | | 23.8 | 100.0 | 1.01 × 10⁻⁵ | 1 to 1 | N/A |
| | | | | | | fitting | |
| I-401 | | | 99.6 | 86.4 | 1.89 × 10⁻⁶ | 2.47 × 10⁻⁶ | 7.84 × 10⁻⁵ |
| I-401 | | | 52.3 | 101.9 | 2.37 × 10⁻⁷ | 1.81 × 10⁻⁵ | 9.05 × 10⁻⁷ |
| I-153 | | | −20.1 | 99.9 | 6.88 × 10⁻⁸ | 1 to 1 | N/A |
| | | | | | | fitting | |
| I-774 | | | −0.1 | 94.4 | 2.52 × 10⁻⁷ | 1 to 1 | 2.75 × 10⁻⁸ |
| | | | | | | fitting | |
| I-205 | | | 14.3 | 95.6 | 1.09 × 10⁻⁶ | 1.10 × 10⁻⁶ | 2.08 × 10⁻⁷ |
| I-721 | Borderline | | 120.1 | 84.3 | 2.24 × 10⁻⁷ | 1.76 × 10⁻⁶ | 3.72 × 10⁻⁵ |
| | Toxic @ 10 nM | | | | | | |
| I-740 | | | 39.1 | 89.8 | 1.57 × 10⁻⁷ | 3.38 × 10⁻⁶ | 5.44 × 10⁻⁷ |
| I-735 | | | 38.8 | 96.9 | 4.69 × 10⁻⁸ | 2.80 × 10⁻⁶ | 4.98 × 10⁻¹⁰ |
| I-736 | | | 7.5 | 96.0 | 9.93 × 10⁻⁸ | 1.94 × 10⁻⁶ | 2.06 × 10⁻⁹ |
| I-737 | | | −4.8 | 93.0 | 4.89 × 10⁻¹² | 4.00 × 10⁻⁶ | 1.70 × 10⁻⁹ |
| I-503 | | | −94.1 | 90.7 | 2.56 × 10⁻⁷ | 3.02 × 10⁻⁶ | 5.11 × 10⁻¹⁰ |
| I-702 | Toxic @ 10 nM | | 147.7 | 96.4 | 6.05 × 10⁻⁷ | 2.72 × 10⁻⁶ | 7.87 × 10⁻⁷ |
| I-216 | Not active | | 176.4 | 87.1 | 1.24 × 10⁻⁷ | 8.29 × 10⁻⁷ | 8.71 × 10⁻¹² |
| I-746 | | | 194.3 | 101.6 | 7.96 × 10⁻⁷ | 1 to 1 | N/A |
| | | | | | | fitting | |
| I-738 | | | −9.9 | 100.4 | No Interaction | | |
| I-747 | | | 179.3 | 86.9 | 8.91 × 10⁻⁸ | 1.56 × 10⁻⁶ | 2.57 × 10⁻⁴ |
| I-748 | | | 22.3 | 94.8 | 4.15 × 10⁻⁶ | 1 to 1 | N/A |
| | | | | | | fitting | |
| I-749 | | | 64.1 | 78.7 | 1.26 × 10⁻⁹ | 7.55 × 10⁻⁷ | 4.02 × 10⁻⁷ |
| I-750 | | | 63.7 | 93.0 | 1.38 × 10⁻¹⁰ | 5.72 × 10⁻¹⁰ | 1.16 × 10⁻⁶ |
| I-751 | | | 22.3 | 101.1 | 5.60 × 10⁻⁷ | 1 to 1 | N/A |
| | | | | | | fitting | |
| I-752 | | | 14.7 | 97.7 | 2.39 × 10⁻¹⁴ | 1 to 1 | N/A |
| | | | | | | fitting | |
| I-803 | | | 3.2 | 100.4 | 8.17 × 10⁻⁷ | 1 to 1 | N/A |
| | | | | | | fitting | |
| I-804 | | | 50.0 | 97.5 | 9.08 × 10⁻⁸ | 1 to 1 | N/A |
| | | | | | | fitting | |
| I-802 | | | 4.4 | 99.3 | 4.47 × 10⁻⁷ | 1 to 1 | 1.82 × 10⁻⁷ |
| | | | | | | fitting | |
| I-213 | | | −4.6 | 97.6 | 1.60 × 10⁻⁶ | 1 to 1 | N/A |
| | | | | | | fitting | |
| I-759 | | | −7.6 | 98.2 | 2.21 × 10⁻⁷ | 1 to 1 | N/A |
| | | | | | | fitting | |
| I-760 | | | 46.5 | 96.8 | 7.80 × 10⁻⁷ | 1 to 1 | N/A |
| | | | | | | fitting | |
| I-763 | | | 11.0 | 97.6 | 4.22 × 10⁻⁷ | 1 to 1 | N/A |
| | | | | | | fitting | |
| I-761 | | | −3.8 | 101.5 | 8.54 × 10⁻⁷ | 1 to 1 | N/A |
| | | | | | | fitting | |
| I-762 | | | 25.7 | 98.9 | 2.77 × 10⁻⁷ | 7.46 × 10⁻⁶ | 3.98 × 10⁻⁹ |
| I-219 | | | 108.7 | 79.2 | 6.86 × 10⁻¹³ | 3.29 × 10⁻⁶ | 4.93 × 10⁻⁸ |
| I-306 | | | −5.2 | 95.7 | 1.36 × 10⁻⁸ | 5.43 × 10⁻⁶ | 7.31 × 10⁻⁷ |
| I-166 | Not active | | 24.6 | 97.0 | 6.03 × 10⁻⁹ | 1 to 1 | N/A |
| | Toxic @ 1 μM | | | | | fitting | |
| I-133 | | | 16.0 | 102.5 | 7.79 × 10⁻⁷ | 6.53 × 10⁻⁵ | 1.51 × 10⁻⁹ |
| I-162 | | | 77.5 | 90.8 | 1.86 × 10⁻⁹ | 1 to 1 | N/A |
| | | | | | | fitting | |
| I-162 | Not active | | 20.6 | 99.9 | No Interaction | | |
| | Toxic @ 1 μM | | | | | | |
| I-220 | | | 9.8 | 103.6 | 2.83 × 10⁻⁷ | 3.82 × 10⁻⁶ | 3.95 × 10⁻⁶ |
| I-134 | | | 45.8 | 100.5 | No Interaction | | |
| I-753 | | | 76.5 | 95.8 | 2.04 × 10⁻⁸ | 1 to 1 | N/A |
| | | | | | | fitting | |
| I-805 | | | 33.9 | 93.7 | 5.96 × 10⁻⁷ | 1 to 1 | N/A |
| | | | | | | fitting | |
| I-1003 | | | 42.3 | 100.9 | 1.81 × 10⁻⁶ | 1 to 1 | N/A |
| | | | | | | fitting | |
| I-756 | | | 34.3 | 101.9 | No Interaction | | |
| I-757 | | | 17.2 | 102.8 | 2.39 × 10⁻⁶ | 1 to 1 | N/A |
| | | | | | | fitting | |
| I-758 | | | 54.9 | 99.8 | 6.31 × 10⁻⁸ | 1 to 1 | N/A |
| | | | | | | fitting | |
| I-310 | | | 36.6 | 105.6 | 5.02 × 10⁻⁸ | 1 to 1 | N/A |
| | | | | | | fitting | |
| I-156 | | | 15.4 | 101.5 | 8.56 × 10⁻⁹ | 1 to 1 | N/A |
| | | | | | | fitting | |
| I-217 | | | 0.1 | 104.9 | No Interaction | | |
| I-720 | | | 92.2 | 71.4 | 1.19 × 10⁻⁶ | 1 to 1 | 1.81 × 10⁻⁶ |
| | | | | | | fitting | |
| I-806 | | | 20.6 | 99.1 | 9.90 × 10⁻⁶ | 1 to 1 | N/A |
| | | | | | | fitting | |
| I-504 | | | −43.7 | 101.3 | 1.06 × 10⁻⁵ | 1 to 1 | N/A |
| | | | | | | fitting | |
| I-135 | | | 35.7 | 91.9 | 6.36 × 10⁻⁷ | 1 to 1 | 1.47 × 10⁻⁸ |
| | | | | | | fitting | |
| I-127 | | | 2.4 | 100.0 | 2.10 × 10⁻⁶ | 1 to 1 | N/A |
| | | | | | | fitting | |
| I-137 | | | −1.4 | 99.4 | 2.43 × 10⁻⁷ | 1 to 1 | N/A |
| | | | | | | fitting | |
| I-138 | | | 48.2 | 95.8 | 3.96 × 10⁻⁶ | 1 to 1 | N/A |
| | | | | | | fitting | |
| I-770 | | | 49.2 | 99.7 | 1.05 × 10⁻⁹ | 1 to 1 | N/A |
| | | | | | | fitting | |
| I-553 | | | 74.3 | 62.3 | 1.23 × 10⁻⁶ | 1 to 1 | N/A |
| | | | | | | fitting | |
| I-214 | | | −5.5 | 99.9 | 2.08 × 10⁻⁸ | 1 to 1 | N/A |
| | | | | | | fitting | |
| I-772 | | | 49.6 | 100.1 | 8.58 × 10⁻⁷ | 1 to 1 | N/A |
| | | | | | | fitting | |
| I-771 | | | 13.1 | 100.1 | 9.65 × 10⁻⁷ | 1 to 1 | N/A |
| | | | | | | fitting | |
| I-1004 | | | −12.3 | 101.3 | 3.54 × 10⁻⁷ | 1 to 1 | N/A |
| | | | | | | fitting | |
| I-769 | | | 26.5 | 98.8 | 7.98 × 10⁻⁷ | 1 to 1 | N/A |
| | | | | | | fitting | |
| I-777 | | | −3.6 | 97.0 | 9.97 × 10⁻⁷ | 1 to 1 | N/A |
| | | | | | | fitting | |
| I-768 | | | −34.5 | 99.8 | 3.84 × 10⁻⁶ | 1 to 1 | N/A |
| | | | | | | fitting | |
| I-556 | | | 18.1 | 86.1 | 7.83 × 10⁻⁷ | 1 to 1 | N/A |
| | | | | | | fitting | |
| I-767 | | | 18.9 | 97.6 | 4.25 × 10⁻⁷ | 1 to 1 | N/A |
| | | | | | | fitting | |
| I-766 | | | 46.9 | 99.5 | 4.81 × 10⁻⁷ | 1 to 1 | N/A |
| | | | | | | fitting | |
| I-765 | | | 13.5 | 99.3 | No Interaction | | |
| I-206 | | | 35.2 | 90.6 | No Interaction | | |
| I-807 | | | 40.4 | 99.0 | No Interaction | | |
| I-808 | | | 12.8 | 102.6 | No Interaction | | |
| I-809 | | | 14.1 | 97.9 | 2.74 × 10⁻⁶ | 1 to 1 | N/A |
| | | | | | | fitting | |
| I-764 | | | 47.4 | 100.6 | No Interaction | | |
| I-773 | | | 25.5 | 101.8 | 5.59 × 10⁻⁸ | 1 to 1 | N/A |
| | | | | | | fitting | |
| I-207 | ≥1 | μM | −9.4 | 99.3 | 1.49 × 10⁻⁹ | 8.09 × 10⁻⁷ | 1.80 × 10⁻¹³ |

| I-810 | | 42.8 | 101.3 | 7.07 × 10−8 | 3.19 × 10⁻⁶ | 3.58 × 10⁻⁶ |
|---|---|---|---|---|---|---|
| I-811 | | 8.3 | 100.7 | 5.59 × 10⁻⁷ | 2.38 × 10⁻⁶ | 1.26 × 10⁻⁵ |
| I-155 | | −5.9 | 98.2 | 3.40 × 10−6 | 1 to 1 | N/A |
| | | | | | fitting | |
| I-211 | | −19.4 | 103.5 | 7.22 × 10⁻⁶ | 1 to 1 | N/A |
| | | | | | fitting | |
| I-1005 | | 49.2 | 93.5 | 9.10 × 10⁻⁸ | 1 to 1 | N/A |
| | | | | | fitting | |
| I-718 | | 56.3 | 89.1 | 5.97 × 10⁻⁷ | 1 to 1 | N/A |
| | | | | | fitting | |
| I-404 | | 1.3 | 100.5 | 1.28 × 10⁻⁷ | 1 to 1 | N/A |
| | | | | | fitting | |
| I-309 | Not active | 92.2 | 99.1 | 5.76 × 10⁻¹⁰ | 1 to 1 | N/A |
| | Toxic @ 1 μM | | | | fitting | |
| I-221 | | 10.6 | 103.1 | 1.28 × 10⁻⁵ | 1 to 1 | N/A |
| | | | | | fitting | |
| I-163 | | 0.3 | 96.6 | 1.01 × 10⁻⁶ | 1 to 1 | 5.82 × 10⁻⁶ |
| | | | | | fitting | |
| I-139 | | 44.1 | 80.8 | 5.56 × 10⁻⁷ | 1 to 1 | 2.07 × 10⁻⁶ |
| | | | | | fitting | |
| I-140 | | 57.3 | 98.3 | 9.15 × 10⁻⁷ | 1 to 1 | N/A |
| | | | | | fitting | |
| I-713 | | 27.8 | 76.8 | 3.78 × 10⁻⁴ | 1 to 1 | N/A |
| | | | | | fitting | |
| I-141 | | 8.7 | 99.5 | No Interaction | | |
| I-164 | | 64.9 | 93.6 | No Interaction | | |
| I-142 | | −7.8 | 100.2 | 3.28 × 10⁻⁵ | 1 to 1 | N/A |
| | | | | | fitting | |
| I-143 | | −19.6 | 98.9 | 4.26 × 10⁻⁷ | 1 to 1 | N/A |
| | | | | | fitting | |
| I-144 | | −6.1 | 99.7 | No Interaction | | |
| I-302 | | −2.4 | 98.9 | 3.61 × 10⁻⁶ | 1 to 1 | N/A |
| | | | | | fitting | |
| I-303 | | 40.6 | 98.3 | 3.96 × 10⁻⁷ | 1 to 1 | N/A |
| | | | | | fitting | |
| I-145 | Not active | −11.7 | 98.2 | 2.11 × 10⁻¹⁴ | 1 to 1 | N/A |
| | | | | | fitting | |
| I-146 | | 5.7 | 98.5 | 1.65 × 10⁻⁶ | 1 to 1 | N/A |
| | | | | | fitting | |
| I-147 | | 59.3 | 97.6 | 7.52 × 10⁻¹¹ | 1 to 1 | N/A |
| | | | | | fitting | |
| I-714 | | 63.6 | 98.3 | 1.11 × 10⁻⁶ | 1 to 1 | N/A |
| | | | | | fitting | |
| I-715 | | 25.2 | 99.1 | 6.86 × 10⁻⁷ | 1.33 × 10⁻⁶ | 2.51 × 10⁻⁶ |
| I-703 | | −8.1 | 99.2 | 1.54 × 10⁻⁶ | 1 to 1 | N/A |
| | | | | | fitting | |
| I-701 | Toxic @ 10 nM | 106.4 | 98.7 | 2.64 × 10⁻⁸ | 4.02 × 10⁻⁶ | 2.88 × 10⁻⁶ |
| I-704 | | 6.6 | 99.4 | 3.62 × 10⁻⁶ | 1 to 1 | N/A |
| | | | | | fitting | |
| I-705 | | −13.8 | 100.9 | 1.11 × 10⁻⁶ | 1 to 1 | N/A |
| | | | | | fitting | |
| I-716 | | 22.8 | 97.1 | 1.97 × 10⁻⁶ | 1 to 1 | 4.37 × 10⁻⁶ |
| | | | | | fitting | |
| I-706 | | 9.8 | 98.6 | 1.17 × 10⁻⁶ | 1 to 1 | N/A |
| | | | | | fitting | |
| I-555 | | −118.0 | 104.6 | 8.75 × 10⁻⁷ | 1 to 1 | N/A |
| | | | | | fitting | |
| I-707 | | −10.6 | 109.3 | 1.98 × 10⁻⁶ | 1 to 1 | N/A |
| | | | | | fitting | |
| I-167 | Not active | 52.1 | 81.2 | 2.99 × 10⁻⁷ | 1.74 × 10⁻⁶ | 2.02 × 10⁻⁹ |
| I-717 | | −1.8 | 104.1 | No Interaction | | |
| I-709 | | 66.7 | 74.0 | 2.63 × 10⁻⁸ | 1 to 1 | N/A |
| | | | | | fitting | |
| I-708 | | 117.0 | 77.8 | 1.40 × 10⁻⁹ | 1 to 1 | N/A |
| | | | | | fitting | |
| I-775 | | 62.1 | 72.9 | 3.16 × 10⁻⁷ | 1 to 1 | N/A |
| | | | | | fitting | |
| I-406 | | −4.4 | 109.7 | 5.49 × 10⁻⁷ | 1.23 × 10⁻⁶ | 2.67 × 10⁻⁶ |
| I-148 | Not active | 98.1 | 75.5 | 6.56 × 10⁻⁷ | 1.77 × 10⁻⁶ | 3.33 × 10⁻¹⁰ |
| | Toxic @ 1 μM | | | | | |
| I-201 | Not active | 53.4 | 70.5 | 7.34 × 10⁻⁸ | 5.80 × 10⁻⁶ | 3.94 × 10⁻⁷ |
| | Toxic @ 1 μM | | | | | |
| I-201 | | 110.5 | 74.6 | 9.36 × 10⁻¹⁰ | 1.50 × 10⁻⁵ | 1.93 × 10⁻⁶ |
| I-208 | | 39.2 | 90.6 | 1.38 × 10⁻⁵ | 1 to 1 | 6.78 × 10⁻⁵ |
| | | | | | fitting | |
| I-1101 | | −9.9 | 99.3 | 3.76 × 10⁻⁷ | 1 to 1 | N/A |
| | | | | | fitting | |
| I-215 | Not active | 53.5 | 74.5 | 4.24 × 10⁻⁷ | 2.08 × 10⁻⁶ | 1.62 × 10⁻⁷ |
| | Toxic @ 1 μM | | | | | |
| I-215 | | 73.6 | 76.0 | 5.10 × 10⁻¹⁰ | 2.82 × 10⁻⁵ | 2.71 × 10⁻¹⁰ |
| I-550 | | −12.9 | 98.8 | 2.82 × 10⁻⁷ | 6.20 × 10⁻⁶ | 8.54 × 10⁻⁹ |
| I-149 | | 54.5 | 98.5 | 2.33 × 10⁻⁶ | 1 to 1 | N/A |
| | | | | | fitting | |
| I-168 | | 19.2 | 97.9 | 7.51 × 10⁻⁷ | 4.97 × 10⁻⁶ | 2.73 × 10⁻⁶ |
| I-719 | | −8.3 | 98.0 | 2.20 × 10⁻⁶ | 1 to 1 | 4.51 × 10⁻⁷ |
| | | | | | fitting | |
| I-169 | | −4.8 | 98.7 | 4.65 × 10⁻⁷ | 1.81 × 10⁻⁶ | 7.40 × 10−6 |
| I-1201 | | 11.8 | 95.4 | 3.69 × 10⁻⁷ | 2.51 × 10⁻⁶ | 7.40 × 10⁻⁷ |
| I-307 | | 47.5 | 98.2 | 8.90 × 10⁻⁷ | 1 to 1 | N/A |
| | | | | | fitting | |
| I-218 | | −7.0 | 97.9 | 1.12 × 10⁻⁶ | 1 to 1 | 6.92 × 10⁻⁷ |
| | | | | | fitting | |
| I-170 | | 67.1 | 98.8 | 1.86 × 10⁻¹³ | 1.99 × 10⁻⁶ | 1.25 × 10⁻⁸ |
| I-171 | | 131.2 | 75.8 | 8.92 × 10⁻⁷ | 1 to 1 | 1.33 × 10⁻⁸ |
| | | | | | fitting | |
| I-554 | | 12.2 | 95.0 | 5.62 × 10⁻⁸ | 2.13 × 10⁻⁶ | 3.15 × 10⁻¹² |
| I-172 | | 56.0 | 96.5 | 2.50 × 10⁻⁷ | 1 to 1 | 6.93 × 10⁻⁸ |
| | | | | | fitting | |
| I-165 | | 25.3 | 89.6 | 6.89 × 10⁻⁷ | 1 to 1 | 6.36 × 10⁻⁷ |
| | | | | | fitting | |
| I-209 | | 4.2 | 100.1 | 7.76 × 10⁻⁷ | 1 to 1 | 5.83 × 10⁻⁶ |
| | | | | | fitting | |
| I-710 | | 2.5 | 106.9 | 6.96 × 10⁻¹⁴ | 9.78 × 10⁻⁶ | 2.46 × 10⁻⁶ |
| I-711 | | −0.4 | 97.2 | 2.34 × 10⁻¹³ | 8.64 × 10⁻⁶ | 3.29 × 10⁻⁶ |
| I-712 | | 52.6 | 104.4 | 6.85 × 10⁻⁸ | 1.03 × 10⁻⁵ | 1.99 × 10⁻⁸ |
| I-901 | | 14.3 | 105.0 | 4.81 × 10⁻⁷ | 1 to 1 | 1.53 × 10⁻¹¹ |
| | | | | | fitting | |
| I-902 | | 13.8 | 106.5 | 6.58 × 10⁻⁹ | 1 to 1 | 3.67 × 10⁻¹² |
| | | | | | fitting | |
| I-210 | | 95.4 | 83.7 | 8.86 × 10⁻⁷ | 8.12 × 10⁻⁶ | 4.56 × 10⁻⁶ |
| I-776 | | 19.3 | 97.1 | 3.93 × 10⁻⁷ | 1 to 1 | 1.20 × 10⁻⁶ |
| | | | | | fitting | |
| I-150 | | 53.9 | 77.7 | 6.75 × ⁻¹⁰ | 1.04 × 10⁻⁵ | 1.07 × 10⁻⁶ |
| I-304 | | −2.0 | 98.7 | 1.38 × 10⁻⁷ | 4.03 × 10⁻⁵ | 3.15 × 10⁻⁶ |
| I-308 | | 61.4 | 100.1 | 9.41 × 10⁻⁷ | 2.21 × 10⁻⁶ | 4.06 × 10⁻⁶ |
| I-722 | | 53.4 | 86.8 | 2.24 × 10⁻⁶ | 7.20 × 10⁻⁶ | 5.49 × 10⁻⁶ |
| I-723 | | −18.1 | 92.2 | 2.10 × 10⁻⁶ | 1.22 × 10⁻⁵ | 9.09 × 10⁻⁶ |
| I-724 | | 55.8 | 98.6 | No Interaction | | |
| I-154 | | 45.4 | 69.3 | 2.88 × 10⁻⁵ | 1 to 1 | N/A |
| | | | | | fitting | |
| I-739 | | −7.0 | 100.0 | No Interaction | | |
| I-305 | | 59.3 | 96.8 | 7.44 × 10⁻⁷ | 1 to 1 | N/A |
| | | | | | fitting | |
| I-403 | | 4.1 | 99.9 | 2.40 × 10⁻⁶ | 1 to 1 | N/A |
| | | | | | fitting | |
| I-405 | | −9.6 | 95.7 | 4.39 × 10⁻⁶ | 1 to 1 | N/A |
| | | | | | fitting | |
| I-151 | | 106.1 | 83.0 | 5.34 × 10⁻⁷ | 4.65 × 10⁻⁵ | 5.65 × 10⁻⁶ |
| I-725 | | 37.4 | 95.6 | 3.29 × 10⁻⁶ | 1 to 1 | N/A |
| | | | | | fitting | |
| I-1401 | | −34.5 | 99.7 | No Interaction | | |
| I-174 | | 11.9 | 101.5 | 1.37 × 10⁻⁶ | 1 to 1 | N/A |
| | | | | | fitting | |
| I-177 | | −45.5 | 102.1 | No Interaction | | |
| I-311 | | 29.8 | 94.5 | 1.01 × 10⁻⁶ | 1 to 1 | N/A |
| | | | | | fitting | |
| I-312 | | −33.7 | 100.1 | No Interaction | | |
| I-313 | | 33.2 | 96.7 | 4.80 × 10⁻⁷ | 1 to 1 | N/A |
| | | | | | fitting | |
| I-314 | | −1.0 | 100.1 | 7.05 × 10⁻⁷ | 1 to 1 | N/A |
| | | | | | fitting | |
| I-315 | | 28.5 | 100.6 | 1.88 × 10⁻⁶ | 1 to 1 | N/A |
| | | | | | fitting | |

N/A means “not applicable”

TABLE 2

| | HIV | HIV-1 | Percent | | | |
|---|---|---|---|---|---|---|
| | Replication | Infectivity % | Viability | Nef NL4-3 Binding, SPR | | |
| | PBMC IC₅₀ | Inhibition | @ 1 μM | 1:1 or 2-site | | 2-state |
| Cpd. | vs. HIV | Norm. to Δ- | (TZM-bl | fitting | | fitting |
| No. | NL4-3 | Nef @ 1 μM | cells) | K_{D}1, M | K_{D}2, M | K_{D}, M |
| I-601 | ≤10 nM | 67.6 | 101.3 | 1.72 × 10⁻⁶ | 2.64 × 10⁻⁵ | 8.96 × 10⁻⁹ |
| I-602 | | 73.8 | 97.2 | No interaction | | |
| I-603 | | 57.8 | 106.0 | 1.83 × 10⁻⁶ | 3.60 × 10⁻⁶ | 5.80 × 10⁻⁸ |
| I-604 | | 25.5 | 110.4 | 6.08 × 10⁻⁶ | 1 to 1 | 2.80 × 10⁻⁵ |
| | | | | | fitting | |
| I-605 | | 67.8 | 106.0 | 2.45 × 10⁻⁶ | 4.64 × 10⁻⁶ | 9.70 × 10⁻⁶ |
| I-606 | | 0.0 | 95.5 | 7.67 × 10⁻⁷ | 2.24 × 10⁻⁶ | 1.15 × 10⁻⁶ |
| I-607 | | 57.3 | 100.0 | 6.92 × 10⁻⁶ | 1 to 1 | N/A |
| | | | | | fitting | |
| I-608 | | 5.3 | 101.2 | No interaction | | |
| I-609 | | 67.3 | 99.6 | 1.15 × 10⁻⁶ | 1 to 1 | N/A |
| | | | | | fitting | |
| I-610 | | 25.7 | 101.1 | 3.93 × 10⁻⁶ | 1 to 1 | N/A |
| | | | | | fitting | |
| I-611 | | 65.7 | 99.6 | No interaction | | |
| I-612 | | 13.8 | 100.7 | 2.43 × 10⁻⁶ | 8.71 × 10⁻⁶ | 3.82 × 10⁻⁶ |
| I-613 | | 51.3 | 99.7 | 2.46 × 10⁻⁶ | 1 to 1 | N/A |
| | | | | | fitting | |
| I-614 | | 0.0 | 101.3 | 3.56 × 10⁻⁶ | 1 to 1 | N/A |
| | | | | | fitting | |
| I-615 | | 47.9 | 98.9 | No interaction | | |
| I-616 | | 0.0 | 95.8 | 4.91 × 10⁻⁶ | 1.88 × 10⁻⁵ | 1.30 × 10⁻⁶ |
| I-617 | | 0.0 | 99.8 | 9.64 × 10⁻⁶ | 1 to 1 | N/A |
| | | | | | fitting | |
| I-618 | | 0.0 | 93.8 | No interaction | | |
| I-619 | | 0.0 | 105.5 | No interaction | | |
| I-620 | | 12.0 | 100.0 | 4.16 × 10⁻⁶ | 1 to 1 | N/A |
| | | | | | fitting | |
| I-1402 | | 8.7 | 102.2 | 4.75 × 10⁻⁶ | 1 to 1 | N/A |
| | | | | | fitting | |
| I-1501 | | −21.4 | 102.5 | 9.42 × 10⁻⁷ | 1 to 1 | N/A |
| | | | | | fitting | |
| I-621 | | 42.4 | 95.3 | No interaction | | |
| I-622 | | 4.9 | 98.5 | No interaction | | |
| I-623 | | 17.4 | 100.7 | 2.21 × 10⁻⁴ | 1 to 1 | N/A |
| | | | | | fitting | |

N/A means “not applicable”

TABLE 3

| | Inhibition | | HIV-1 | | Reichert | Reichert | Reichert |
|---|---|---|---|---|---|---|---|
| | of HIV-1 | HIV | Infectivity % | Toxicity % | SPR | SPR | SPR |
| | NL4-3 | Infectivity % | Inhibition | Viability @ | Nef SF2 | Nef NL43 | SIVNef mac239 |
| Patent | Replication | Control @ | norm. to Δ- | 1 μM | (M) two- | (M) two- | (M) two- |
| Cpd No. | in PBMCs | 1 μM | Nef @ 1 μM | (TZM-bl) | step fitting | step fitting | step fitting |
| I-101 | X | 23.0 | 128.4 | 76.3 | 1.43 × 10⁻¹⁰ | 2.91 × 10⁻¹⁰ | 2.54 × 10⁻⁸ |
| I-148 | | 37.7 | 98.1 | 75.5 | 2.07 × 10⁻¹¹ | 3.33 × 10⁻¹⁰ | 1.08 × 10⁻⁹ |
| I-151 | | 42.3 | 106.1 | 83.0 | 3.50 × 10⁻⁶ | 5.65 × 10⁻⁶ | 1.46 × 10⁻⁶ |
| I-170 | | 65.9 | 67.1 | 98.8 | 4.90 × 10⁻⁷ | 1.25 × 10⁻⁸ | 2.87 × 10⁻⁹ |
| I-201 | | 67.2 | 53.4 | 70.5 | 5.01 × 10⁻⁷ | 3.94 × 10⁻⁷ | 2.38 × 10⁻⁸ |
| I-210 | | 42.1 | 95.4 | 83.7 | 4.31 × 10⁻⁶ | 4.56 × 10⁻⁶ | 7.09 × 10⁻⁷ |
| I-301 | X | 50.7 | 81.2 | 90.7 | 3.69 × 10⁻⁸ | 6.00 × 10⁻¹¹ | 1.74 × 10⁻⁹ |
| I-308 | | 63.5 | 61.4 | 100.1 | 2.91 × 10⁻⁶ | 4.06 × 10⁻⁶ | 3.85 × 10⁻⁶ |
| I-601 | X | 59.5 | 67.6 | 101.3 | 4.55 × 10⁻⁵ | 8.96 × 10⁻⁹ | 2.20 × 10⁻⁵ |
| I-604 | | 84.5 | 25.5 | 110.4 | 2.26 × 10⁻⁵ | 2.80 × 10⁻⁵ | 2.72 × 10⁻⁵ |
| I-701 | | 29.1 | 106.4 | 98.7 | 1.37 × 10⁻⁷ | 2.88 × 10⁻⁶ | 1.93 × 10⁻⁵ |
| I-712 | | 68.1 | 52.6 | 104.4 | 5.90 × 10⁻⁹ | 1.99 × 10⁻⁸ | 2.19 × 10⁻⁹ |
| I-720 | | 52.0 | 92.2 | 71.4 | N/A | 1.81 × 10⁻⁶ | 2.18 × 10⁻⁸ |
| I-721 | | 36.5 | 120.1 | 84.3 | 5.88 × 10⁻⁶ | 3.72 × 10⁻⁵ | 8.26 × 10⁻⁸ |
| I-722 | | 68.2 | 53.4 | 86.8 | 1.28 × 10⁻⁷ | 5.49 × 10⁻⁶ | 4.90 × 10⁻⁶ |
| I-746 | | 85.1 | 194.3 | 101.6 | 6.84 × 10⁻⁶ | N/A | 4.93 × 10⁻⁸ |
| I-747 | | 30.9 | 179.3 | 86.9 | 4.37 × 10⁻¹⁴ | 2.57 × 10⁻⁴ | 2.02 × 10⁻¹³ |

TABLE 4
Each of 216 unique Nef inhibitor compounds were tested for inhibition of HIV-1 infectivity at 1.0
μM using the TZM-bl reporter cell line and for direct interaction with recombinant purified HIV-1 Nef_{NL4-3}
by SPR. Uninfected TZM-bl cell viability at 1.0 μM is also shown (CellTiter-Blue Assay; Promega). SPR
data were best-fit by either a 1:1 Langmuir model (K_{D} = k_{off}/kₒₙ) or by a 2-step, induced fit model (K_{D} =
k_{off}/kₒₙ × k_{off2}/(k_{off2} + kₒₙ₂) as indicted. Residence time (T) is defined as 1/k_{off} for the 1:1 fit and 1/k_{off2} for
the 2-step induced fit model. Extent of bonding is defined as the ratio of the response units observed at 30
μM (RU₃₀) to the amount of Nef immobilized on the SPR chip (RU_{Nef} values varied by less than 2-fold
across all of the SPR experiments).

| | | | | | | % | |
|---|---|---|---|---|---|---|---|
| | | | | | | Infec- | |
| Com- | | | | | | tivity | % Cell |
| pound | | | T, | RU₃₀/ | SPR | Inhibi- | Via- |
| No. | Structure | K_{D}, M | minutes | RU_{Nef} | fit | tion | bility |
| I-202 | 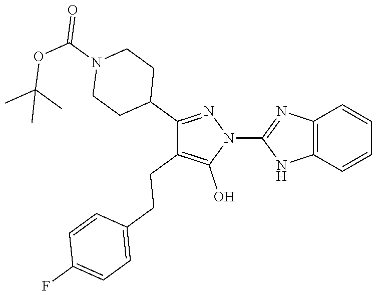 [SMILES: CC(C)(C)OC(=O)N1CCC(c2nn(-c3nc4ccccc4[nH]3)c(O)c2CCc2ccc(F)cc2)CC1] | 6.05E- 12 | 6.36E + 06 | 4.09E- 03 | 2- Step | 100 | 79.2 |
| I-742 | 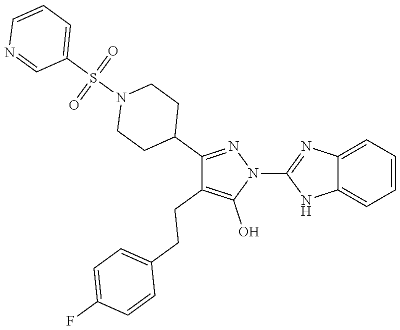 [SMILES: O=S(=O)(c1cccnc1)N1CCC(c2nn(-c3nc4ccccc4[nH]3)c(O)c2CCc2ccc(F)cc2)CC1] | 1.43E- 12 | 2.20E + 07 | 2.76E- 03 | 2- Step | 100 | 77.3 |
| I-160 | 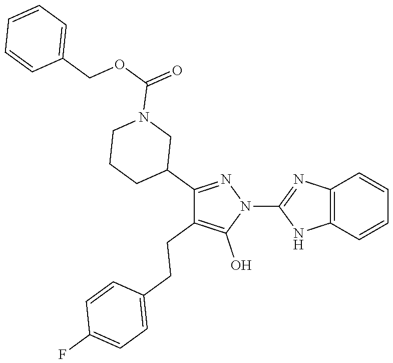 [SMILES: O=C(OCc1ccccc1)N1CCCC(c2nn(-c3nc4ccccc4[nH]3)c(O)c2CCc2ccc(F)cc2)C1] | 2.38E- 12 | 7.41E + 06 | 1.74E- 02 | 2- Step | 33.4 | 93.7 |
| I-161 | 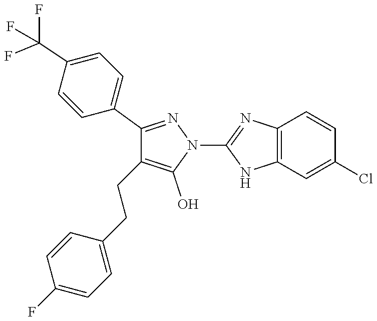 [SMILES: Oc1c(CCc2ccc(F)cc2)c(-c2ccc(C(F)(F)F)cc2)nn1-c1nc2ccc(Cl)cc2[nH]1] | 1.06E- 15 | 4.41E + 10 | 1.44E- 02 | 2- Step | 38.7 | 89.9 |
| I-119 | 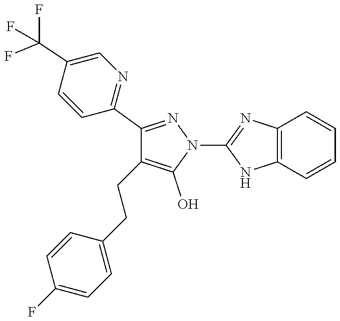 [SMILES: Oc1c(CCc2ccc(F)cc2)c(-c2ccc(C(F)(F)F)cn2)nn1-c1nc2ccccc2[nH]1] | 8.08E- 13 | 3.09E + 07 | 7.52E- 03 | 2- Step | 80.7 | 77.1 |
| I-121 | 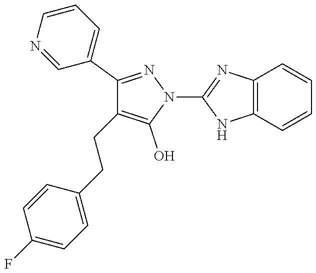 [SMILES: Oc1c(CCc2ccc(F)cc2)c(-c2cccnc2)nn1-c1nc2ccccc2[nH]1] | 4.74E- 07 | 2.58E + 01 | 7.40E- 03 | 2- Step | 29.6 | 97.8 |
| I-122 | 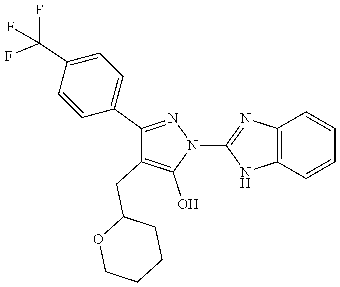 [SMILES: Oc1c(CC2CCCCO2)c(-c2ccc(C(F)(F)F)cc2)nn1-c1nc2ccccc2[nH]1] | 2.25E- 06 | 1.02E + 01 | 1.07E- 02 | 2- Step | 95.4 | 75.2 |
| I-552 | 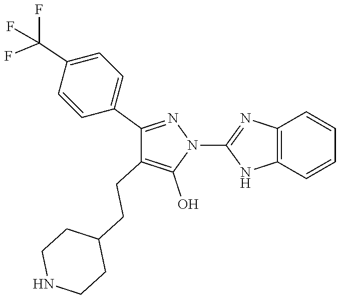 [SMILES: Oc1c(CCC2CCNCC2)c(-c2ccc(C(F)(F)F)cc2)nn1-c1nc2ccccc2[nH]1] | 1.04E- 06 | 2.17E + 01 | 1.04E- 02 | 2- Step | 50.5 | 91.4 |
| I-123 | 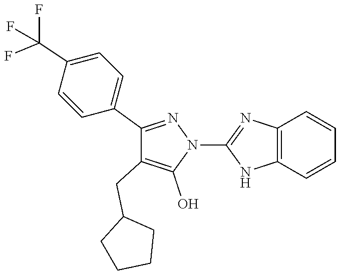 [SMILES: Oc1c(CC2CCCC2)c(-c2ccc(C(F)(F)F)cc2)nn1-c1nc2ccccc2[nH]1] | 9.36E- 11 | 3.79E + 05 | 2.57E- 02 | 2- Step | 96.1 | 85.3 |
| I-204 | 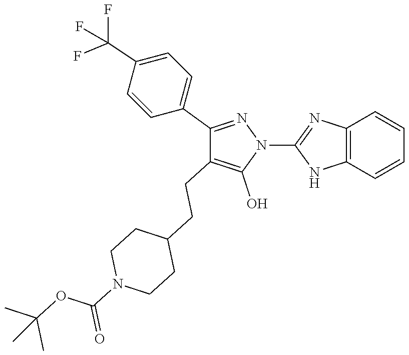 [SMILES: CC(C)(C)OC(=O)N1CCC(CCc2c(-c3ccc(C(F)(F)F)cc3)nn(-c3nc4ccccc4[nH]3)c2O)CC1] | 2.29E- 11 | 1.02E + 05 | 8.83E- 03 | 2- Step | 5.8 | 95.1 |
| I-125 | 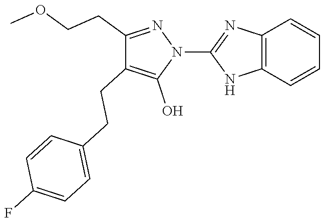 [SMILES: COCCc1nn(-c2nc3ccccc3[nH]2)c(O)c1CCc1ccc(F)cc1] | 3.13E- 06 | 8.87E + 00 | 2.86E- 03 | 2- Step | 21.2 | 100 |
| I-1006 | 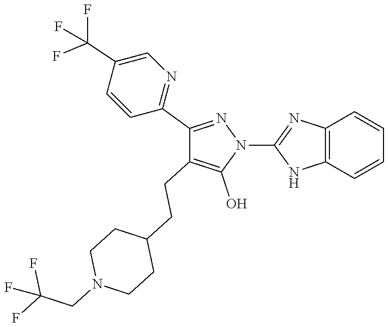 [SMILES: Oc1c(CCC2CCN(CC(F)(F)F)CC2)c(-c2ccc(C(F)(F)F)cn2)nn1-c1nc2ccccc2[nH]1] | 1.24E- 09 | 1.80E + 04 | 5.08E- 03 | 2- Step | 83.1 | 85.2 |
| I-301 | 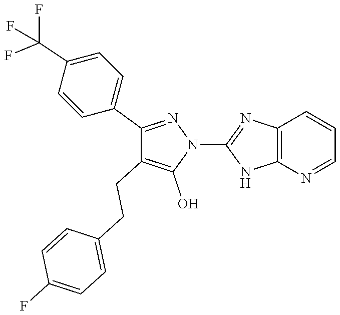 [SMILES: Oc1c(CCc2ccc(F)cc2)c(-c2ccc(C(F)(F)F)cc2)nn1-c1nc2cccnc2[nH]1] | 6.00E- 11 | 6.49E + 07 | 1.34E- 02 | 2- Step | 81.2 | 90.7 |
| I-601 | 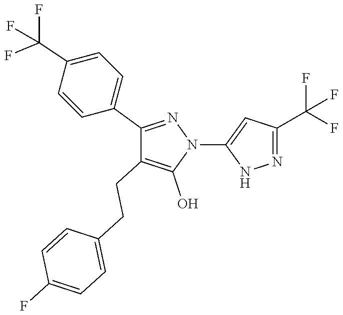 [SMILES: Oc1c(CCc2ccc(F)cc2)c(-c2ccc(C(F)(F)F)cc2)nn1-c1cc(C(F)(F)F)n[nH]1] | 2.64E- 05 | 5.59E + 00 | 4.19E- 02 | 2- Step | 67.6 | 100 |
| I-128 | 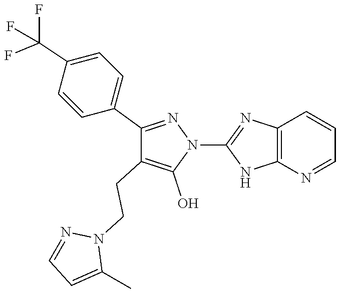 [SMILES: Cc1ccnn1CCc1c(-c2ccc(C(F)(F)F)cc2)nn(-c2nc3cccnc3[nH]2)c1O] | 7.64E- 09 | 1.20E + 03 | 5.56E- 03 | 2- Step | 33.5 | 97.9 |
| I-136 | 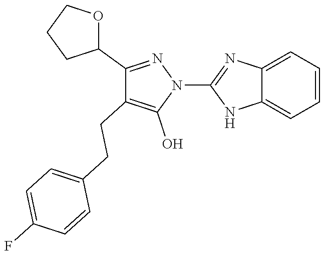 [SMILES: Oc1c(CCc2ccc(F)cc2)c(C2CCCO2)nn1-c1nc2ccccc2[nH]1] | 2.46E- 06 | 3.32E + 00 | 5.11E- 03 | 2- Step | 26.5 | 99.9 |
| I-129 | 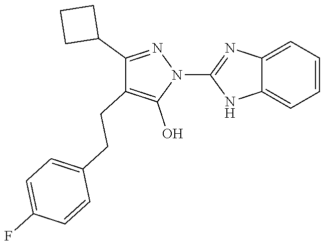 [SMILES: Oc1c(CCc2ccc(F)cc2)c(C2CCC2)nn1-c1nc2ccccc2[nH]1] | 3.71E- 11 | 5.19E + 05 | 1.28E- 02 | 2- Step | 60.9 | 99.3 |
| I-130 | 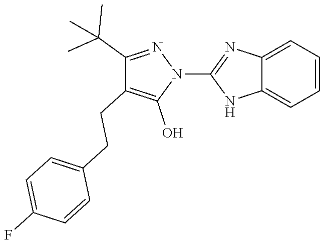 [SMILES: CC(C)(C)c1nn(-c2nc3ccccc3[nH]2)c(O)c1CCc1ccc(F)cc1] | 3.80E- 06 | 1.15E + 01 | 9.49E- 03 | 2- Step | 43.6 | 98.7 |
| I-101 | 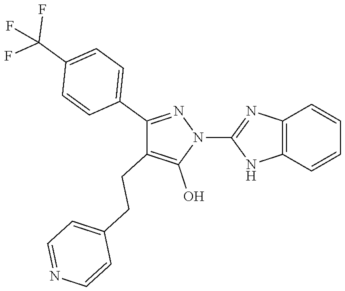 [SMILES: Oc1c(CCc2ccncc2)c(-c2ccc(C(F)(F)F)cc2)nn1-c1nc2ccccc2[nH]1] | 2.91E- 10 | 7.00E + 04 | 1.33E- 02 | 2- Step | 100 | 76.3 |
| I-754 | 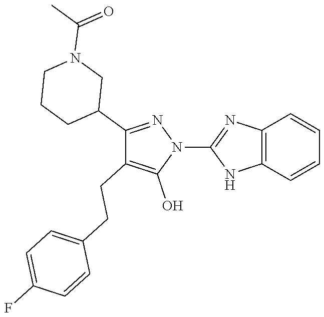 [SMILES: CC(=O)N1CCCC(c2nn(-c3nc4ccccc4[nH]3)c(O)c2CCc2ccc(F)cc2)C1] | 2.33E- 11 | 8.05E + 05 | 5.14E- 03 | 2- Step | 37.4 | 97.2 |
| I-131 | 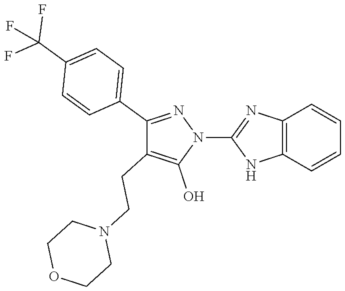 [SMILES: Oc1c(CCN2CCOCC2)c(-c2ccc(C(F)(F)F)cc2)nn1-c1nc2ccccc2[nH]1] | 4.93E- 06 | 5.16E + 00 | 1.06E- 02 | 2- Step | 14.2 | 96.4 |
| I-744 | 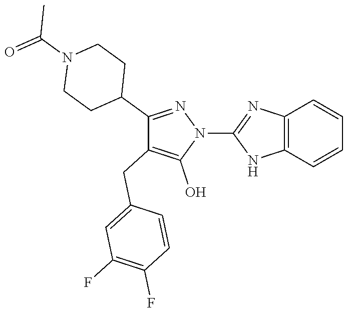 [SMILES: CC(=O)N1CCC(c2nn(-c3nc4ccccc4[nH]3)c(O)c2Cc2ccc(F)c(F)c2)CC1] | 1.95E- 06 | 5.61E + 00 | 4.92E- 03 | 2- Step | 51.2 | 98.2 |
| I-132 | 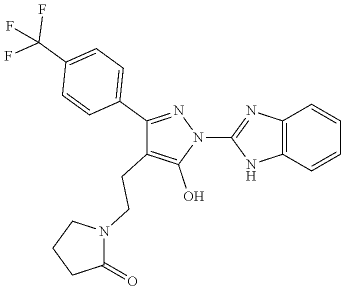 [SMILES: O=C1CCCN1CCc1c(-c2ccc(C(F)(F)F)cc2)nn(-c2nc3ccccc3[nH]2)c1O] | 1.70E- 06 | 5.46E + 00 | 5.62E- 03 | 2- Step | 15.7 | 96.5 |
| I-732 | 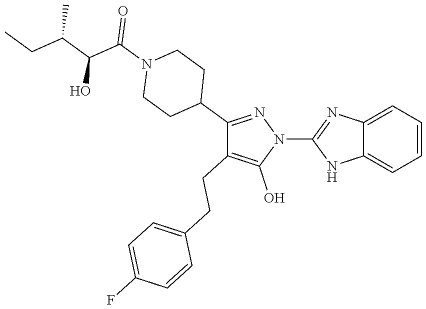 [SMILES: CC[C@H](C)[C@H](O)C(=O)N1CCC(c2nn(-c3nc4ccccc4[nH]3)c(O)c2CCc2ccc(F)cc2)CC1] | 4.21E- 06 | 3.78E + 00 | 3.09E- 03 | 2- Step | 85.5 | 91.0 |
| I-401 | 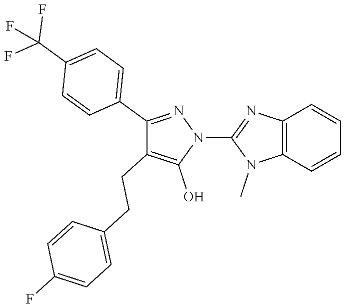 [SMILES: Cn1c(-n2nc(-c3ccc(C(F)(F)F)cc3)c(CCc3ccc(F)cc3)c2O)nc2ccccc21] | 7.84E- 05 | 1.45E + 03 | 2.01E- 02 | 2- Step | 99.6 | 86.4 |
| I-153 | 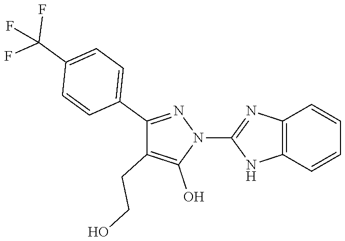 [SMILES: OCCc1c(-c2ccc(C(F)(F)F)cc2)nn(-c2nc3ccccc3[nH]2)c1O] | 6.88E- 08 | 9.31E + 00 | 9.06E- 04 | 1:1 | 0 | 99.9 |
| I-774 | 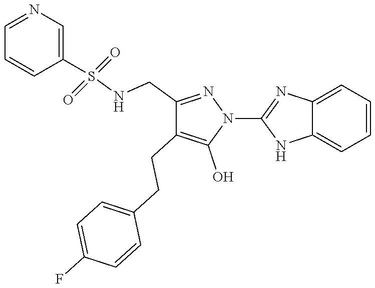 [SMILES: O=S(=O)(NCc1nn(-c2nc3ccccc3[nH]2)c(O)c1CCc1ccc(F)cc1)c1cccnc1] | 2.75E- 08 | 1.09E + 02 | 2.56E- 03 | 2- Step | 0 | 94.4 |
| I-205 | 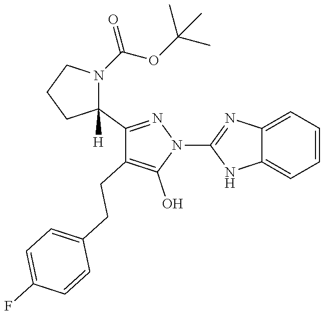 [SMILES: CC(C)(C)OC(=O)N1CCC[C@H]1c1nn(-c2nc3ccccc3[nH]2)c(O)c1CCc1ccc(F)cc1] | 2.08E- 07 | 9.98E + 01 | 5.81E- 03 | 2- Step | 14.3 | 95.6 |
| I-721 | 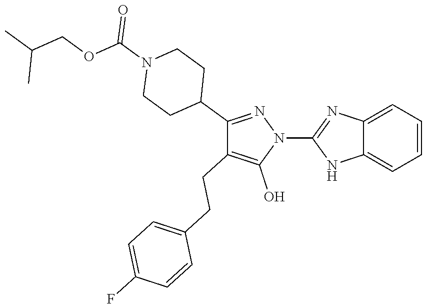 [SMILES: CC(C)COC(=O)N1CCC(c2nn(-c3nc4ccccc4[nH]3)c(O)c2CCc2ccc(F)cc2)CC1] | 3.72E- 05 | 4.69E + 02 | 1.20E- 02 | 2- Step | 100 | 84.3 |
| I-740 | 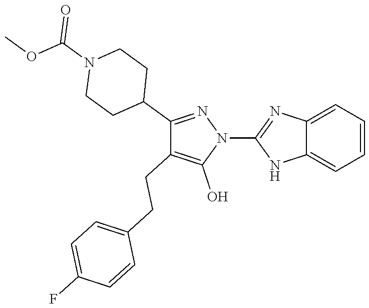 [SMILES: COC(=O)N1CCC(c2nn(-c3nc4ccccc4[nH]3)c(O)c2CCc2ccc(F)cc2)CC1] | 5.44E- 07 | 1.26E + 01 | 7.81E- 03 | 2- Step | 39.1 | 89.8 |
| I-735 | 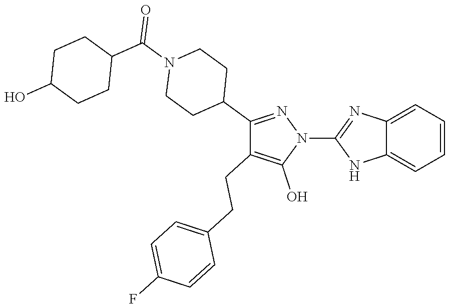 [SMILES: O=C(C1CCC(O)CC1)N1CCC(c2nn(-c3nc4ccccc4[nH]3)c(O)c2CCc2ccc(F)cc2)CC1] | 4.98E- 10 | 6.44E + 03 | 6.53E- 03 | 2- Step | 38.8 | 96.9 |
| I-736 | 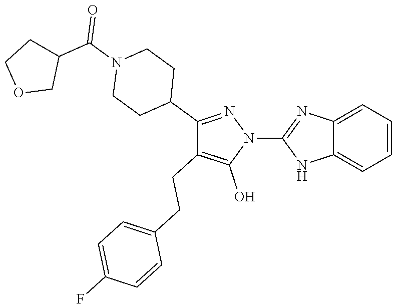 [SMILES: O=C(C1CCOC1)N1CCC(c2nn(-c3nc4ccccc4[nH]3)c(O)c2CCc2ccc(F)cc2)CC1] | 2.06E- 09 | 1.97E + 03 | 1.02E- 02 | 2- Step | 7.5 | 96.0 |
| I-737 | 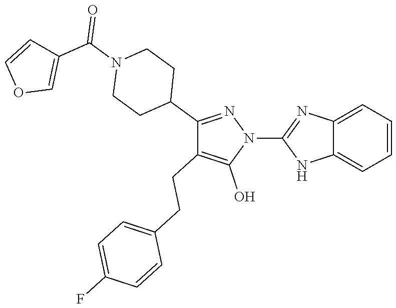 [SMILES: O=C(c1ccoc1)N1CCC(c2nn(-c3nc4ccccc4[nH]3)c(O)c2CCc2ccc(F)cc2)CC1] | 1.70E- 09 | 9.06E + 03 | 1.36E- 02 | 2- Step | 0 | 93.0 |
| I-503 | 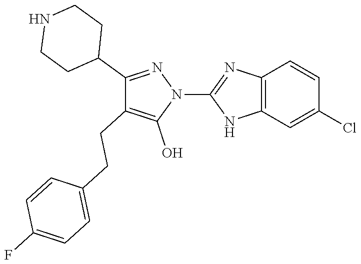 [SMILES: Oc1c(CCc2ccc(F)cc2)c(C2CCNCC2)nn1-c1nc2ccc(Cl)cc2[nH]1] | 6.17E- 10 | 3.18E + 04 | 1.05E- 02 | 2- Step | 0 | 90.7 |
| I-702 | 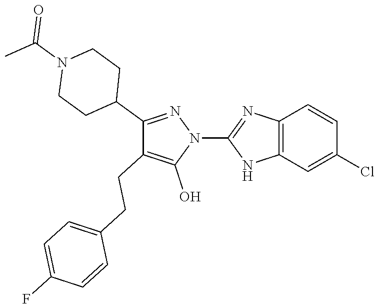 [SMILES: CC(=O)N1CCC(c2nn(-c3nc4ccc(Cl)cc4[nH]3)c(O)c2CCc2ccc(F)cc2)CC1] | 3.59E- 06 | 5.05E + 00 | 1.56E- 02 | 2- Step | 100 | 96.4 |
| I-216 | 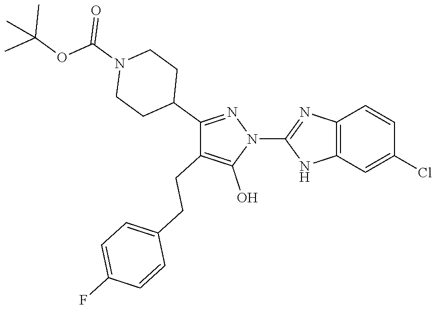 [SMILES: CC(C)(C)OC(=O)N1CCC(c2nn(-c3nc4ccc(Cl)cc4[nH]3)c(O)c2CCc2ccc(F)cc2)CC1] | 8.71E- 12 | 4.71E + 05 | 1.60E- 02 | 2- Step | 100 | 87.1 |
| I-803 | 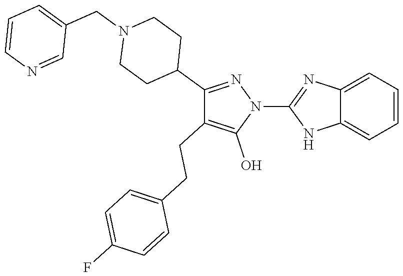 [SMILES: Oc1c(CCc2ccc(F)cc2)c(C2CCN(Cc3cccnc3)CC2)nn1-c1nc2ccccc2[nH]1] | 8.17E- 07 | 1.59E + 01 | 1.69E- 03 | 1:1 | 3.2 | 100 |
| I-802 | 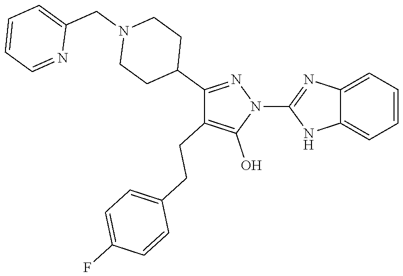 [SMILES: Oc1c(CCc2ccc(F)cc2)c(C2CCN(Cc3ccccn3)CC2)nn1-c1nc2ccccc2[nH]1] | 1.82E- 07 | 2.21E + 01 | 2.48E- 03 | 2- Step | 4.4 | 99.3 |
| I-760 | 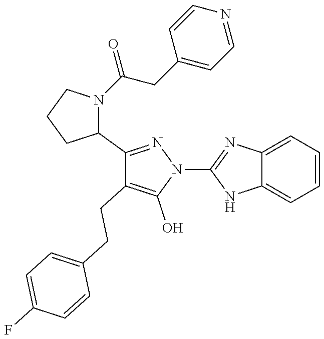 [SMILES: O=C(Cc1ccncc1)N1CCCC1c1nn(-c2nc3ccccc3[nH]2)c(O)c1CCc1ccc(F)cc1] | 7.80E- 07 | 7.31E + 00 | 1.38E- 03 | 1:1 | 46.5 | 96.8 |
| I-762 | 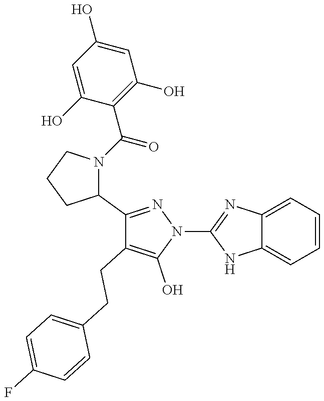 [SMILES: O=C(c1c(O)cc(O)cc1O)N1CCCC1c1nn(-c2nc3ccccc3[nH]2)c(O)c1CCc1ccc(F)cc1] | 3.98E- 09 | 6.59E + 03 | 2.87E- 03 | 2- Step | 25.7 | 98.6 |
| I-219 | 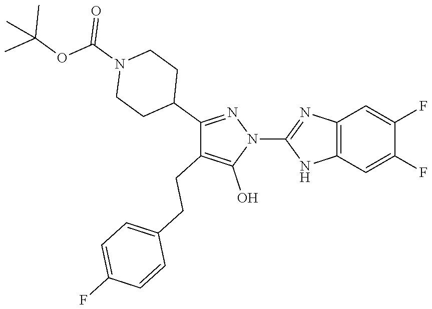 [SMILES: CC(C)(C)OC(=O)N1CCC(c2nn(-c3nc4cc(F)c(F)cc4[nH]3)c(O)c2CCc2ccc(F)cc2)CC1] | 4.93E- 08 | 4.93E + 02 | 4.76E- 03 | 2- Step | 100 | 79.2 |
| I-306 | 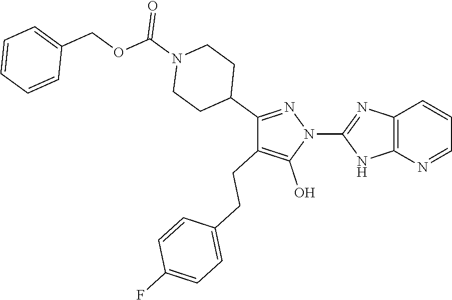 [SMILES: O=C(OCc1ccccc1)N1CCC(c2nn(-c3nc4cccnc4[nH]3)c(O)c2CCc2ccc(F)cc2)CC1] | 7.31E- 07 | 3.52E + 01 | 9.61E- 03 | 2- Step | 0 | 95.7 |
| I-166 | 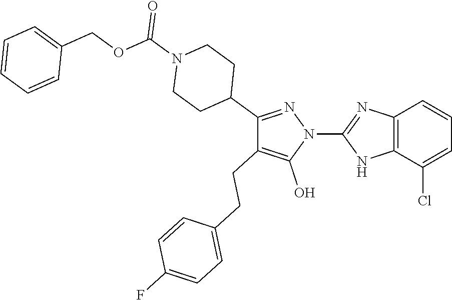 [SMILES: O=C(OCc1ccccc1)N1CCC(c2nn(-c3nc4cccc(Cl)c4[nH]3)c(O)c2CCc2ccc(F)cc2)CC1] | 6.03E- 09 | 2.07E + 03 | 4.21E- 03 | 1:1 | 24.6 | 97.0 |
| I-133 | 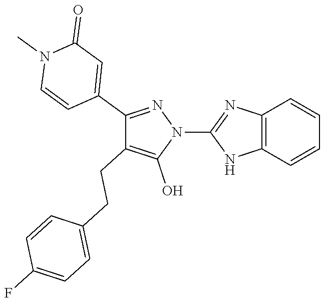 [SMILES: Cn1ccc(-c2nn(-c3nc4ccccc4[nH]3)c(O)c2CCc2ccc(F)cc2)cc1=O] | 1.51E- 09 | 8.59E + 03 | 2.76E- 03 | 2- Step | 16 | 100 |
| I-162 | 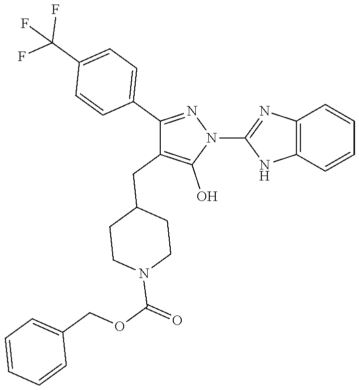 [SMILES: O=C(OCc1ccccc1)N1CCC(Cc2c(-c3ccc(C(F)(F)F)cc3)nn(-c3nc4ccccc4[nH]3)c2O)CC1] | 1.86E- 09 | 8.05E + 03 | 4.69E- 03 | 1:1 | 77.5 | 90.8 |
| I-220 | 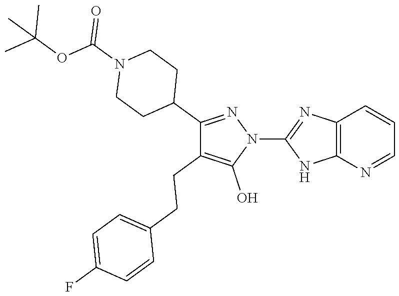 [SMILES: CC(C)(C)OC(=O)N1CCC(c2nn(-c3nc4cccnc4[nH]3)c(O)c2CCc2ccc(F)cc2)CC1] | 3.95E- 06 | 6.92E + 00 | 7.24E- 03 | 2- Step | 9.8 | 100 |
| I-758 | 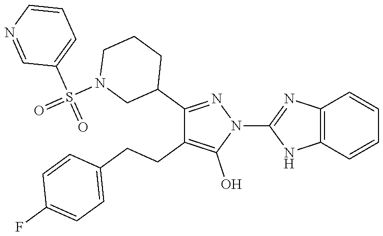 [SMILES: O=S(=O)(c1cccnc1)N1CCCC(c2nn(-c3nc4ccccc4[nH]3)c(O)c2CCc2ccc(F)cc2)C1] | 6.81E- 08 | 9.21E + 01 | 2.95E- 03 | 1:1 | 54.9 | 99.8 |
| I-310 | 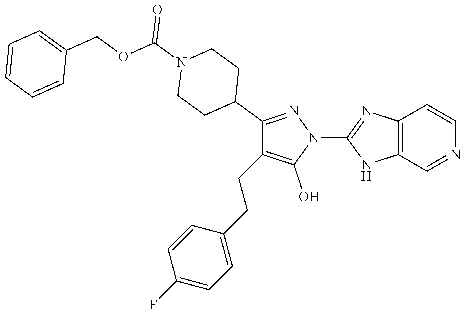 [SMILES: O=C(OCc1ccccc1)N1CCC(c2nn(-c3nc4ccncc4[nH]3)c(O)c2CCc2ccc(F)cc2)CC1] | 5.02E- 08 | 3.56E + 02 | 2.36E- 03 | 1:1 | 36.6 | 100 |
| I-156 | 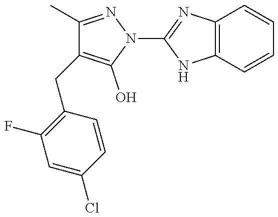 [SMILES: Cc1nn(-c2nc3ccccc3[nH]2)c(O)c1Cc1ccc(Cl)cc1F] | 8.56E- 09 | 7.34E + 02 | 3.62E- 03 | 1:1 | 15.4 | 100 |
| I-720 | 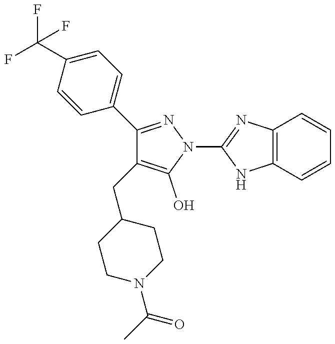 [SMILES: CC(=O)N1CCC(Cc2c(-c3ccc(C(F)(F)F)cc3)nn(-c3nc4ccccc4[nH]3)c2O)CC1] | 1.81E- 06 | 1.32E + 00 | 2.80E- 03 | 2- Step | 92.2 | 71.4 |
| I-806 | 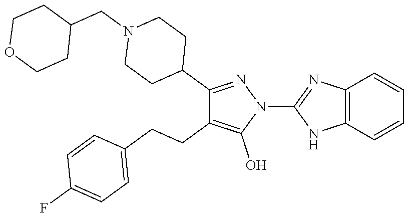 [SMILES: Oc1c(CCc2ccc(F)cc2)c(C2CCN(CC3CCOCC3)CC2)nn1-c1nc2ccccc2[nH]1] | 9.90E- 06 | 1.79E + 00 | 7.48E- 04 | 1:1 | 20.6 | 99.1 |
| I-135 | 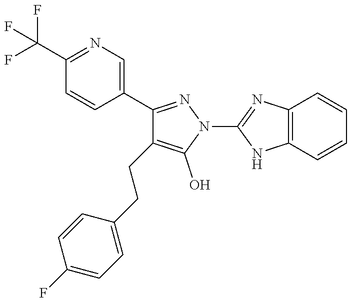 [SMILES: Oc1c(CCc2ccc(F)cc2)c(-c2ccc(C(F)(F)F)nc2)nn1-c1nc2ccccc2[nH]1] | 1.47E- 08 | 1.47E + 03 | 2.87E- 03 | 2- Step | 35.7 | 91.9 |
| I-137 | 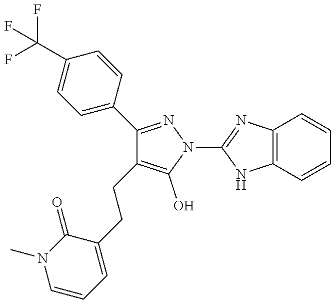 [SMILES: Cn1cccc(CCc2c(-c3ccc(C(F)(F)F)cc3)nn(-c3nc4ccccc4[nH]3)c2O)c1=O] | 2.43E- 07 | 5.36E + 00 | 1.02E- 03 | 1:1 | 0 | 99.4 |
| I-770 | 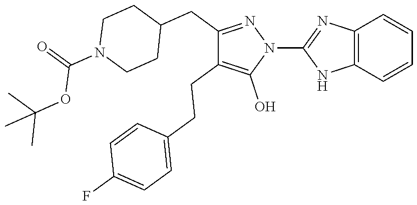 [SMILES: CC(C)(C)OC(=O)N1CCC(Cc2nn(-c3nc4ccccc4[nH]3)c(O)c2CCc2ccc(F)cc2)CC1] | 1.05E- 09 | 1.65E + 04 | 3.04E- 03 | 1:1 | 49.2 | 99.7 |
| I-124 | 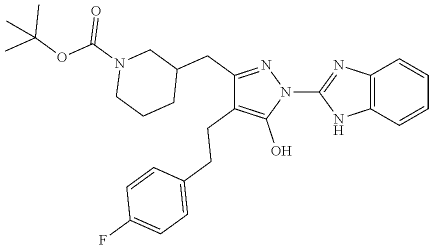 [SMILES: CC(C)(C)OC(=O)N1CCCC(Cc2nn(-c3nc4ccccc4[nH]3)c(O)c2CCc2ccc(F)cc2)C1] | 2.08E- 08 | 4.68E + 02 | 2.46E- 03 | 1:1 | 0 | 99.9 |
| I-1004 | 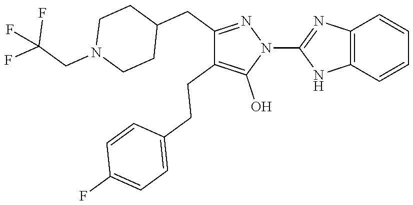 [SMILES: Oc1c(CCc2ccc(F)cc2)c(CC2CCN(CC(F)(F)F)CC2)nn1-c1nc2ccccc2[nH]1] | 3.54E- 07 | 2.82E + 01 | 2.27E- 03 | 1:1 | 0 | 100 |
| I-769 | 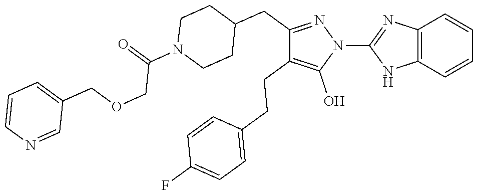 [SMILES: O=C(COCc1cccnc1)N1CCC(Cc2nn(-c3nc4ccccc4[nH]3)c(O)c2CCc2ccc(F)cc2)CC1] | 7.98E- 07 | 9.47E + 00 | 4.88E- 03 | 1:1 | 26.5 | 98.8 |
| I-777 | 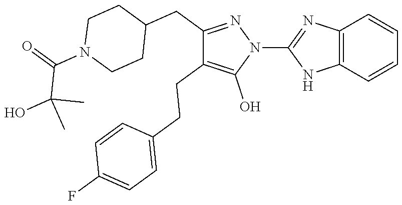 [SMILES: CC(C)(O)C(=O)N1CCC(Cc2nn(-c3nc4ccccc4[nH]3)c(O)c2CCc2ccc(F)cc2)CC1] | 9.97E- 07 | 3.50E + 00 | 2.23E- 03 | 1:1 | 0 | 97.0 |
| I-207 | 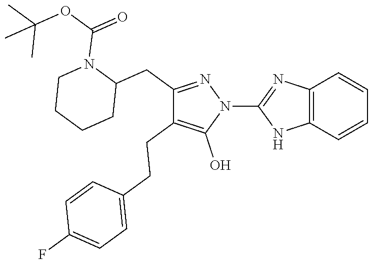 [SMILES: CC(C)(C)OC(=O)N1CCCCC1Cc1nn(-c2nc3ccccc3[nH]2)c(O)c1CCc1ccc(F)cc1] | 1.80E- 13 | 1.33E + 08 | 4.14E- 03 | 2- Step | 0 | 99.3 |
| I-810 | 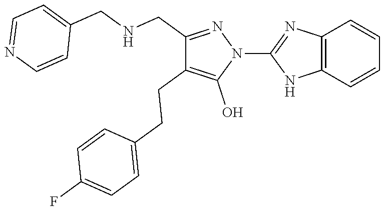 [SMILES: Oc1c(CCc2ccc(F)cc2)c(CNCc2ccncc2)nn1-c1nc2ccccc2[nH]1] | 3.58E- 06 | 5.97E + 00 | 3.75E- 03 | 2- Step | 42.8 | 100 |
| I-811 | 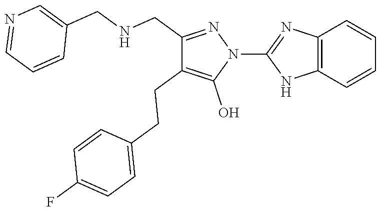 [SMILES: Oc1c(CCc2ccc(F)cc2)c(CNCc2cccnc2)nn1-c1nc2ccccc2[nH]1] | 1.26E- 05 | 3.83E- 01 | 3.57E- 03 | 2- Step | 8.3 | 100 |
| I-211 | 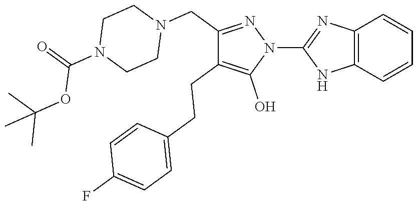 [SMILES: CC(C)(C)OC(=O)N1CCN(Cc2nn(-c3nc4ccccc4[nH]3)c(O)c2CCc2ccc(F)cc2)CC1] | 7.22E- 06 | 1.65E + 00 | 2.89E- 03 | 1:1 | 0 | 100 |
| I-1005 | 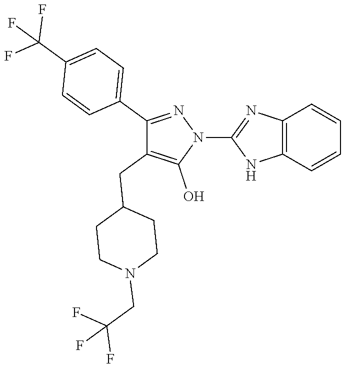 [SMILES: Oc1c(CC2CCN(CC(F)(F)F)CC2)c(-c2ccc(C(F)(F)F)cc2)nn1-c1nc2ccccc2[nH]1] | 9.10E- 08 | 9.47E + 00 | 1.26E- 03 | 1:1 | 49.2 | 93.5 |
| I-718 | 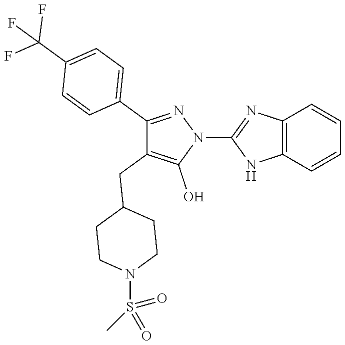 [SMILES: CS(=O)(=O)N1CCC(Cc2c(-c3ccc(C(F)(F)F)cc3)nn(-c3nc4ccccc4[nH]3)c2O)CC1] | 5.97E- 07 | 3.62E + 00 | 1.22E- 03 | 1:1 | 56.3 | 89.1 |
| I-309 | 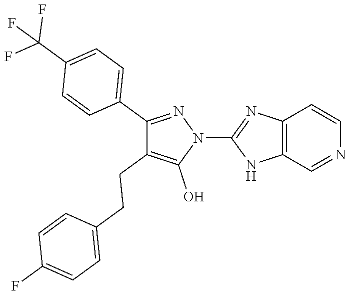 [SMILES: Oc1c(CCc2ccc(F)cc2)c(-c2ccc(C(F)(F)F)cc2)nn1-c1nc2ccncc2[nH]1] | 5.76E- 10 | 1.78E + 04 | 1.59E- 03 | 1:1 | 92.2 | 99.1 |
| I-163 | 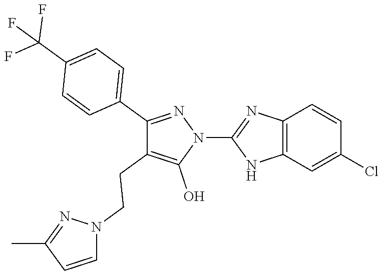 [SMILES: Cc1ccn(CCc2c(-c3ccc(C(F)(F)F)cc3)nn(-c3nc4ccc(Cl)cc4[nH]3)c2O)n1] | 5.82E- 06 | 1.44E + 01 | 4.48E- 03 | 2- step | 0.3 | 96.6 |
| I-139 | 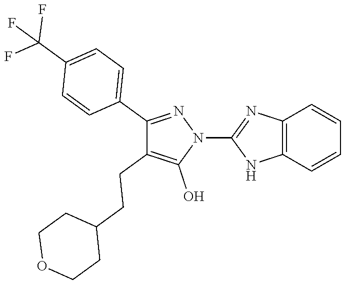 [SMILES: Oc1c(CCC2CCOCC2)c(-c2ccc(C(F)(F)F)cc2)nn1-c1nc2ccccc2[nH]1] | 2.07E- 06 | 1.77E + 01 | 3.41E- 03 | 2- step | 44.1 | 80.8 |
| I-143 | 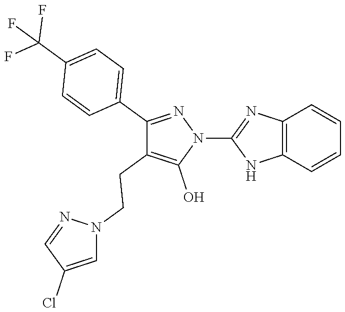 [SMILES: Oc1c(CCn2cc(Cl)cn2)c(-c2ccc(C(F)(F)F)cc2)nn1-c1nc2ccccc2[nH]1] | 4.26E- 07 | 1.75E + 01 | 1.02E- 03 | 1:1 | 0 | 98.9 |
| I-302 | 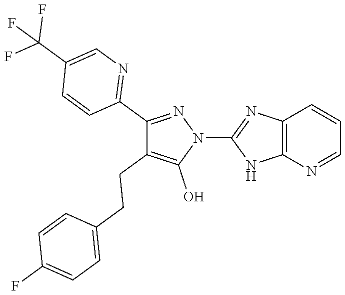 [SMILES: Oc1c(CCc2ccc(F)cc2)c(-c2ccc(C(F)(F)F)cn2)nn1-c1nc2cccnc2[nH]1] | 3.61E- 06 | 8.82E + 00 | 1.66E- 03 | 1:1 | 0 | 98.9 |
| I-303 | 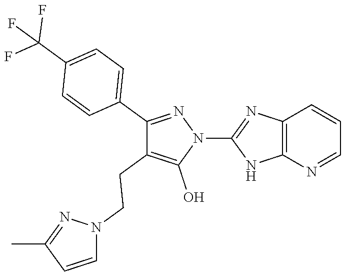 [SMILES: Cc1ccn(CCc2c(-c3ccc(C(F)(F)F)cc3)nn(-c3nc4cccnc4[nH]3)c2O)n1] | 3.96E- 07 | 9.47E + 01 | 3.06E- 03 | 1:1 | 40.1 | 98.3 |
| I-145 | 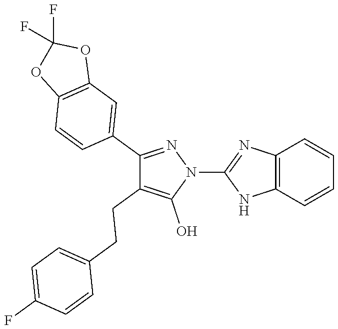 [SMILES: Oc1c(CCc2ccc(F)cc2)c(-c2ccc3c(c2)OC(F)(F)O3)nn1-c1nc2ccccc2[nH]1] | 2.11E- 14 | 7.22E + 08 | 4.38E- 03 | 1:1 | 0 | 98.2 |
| I-146 | 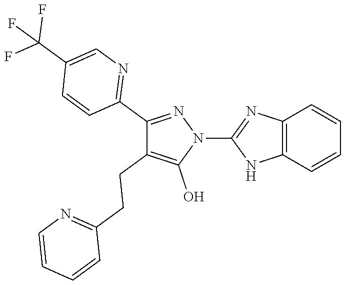 [SMILES: Oc1c(CCc2ccccn2)c(-c2ccc(C(F)(F)F)cn2)nn1-c1nc2ccccc2[nH]1] | 1.65E- 06 | 3.67E + 00 | 6.42E- 04 | 1:1 | 5.7 | 98.5 |
| I-147 | 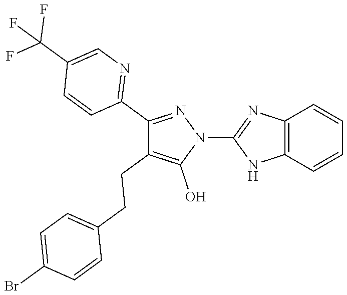 [SMILES: Oc1c(CCc2ccc(Br)cc2)c(-c2ccc(C(F)(F)F)cn2)nn1-c1nc2ccccc2[nH]1] | 7.52E- 11 | 4.30E + 04 | 2.11E- 03 | 1:1 | 59.3 | 97.6 |
| I-714 | 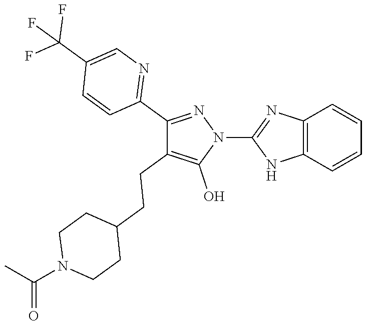 [SMILES: CC(=O)N1CCC(CCc2c(-c3ccc(C(F)(F)F)cn3)nn(-c3nc4ccccc4[nH]3)c2O)CC1] | 1.11E- 06 | 3.64E + 00 | 1.32E- 03 | 1:1 | 63.6 | 98.3 |
| I-715 | 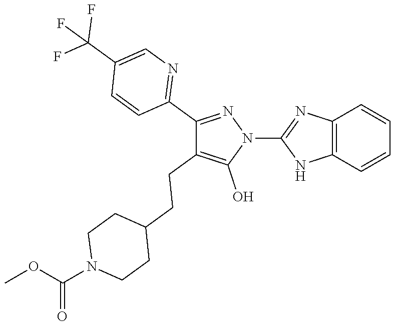 [SMILES: COC(=O)N1CCC(CCc2c(-c3ccc(C(F)(F)F)cn3)nn(-c3nc4ccccc4[nH]3)c2O)CC1] | 2.51E- 06 | 3.76E + 00 | 5.47E- 03 | 2- step | 25.2 | 99.1 |
| I-701 | 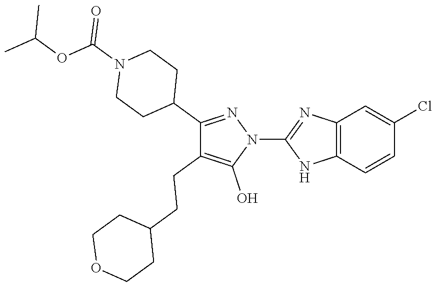 [SMILES: CC(C)OC(=O)N1CCC(c2nn(-c3nc4cc(Cl)ccc4[nH]3)c(O)c2CCC2CCOCC2)CC1] | 2.88E- 06 | 4.73E + 00 | 5.85E- 03 | 2- step | 100 | 98.7 |
| I-704 | 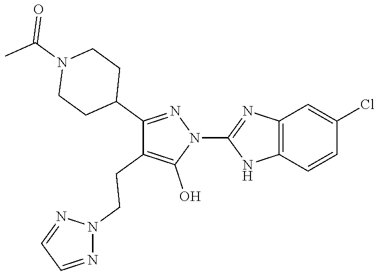 [SMILES: CC(=O)N1CCC(c2nn(-c3nc4cc(Cl)ccc4[nH]3)c(O)c2CCn2nccn2)CC1] | 3.62E- 06 | 7.61E + 00 | 1.92E- 03 | 1:1 | 6.6 | 99.4 |
| I-716 | 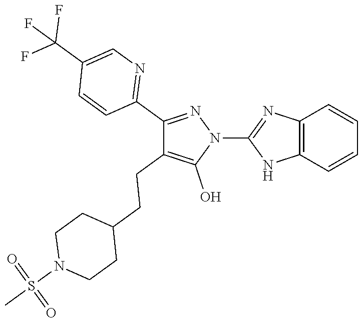 [SMILES: CS(=O)(=O)N1CCC(CCc2c(-c3ccc(C(F)(F)F)cn3)nn(-c3nc4ccccc4[nH]3)c2O)CC1] | 4.37E- 06 | 3.22E + 00 | 5.13E- 03 | 2- step | 22.8 | 97.1 |
| I-706 | 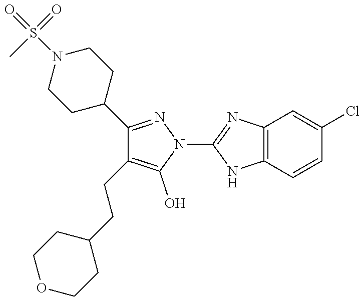 [SMILES: CS(=O)(=O)N1CCC(c2nn(-c3nc4cc(Cl)ccc4[nH]3)c(O)c2CCC2CCOCC2)CC1] | 1.17E- 06 | 3.95E + 00 | 2.34E- 03 | 1:1 | 9.8 | 98.6 |
| I-707 | 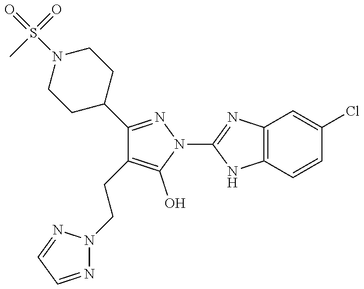 [SMILES: CS(=O)(=O)N1CCC(c2nn(-c3nc4cc(Cl)ccc4[nH]3)c(O)c2CCn2nccn2)CC1] | 1.98E- 06 | 4.33E + 00 | 1.81E- 03 | 1:1 | 0 | 100 |
| I-167 | 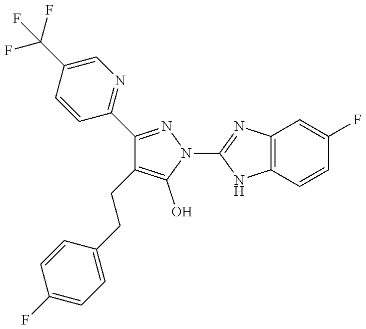 [SMILES: Oc1c(CCc2ccc(F)cc2)c(-c2ccc(C(F)(F)F)cn2)nn1-c1nc2cc(F)ccc2[nH]1] | 2.02E- 09 | 5.05E + 03 | 1.11E- 02 | 2- step | 52.1 | 81.2 |
| I-709 | 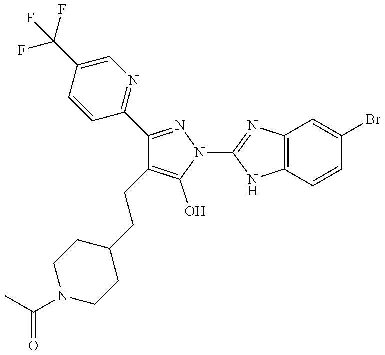 [SMILES: CC(=O)N1CCC(CCc2c(-c3ccc(C(F)(F)F)cn3)nn(-c3nc4cc(Br)ccc4[nH]3)c2O)CC1] | 2.63E- 08 | 5.18E + 02 | 8.85E- 04 | 1:1 | 66.7 | 74.0 |
| I-708 | 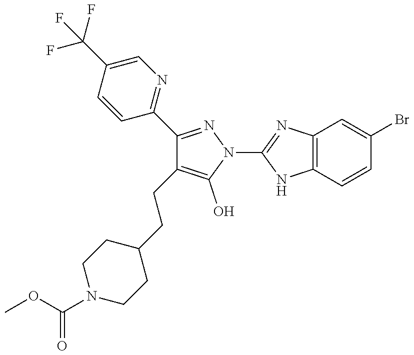 [SMILES: COC(=O)N1CCC(CCc2c(-c3ccc(C(F)(F)F)cn3)nn(-c3nc4cc(Br)ccc4[nH]3)c2O)CC1] | 1.40E- 09 | 1.00E + 04 | 2.04E- 03 | 1:1 | 100 | 77.8 |
| I-775 | 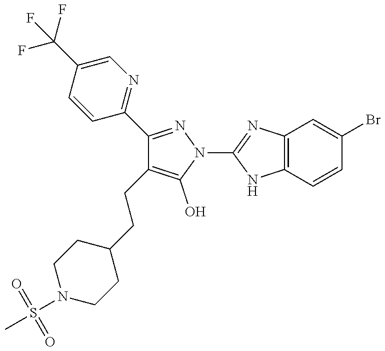 [SMILES: CS(=O)(=O)N1CCC(CCc2c(-c3ccc(C(F)(F)F)cn3)nn(-c3nc4cc(Br)ccc4[nH]3)c2O)CC1] | 3.16E- 07 | 4.26E + 01 | 1.62E- 03 | 1:1 | 62.1 | 72.9 |
| I-406 | 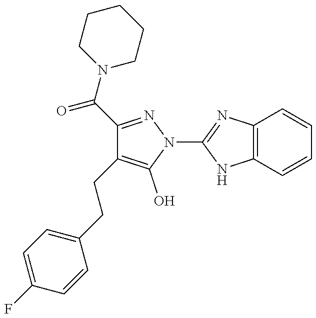 [SMILES: O=C(c1nn(-c2nc3ccccc3[nH]2)c(O)c1CCc1ccc(F)cc1)N1CCCCC1] | 2.67E- 06 | 2.85E + 00 | 7.13E- 03 | 2- step | 0 | 100 |
| I-148 | 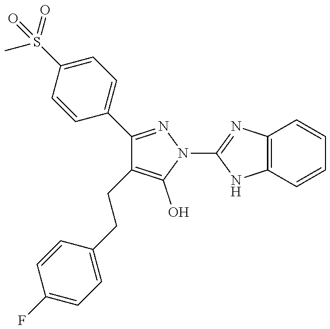 [SMILES: CS(=O)(=O)c1ccc(-c2nn(-c3nc4ccccc4[nH]3)c(O)c2CCc2ccc(F)cc2)cc1] | 3.33E- 10 | 3.26E + 04 | 1.32E- 02 | 2- step | 98.1 | 75.5 |
| I-201 | 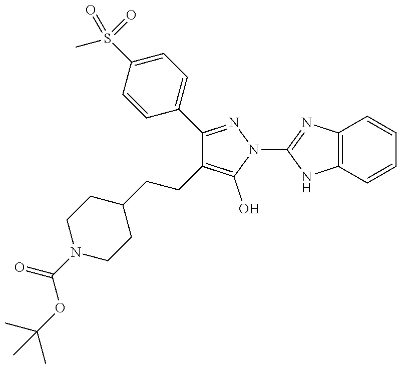 [SMILES: CC(C)(C)OC(=O)N1CCC(CCc2c(-c3ccc(S(C)(=O)=O)cc3)nn(-c3nc4ccccc4[nH]3)c2O)CC1] | 3.94E- 07 | 2.62E + 01 | 1.83E- 02 | 2- step | 53.4 | 70.5 |
| I-208 | 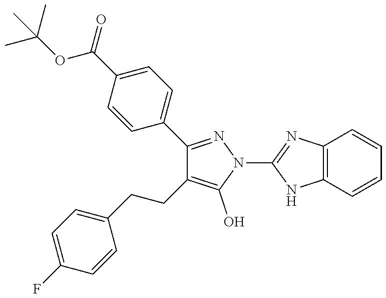 [SMILES: CC(C)(C)OC(=O)c1ccc(-c2nn(-c3nc4ccccc4[nH]3)c(O)c2CCc2ccc(F)cc2)cc1] | 6.78E- 05 | 3.95E- 02 | 1.63E- 02 | 2- step | 39.2 | 90.6 |
| I-215 | 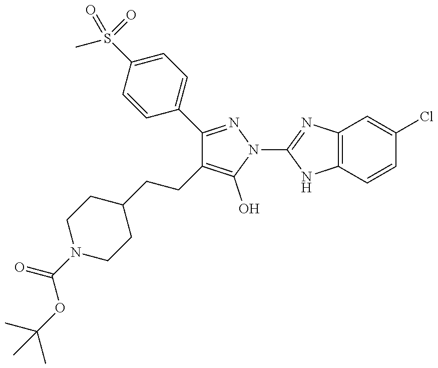 [SMILES: CC(C)(C)OC(=O)N1CCC(CCc2c(-c3ccc(S(C)(=O)=O)cc3)nn(-c3nc4cc(Cl)ccc4[nH]3)c2O)CC1] | 1.62E- 07 | 5.87E + 01 | 1.00E- 02 | 2- step | 53.5 | 74.5 |
| I-550 | 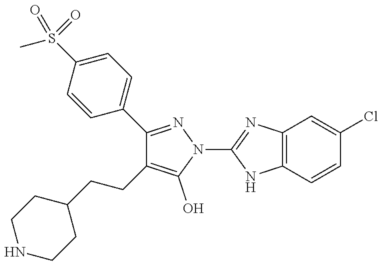 [SMILES: CS(=O)(=O)c1ccc(-c2nn(-c3nc4cc(Cl)ccc4[nH]3)c(O)c2CCC2CCNCC2)cc1] | 8.54E- 09 | 3.33E + 03 | 1.75E- 02 | 2- step | 0 | 98.8 |
| I-168 | 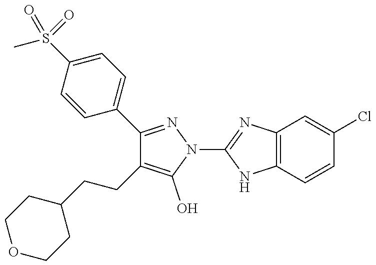 [SMILES: CS(=O)(=O)c1ccc(-c2nn(-c3nc4cc(Cl)ccc4[nH]3)c(O)c2CCC2CCOCC2)cc1] | 2.73E- 06 | 4.96E + 00 | 6.97E- 03 | 2- step | 19.2 | 97.9 |
| I-719 | 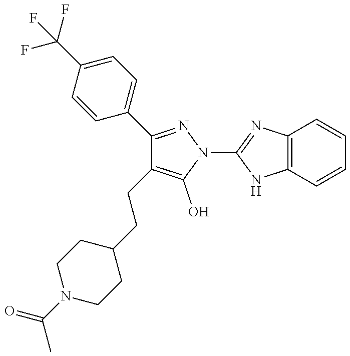 [SMILES: CC(=O)N1CCC(CCc2c(-c3ccc(C(F)(F)F)cc3)nn(-c3nc4ccccc4[nH]3)c2O)CC1] | 4.51E- 07 | 4.44E + 01 | 9.42E- 03 | 2- step | 0 | 98.0 |
| I-1201 | 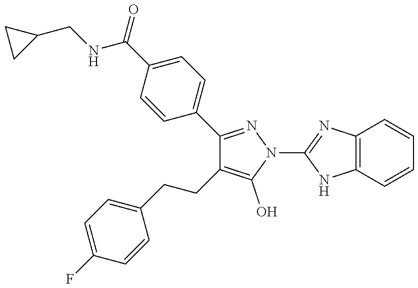 [SMILES: O=C(NCC1CC1)c1ccc(-c2nn(-c3nc4ccccc4[nH]3)c(O)c2CCc2ccc(F)cc2)cc1] | 7.40E- 07 | 2.06E + 03 | 2.17E- 02 | 2- step | 11.8 | 95.4 |
| I-307 | 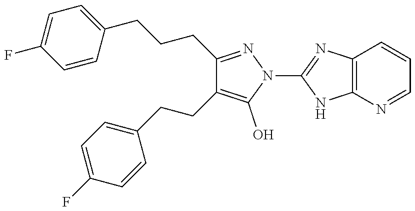 [SMILES: Oc1c(CCc2ccc(F)cc2)c(CCCc2ccc(F)cc2)nn1-c1nc2cccnc2[nH]1] | 8.90E- 07 | 1.14E + 01 | 6.84E- 03 | 1:1 | 47.5 | 98.2 |
| I-218 | 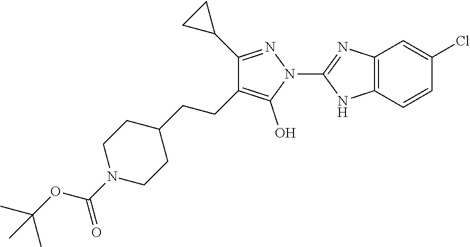 [SMILES: CC(C)(C)OC(=O)N1CCC(CCc2c(C3CC3)nn(-c3nc4cc(Cl)ccc4[nH]3)c2O)CC1] | 6.92E- 07 | 1.02E + 01 | 1.32E- 02 | 2- step | 0 | 97.9 |
| I-170 | 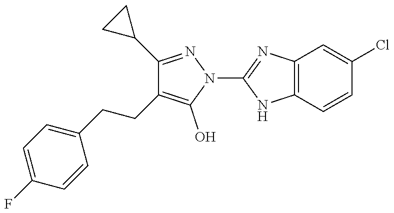 [SMILES: Oc1c(CCc2ccc(F)cc2)c(C2CC2)nn1-c1nc2cc(Cl)ccc2[nH]1] | 1.25E- 08 | 8.01E + 02 | 2.34E- 02 | 2- step | 67.1 | 98.8 |
| I-171 | 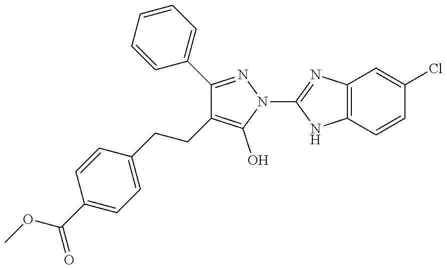 [SMILES: COC(=O)c1ccc(CCc2c(-c3ccccc3)nn(-c3nc4cc(Cl)ccc4[nH]3)c2O)cc1] | 1.33E- 08 | 7.82E + 02 | 2.77E- 02 | 2- step | 100 | 75.8 |
| I-554 | 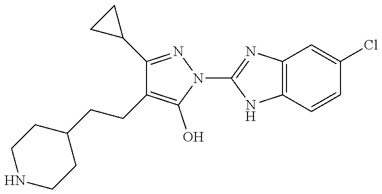 [SMILES: Oc1c(CCC2CCNCC2)c(C2CC2)nn1-c1nc2cc(Cl)ccc2[nH]1] | 3.15E- 12 | 3.33E + 06 | 3.65E- 02 | 2- step | 12.2 | 95.0 |
| I-172 | 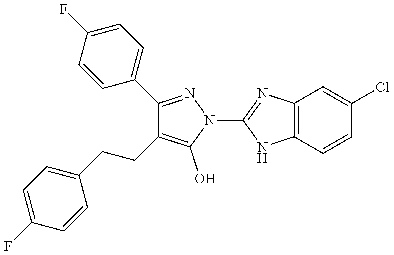 [SMILES: Oc1c(CCc2ccc(F)cc2)c(-c2ccc(F)cc2)nn1-c1nc2cc(Cl)ccc2[nH]1] | 6.93E- 08 | 1.73E + 01 | 4.28E- 02 | 2- step | 56 | 96.5 |
| I-165 | 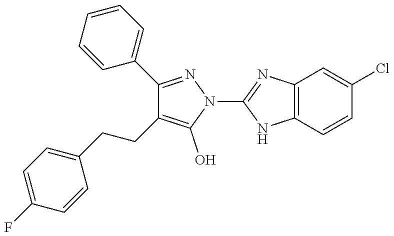 [SMILES: Oc1c(CCc2ccc(F)cc2)c(-c2ccccc2)nn1-c1nc2cc(Cl)ccc2[nH]1] | 6.36E- 07 | 5.10E + 00 | 4.14E- 02 | 2- step | 25.3 | 89.6 |
| I-209 | 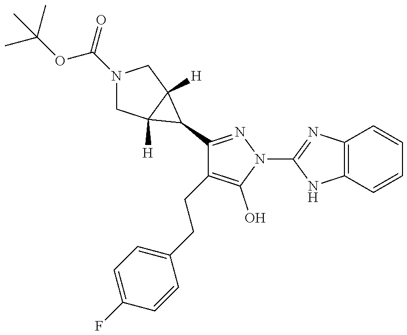 [SMILES: CC(C)(C)OC(=O)N1C[C@@H]2[C@H](C1)[C@H]2c1nn(-c2nc3ccccc3[nH]2)c(O)c1CCc1ccc(F)cc1] | 4.83E- 06 | 3.81E- 01 | 8.59E- 03 | 2- step | 4.2 | 100 |
| I-710 | 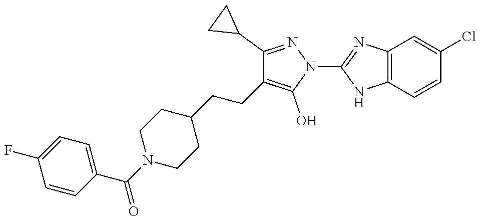 [SMILES: O=C(c1ccc(F)cc1)N1CCC(CCc2c(C3CC3)nn(-c3nc4cc(Cl)ccc4[nH]3)c2O)CC1] | 2.46E- 06 | 4.16E + 00 | 2.12E- 02 | 2- step | 2.5 | 100 |
| I-711 | 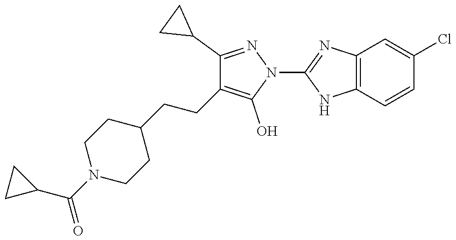 [SMILES: O=C(C1CC1)N1CCC(CCc2c(C3CC3)nn(-c3nc4cc(Cl)ccc4[nH]3)c2O)CC1] | 3.29E- 06 | 5.97E + 00 | 1.96E- 02 | 2- step | 0 | 97.2 |
| I-712 | 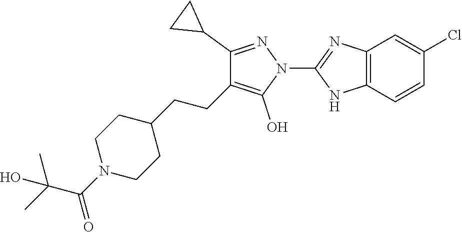 [SMILES: CC(C)(O)C(=O)N1CCC(CCc2c(C3CC3)nn(-c3nc4cc(Cl)ccc4[nH]3)c2O)CC1] | 1.99E- 08 | 7.54E + 02 | 1.07E- 02 | 2- step | 52.3 | 100 |
| I-901 | 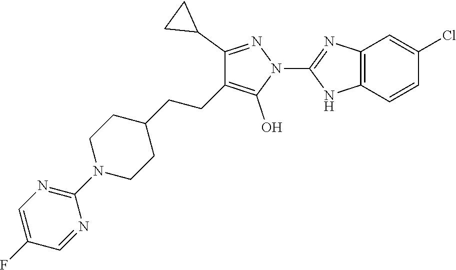 [SMILES: Oc1c(CCC2CCN(c3ncc(F)cn3)CC2)c(C2CC2)nn1-c1nc2cc(Cl)ccc2[nH]1] | 1.53E- 11 | 2.46E + 05 | 2.23E- 03 | 2- step | 14.3 | 100 |
| I-603 | 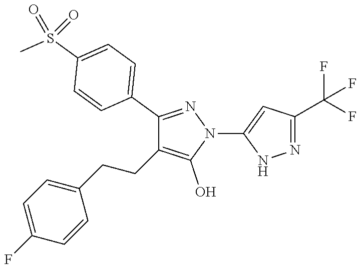 [SMILES: CS(=O)(=O)c1ccc(-c2nn(-c3cc(C(F)(F)F)n[nH]3)c(O)c2CCc2ccc(F)cc2)cc1] | 3.51E- 05 | 2.18E + 00 | 1.19E- 02 | 2- step | 57.8 | 100 |
| I-604 | 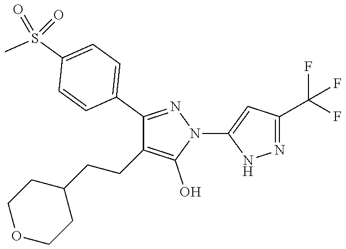 [SMILES: CS(=O)(=O)c1ccc(-c2nn(-c3cc(C(F)(F)F)n[nH]3)c(O)c2CCC2CCOCC2)cc1] | 4.52E- 05 | 5.48E + 00 | 1.24E- 02 | 2- step | 25.5 | 100 |
| I-605 | 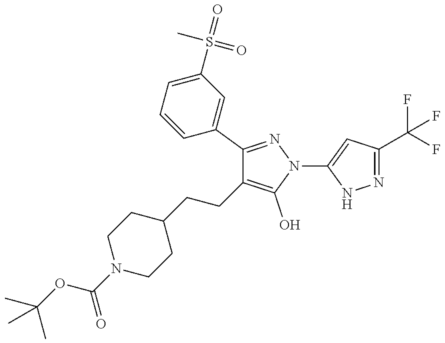 [SMILES: CC(C)(C)OC(=O)N1CCC(CCc2c(-c3cccc(S(C)(=O)=O)c3)nn(-c3cc(C(F)(F)F)n[nH]3)c2O)CC1] | 1.52E- 05 | 4.16E + 00 | 1.91E- 02 | 2- step | 68.7 | 100 |
| I-902 | 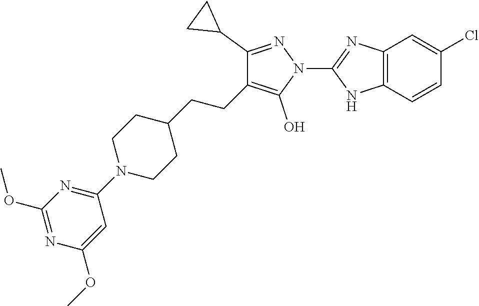 [SMILES: COc1cc(N2CCC(CCc3c(C4CC4)nn(-c4nc5cc(Cl)ccc5[nH]4)c3O)CC2)nc(OC)n1] | 3.67E- 12 | 1.46E + 06 | 7.18E- 03 | 2- step | 13.8 | 100 |
| I-210 | 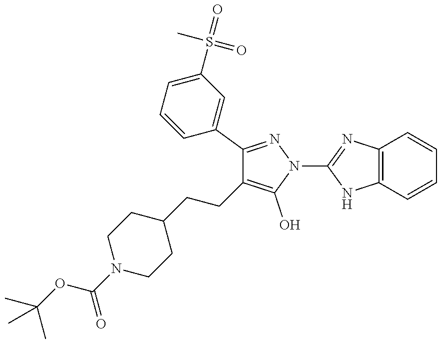 [SMILES: CC(C)(C)OC(=O)N1CCC(CCc2c(-c3cccc(S(C)(=O)=O)c3)nn(-c3nc4ccccc4[nH]3)c2O)CC1] | 4.56E- 06 | 1.06E + 01 | 5.18E- 03 | 2- step | 95.4 | 83.7 |
| I-606 | 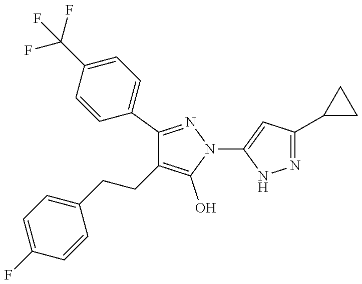 [SMILES: Oc1c(CCc2ccc(F)cc2)c(-c2ccc(C(F)(F)F)cc2)nn1-c1cc(C2CC2)n[nH]1] | 4.67E- 06 | 5.79E + 00 | 1.06E- 02 | 2- step | 0 | 95.5 |
| I-607 | 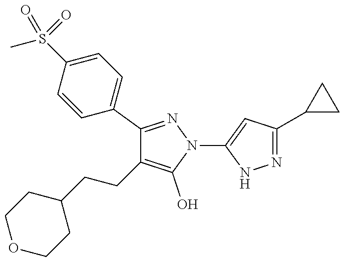 [SMILES: CS(=O)(=O)c1ccc(-c2nn(-c3cc(C4CC4)n[nH]3)c(O)c2CCC2CCOCC2)cc1] | 8.86E- 05 | 3.74E- 01 | 7.73E- 03 | 1:1 | 57.3 | 100 |
| I-776 | 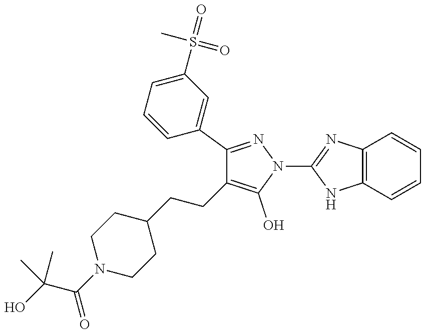 [SMILES: CC(C)(O)C(=O)N1CCC(CCc2c(-c3cccc(S(C)(=O)=O)c3)nn(-c3nc4ccccc4[nH]3)c2O)CC1] | 1.20E- 06 | 1.06E + 01 | 5.14E- 03 | 2- step | 19.3 | 97.1 |
| I-609 | 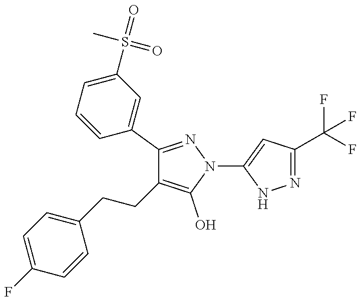 [SMILES: CS(=O)(=O)c1cccc(-c2nn(-c3cc(C(F)(F)F)n[nH]3)c(O)c2CCc2ccc(F)cc2)c1] | 6.49E- 06 | 1.71E + 00 | 1.95E- 03 | 1:1 | 67.3 | 99.6 |
| I-610 | 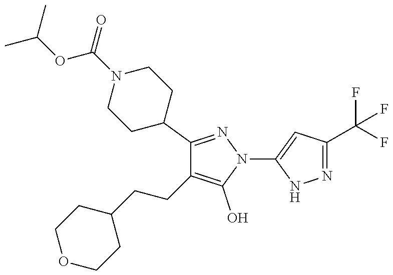 [SMILES: CC(C)OC(=O)N1CCC(c2nn(-c3cc(C(F)(F)F)n[nH]3)c(O)c2CCC2CCOCC2)CC1] | 514E- 05 | 3.66E- 01 | 6.91E- 03 | 1:1 | 25.7 | 100 |
| I-612 | 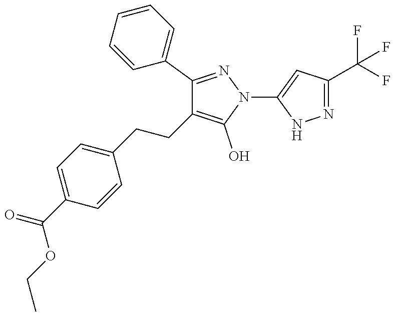 [SMILES: CCOC(=O)c1ccc(CCc2c(-c3ccccc3)nn(-c3cc(C(F)(F)F)n[nH]3)c2O)cc1] | 3.82E- 06 | 8.96E + 00 | 1.39E- 02 | 2- step | 13.8 | 100 |
| I-613 | 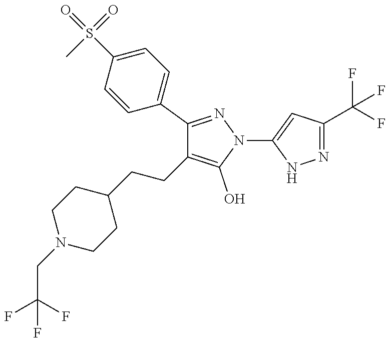 [SMILES: CS(=O)(=O)c1ccc(-c2nn(-c3cc(C(F)(F)F)n[nH]3)c(O)c2CCC2CCN(CC(F)(F)F)CC2)cc1] | 2.46E- 06 | 2.22E + 00 | 2.05E- 03 | 1:1 | 51.3 | 99.7 |
| I-614 | 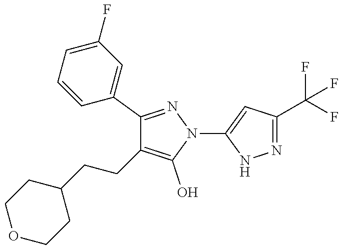 [SMILES: Oc1c(CCC2CCOCC2)c(-c2cccc(F)c2)nn1-c1cc(C(F)(F)F)n[nH]1] | 1.87E- 05 | 2.26E + 00 | 4.94E- 03 | 1:1 | 0 | 100 |
| I-150 | 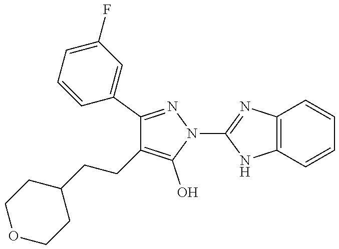 [SMILES: Oc1c(CCC2CCOCC2)c(-c2cccc(F)c2)nn1-c1nc2ccccc2[nH]1] | 1.07E- 06 | 9.69E + 00 | 4.75E- 03 | 2- step | 53.9 | 77.7 |
| I-304 | 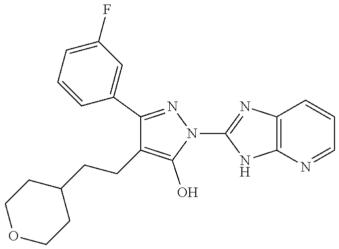 [SMILES: Oc1c(CCC2CCOCC2)c(-c2cccc(F)c2)nn1-c1nc2cccnc2[nH]1] | 3.15E- 06 | 1.25E + 01 | 5.25E- 03 | 2- step | 0 | 98.7 |
| I-616 | 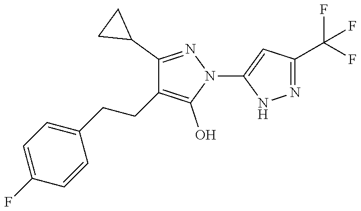 [SMILES: Oc1c(CCc2ccc(F)cc2)c(C2CC2)nn1-c1cc(C(F)(F)F)n[nH]1] | 4.58E- 05 | 7.37E + 00 | 3.07E- 02 | 2- step | 0 | 95.8 |
| I-308 | 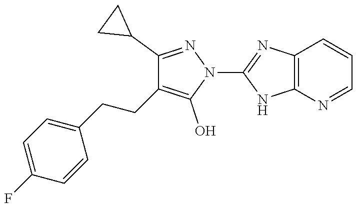 [SMILES: Oc1c(CCc2ccc(F)cc2)c(C2CC2)nn1-c1nc2cccnc2[nH]1] | 4.06E- 06 | 5.81E + 00 | 6.06E- 03 | 2- step | 61.4 | 100 |
| I-723 | 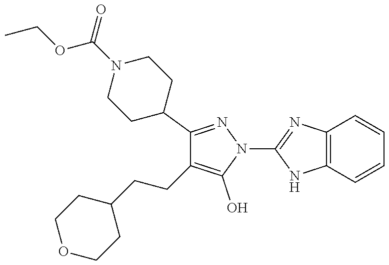 [SMILES: CCOC(=O)N1CCC(c2nn(-c3nc4ccccc4[nH]3)c(O)c2CCC2CCOCC2)CC1] | 9.09E- 06 | 5.21E + 00 | 9.19E- 03 | 2- step | 0 | 92.2 |
| I-617 | 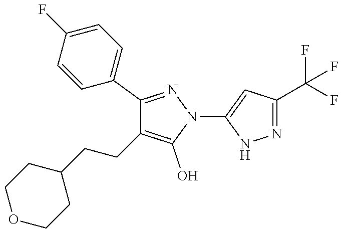 [SMILES: Oc1c(CCC2CCOCC2)c(-c2ccc(F)cc2)nn1-c1cc(C(F)(F)F)n[nH]1] | 9.64E- 06 | 3.96E + 00 | 1.31E- 03 | 1:1 | 0 | 99.8 |
| I-154 | 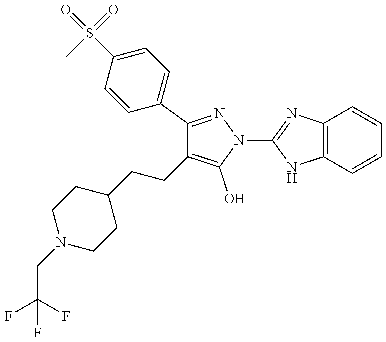 [SMILES: CS(=O)(=O)c1ccc(-c2nn(-c3nc4ccccc4[nH]3)c(O)c2CCC2CCN(CC(F)(F)F)CC2)cc1] | 2.88E- 05 | 6.80E + 00 | 4.50E- 03 | 1:1 | 45.4 | 69.3 |
| I-305 | 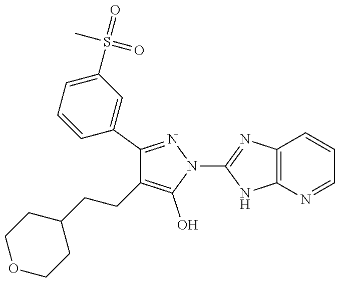 [SMILES: CS(=O)(=O)c1cccc(-c2nn(-c3nc4cccnc4[nH]3)c(O)c2CCC2CCOCC2)c1] | 7.44E- 07 | 1.13E + 01 | 2.13E- 03 | 1:1 | 59.3 | 96.8 |
| I-403 | 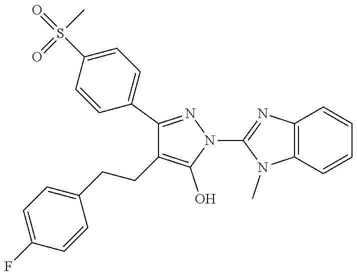 [SMILES: Cn1c(-n2nc(-c3ccc(S(C)(=O)=O)cc3)c(CCc3ccc(F)cc3)c2O)nc2ccccc21] | 2.40E- 06 | 3.63E + 00 | 2.88E- 03 | 1:1 | 4.1 | 99.9 |
| I-405 | 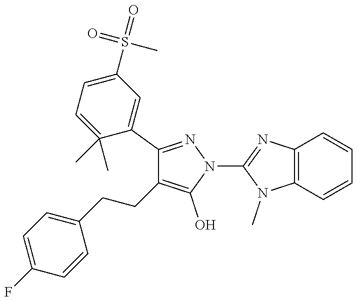 | 4.39E- 06 | 2.32E + 00 | 3.25E- 03 | 1:1 | 0 | 95.7 |
| I-151 | 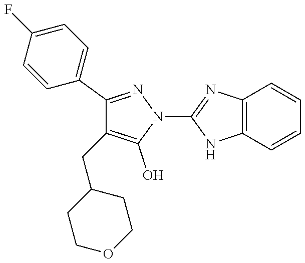 [SMILES: Oc1c(CC2CCOCC2)c(-c2ccc(F)cc2)nn1-c1nc2ccccc2[nH]1] | 6.26E- 06 | 4.68E + 00 | 8.50E- 03 | 2- step | 100 | 83.0 |
| I-620 | 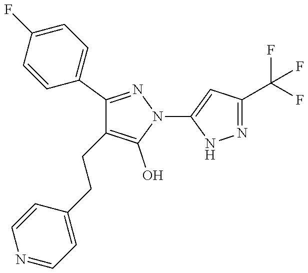 [SMILES: Oc1c(CCc2ccncc2)c(-c2ccc(F)cc2)nn1-c1cc(C(F)(F)F)n[nH]1] | 7.47E- 06 | 1.63E + 00 | 1.44E- 03 | 1:1 | 12 | 100 |
| I-725 | 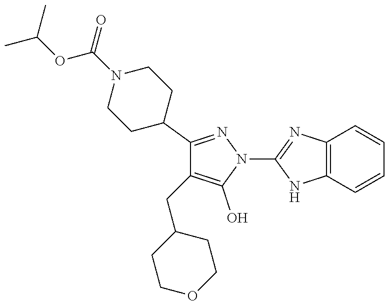 [SMILES: CC(C)OC(=O)N1CCC(c2nn(-c3nc4ccccc4[nH]3)c(O)c2CC2CCOCC2)CC1] | 3.29E- 06 | 4.73E + 00 | 3.88E- 03 | 1:1 | 37.4 | 95.6 |
| I-402 | 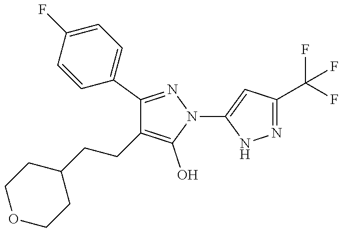 [SMILES: Oc1c(CCC2CCOCC2)c(-c2ccc(F)cc2)nn1-c1cc(C(F)(F)F)n[nH]1] | 4.75E- 06 | 1.47E + 00 | 2.00E- 03 | 1:1 | 8.7 | 100 |
| I-1501 | 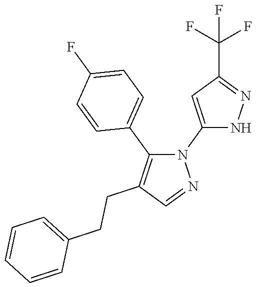 [SMILES: Fc1ccc(-c2c(CCc3ccccc3)cnn2-c2cc(C(F)(F)F)n[nH]2)cc1] | 1.98E- 07 | 2.36E + 02 | 1.15E- 02 | 1:1 | 3 | 97.9 |
| I-177 | 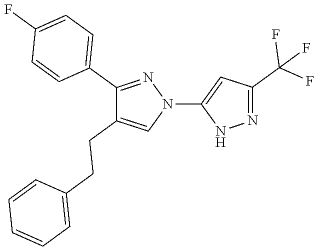 [SMILES: Fc1ccc(-c2nn(-c3cc(C(F)(F)F)n[nH]3)cc2CCc2ccccc2)cc1] | 9.42E- 07 | 3.64E + 00 | 6.00E- 03 | 1:1 | 0 | 100 |
| I-313 | 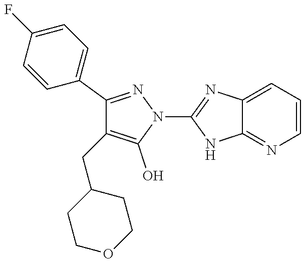 [SMILES: Oc1c(CC2CCOCC2)c(-c2ccc(F)cc2)nn1-c1nc2cccnc2[nH]1] | 4.80E- 07 | 2.92E + 01 | 5.15E- 03 | 1:1 | 33.2 | 96.7 |
| I-314 | 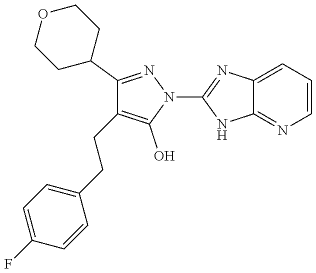 [SMILES: Oc1c(CCc2ccc(F)cc2)c(C2CCOCC2)nn1-c1nc2cccnc2[nH]1] | 7.05E- 07 | 1.79E + 01 | 2.97E- 03 | 1:1 | 0 | 100 |
| I-174 | 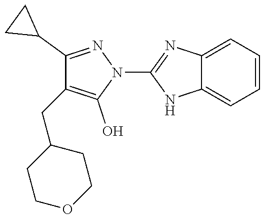 [SMILES: Oc1c(CC2CCOCC2)c(C2CC2)nn1-c1nc2ccccc2[nH]1] | 1.37E- 06 | 6.54E + 00 | 3.94E- 03 | 1:1 | 11.9 | 100 |
| I-623 | 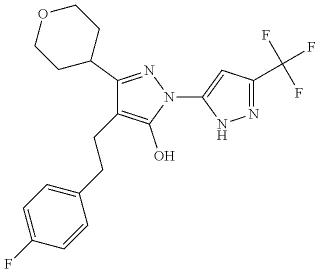 [SMILES: Oc1c(CCc2ccc(F)cc2)c(C2CCOCC2)nn1-c1cc(C(F)(F)F)n[nH]1] | 2.21E- 04 | 2.88E + 00 | 1.67E- 02 | 1:1 | 17.4 | 100 |
| I-780 | 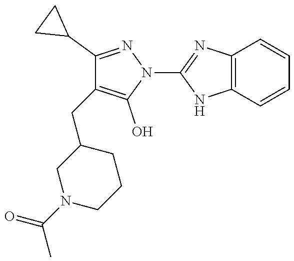 [SMILES: CC(=O)N1CCCC(Cc2c(C3CC3)nn(-c3nc4ccccc4[nH]3)c2O)C1] | 4.07E- 06 | 8.13E + 00 | 4.47E- 03 | 1:1 | 0 | 99 |
| I-782 | 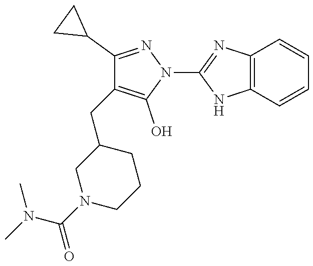 [SMILES: CN(C)C(=O)N1CCCC(Cc2c(C3CC3)nn(-c3nc4ccccc4[nH]3)c2O)C1] | 9.41E- 06 | 3.62E + 00 | 5.13E- 03 | 1:1 | 61.1 | 98.8 |
| I-175 | 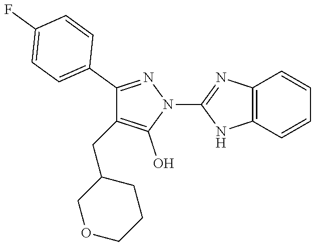 [SMILES: Oc1c(CC2CCCOC2)c(-c2ccc(F)cc2)nn1-c1nc2ccccc2[nH]1] | 6.05E- 07 | 7.72E + 00 | 6.67E- 04 | 1:1 | 67.2 | 88.8 |
| I-783 | 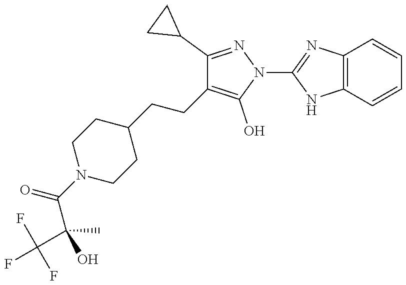 [SMILES: C[C@@](O)(C(=O)N1CCC(CCc2c(C3CC3)nn(-c3nc4ccccc4[nH]3)c2O)CC1)C(F)(F)F] | 3.37E- 06 | 4.08E + 00 | 1.38E- 03 | 1:1 | 5.4 | 100 |
| I-176 | 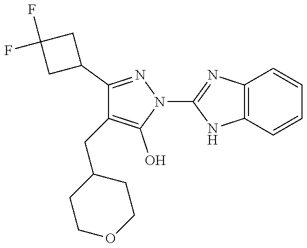 [SMILES: Oc1c(CC2CCOCC2)c(C2CC(F)(F)C2)nn1-c1nc2ccccc2[nH]1] | 2.24E- 06 | 5.08E + 00 | 1.52E- 03 | 1:1 | 0 | 98.4 |
| I-316 | 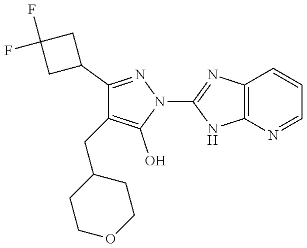 [SMILES: Oc1c(CC2CCOCC2)c(C2CC(F)(F)C2)nn1-c1nc2cccnc2[nH]1] | 4.49E- 06 | 5.19E + 00 | 1.19E- 03 | 1:1 | 32.8 | 99.7 |
| I-173 | 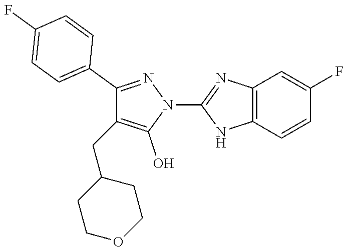 [SMILES: Oc1c(CC2CCOCC2)c(-c2ccc(F)cc2)nn1-c1nc2cc(F)ccc2[nH]1] | 3.48E- 10 | 7.12E + 04 | 5.24E- 03 | 2- step | 97.2 | 85.7 |
| I-178 | 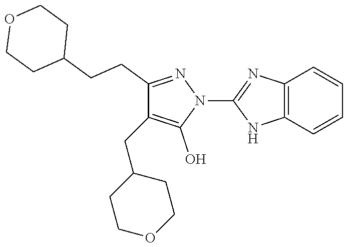 [SMILES: Oc1c(CC2CCOCC2)c(CCC2CCOCC2)nn1-c1nc2ccccc2[nH]1] | 1.09E- 05 | 7.72E + 00 | 8.86E- 03 | 2- step | 0 | 97.9 |
| I-180 | 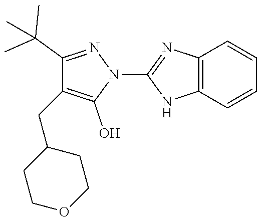 [SMILES: CC(C)(C)c1nn(-c2nc3ccccc3[nH]2)c(O)c1CC1CCOCC1] | 7.19E- 06 | 2.48E + 00 | 2.62E- 03 | 1:1 | 74.8 | 100 |
| I-182 | 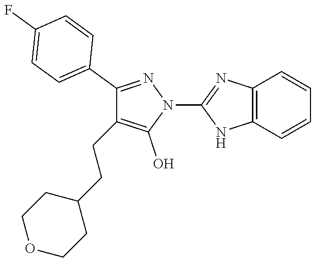 [SMILES: Oc1c(CCC2CCOCC2)c(-c2ccc(F)cc2)nn1-c1nc2ccccc2[nH]1] | 4.42E- 06 | 9.98E + 00 | 1.11E- 02 | 2- step | 100 | 90 |
| I-183 | 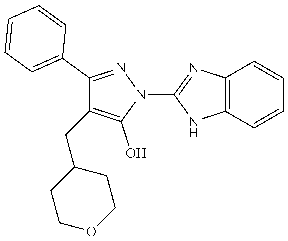 [SMILES: Oc1c(CC2CCOCC2)c(-c2ccccc2)nn1-c1nc2ccccc2[nH]1] | 2.27E- 06 | 7.61E + 00 | 1.71E- 03 | 1:1 | 37.8 | 100 |
| I-184 | 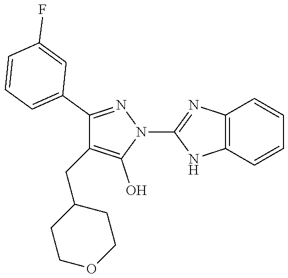 [SMILES: Oc1c(CC2CCOCC2)c(-c2cccc(F)c2)nn1-c1nc2ccccc2[nH]1] | 3.52E- 06 | 6.46E + 00 | 2.48E- 03 | 1:1 | 100 | 100 |
| I-185 | 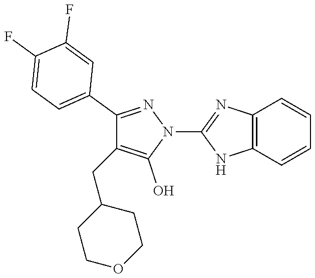 [SMILES: Oc1c(CC2CCOCC2)c(-c2ccc(F)c(F)c2)nn1-c1nc2ccccc2[nH]1] | 3.40E- 06 | 1.04E + 01 | 4.57E- 03 | 2- step | 100 | 95.1 |
| I-186 | 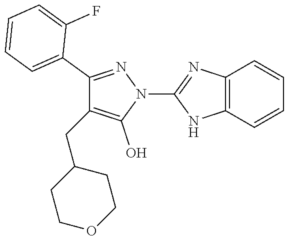 [SMILES: Oc1c(CC2CCOCC2)c(-c2ccccc2F)nn1-c1nc2ccccc2[nH]1] | 1.17E- 05 | 7.72E + 00 | 1.62E- 02 | 2- step | 61.5 | 99.3 |
| I-187 | 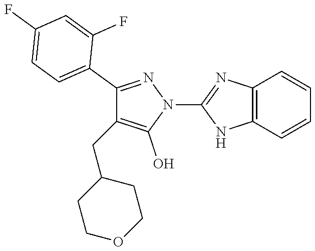 [SMILES: Oc1c(CC2CCOCC2)c(-c2ccc(F)cc2F)nn1-c1nc2ccccc2[nH]1] | 3.07E- 11 | 1.02E + 06 | 8.62E- 03 | 2- step | 20.2 | 100 |

In view of the many possible embodiments to which the principles of the disclosed invention may be applied, it should be recognized that the illustrated embodiments are only preferred examples of the invention and should not be taken as limiting the scope of the invention.

1. A compound of formula I, or a stereoisomer, tautomer, or pharmaceutically acceptable salt thereof: 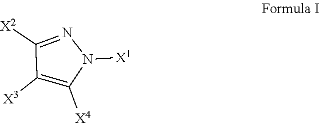
wherein X¹ is benzimidazolyl, substituted benzimidazolyl, azabenzimidazolyl, substituted azabenzimidazolyl, pyrazolyl, or substituted pyrazolyl;
X² is aryl, substituted aryl, arylalkyl, substituted arylalkyl, heteroaryl, substituted heteroaryl, heteroarylalkyl, substituted heteroarylalkyl, cycloalkyl, substituted cycloalkyl, (cycloalkyl)alkyl, substituted (cycloalkyl)alkyl, heterocycloalkyl, substituted heterocycloalkyl, (heterocycloalkyl)alkyl, substituted (heterocycloalkyl)alkyl, alkyl, substituted alkyl, alkoxycarbonyl, substituted alkoxycarbonyl, dialkylaminocarbonyl, substituted dialkylaminocarbonyl, (heterocycloalkyl)carbonyl, or substituted (heterocycloalkyl)carbonyl;
X³ is alkyl, substituted alkyl, alkenyl, substituted alkenyl, heterocycloalkyl, substituted heterocycloalkyl, (heterocycloalkyl)alkyl, substituted (heterocycloalkyl)alkyl, aryl, substituted aryl, arylalkyl, substituted arylalkyl, heteroaryl, substituted heteroaryl, heteroarylalkyl, substituted heteroarylalkyl, cycloalkyl, substituted cycloalkyl, (cycloalkyl)alkyl, or substituted (cycloalkyl)alkyl; and
X⁴ is hydroxy, amino, alkyl, or hydrogen;
provided that if X¹ is benzimidazolyl, substituted benzimidazolyl, pyrazolyl, or substituted pyrazolyl, then X² is aryl, substituted aryl, arylalkyl, substituted arylalkyl, heteroaryl, substituted heteroaryl, heteroarylalkyl, substituted heteroarylalkyl, cycloalkyl, substituted cycloalkyl, (cycloalkyl)alkyl, substituted (cycloalkyl)alkyl, heterocycloalkyl, substituted heterocycloalkyl, (heterocycloalkyl)alkyl, or substituted (heterocycloalkyl)alkyl; X³ is heterocycloalkyl, substituted heterocycloalkyl, (heterocycloalkyl)alkyl, substituted (heterocycloalkyl)alkyl, aryl, substituted aryl, arylalkyl, substituted arylalkyl, heteroaryl, substituted heteroaryl, heteroarylalkyl, substituted heteroarylalkyl, cycloalkyl, substituted cycloalkyl, (cycloalkyl)alkyl, or substituted (cycloalkyl)alkyl; and further provided that at least one of X² or X³ is substituted aryl, substituted arylalkyl, substituted heteroaryl, substituted heteroarylalkyl, substituted cycloalkyl, substituted (cycloalkyl)alkyl, substituted heterocycloalkyl, or substituted (heterocycloalkyl)alkyl.

2. A compound of formula I, or a stereoisomer, tautomer, or pharmaceutically acceptable salt thereof: 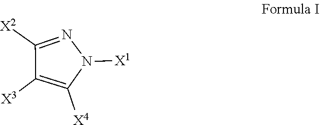
wherein X¹ is benzimidazolyl, substituted benzimidazolyl, azabenzimidazolyl, substituted azabenzimidazolyl, pyrazolyl, or substituted pyrazolyl;
X² is aryl, substituted aryl, arylalkyl, substituted arylalkyl, heteroaryl, substituted heteroaryl, heteroarylalkyl, substituted heteroarylalkyl, cycloalkyl, substituted cycloalkyl, (cycloalkyl)alkyl, substituted (cycloalkyl)alkyl, heterocycloalkyl, substituted heterocycloalkyl, (heterocycloalkyl)alkyl, substituted (heterocycloalkyl)alkyl, alkoxycarbonyl, substituted alkoxycarbonyl, dialkylaminocarbonyl, substituted dialkylaminocarbonyl, (heterocycloalkyl)carbonyl, or substituted (heterocycloalkyl)carbonyl;
X³ is alkyl, substituted alkyl, alkenyl, substituted alkenyl, heterocycloalkyl, substituted heterocycloalkyl, (heterocycloalkyl)alkyl, substituted (heterocycloalkyl)alkyl, aryl, substituted aryl, arylalkyl, substituted arylalkyl, heteroaryl, substituted heteroaryl, heteroarylalkyl, substituted heteroarylalkyl, cycloalkyl, substituted cycloalkyl, (cycloalkyl)alkyl, or substituted (cycloalkyl)alkyl; and
X⁴ is hydroxy, amino, alkyl, or hydrogen;
provided that if X¹ is benzimidazolyl, substituted benzimidazolyl, pyrazolyl, or substituted pyrazolyl, then at least one of X² or X³ is heteroaryl, substituted heteroaryl, heteroarylalkyl, substituted heteroarylalkyl, cycloalkyl, substituted cycloalkyl, (cycloalkyl)alkyl, substituted (cycloalkyl)alkyl, heterocycloalkyl, substituted heterocycloalkyl, (heterocycloalkyl)alkyl, or substituted (heterocycloalkyl)alkyl.

3. The compound of claim 1, wherein X¹ is a benzimidazolyl or a substituted benzimidazolyl.

4. The compound of claim 1, wherein X¹ is: 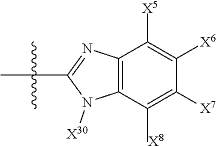
wherein each of X⁵, X⁶, X⁷, and X⁸ is independently selected from hydrogen, halogen, cyano, alkyl, haloalkyl, alkoxy, haloalkoxy or cycloalkyl; and X³⁰ is selected from hydrogen, alkyl, haloalkyl, or cycloalkyl.

5. The compound of claim 4, wherein each of X⁵, X⁶, X⁷, X⁸, and X³⁰ is hydrogen.

6. The compound of claim 4, wherein X⁶ is halogen.

7. The compound of claim 4, wherein X⁶ is halogen, and each of X⁵, X⁷, X⁸, and X³⁰ is hydrogen.

8. The compound of claim 1, wherein X¹ is azabenzimidazolyl or substituted azabenzimidazolyl.

9. The compound of claim 1, wherein X¹ is: 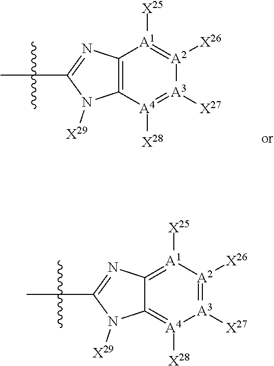
wherein each of A¹, A², A³ and A⁴ is independently C or N, provided at least one of A¹, A², A³ and A⁴ is N; each of X²⁵, X²⁶, X²⁷, and X²⁸ is independently selected from hydrogen, halogen, cyano, alkyl, haloalkyl, alkoxy, haloalkoxy or cycloalkyl; and X²⁹ is hydrogen, alkyl, haloalkyl, or cycloalkyl; and also provided that if A¹ is N then X²⁵ is not present, if A² is N then X²⁶ is not present, if A³ is N then X²⁷ is not present, and if A⁴ is N then X²⁸ is not present.

10. The compound of claim 9, wherein A⁴ is N and X²⁸ is not present, and A¹, A² and A³ are each C.

11. The compound of claim 10, wherein each of X²⁵, X²⁶, X²⁷, and X²⁹ is H.

12. The compound of claim 1, wherein X¹ is pyrazolyl or substituted pyrazolyl.

13. The compound of claim 1, wherein X¹ is: 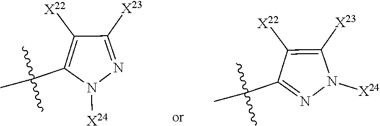
wherein each of X²² and X²³ and X²⁴ is independently selected from hydrogen, halogen, cyano, alkyl, haloalkyl, alkoxy, haloalkoxy, and cycloalkyl; and X²⁴ is selected from hydrogen, alkyl, haloalkyl, alkylsulfonyl and cycloalkyl.

14. The compound of claim 13, wherein X²² and X²⁴ are each hydrogen, and X²³ is alkyl, haloalkyl or cycloalkyl.

15. The compound of claim 13, wherein X²² and X²⁴ are each hydrogen, and X²³ is —CF₃.

16. The compound of claim 1, wherein X² is 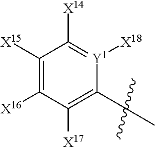
wherein each of X¹⁴, X¹⁵, X¹⁶, X¹⁷ and X¹⁸ is independently selected from hydrogen, halogen, cyano, alkyl, haloalkyl, alkoxy, haloalkoxy, alkylsulfonyl or substituted alkyl, or X¹⁵ and X¹⁶ taken together, or X¹⁶ and X¹⁷ taken together, comprise a methylenedioxy or difluoromethylenedioxy group; and
Y¹ is C or N, provided that if Y¹ then X¹⁸ is not present.

17. The compound of claim 16, wherein X¹⁵ is a haloalkyl.

18. The compound of claim 16, wherein X¹⁵ is —CF₃.

19. The compound of claim 16, wherein X¹⁵ is alkylsulfonyl.

20. The compound of claim 16, wherein X¹⁵ is MeSO₂—.

21. The compound of claim 16, wherein X¹⁶ is alkylsulfonyl.

22. The compound of claim 16, wherein X¹⁶ is MeSO₂—.

23. The compound of claim 16, wherein at least one of X¹⁴, X¹⁵, X¹⁶, X¹⁷ or X¹⁸ is not hydrogen.

24. The compound of claim 1, wherein X² is a N-heterocycloalkyl.

25. The compound of claim 1, wherein X² is 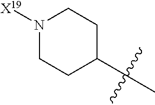
wherein X¹⁹ is alkoxycarbonyl, substituted alkoxycarbonyl, heteroaryl, substituted heteroaryl, alkylcarbonyl, substituted alkylcarbonyl alkoxycarbonylamino, substituted alkoxycarbonylamino alkyl, substituted alkyl, haloalkyl, substituted haloalkyl, cycloalkyl, substituted cycloalkyl, arylalkyl, substituted arylalkyl, heteroarylalkyl, substituted heteroarylalkyl, (heterocycloalkyl)alkyl, substituted (heterocycloalkyl)alkyl, cycloalkylcarbonyl, substituted cycloalkylcarbonyl, hydroxycycloalkylcarbonyl, substituted hydroxycycloalkylcarbonyl, hydroxyalkylcarbonyl, substituted hydroxyalkylcarbonyl, alkoxyalkylcarbonyl, substituted alkoxyalkylcarbonyl, arylalkoxycarbonyl, substituted arylalkoxycarbonyl, arylcarbonyl, substituted arylcarbonyl, heterocycloalkyl, substituted heterocycloalkyl, heterocycloalkylcarbonyl, substituted heterocycloalkylcarbonyl, heteroarylcarbonyl, substituted heteroarylcarbonyl, (arylalkyl)carbonyl, substituted (arylalkyl)carbonyl, (heterocycloalkyl)alkylcarbonyl, substituted (heterocycloalkyl)alkylcarbonyl, heteroarylalkylcarbonyl, substituted heteroarylalkylcarbonyl, heteroarylalkoxyalkylcarbonyl, substituted heteroarylalkoxyalkylcarbonyl, alkylsulfonyl, substituted alkylsulfonyl, cycloalkylsulfonyl, substituted cycloalkylsulfonyl, arylsulfonyl, substituted arylsulfonyl, heteroarylsulfonyl, substituted heteroarylsulfonyl, alkylaminocarbonyl, substituted alkylaminocarbonyl, dialkylaminocarbonyl, substituted dialkylaminocarbonyl, dialkylaminosulfonyl, or substituted dialkylaminosulfonyl.

26. The compound of claim 25, wherein X¹⁹ is alkoxycarbonyl, dialkylaminosulfonyl, or cycloalkylsulfonyl.

27. The compound of claim 1, wherein X² is 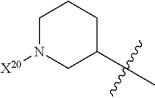
wherein X²⁰ is alkoxycarbonyl, substituted alkoxycarbonyl, heteroaryl, substituted heteroaryl, alkylcarbonyl, substituted alkylcarbonyl alkoxycarbonylamino, substituted alkoxycarbonylamino alkyl, substituted alkyl, haloalkyl, substituted haloalkyl, cycloalkyl, substituted cycloalkyl, arylalkyl, substituted arylalkyl, heteroarylalkyl, substituted heteroarylalkyl, (heterocycloalkyl)alkyl, substituted (heterocycloalkyl)alkyl, cycloalkylcarbonyl, substituted cycloalkylcarbonyl, hydroxycycloalkylcarbonyl, substituted hydroxycycloalkylcarbonyl, hydroxyalkylcarbonyl, substituted hydroxyalkylcarbonyl, alkoxyalkylcarbonyl, substituted alkoxyalkylcarbonyl, arylalkoxycarbonyl, substituted arylalkoxycarbonyl, arylcarbonyl, substituted arylcarbonyl, heterocycloalkyl, substituted heterocycloalkyl, heterocycloalkylcarbonyl, substituted heterocycloalkylcarbonyl, heteroarylcarbonyl, substituted heteroarylcarbonyl, (arylalkyl)carbonyl, substituted (arylalkyl)carbonyl, (heterocycloalkyl)alkylcarbonyl, substituted (heterocycloalkyl)alkylcarbonyl, heteroarylalkylcarbonyl, substituted heteroarylalkylcarbonyl, heteroarylalkoxyalkylcarbonyl, substituted heteroarylalkoxyalkylcarbonyl, alkylsulfonyl, substituted alkylsulfonyl, cycloalkylsulfonyl, substituted cycloalkylsulfonyl, arylsulfonyl, substituted arylsulfonyl, heteroarylsulfonyl, substituted heteroarylsulfonyl, alkylaminocarbonyl, substituted alkylaminocarbonyl, dialkylaminocarbonyl, substituted dialkylaminocarbonyl, dialkylaminosulfonyl, or substituted dialkylaminosulfonyl.

28. The compound of claim 1, wherein X² is O-heterocycloalkyl.

29. The compound of claim 1, wherein X² is cycloalkyl.

30. The compound of claim 1, wherein X² is cyclopropyl.

31. The compound of claim 1, wherein X³ is alkenyl, arylalkyl, substituted arylalkyl, heteroarylalkyl, substituted heteroarylalkyl, (heterocycloalkyl)alkyl, substituted (heterocycloalkyl)alkyl, (cycloalkyl)alkyl, substituted (cycloalkyl)alkyl, or hydroxyalkyl.

32. A compound of formula I, or a stereoisomer, tautomer, or pharmaceutically acceptable salt thereof: 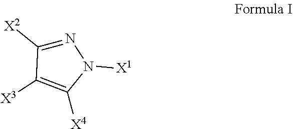
wherein X¹ is benzimidazolyl, substituted benzimidazolyl, azabenzimidazolyl, substituted azabenzimidazolyl, pyrazolyl, or substituted pyrazolyl;
X² is aryl, substituted aryl, arylalkyl, substituted arylalkyl, heteroaryl, substituted heteroaryl, heteroarylalkyl, substituted heteroarylalkyl, cycloalkyl, substituted cycloalkyl, (cycloalkyl)alkyl, substituted (cycloalkyl)alkyl, heterocycloalkyl, substituted heterocycloalkyl, (heterocycloalkyl)alkyl, substituted (heterocycloalkyl)alkyl, alkyl, substituted alkyl, alkoxycarbonyl, substituted alkoxycarbonyl, dialkylaminocarbonyl, substituted dialkylaminocarbonyl, (heterocycloalkyl)carbonyl, or substituted (heterocycloalkyl)carbonyl;
X³ is 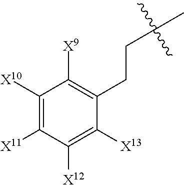
wherein each of X⁹, X¹⁰, X¹¹, X¹² and X¹³ is independently selected from hydrogen, halogen cyano, alkyl, alkoxy, haloalkyl or alkylsulfonyl; or 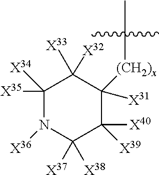
wherein x is 1 or 2; each of X³¹, X³², X³³, X³⁴, X³⁵, X³⁷, X³⁸, X³⁹, and X⁴⁰ is independently selected from hydrogen, hydroxy, cyano, aminocarbonyl, alkyl or haloalkyl; and X³⁶ is H, haloalkyl, alkoxycarbonyl, arylalkoxycarbonyl, substituted arylalkoxycarbonyl, alkylcarbonyl, hydroxyalkylcarbonyl, cycloalkylcarbonyl, arylcarbonyl, substituted arylcarbonyl, alkylsulfonyl, heteroaryl, or substituted heteroaryl; or 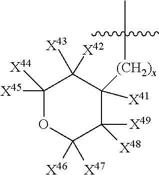
wherein x is 1 or 2; and each of X⁴¹, X⁴², X⁴³, X⁴⁴, X⁴⁵, X⁴⁶, X⁴⁷, X⁴⁸, and X⁴⁹ is independently selected from hydrogen, hydroxy, cyano, aminocarbonyl, alkyl or haloalkyl; and
X⁴ is hydroxy, amino, alkyl, or hydrogen.

33. The compound of claim 1, wherein X³ is 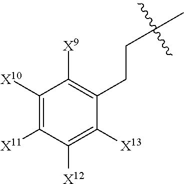
wherein each of X⁹, X¹⁰, X¹¹, X¹² and X¹³ is independently selected from hydrogen, halogen cyano, alkyl, alkoxy, haloalkyl or alkylsulfonyl.

34. The compound of claim 33, wherein X¹¹ is halogen.

35. The compound of claim 33, wherein X¹¹ is —F.

36. The compound of claim 34, wherein each of X⁹, X¹⁰, X¹² and X¹³ is hydrogen.

37. The compound of claim 1, wherein X³ is 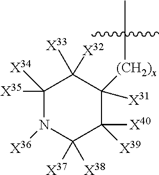
wherein x is 1 or 2; each of X³¹, X³², X³³, X³⁴, X³⁵, X³⁷, X³⁸, X³⁹, and X⁴⁰ is independently selected from hydrogen, hydroxy, cyano, aminocarbonyl, alkyl or haloalkyl; and X³⁶ is H, haloalkyl, alkoxycarbonyl, arylalkoxycarbonyl, substituted arylalkoxycarbonyl, alkylcarbonyl, hydroxyalkylcarbonyl, cycloalkylcarbonyl, arylcarbonyl, substituted arylcarbonyl, alkylsulfonyl, heteroaryl, or substituted heteroaryl.

38. The compound of claim 37, wherein each of X³¹, X³², X³³, X³⁴, X³⁵, X³⁷, X³⁸, X³⁹, and X⁴⁰ is hydrogen, and X³⁶ is alkoxycarbonyl.

39. The compound of claim 1, wherein X³ is 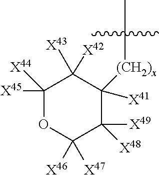
wherein x is 1 or 2; and each of X⁴¹, X⁴², X⁴³, X⁴⁴, X⁴⁵, X⁴⁶, X⁴⁷, X⁴⁸, and X⁴⁹ is independently selected from hydrogen, hydroxy, cyano, aminocarbonyl, alkyl or haloalkyl; and
X⁴ is hydroxy, amino, alkyl, or hydrogen.

40. The compound of claim 39, wherein each of X⁴¹, X⁴², X⁴³, X⁴⁴, X⁴⁵, X⁴⁶, X⁴⁷, X⁴⁸, and X⁴⁹ is hydrogen.

41. The compound of claim 1, wherein X⁴ is hydroxy.

42. The compound of claim 1, wherein the compound is:

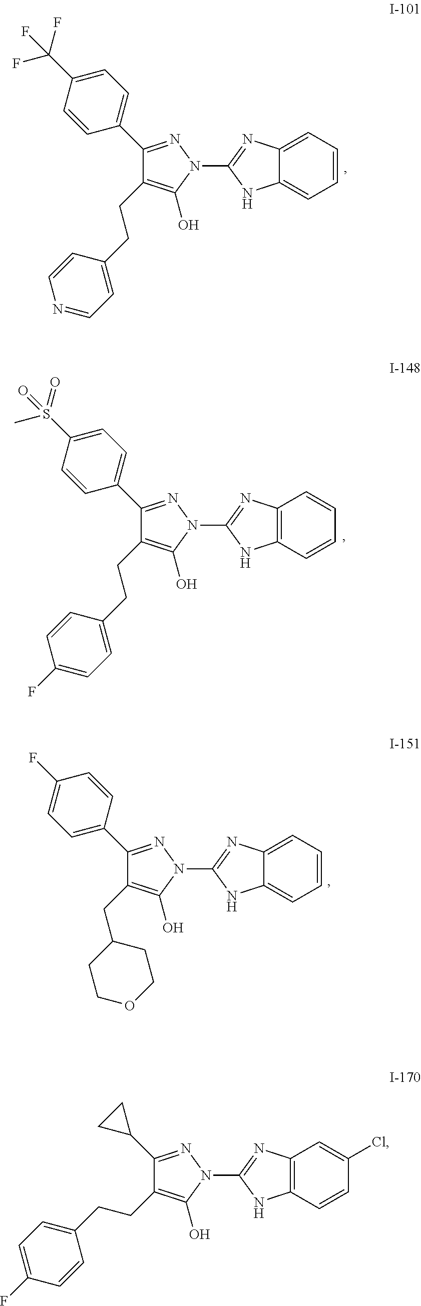 [SMILES: Oc1c(CCc2ccncc2)c(-c2ccc(C(F)(F)F)cc2)nn1-c1nc2ccccc2[nH]1 ; CS(=O)(=O)c1ccc(-c2nn(-c3nc4ccccc4[nH]3)c(O)c2CCc2ccc(F)cc2)cc1 ; Oc1c(CC2CCOCC2)c(-c2ccc(F)cc2)nn1-c1nc2ccccc2[nH]1 ; Oc1c(CCc2ccc(F)cc2)c(C2CC2)nn1-c1nc2cc(Cl)ccc2[nH]1]

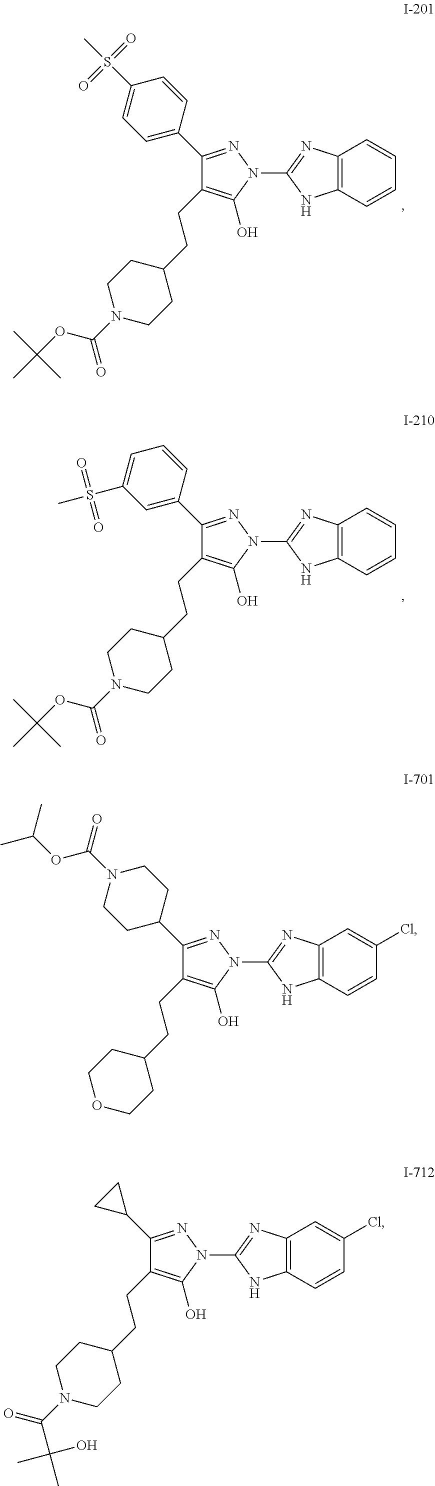 [SMILES: CC(C)(C)OC(=O)N1CCC(CCc2c(-c3ccc(S(C)(=O)=O)cc3)nn(-c3nc4ccccc4[nH]3)c2O)CC1 ; CC(C)(C)OC(=O)N1CCC(CCc2c(-c3cccc(S(C)(=O)=O)c3)nn(-c3nc4ccccc4[nH]3)c2O)CC1 ; CC(C)OC(=O)N1CCC(c2nn(-c3nc4cc(Cl)ccc4[nH]3)c(O)c2CCC2CCOCC2)CC1 ; CC(C)(O)C(=O)N1CCC(CCc2c(C3CC3)nn(-c3nc4cc(Cl)ccc4[nH]3)c2O)CC1]

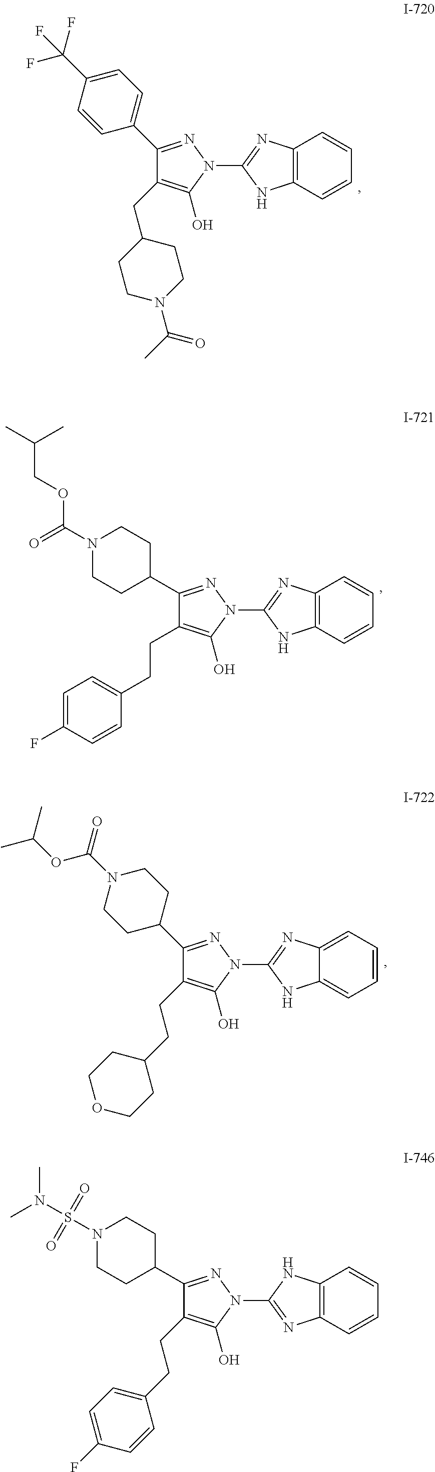 [SMILES: CC(=O)N1CCC(Cc2c(-c3ccc(C(F)(F)F)cc3)nn(-c3nc4ccccc4[nH]3)c2O)CC1 ; CC(C)COC(=O)N1CCC(c2nn(-c3nc4ccccc4[nH]3)c(O)c2CCc2ccc(F)cc2)CC1 ; CC(C)OC(=O)N1CCC(c2nn(-c3nc4ccccc4[nH]3)c(O)c2CCC2CCOCC2)CC1 ; CN(C)S(=O)(=O)N1CCC(c2nn(-c3nc4ccccc4[nH]3)c(O)c2CCc2ccc(F)cc2)CC1]

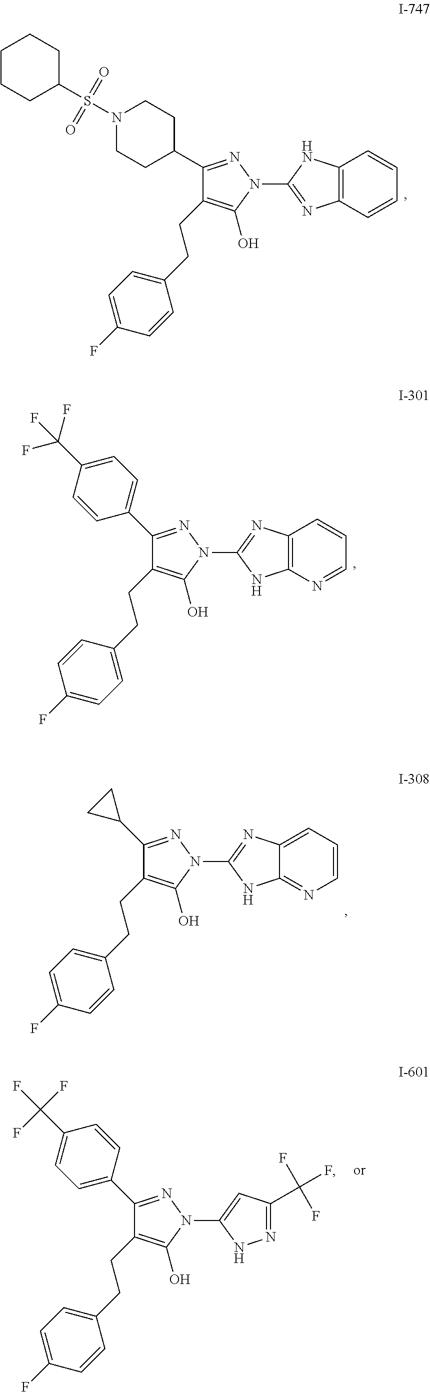 [SMILES: O=S(=O)(C1CCCCC1)N1CCC(c2nn(-c3nc4ccccc4[nH]3)c(O)c2CCc2ccc(F)cc2)CC1 ; Oc1c(CCc2ccc(F)cc2)c(-c2ccc(C(F)(F)F)cc2)nn1-c1nc2cccnc2[nH]1 ; Oc1c(CCc2ccc(F)cc2)c(C2CC2)nn1-c1nc2cccnc2[nH]1 ; Oc1c(CCc2ccc(F)cc2)c(-c2ccc(C(F)(F)F)cc2)nn1-c1cc(C(F)(F)F)n[nH]1]

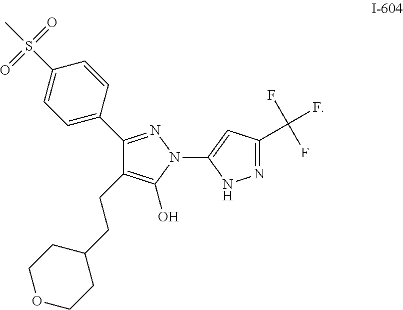 [SMILES: CS(=O)(=O)c1ccc(-c2nn(-c3cc(C(F)(F)F)n[nH]3)c(O)c2CCC2CCOCC2)cc1]

43-44. (canceled)

45. A pharmaceutical composition comprising at least one compound of claim 1 and at least one pharmaceutically acceptable excipient.

46. A pharmaceutical composition comprising at least one compound of claim 1 and at least one antiretroviral agent.

47. The pharmaceutical composition of claim 46, wherein the at least one antiretroviral agent is selected from an entry inhibitor, a CCR5 receptor antagonist, a nucleoside reverse transcriptase inhibitor, a non-nucleoside reverse transcriptase inhibitor, a protease inhibitor, an integrase inhibitor, a maturation inhibitor, or combinations thereof.

48. The pharmaceutical composition of claim 46, wherein the at least one antiretroviral agent is selected from maraviroc, enfuvirtide, aplaviroc, vicriviroc, zidovudine, didanosine, zalcitabine, stavudine, lamivudine, abacavir, emtricitabine, entecavir, apricitabine, tenofovir, adefovir, efavirenz, nevirapine, delavirdine, etravirine, rilpivirine, saquinavir, ritonavir, indinavir, nelfinavir, amprenavir, lopinavir, atazanavir, fosamprenavir, tipranavir, darunavir, MK-2048, elvitegravir, bevirimat, MPC-9055, or a combination thereof.

49. A method comprising administering to a subject having, suspected of having, or at risk of developing, HIV an effective amount of at least one compound selected from claim 1.

50. A method comprising administering to a subject having, suspected of having, or at risk of developing, HIV an effective amount of a pharmaceutical composition of claim 45.

51. A method of treating an HIV-related condition in a subject comprising administering to a subject in need thereof an effective amount of at least one compound selected from claim 1.

52. The method according to claim 50 wherein the HIV-related condition is selected from HIV replication, HIV-associated CD₄+ T-cell loss and immunodeficiency, HIV-induced infection, Kaposi's sarcoma, HIV-associated nephropathy, AIDS dementia complex, or a combination thereof.

53. The method of claim 49 wherein the subject is administered the compound post-exposure prophylactically.

54. The method of claim 49, further comprising co-administering to the subject at least one antiretroviral agent.

55. A method for inhibiting a biological function of Nef, comprising contacting Nef with an effective amount of at least one compound of claim 1.

56. The method of claim 55 wherein the biological function of Nef is selected from HIV infectivity, HIV replication, MHC-I downregulation or AIDS progression.

57. A method of inhibiting an activity of a Nef-dependent kinase comprising contacting the Nef-dependent kinase with an effective amount of at least one compound of claim 1.

58. The compound of claim 1, wherein X¹ is: 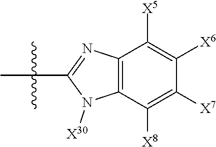
wherein each of X⁵, X⁶, X⁷, and X⁸ is independently selected from hydrogen, halogen, cyano, alkyl, haloalkyl, alkoxy, haloalkoxy or cycloalkyl; and X³⁰ is selected from aminoalkyl, alkylaminoalkyl, dialkylaminoalkyl, heterocycloalkyl, or heteroarylalkyl.
